(19) United States
(12) Patent Application Publication
(10) Pub. No.: US 2025/0382289 A1
(43) Pub. Date: Dec. 18, 2025

(54) PYRAZOLOPYRIDINE DERIVATIVE AND APPLICATION THEREOF IN MEDICINE

(71) Applicant: XIZANG HAISCO PHARMACEUTICAL CO., LTD., Lhoka, Tibet (CN)

(72) Inventors: Chen ZHANG, Chengdu City (CN); Jianmin WANG, Chengdu City (CN); Guofei QIAN, Chengdu City (CN); Yan YU, Chengdu City (CN); Pingming TANG, Chengdu City (CN); Tengfei CHEN, Chengdu City (CN); Yao LI, Chengdu City (CN); Pangke YAN, Chengdu City (CN)

(73) Assignee: XIZANG HAISCO PHARMACEUTICAL CO., LTD., Lhoka, Tibet (CN)

(21) Appl. No.: 18/839,389

(22) PCT Filed: Feb. 17, 2023
(86) PCT No.: PCT/CN2023/076700

§ 371 Date: Aug. 16, 2024

(30) Foreign Application Priority Data
Feb. 18, 2022 (CN) 202210137155.4
Mar. 25, 2022 (CN) 202210284775.0
Nov. 9, 2022 (CN) 202211396761.4

(51) Int. Cl. C07D 471/04; A61K 31/437; A61K 31/444; A61K 31/506; C07B 59/00; C07D 519/00

(52) U.S. Cl. CPC C07D 471/04; A61K 31/437; A61K 31/444; A61K 31/506; C07B 59/002; C07D 519/00

(57) ABSTRACT

The present invention relates to the compound shown in general formula (I) or to a stereoisomer, tautomer, deuterated substance, solvate, prodrug, metabolite, pharmaceutically acceptable salt or eutectic crystal thereof, and to an intermediate and a preparation method thereof, and also to an application thereof in the preparation of a medicine for treating diseases related to USP1 activity or expression.

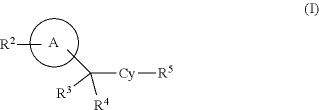

### CROSS-REFERENCE TO RELATED APPLICATIONS

This application is a 35 U.S.C. § 371 National Stage of International Patent Application No. PCT/CN2023/076700, filed Feb. 17, 2023, designating the United States, which claims priority to and the benefits of Chinese Patent Application No. 202210137155.4, filed Feb. 18, 2022, Chinese Patent Application No. 202210284775.0, filed Mar. 25, 2022, Chinese Patent Application No. 202211396761.4, filed Nov. 9, 2022, the disclosures of which are incorporated herein in their entirety by reference, and priority is claimed to each of the foregoing.

### TECHNICAL FIELD

The present invention relates to a compound shown in general formula (I) or to a stereoisomer, tautomer, deuterated substance, solvate, prodrug, metabolite, pharmaceutically acceptable salt or eutectic crystal thereof, and to an intermediate and a preparation method thereof, and also to an application thereof in the preparation of a medicine for treating diseases related to USP1 activity or expression.

### BACKGROUND

Protein ubiquitination in cells is a key protein modification that can regulate multiple cellular processes. Protein ubiquitination is controlled by E3 ubiquitin ligase and deubiquitinating enzymes (DUBs) in a synergistic manner. DUBs can cleave an isopeptide bond between ubiquitin and a modified protein, and is responsible for removing ubiquitin from the target protein and rescuing it from the degradation pathway; they are also involved in editing, maturation and recycling of ubiquitin molecules after degradation. At present, more than 100 deubiquitinating enzymes are known, and these proteins are subdivided into six subfamilies. Ubiquitin-specific protease (USP) subfamily is the largest subfamily among them and is currently known to have 58 members. USP is a cysteine protease having a highly conserved catalytic domain, and USP1 is one of USP subfamilies among DUBs (Cancers, 2020, 12, 1579; Molecular Cell, 2018, 72, 925-941).

Fanconi Anemia (FA) and DNA Translesion Synthesis (TLS) pathways are the first DNA damage tolerance and repair pathways found to be regulated by reversible ubiquitination. USP1 can participate in the regulation of DNA damage-repair pathway by regulating deubiquitination of specific proteins in FA and TLS pathways (Nature Chemical Biology, 2014, 10, 298-304). USP1 plays an important role in DNA repair in tumor cells. It has been reported that the deletion of USP1 leads to the decrease of survival rate of BRCA1-deficient cells and replication fork degradation (Molecular Cell, 2018, 72, 925-941). UAF1 (USP1-associated factor 1), as an auxiliary factor for USP1, USP12 and USP46, can enhance their activity as deubiquitinating enzymes by forming a stable USP/UAF1 protein complex. The USP1/UAF1 complex deubiquitinates various substrates and is related to DNA repair process, tumor pathogenesis and regulation of antiviral innate immunity (Nat Commun, 2020, 11, 6042). At present, there are no drugs targeting USP1 protein on the market, and the research on USP1 inhibitors has broad application prospects.

### SUMMARY

An object of the present invention is to provide a compound capable of inhibiting USP1 or a stereoisomer, tautomer, deuterated substance, solvate, prodrug, metabolite, pharmaceutically acceptable salt or eutectic crystal thereof, an intermediate and a preparation method thereof, and an application thereof in the preparation of a medicine for treating a disease related to USP1 activity or expression.

The present invention provides a compound shown in general formula (I) or a stereoisomer, tautomer, deuterated substance, solvate, prodrug, metabolite, pharmaceutically acceptable salt or eutectic crystal thereof, wherein

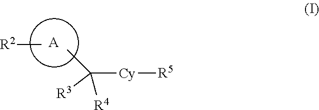

in some embodiments, ring A is selected from

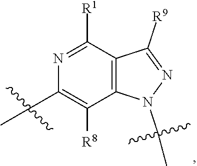

wherein the left side is connected to R²;
in some embodiments, ring A is selected from

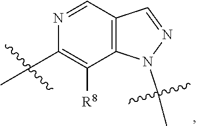

wherein the left side is connected to R²;
in some embodiments, ring A is selected from

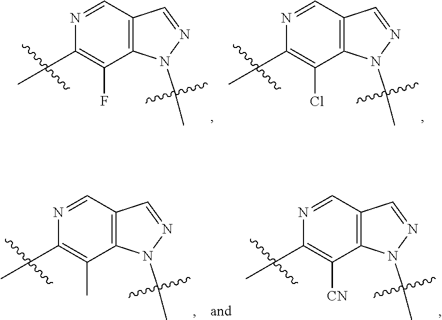 [SMILES: *c1ncc2cnn(*)c2c1F ; *c1ncc2cnn(*)c2c1Cl ; *c1ncc2cnn(*)c2c1C ; *c1ncc2cnn(*)c2c1C#N]

wherein the left side is connected to R²;
in some embodiments, R¹ and R⁹ are each independently selected from H, halogen, cyano, C₁₋₆ alkyl, C₂₋₆ alkenyl, C₂₋₆ alkynyl, or C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, or cyano-substituted C₁₋₆ alkyl;
in some embodiments, R¹ and R⁹ are each independently selected from H, halogen, cyano, C₁₋₄ alkyl, C₂₋₄ alkenyl, C₂₋₄ alkynyl, or C₁₋₄ alkoxy, wherein the alkyl, alkenyl, alkynyl, or alkoxy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, or cyano-substituted C₁₋₄ alkyl;
in some embodiments, R¹ and R⁹ are each independently selected from H, F, Cl, Br, I, cyano, methyl, or ethyl;
in some embodiments, R¹ is H;
in some embodiments, R⁹ is H;
in some embodiments, R⁸ is selected from halogen, cyano, C₁₋₆ alkyl, C₂₋₆ alkenyl, C₂₋₆ alkynyl, or C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, or cyano-substituted C₁₋₆ alkyl;
in some embodiments, R⁸ is selected from halogen, cyano, C₁₋₄ alkyl, C₂₋₄ alkynyl, and C₁₋₄ alkoxy, wherein the alkyl, alkynyl, or alkoxy is optionally further substituted with 0 to 4 substituents selected from H, halogen, OH, cyano, NH₂, C₁₋₄ alkyl, C₂₋₄ alkynyl, and C₁₋₄ alkoxy;
in some embodiments, R⁸ is selected from F, Cl, Br, I, CN, methyl, ethyl, ethynyl, methoxy, or ethoxy;
in some embodiments, R⁸ is selected from F, Cl, Br, CN, methyl, ethynyl, or methoxy;
in some embodiments, R⁸ is selected from F or Cl;
in some embodiments, R² is selected from C₆₋₁₀ carbocycle or 5- to 10-membered heterocycle, wherein the carbocycle or heterocycle is optionally further substituted with 0 to 4 R^{2a};
in some embodiments, R² is selected from phenyl, 6-membered heteroaryl, benzo C₄₋₆ carbocycle, benzo 4- to 6-membered heterocycle, pyridino C₄₋₆ carbocycle, and pyridino 4- to 6-membered heterocycle;
in some embodiments, R² is selected from phenyl, pyridine, pyrimidine, pyrrole, pyrazole, imidazole, furan, thiophene, thiazole, or oxazole, wherein the phenyl, pyridine, pyrimidine, pyrrole, pyrazole, imidazole, furan, thiophene, thiazole, or oxazole is optionally further substituted with 0 to 4 R^{2a};
in some embodiments, R² is selected from

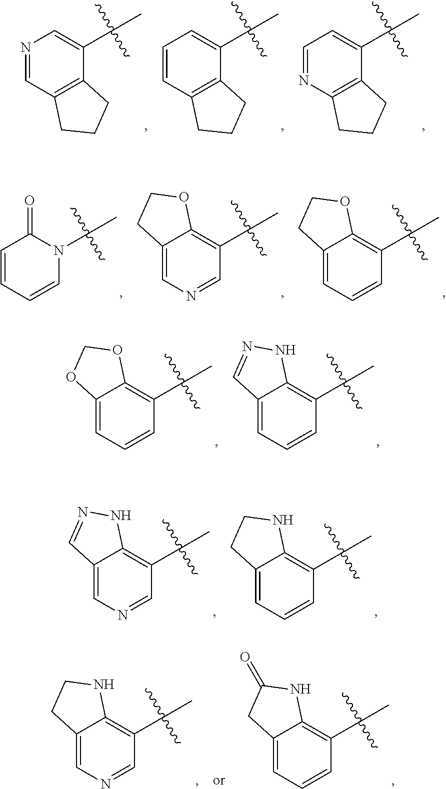

and R² is optionally further substituted with 0 to 4 R^{2a};
in some embodiments, R² is selected from pyrrole, pyrazole, imidazole, pyrimidine,

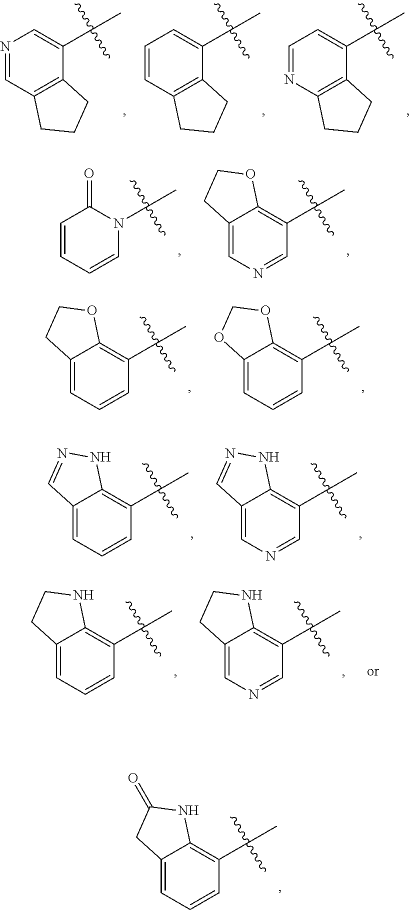

wherein the pyrrole, pyrazole, imidazole, pyrimidine,

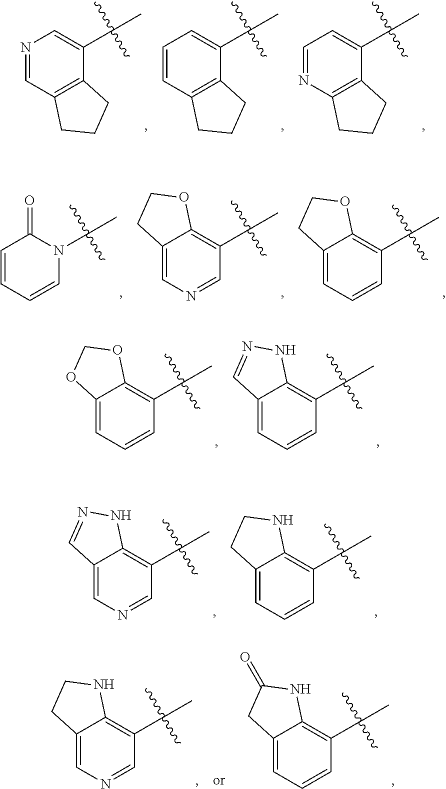

is optionally further substituted with 0 to 4 substituents selected from H, F, Cl, Br, I, cyano, —SO₂-methyl, —SO₂-ethyl, —SO₂-propyl, —SO₂-isopropyl, —SO₂-cyclopropyl, CH₂F, CHF₂, CF₃, —OCH₂F, —OCHF₂, —OCF₃, methyl, ethyl, propyl, isopropyl, methoxy, ethoxy, propoxy, —OCD₃, methylthio, ethylthio, propylthio, cyclopropyl, cyclobutyl, cyclopentyl, cyclohexyl, azetidinyl, azacyclopentyl, azacyclohexyl, piperazine, oxetanyl, oxacyclopentyl, oxacyclohexyl, morpholine, —O-azetidinyl, —O-azacyclopentyl, —O-azacyclohexyl, —O-oxetanyl, —O-oxacyclopentyl, —O-oxacyclohexyl, —OCH₂-phenyl, —OCH₂-cyclopropyl, —OCH₂-cyclobutyl, —OCH₂-cyclopentyl, —OCH₂-cyclohexyl,

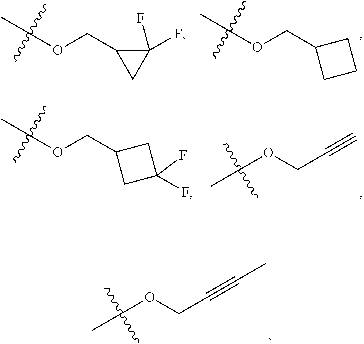

pyrrole, pyrazole, imidazole, thiazole, thiophene, furan, oxazole,

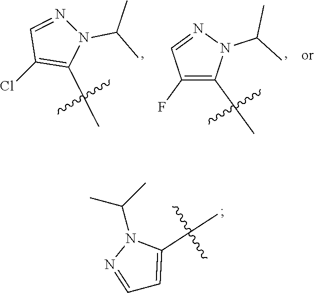

in some embodiments, R² is selected from

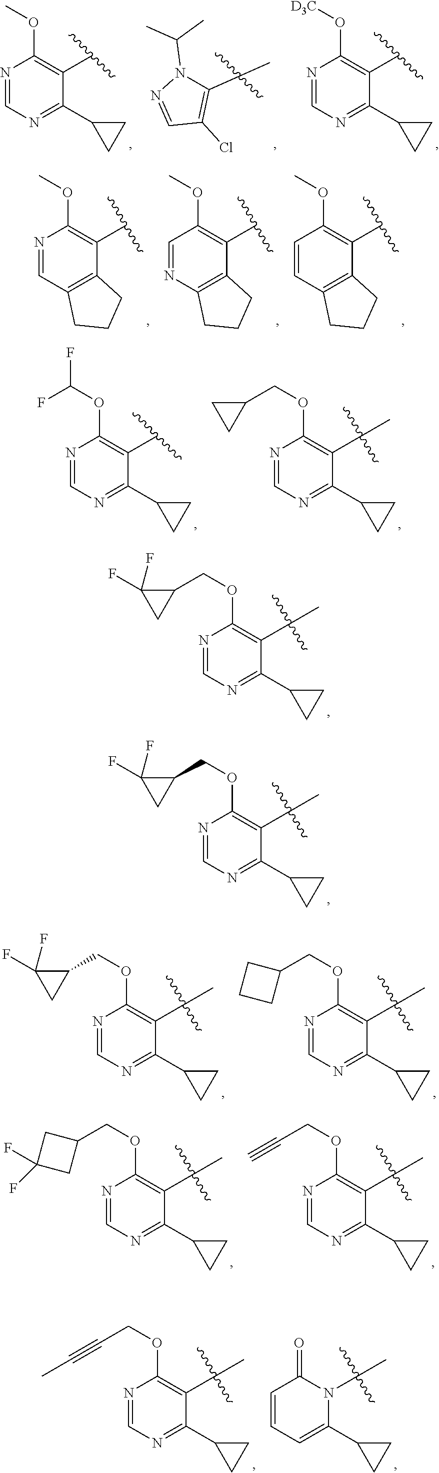

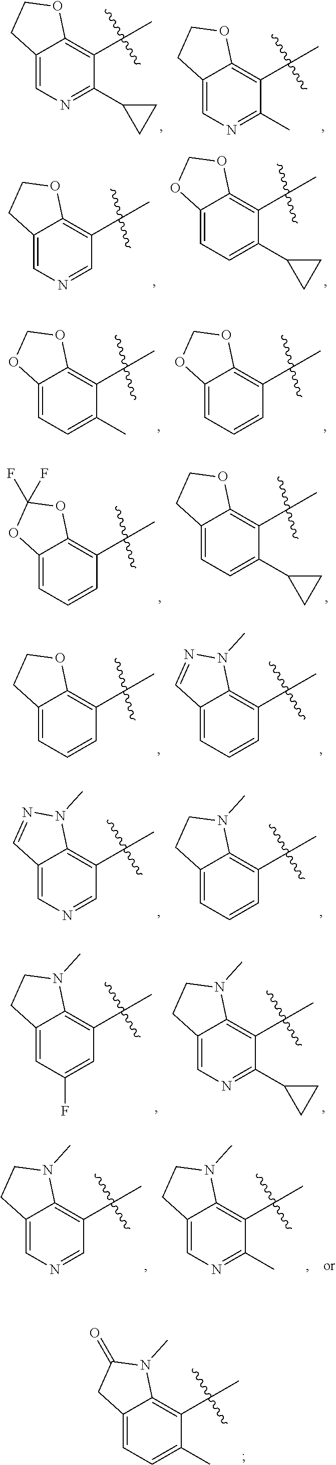

in some embodiments, R² is selected from

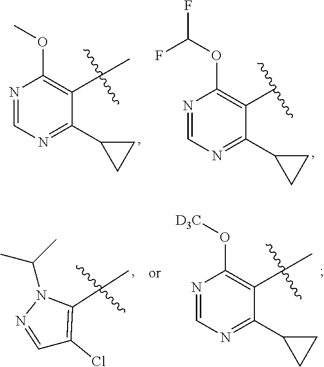

in some embodiments, each R^{2a} is independently selected from H, halogen, OH, ═O, cyano, NH₂, NH(C₁₋₆ alkyl), N(C₁₋₆ alkyl)₂, —SO₂—C₁₋₆ alkyl, —SO₂—C₃₋₆ cycloalkyl, C₁₋₆ alkyl, C₁₋₆ alkoxy, C₁₋₆ alkylthio, —Z—C₃₋₆ cycloalkyl, or —Z-3- to 8-membered heterocycle, wherein the alkyl, cycloalkyl, alkoxy, alkylthio, or heterocycle is optionally further substituted with 0 to 4 substituents selected from H, D, halogen, OH, ═O, cyano, NH₂, C₁₋₆ alkyl, C₂₋₆ alkynyl, or C₁₋₆ alkoxy, and the heterocycle contains 1 to 4 heteroatoms selected from O, S, and N;
in some embodiments, each R^{2a} is independently selected from H, halogen, OH, ═O, cyano, NH₂, NH(C₁₋₄ alkyl), N(C₁₋₄ alkyl)₂, —SO₂—C₁₋₄ alkyl, —SO₂—C₃₋₆ cycloalkyl, C₁₋₄ alkyl, C₁₋₄ alkoxy, C₁₋₄ alkylthio, —Z—C₃₋₆ cycloalkyl, or —Z-3- to 6-membered heterocycle, wherein the alkyl, cycloalkyl, alkoxy, alkylthio, or heterocycle is optionally further substituted with 0 to 4 substituents selected from H, D, halogen, OH, ═O, cyano, NH₂, C₁₋₄ alkyl, C₂₋₄ alkynyl, C₁₋₄ alkoxy, and the heterocycle contains 1 to 4 heteroatoms selected from O, S, and N;
in some embodiments, each R^{2a} is independently selected from H, F, Cl, Br, I, or cyano, or each R^{2a} is independently selected from one of the following substituted or unsubstituted groups: —SO₂-methyl, —SO₂-ethyl, —SO₂-propyl, —SO₂-isopropyl, —SO₂-cyclopropyl, methyl, ethyl, propyl, isopropyl, methoxy, ethoxy, propoxy, methylthio, ethylthio, propylthio, cyclopropyl, cyclobutyl, cyclopentyl, cyclohexyl, azetidinyl, azacyclopentyl, azacyclohexyl, piperazine, oxetanyl, oxacyclopentyl, oxacyclohexyl, morpholine, —O-azetidinyl, —O-azacyclopentyl, —O-azacyclohexyl, —O-oxetanyl, —O-oxacyclopentyl, —O-oxacyclohexyl, —OCH₂-phenyl, pyrrole, pyrazole, imidazole, thiazole, thiophene, furan, and oxazole, which, when substituted, are optionally further substituted with 0 to 4 substituents selected from H, D, halogen, OH, ═O, cyano, NH₂, C₁₋₄ alkyl, C₂₋₄ alkynyl, and C₁₋₄ alkoxy;
in some embodiments, Z is selected from a bond, O, S, NH, N(C₁₋₄ alkyl), C(═O)NH, NHC(═O), —OCH₂—, —CH₂O—, or C₁₋₃ alkylene;
in some embodiments, R³ and R⁴ are each independently selected from H, halogen, OH, cyano, NH₂, C₁₋₆ alkyl, C₂₋₆ alkenyl, C₂₋₆ alkynyl, or C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, or 3- to 8-membered heterocyclyl, and the heterocyclyl contains 1 to 4 heteroatoms selected from O, S, and N;
in some embodiments, R³ and R⁴ are each independently selected from H, halogen, OH, cyano, C₁₋₄ alkyl;
in some embodiments, R³ and R⁴ are each independently selected from H, F, Cl, Br, I, methyl, and ethyl;
in some embodiments, R³ and R⁴ are H;
in some embodiments, Cy is selected from C₃₋₁₂ carbocycle or 4- to 12-membered heterocycle, and the Cy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, or 3- to 8-membered heterocyclyl, wherein the heterocycle contains 1 to 4 heteroatoms selected from O, S, and N;
in some embodiments, Cy is selected from C₃₋₆ monocyclic cycloalkyl, C₅₋₁₁ fused cycloalkyl, C₅₋₁₁ spirocycloalkyl, C₅₋₁₁ bridged cycloalkyl, 4- to 7-membered monocyclic heterocycloalkyl, 6- to 11-membered fused heterocycloalkyl, 6- to 11-membered spiroheterocycloalkyl, 6- to 11-membered bridged heterocycloalkyl, benzene ring, benzo C₄₋₆ carbocycle, benzo 4- to 6-membered heterocycle, 5- to 6-membered heteroaromatic ring, or 8- to 10-membered fused heteroaromatic ring, and the Cy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, or 3- to 8-membered heterocycle, wherein the heterocycle contains 1 to 4 heteroatoms selected from O, S, and N;
in some embodiments, Cy is selected from cyclopropyl, cyclobutyl, cyclopentyl, cyclohexyl, bicyclo[2.2.2]octyl, bicyclo[1.1.1]pentyl, cyclopentyl-fused cyclopentyl, cyclobutylspirocyclohexyl, cyclobutylspirocyclopentyl, azetidinyl, azacyclopentyl, azacyclohexyl, azetidinylspirocyclobutyl, azetidinylspirocyclopentyl, azetidinylspirocyclohexyl, azetidinylspiroazetidinyl, azetidinylspiroazacyclopentyl, azetidinylspiroazacyclohexyl,

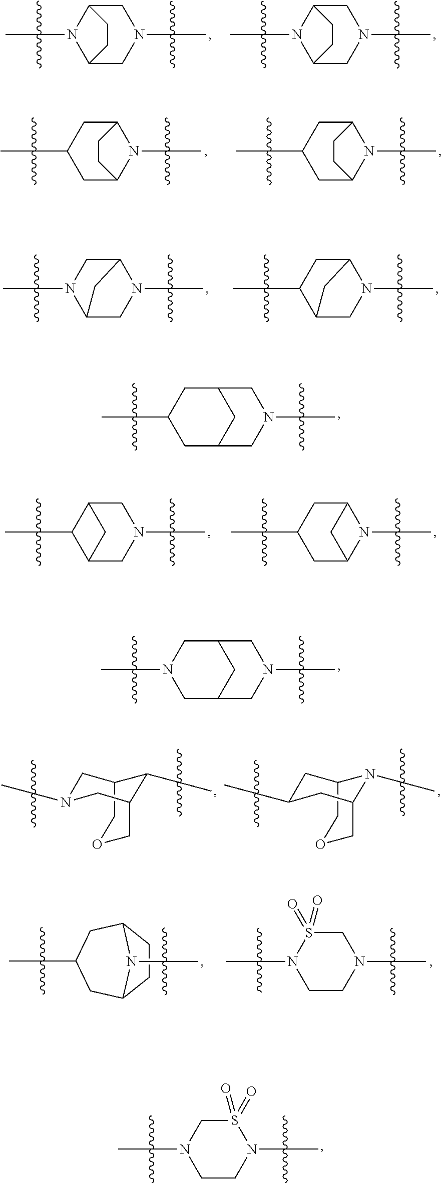

benzene ring, thiophene, furan, pyrrole, thiazole, oxazole, pyrazole, pyrazine, pyrimidine,

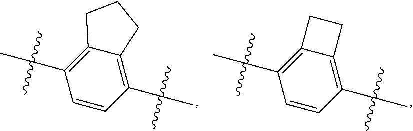 [SMILES: *c1ccc(*)c2c1CCC2 ; *c1ccc(*)c2c1CC2]

benzothiazole, benzothiophene, benzofuran, benzoxazole, benzopyrrole, benzopyrazole, benzimidazole, indolizine, pyridinopyrrole, pyridinoimidazole, pyridinopyrazole, pyrimidoimidazole, pyrimidopyrazole, pyridinotriazole, or 2-pyridone, and the Cy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, or 3- to 8-membered heterocycle, wherein the heterocycle contains 1 to 4 heteroatoms selected from O, S, and N;
in some embodiments, Cy is selected from

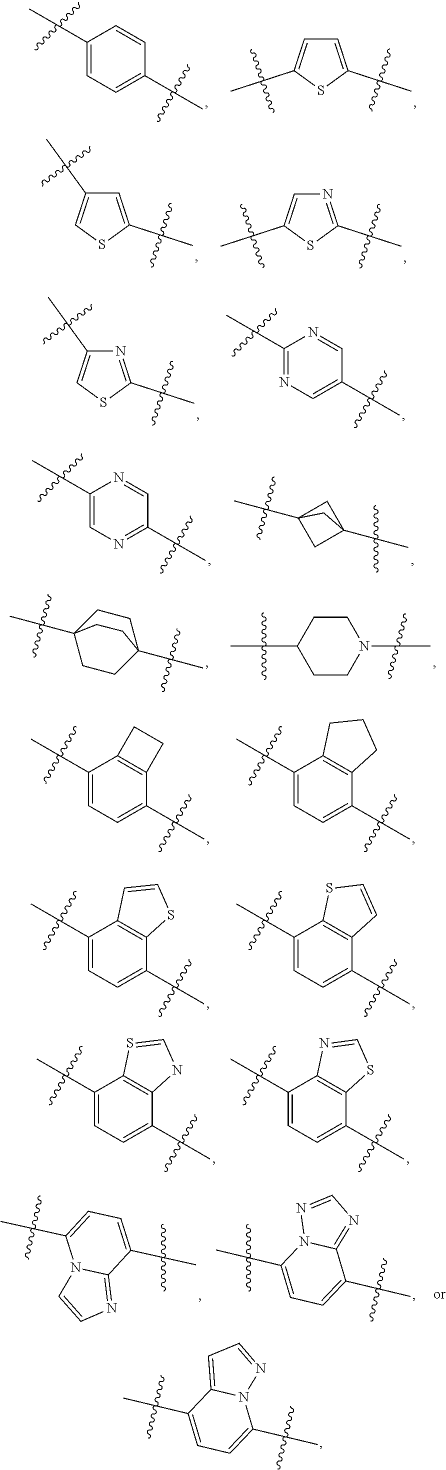

and the Cy is optionally further substituted with 0 to 4 substituents selected from H, F, Cl, Br, I, cyano, CH₂F, CHF₂, CF₃, methyl, ethyl, propyl, isopropyl, methoxy, ethoxy, or cyclopropyl;
in some embodiments, Cy is selected from

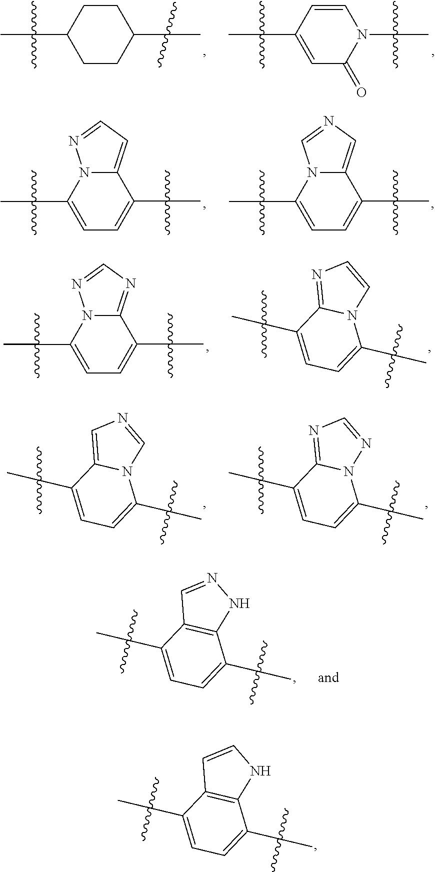

and the Cy is optionally further substituted with 0 to 4 substituents selected from H, F, Cl, Br, I, cyano, CH₂F, CHF₂, CF₃, methyl, ethyl, propyl, isopropyl, methoxy, ethoxy, or cyclopropyl;
in some embodiments, R⁵ is selected from —Z—C₃₋₁₂ carbocycle or —Z-4- to 12-membered heterocycle, and R⁵ is optionally further substituted with 0 to 4 R^{5a};
in some embodiments, R⁵ is selected from —Z—C₆₋₁₂ carbocycle or —Z-5- to 12-membered heterocycle, and R⁵ is optionally further substituted with 0 to 4 R^{5a};
in some embodiments, R⁵ is selected from —Z—C₃₋₁₀ carbocycle or —Z-4- to 10-membered heterocycle, and R⁵ is optionally further substituted with 0 to 4 R^{5a};
in some embodiments, R⁵ is selected from —Z—C₆₋₁₀ carbocycle or —Z-5- to 10-membered heterocycle, and R⁵ is optionally further substituted with 0 to 4 R^{5a};
in some embodiments, Z is selected from a bond, O, S, NH, N(C₁₋₄ alkyl), C(═O)NH, NHC(═O), —OCH₂—, —CH₂O—, or C₁₋₃ alkylene;
in some embodiments, R⁵ is selected from imidazole or imidazo 4- to 9-membered heterocycle, and R⁵ is optionally further substituted with 0 to 4 R^{5a};
in some embodiments, R⁵ is selected from benzene ring, pyrazole, pyrrole, imidazole, triazole, pyridine, pyrimidine, pyrazine, or pyridazine, and R⁵ is optionally further substituted with 0 to 4 R^{5a};
in some embodiments, R⁵ is selected from pyrrole, pyrazole, or imidazole, and the pyrrole, pyrazole, or imidazole is optionally further substituted with 0 to 4 substituents selected from H, F, Cl, Br, I, cyano, CH₂F, CHF₂, CF₃, CD₃, methyl, ethyl, propyl, isopropyl, —CH₂CH₂F, methoxy, ethoxy, cyclopropyl, cyclobutyl, cyclopentyl, cyclohexyl, oxetanyl, oxacyclopentyl, oxacyclohexyl, azetidinyl, N-methylazetidinyl, azacyclopentyl, azacyclohexyl,

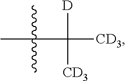 [SMILES: *C([2H])(C([2H])([2H])[2H])C([2H])([2H])[2H]]

—CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, —CH₂-azacyclohexyl,

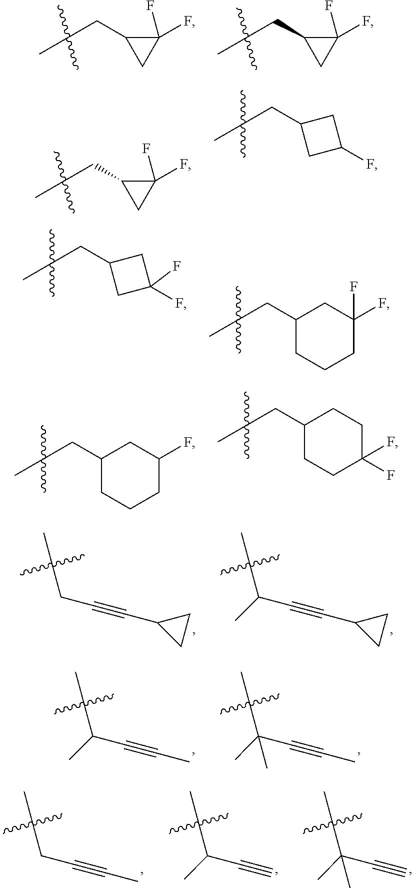

or propargyl;
in some embodiments, R⁵ is selected from

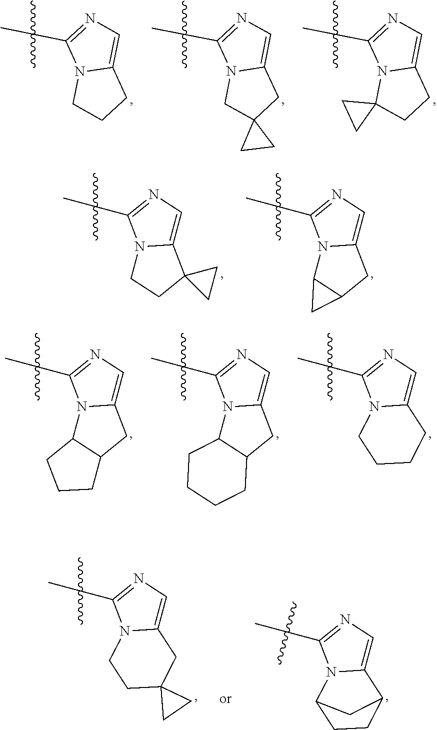

and R⁵ is optionally further substituted with 0 to 4 substituents selected from H, F, Cl, Br, I, cyano, CH₂F, CHF₂, CF₃, CD₃, methyl, ethyl, propyl, isopropyl, —CH₂CH₂F, methoxy, ethoxy, cyclopropyl, cyclobutyl, cyclopentyl, cyclohexyl, oxetanyl, oxacyclopentyl, oxacyclohexyl, azetidinyl, N-methylazetidinyl, azacyclopentyl, azacyclohexyl,

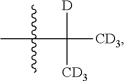 [SMILES: *C([2H])(C([2H])([2H])[2H])C([2H])([2H])[2H]]

—CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, —CH₂-azacyclohexyl,

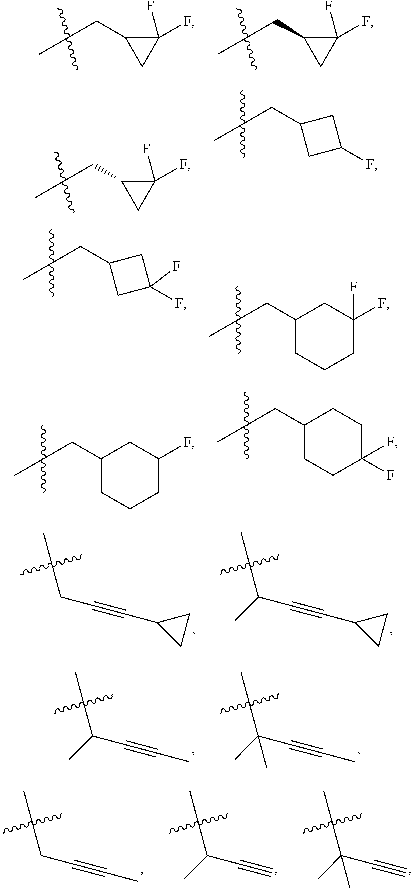

or propargyl;
in some embodiments, R⁵ is selected from pyrrole, pyrazole, or imidazole, wherein the pyrrole, pyrazole, or imidazole is optionally further substituted with 0 to 4 substituents selected from H, F, Cl, Br, I, cyano, CH₂F, CHF₂, CF₃, CD₃, methyl, ethyl, propyl, isopropyl, methoxy, ethoxy, cyclopropyl, cyclobutyl, cyclopentyl, cyclohexyl, oxetanyl, oxacyclopentyl, oxacyclohexyl, azetidinyl, N-methylazetidinyl, azacyclopentyl, or azacyclohexyl;
in some embodiments, R⁵ is selected from pyrrole, pyrazole, or imidazole, wherein the pyrrole, pyrazole, or imidazole is optionally further substituted with 0 to 4 substituents selected from H, F, Cl, Br, I, cyano, CH₂F, CHF₂, CF₃, methyl, ethyl, propyl, isopropyl, methoxy, ethoxy, cyclopropyl, cyclobutyl, cyclopentyl, or cyclohexyl;
in some embodiments, R⁵ is selected from

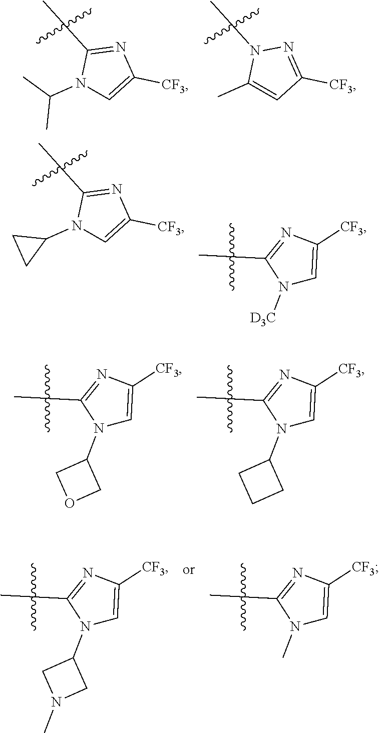

in some embodiments, R⁵ is selected from

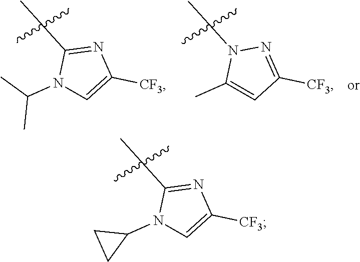 [SMILES: *c1nc(C(F)(F)F)cn1C(C)C ; *n1nc(C(F)(F)F)cc1C ; *c1nc(C(F)(F)F)cn1C1CC1]

in some embodiments, R⁵ is selected from

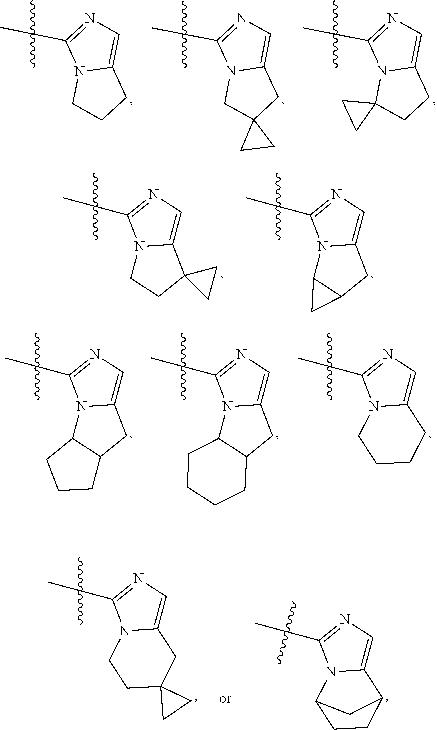

and R⁵ is optionally further substituted with 0 to 4 R^{5a};
in some embodiments, R⁵ is selected from

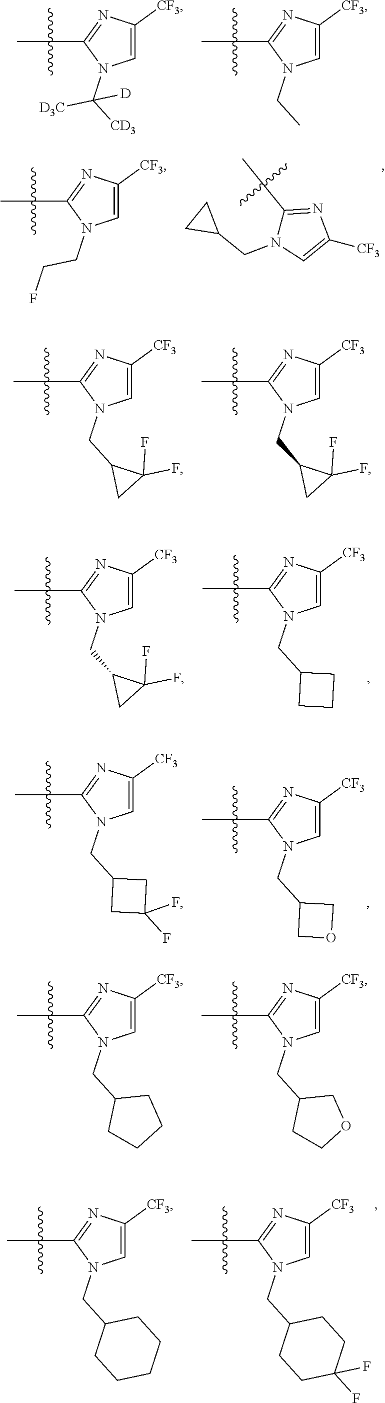

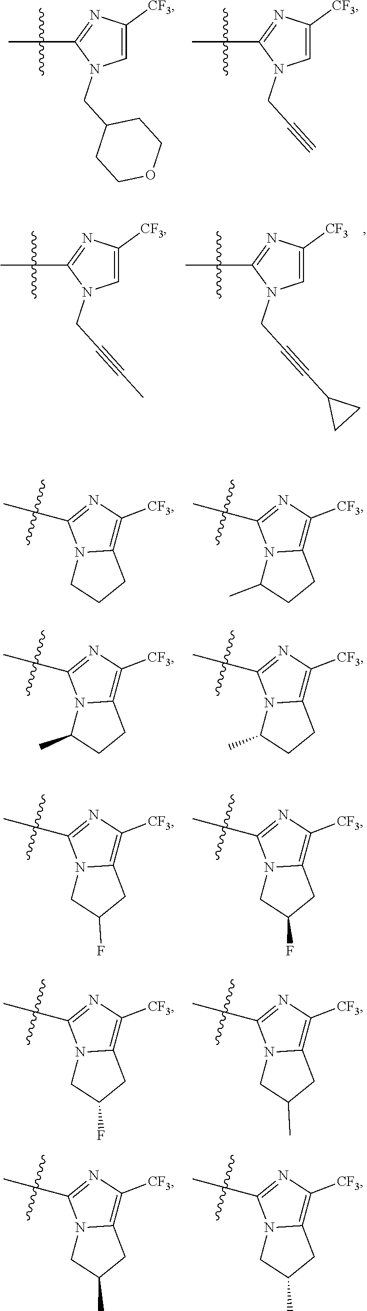

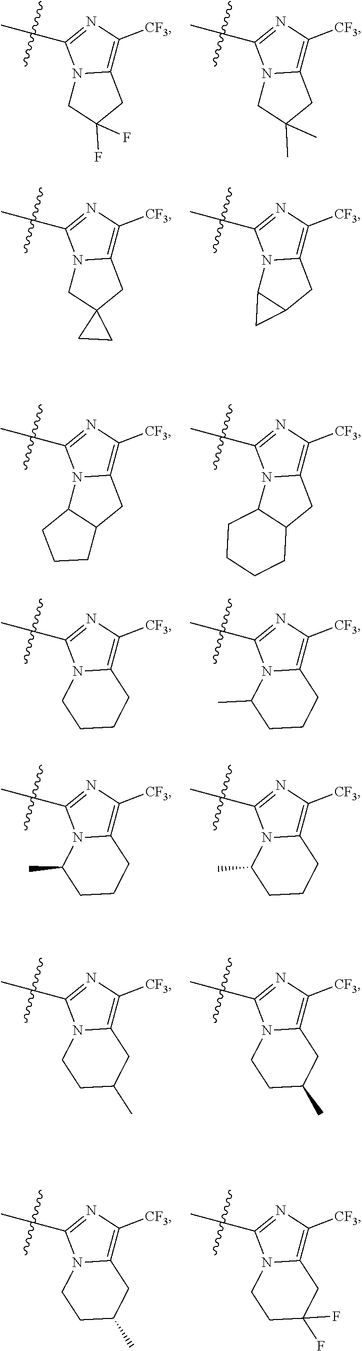

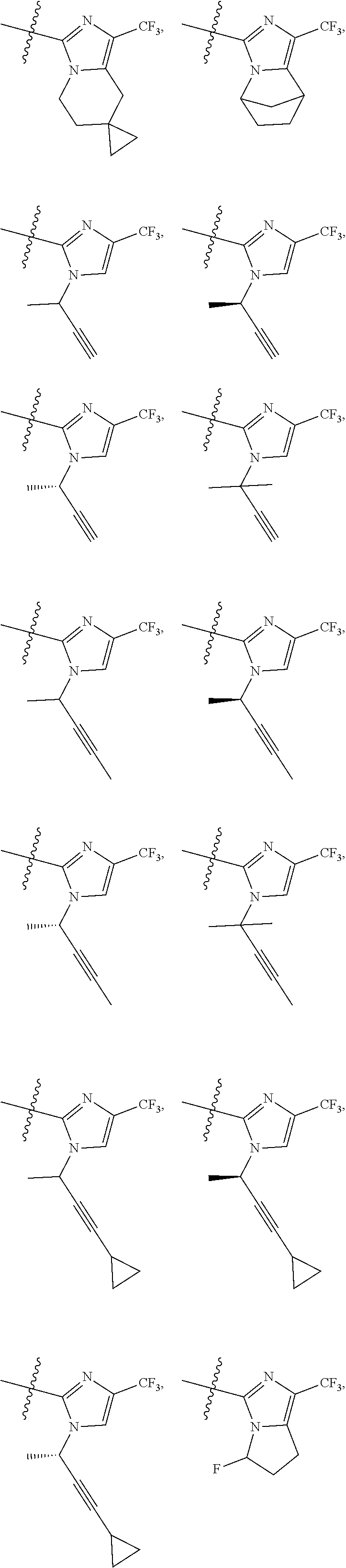

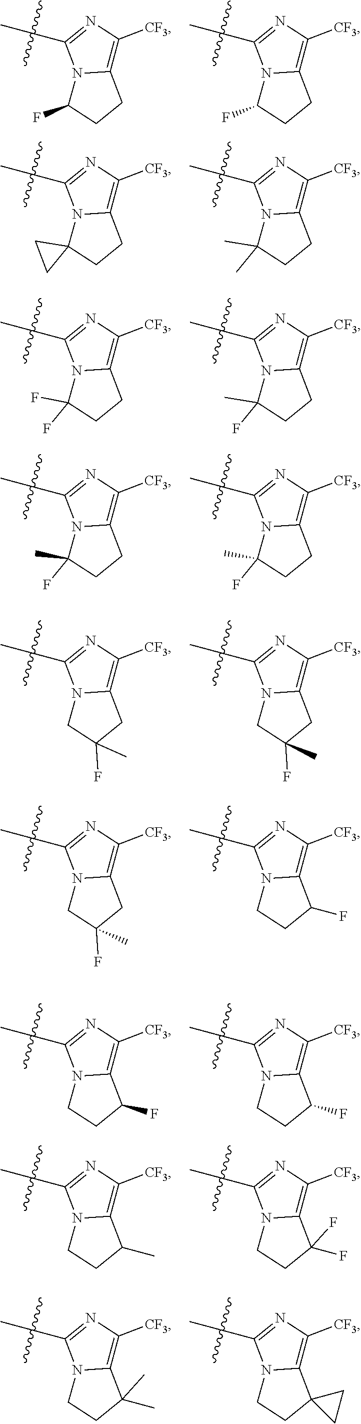

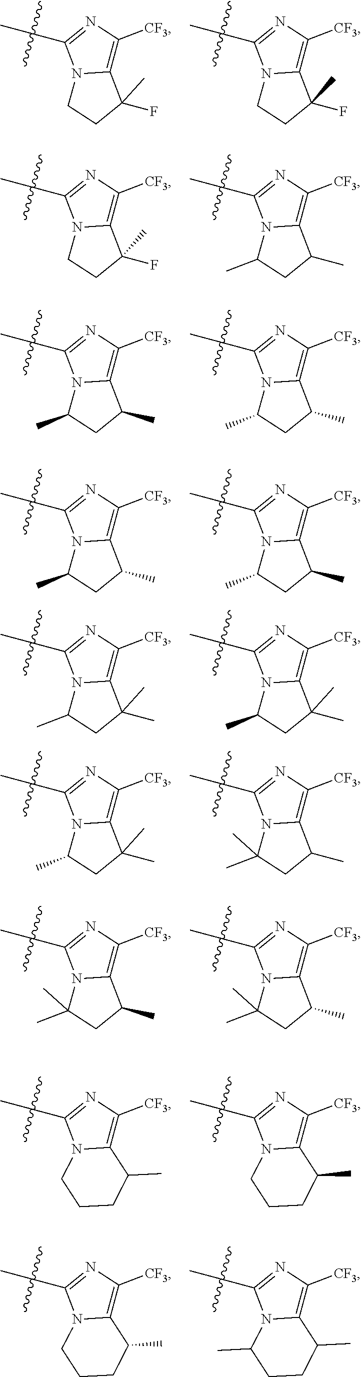

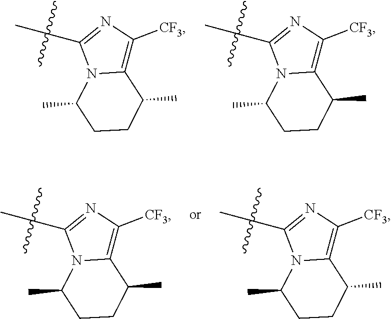

in some embodiments, each R^{5a} is independently selected from H, halogen, OH, cyano, NH₂, NHC₁₋₆ alkyl, N(C₁₋₆ alkyl)₂, C₁₋₆ alkyl, C₂₋₆ alkynyl, C₁₋₆ alkoxy, —C₁₋₄ alkylene-C₃₋₆ cycloalkyl, —C₁₋₄ alkylene-3- to 6-membered heterocycle, C₃₋₆ cycloalkyl, 3- to 8-membered heterocycle, —CONHC₁₋₆ alkyl, —CONHC₁₋₆ alkoxy, —CONHC₃₋₆ cycloalkyl, —CONH 3- to 8-membered heterocycle, —NHCO—C₁₋₆ alkyl, —NHCO—C₁₋₆ alkoxy, —NHCO—C₃₋₆ cycloalkyl, or —NHCO-3- to 8-membered heterocycle, wherein the alkyl, alkylene, alkynyl, alkoxy, cycloalkyl, or heterocycle is optionally further substituted with 0 to 4 substituents selected from H, D, halogen, OH, ═O, cyano, NH₂, —C(═O)NH₂, —C(═O)NHC₁₋₆ alkyl, —C(═O)N(C₁₋₆ alkyl)₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, halogen-substituted C₃₋₆ cycloalkyl, or 3- to 8-membered heterocycle, and the heterocycle contains 1 to 4 heteroatoms selected from O, S, and N;
in some embodiments, each R^{5a} is independently selected from H, halogen, OH, cyano, NH₂, C₁₋₄ alkyl, C₂₋₄ alkynyl, C₁₋₄ alkoxy, —C₁₋₂ alkylene-C₃₋₆ cycloalkyl, —C₁₋₂ alkylene-3- to 6-membered heterocycle, C₃₋₆ cycloalkyl, or 3- to 6-membered heterocycle, wherein the alkyl, alkylene, alkynyl, alkoxy, cycloalkyl, or heterocycle is optionally further substituted with 0 to 4 substituents selected from H, D, halogen, OH, ═O, cyano, NH₂, —C(═O)NH₂, —C(═O)NHC₁₋₄ alkyl, —C(═O)N(C₁₋₄ alkyl)₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, halogen-substituted C₃₋₆ cycloalkyl, or 3- to 8-membered heterocycle, and the heterocycle contains 1 to 4 heteroatoms selected from O, S, and N;
in some embodiments, each R^{5a} is independently selected from H, F, Cl, Br, I, cyano, methyl, ethyl, propyl, isopropyl, propargyl, butargyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, —CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, or —CH₂-azacyclohexyl, wherein the methyl, ethyl, propyl, isopropyl, propargyl, butargyl, methoxy, ethoxy, cyclopropyl, cyclobutyl, cyclopentyl, cyclohexyl, oxetanyl, oxacyclopentyl, oxacyclohexyl, azetidinyl, azacyclopentyl, or azacyclohexyl is optionally further substituted with 0 to 4 substituents selected from H, halogen, OH, ═O, cyano, NH₂, C₁₋₄ alkyl, C₂₋₄ alkynyl, C₁₋₄ alkoxy, C₃₋₆ cycloalkyl, or halogen-substituted C₃₋₆ cycloalkyl;
in some embodiments, the compound shown in general formula (I) is selected from a compound shown in general formula (Ia), and the definitions of the groups in the compound shown in general formula (Ia) are the same as the definitions of those in the compound shown in general formula (I).

A first embodiment of the present invention is a compound shown in the above general formula (I) or a stereoisomer, tautomer, deuterated substance, solvate, prodrug, metabolite, pharmaceutically acceptable salt or eutectic crystal thereof,
wherein ring A is selected from

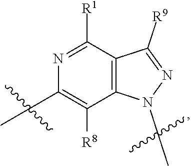

and the left side is connected to R²;
R¹ and R⁹ are each independently selected from H, halogen, cyano, C₁₋₆ alkyl, C₂₋₆ alkenyl, C₂₋₆ alkynyl, or C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, or cyano-substituted C₁₋₆ alkyl;
R⁸ is selected from halogen, cyano, C₁₋₆ alkyl, C₂₋₆ alkenyl, C₂₋₆ alkynyl, or C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, or cyano-substituted C₁₋₆ alkyl;
R² is selected from C₆₋₁₀ carbocycle or 5- to 10-membered heterocycle, wherein the carbocycle or heterocycle is optionally further substituted with 0 to 4 R^{2a};
each R^{2a} is independently selected from H, halogen, OH, ═O, cyano, NH₂, NH(C₁₋₆ alkyl), N(C₁₋₆ alkyl)₂, —SO₂—C₁₋₆ alkyl, —SO₂—C₃₋₆ cycloalkyl, C₁₋₆ alkyl, C₁₋₆ alkoxy, C₁₋₆ alkylthio, —Z—C₃₋₆ cycloalkyl, or —Z-3- to 8-membered heterocycle, wherein the alkyl, cycloalkyl, alkoxy, alkylthio, or heterocycle is optionally further substituted with 0 to 4 substituents selected from H, D, halogen, OH, ═O, cyano, NH₂, C₁₋₆ alkyl, C₂₋₆ alkynyl, C₁₋₆ alkoxy, or C₃₋₆ cycloalkyl, and the heterocycle contains 1 to 4 heteroatoms selected from O, S, and N;
R³ and R⁴ are each independently selected from H, halogen, OH, cyano, NH₂, C₁₋₆ alkyl, C₂₋₆ alkenyl, C₂₋₆ alkynyl, or C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, or 3- to 8-membered heterocyclyl, and the heterocyclyl contains 1 to 4 heteroatoms selected from O, S, and N;
Cy is selected from C₃₋₁₂ carbocycle or 4- to 12-membered heterocycle, and the Cy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, or 3- to 8-membered heterocyclyl, wherein the heterocycle contains 1 to 4 heteroatoms selected from O, S, and N;
R⁵ is selected from —Z—C₃₋₁₂ carbocycle or —Z-4- to 12-membered heterocycle, and the R⁵ is optionally further substituted with 0 to 4 R^{5a};
each R^{5a} is independently selected from H, halogen, OH, cyano, NH₂, NHC₁₋₆ alkyl, N(C₁₋₆ alkyl)₂, C₁₋₆ alkyl, C₂₋₆ alkynyl, C₁₋₆ alkoxy, —C₁₋₄ alkylene-C₃₋₆ cycloalkyl, —C₁₋₄ alkylene-3- to 6-membered heterocycle, C₃₋₆ cycloalkyl, 3- to 8-membered heterocycle, —CONHC₁₋₆ alkyl, —CONHC₁₋₆ alkoxy, —CONHC₃₋₆ cycloalkyl, —CONH 3- to 8-membered heterocycle, —NHCO—C₁₋₆ alkyl, —NHCO—C₁₋₆ alkoxy, —NHCO—C₃₋₆ cycloalkyl, or —NHCO-3- to 8-membered heterocycle, wherein the alkyl, alkylene, alkynyl, alkoxy, cycloalkyl, or heterocycle is optionally further substituted with 0 to 4 substituents selected from H, D, halogen, OH, ═O, cyano, NH₂, —C(═O)NH₂, —C(═O)NHC₁₋₆ alkyl, —C(═O)N(C₁₋₆ alkyl)₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, halogen-substituted C₃₋₆ cycloalkyl, or 3- to 8-membered heterocycle, and the heterocycle contains 1 to 4 heteroatoms selected from O, S, and N;
Z is selected from a bond, O, S, NH, N(C₁₋₄ alkyl), C(═O)NH, NHC(═O), —OCH₂—, —CH₂O—, or C₁₋₃ alkylene.

A second embodiment of the present invention is a compound shown in general formula (Ia) below or a stereoisomer, tautomer, deuterated substance, solvate, prodrug, metabolite, pharmaceutically acceptable salt or eutectic crystal thereof,

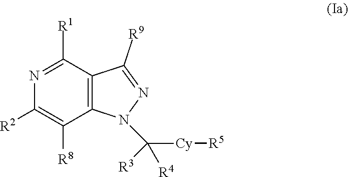

wherein R¹ and R⁹ are each independently selected from H, halogen, cyano, C₁₋₄ alkyl, C₂₋₄ alkenyl, C₂₋₄ alkynyl, or C₁₋₄ alkoxy, wherein the alkyl, alkenyl, alkynyl, or alkoxy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, or cyano-substituted C₁₋₄ alkyl;
R⁸ is selected from halogen, cyano, C₁₋₄ alkyl, C₂₋₄ alkynyl, and C₁₋₄ alkoxy, wherein the alkyl, alkynyl, or alkoxy is optionally further substituted with 0 to 4 substituents selected from H, halogen, OH, cyano, NH₂, C₁₋₄ alkyl, C₂₋₄ alkynyl, and C₁₋₄ alkoxy;
each R^{2a} is independently selected from H, halogen, OH, ═O, cyano, NH₂, NH(C₁₋₄ alkyl), N(C₁₋₄ alkyl)₂, —SO₂—C₁₋₄ alkyl, —SO₂—C₃₋₆ cycloalkyl, C₁₋₄ alkyl, C₁₋₄ alkoxy, C₁₋₄ alkylthio, —Z—C₃₋₆ cycloalkyl, or —Z-3- to 6-membered heterocycle, wherein the alkyl, cycloalkyl, alkoxy, alkylthio, or heterocycle is optionally further substituted with 0 to 4 substituents selected from H, D, halogen, OH, ═O, cyano, NH₂, C₁₋₄ alkyl, C₂₋₄ alkynyl, C₁₋₄ alkoxy, or C₃₋₆ cycloalkyl, and the heterocycle contains 1 to 4 heteroatoms selected from O, S, and N;
R³ and R⁴ are each independently selected from H, halogen, OH, cyano, and C₁₋₄ alkyl;
Cy is selected from C₃₋₆ monocyclic cycloalkyl, C₅₋₁₁ fused cycloalkyl, C₅₋₁₁ spirocycloalkyl, C₅₋₁₁ bridged cycloalkyl, 4- to 7-membered monocyclic heterocycloalkyl, 6- to 11-membered fused heterocycloalkyl, 6- to 11-membered spiroheterocycloalkyl, 6- to 11-membered bridged heterocycloalkyl, benzene ring, benzo C₄₋₆ carbocycle, benzo 4- to 6-membered heterocycle, 5- to 6-membered heteroaromatic ring, or 8- to 10-membered fused heteroaromatic ring, and the Cy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, or 3- to 8-membered heterocycle, wherein the heterocycle contains 1 to 4 heteroatoms selected from O, S, and N;
R⁵ is selected from —Z—C₆₋₁₂ carbocycle or —Z-5- to 12-membered heterocycle, and the R⁵ is optionally further substituted with 0 to 4 R^{5a};
each R^{5a} is independently selected from H, halogen, OH, cyano, NH₂, C₁₋₄ alkyl, C₂₋₄ alkynyl, C₁₋₄ alkoxy, —C₁₋₂ alkylene-C₃₋₆ cycloalkyl, —C₁₋₂ alkylene-3- to 6-membered heterocycle, C₃₋₆ cycloalkyl, or 3- to 6-membered heterocycle, wherein the alkyl, alkylene, alkynyl, alkoxy, cycloalkyl, or heterocycle is optionally further substituted with 0 to 4 substituents selected from H, D, halogen, OH, ═O, cyano, NH₂, —C(═O)NH₂, —C(═O)NHC₁₋₄ alkyl, —C(═O)N(C₁₋₄ alkyl)₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, halogen-substituted C₃₋₆ cycloalkyl, or 3- to 8-membered heterocycle, and the heterocycle contains 1 to 4 heteroatoms selected from O, S, and N;
the definitions of the remaining substituents are the same as those in the first solution of the present invention.

A third embodiment of the present invention is a compound shown in general formula (Ia) above or a stereoisomer, tautomer, deuterated substance, solvate, prodrug, metabolite, pharmaceutically acceptable salt or eutectic crystal thereof, wherein
R¹ and R⁹ are each independently selected from H, F, Cl, Br, I, cyano, methyl, or ethyl;
R⁸ is selected from F, Cl, Br, I, CN, methyl, ethyl, ethynyl, methoxy, or ethoxy;
R² is selected from phenyl, pyridine, pyrimidine, pyrrole, pyrazole, imidazole, furan, thiophene, thiazole, or oxazole, wherein the phenyl, pyridine, pyrimidine, pyrrole, pyrazole, imidazole, furan, thiophene, thiazole, or oxazole is optionally further substituted with 0 to 4 R^{2a};
or R² is selected from

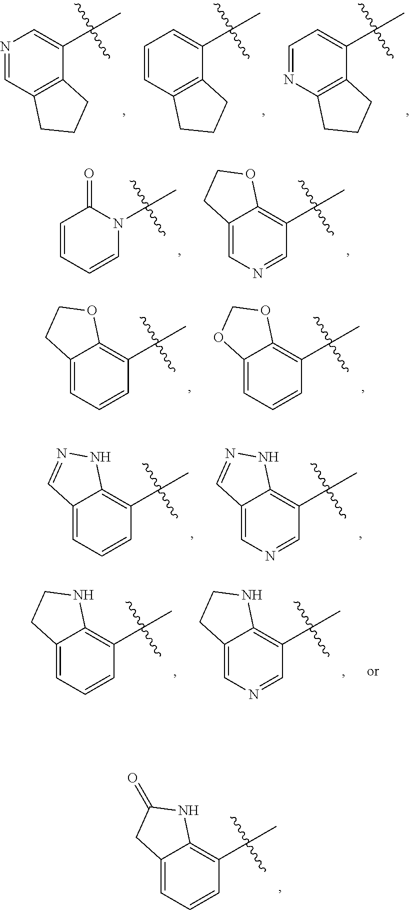

and R² is optionally further substituted with 0 to 4 R^{2a};
each R^{2a} is independently selected from H, F, Cl, Br, I, or cyano, or each R^{2a} is independently selected from one of the following substituted or unsubstituted groups: —SO₂-methyl, —SO₂-ethyl, —SO₂-propyl, —SO₂-isopropyl, —SO₂-cyclopropyl, methyl, ethyl, propyl, isopropyl, methoxy, ethoxy, propoxy, methylthio, ethylthio, propylthio, cyclopropyl, cyclobutyl, cyclopentyl, cyclohexyl, azetidinyl, azacyclopentyl, azacyclohexyl, piperazine, oxetanyl, oxacyclopentyl, oxacyclohexyl, morpholine, —O-azetidinyl, —O-azacyclopentyl, —O-azacyclohexyl, —O-oxetanyl, —O-oxacyclopentyl, —O-oxacyclohexyl, —OCH₂-phenyl, —OCH₂-cyclopropyl, —OCH₂-cyclobutyl, —OCH₂-cyclopentyl, —OCH₂-cyclohexyl, pyrrole, pyrazole, imidazole, thiazole, thiophene, furan, and oxazole, which, when substituted, are optionally further substituted with 0 to 4 substituents selected from H, D, halogen, OH, ═O, cyano, NH₂, C₁₋₄ alkyl, C₂₋₄ alkynyl, C₁₋₄ alkoxy, and C₃₋₆ cycloalkyl;
R³ and R⁴ are each independently selected from H, F, Cl, Br, I, methyl, and ethyl;
Cy is selected from cyclopropyl, cyclobutyl, cyclopentyl, cyclohexyl, bicyclo[2.2.2]octyl, bicyclo[1.1.1]pentyl, cyclopentyl-fused cyclopentyl, cyclobutylspirocyclohexyl, cyclobutylspirocyclopentyl, azetidinyl, azacyclopentyl, azacyclohexyl, azetidinylspirocyclobutyl, azetidinylspirocyclopentyl, azetidinylspirocyclohexyl, azetidinylspiroazetidinyl, azetidinylspiroazacyclopentyl, azetidinylspiroazacyclohexyl,

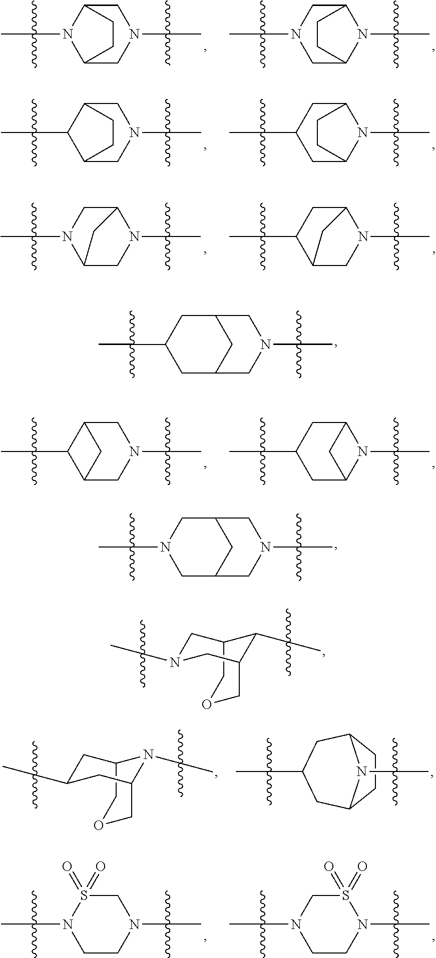

benzene ring, thiophene, furan, pyrrole, thiazole, oxazole, pyrazole, pyrazine, pyrimidine,

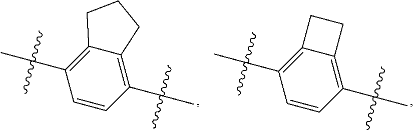 [SMILES: *c1ccc(*)c2c1CCC2 ; *c1ccc(*)c2c1CC2]

benzothiazole, benzothiophene, benzofuran, benzoxazole, benzopyrrole, benzopyrazole, benzimidazole, indolizine, pyridinopyrrole, pyridinoimidazole, pyridinopyrazole, pyrimidoimidazole, pyrimidopyrazole, pyridinotriazole, or 2-pyridone, and the Cy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁. 4 alkyl, C₃₋₆ cycloalkyl, or 3- to 8-membered heterocycle, wherein the heterocycle contains 1 to 4 heteroatoms selected from O, S, and N;
R⁵ is selected from benzene ring, pyrazole, pyrrole, imidazole, triazole, pyridine, pyrimidine, pyrazine, or pyridazine, and the R⁵ is optionally further substituted with 0 to 4 R^{5a};
or R⁵ is selected from

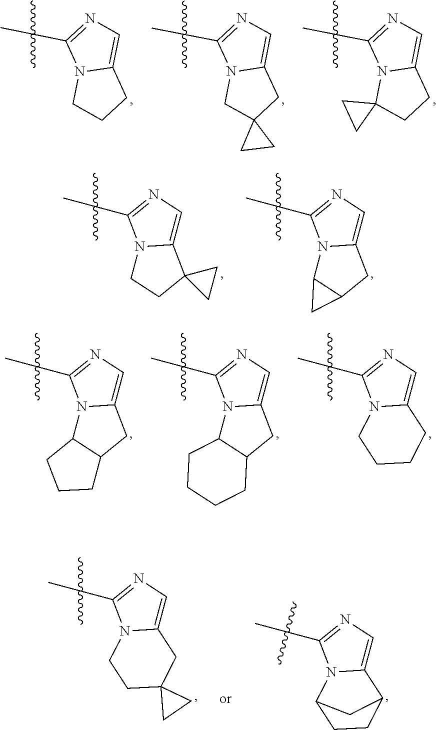

and the R⁵ is optionally further substituted with 0 to 4 R^{5a};
each R^{5a} is independently selected from F, Cl, Br, I, cyano, methyl, ethyl, propyl, isopropyl, propargyl, butargyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, —CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, or —CH₂-azacyclohexyl, wherein the methyl, ethyl, propyl, isopropyl, propargyl, butargyl, methoxy, ethoxy, cyclopropyl, cyclobutyl, cyclopentyl, cyclohexyl, oxetanyl, oxacyclopentyl, oxacyclohexyl, azetidinyl, azacyclopentyl, or azacyclohexyl is optionally further substituted with 0 to 4 substituents selected from H, halogen, OH, ═O, cyano, NH₂, C₁₋₄ alkyl, C₂₋₄ alkynyl, C₁₋₄ alkoxy, C₃₋₆ cycloalkyl, or halogen-substituted C₃₋₆ cycloalkyl;
the definitions of the remaining substituents are the same as those in the first or second solution of the present invention.

A fourth embodiment of the present invention is a compound shown in general formula (Ia) above or a stereoisomer, tautomer, deuterated substance, solvate, prodrug, metabolite, pharmaceutically acceptable salt or eutectic crystal thereof, wherein
R¹ and R⁹ are H;
R⁸ is selected from F, Cl, Br, CN, methyl, ethynyl, or methoxy;
R² is selected from pyrrole, pyrazole, imidazole, pyrimidine,

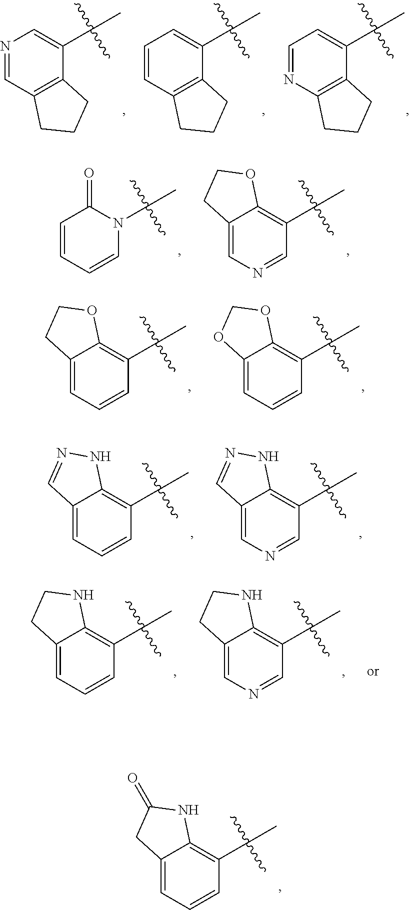

wherein the pyrrole, pyrazole, imidazole, pyrimidine,

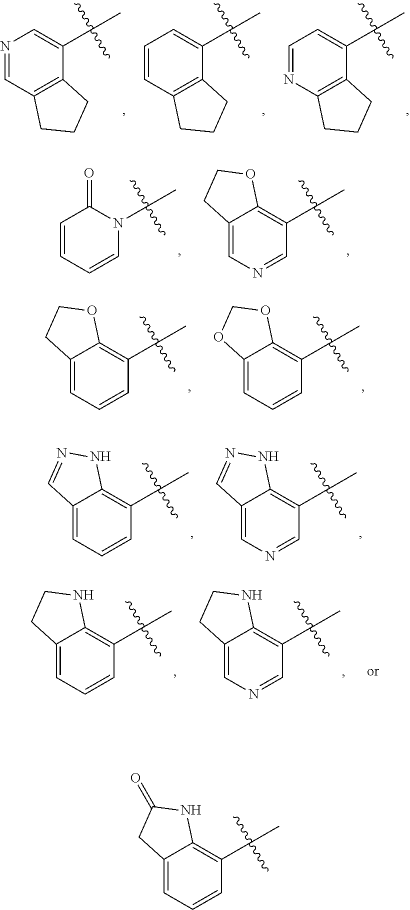

is optionally further substituted with 0 to 4 substituents selected from H, F, Cl, Br, I, cyano, —SO₂-methyl, —SO₂-ethyl, —SO₂-propyl, —SO₂-isopropyl, —SO₂-cyclopropyl, CH₂F, CHF₂, CF₃, —OCH₂F, —OCHF₂, —OCF₃, methyl, ethyl, propyl, isopropyl, methoxy, ethoxy, propoxy, —OCD₃, methylthio, ethylthio, propylthio, cyclopropyl, cyclobutyl, cyclopentyl, cyclohexyl, azetidinyl, azacyclopentyl, azacyclohexyl, piperazine, oxetanyl, oxacyclopentyl, oxacyclohexyl, morpholine, —O-azetidinyl, —O-azacyclopentyl, —O-azacyclohexyl, —O-oxetanyl, —O-oxacyclopentyl, —O-oxacyclohexyl, —OCH₂-phenyl, —OCH₂-cyclopropyl, —OCH₂-cyclobutyl, —OCH₂-cyclopentyl, —OCH₂-cyclohexyl,

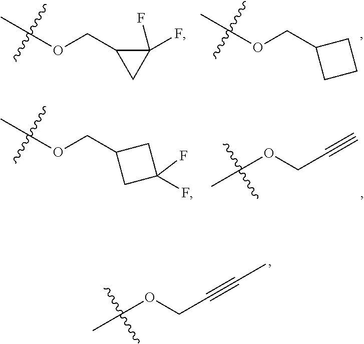

pyrrole, pyrazole, imidazole, thiazole, thiophene, furan, oxazole,

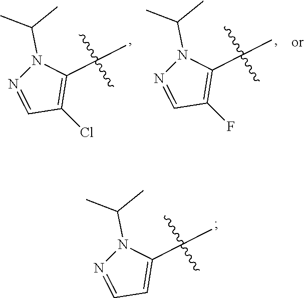

Cy is selected from

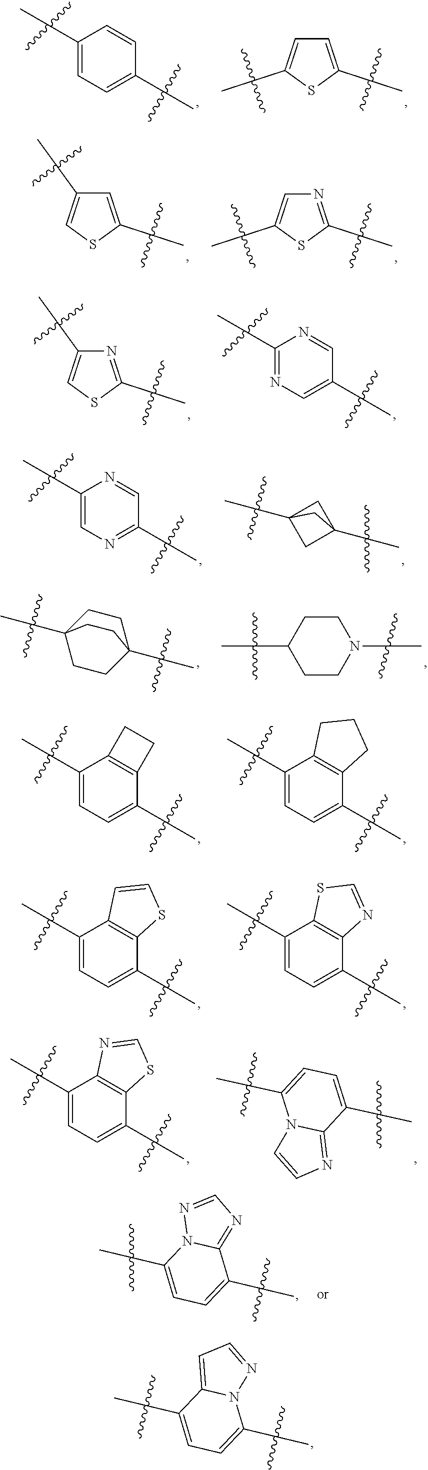

and the Cy is optionally further substituted with 0 to 4 substituents selected from H, F, Cl, Br, I, cyano, CH₂F, CHF₂, CF₃, methyl, ethyl, propyl, isopropyl, methoxy, ethoxy, or cyclopropyl;
alternatively, Cy is selected from

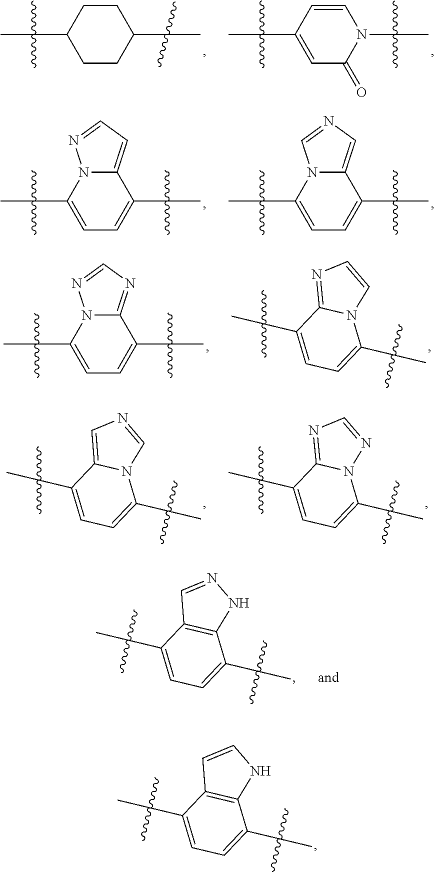

and the Cy is optionally further substituted with 0 to 4 substituents selected from H, F, Cl, Br, I, cyano, CH₂F, CHF₂, CF₃, methyl, ethyl, propyl, isopropyl, methoxy, ethoxy, or cyclopropyl;
R⁵ is selected from pyrrole, pyrazole, or imidazole, and the pyrrole, pyrazole, or imidazole is optionally further substituted with 0 to 4 substituents selected from H, F, Cl, Br, I, cyano, CH₂F, CHF₂, CF₃, CD₃, methyl, ethyl, propyl, isopropyl, —CH₂CH₂F, methoxy, ethoxy, cyclopropyl, cyclobutyl, cyclopentyl, cyclohexyl, oxetanyl, oxacyclopentyl, oxacyclohexyl, azetidinyl, N-methylazetidinyl, azacyclopentyl, azacyclohexyl,

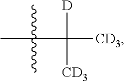 [SMILES: *C([2H])(C([2H])([2H])[2H])C([2H])([2H])[2H]]

—CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, —CH₂-azacyclohexyl,

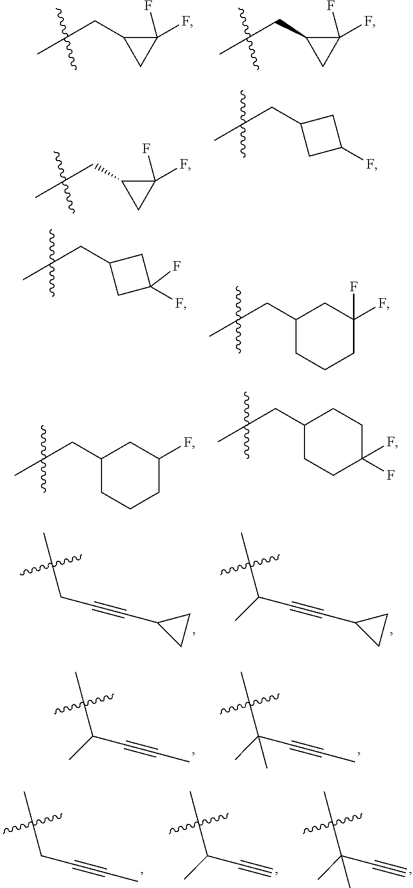

or propargyl;
alternatively, R⁵ is selected from

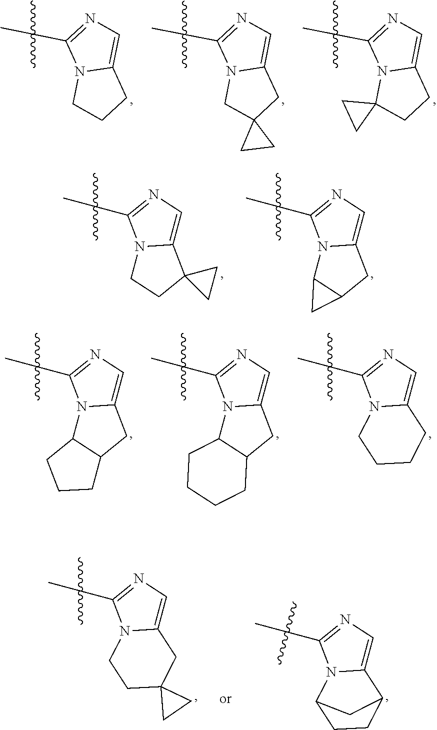

and R⁵ is optionally further substituted with 0 to 4 substituents selected from H, F, Cl, Br, I, cyano, CH₂F, CHF₂, CF₃, CD₃, methyl, ethyl, propyl, isopropyl, —CH₂CH₂F, methoxy, ethoxy, cyclopropyl, cyclobutyl, cyclopentyl, cyclohexyl, oxetanyl, oxacyclopentyl, oxacyclohexyl, azetidinyl, N-methylazetidinyl, azacyclopentyl, azacyclohexyl,

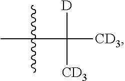 [SMILES: *C([2H])(C([2H])([2H])[2H])C([2H])([2H])[2H]]

—CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, —CH₂-azacyclohexyl,

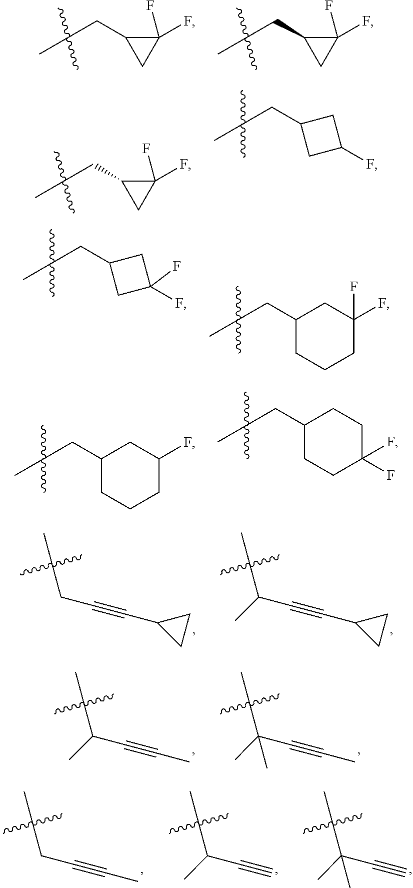

or propargyl;
the definitions of the remaining substituents are the same as those in the first, second, or third solution of the present invention.

A fifth embodiment of the present invention is a compound shown in general formula (Ia) above or a stereoisomer, tautomer, deuterated substance, solvate, prodrug, metabolite, pharmaceutically acceptable salt or eutectic crystal thereof, wherein
R² is selected from

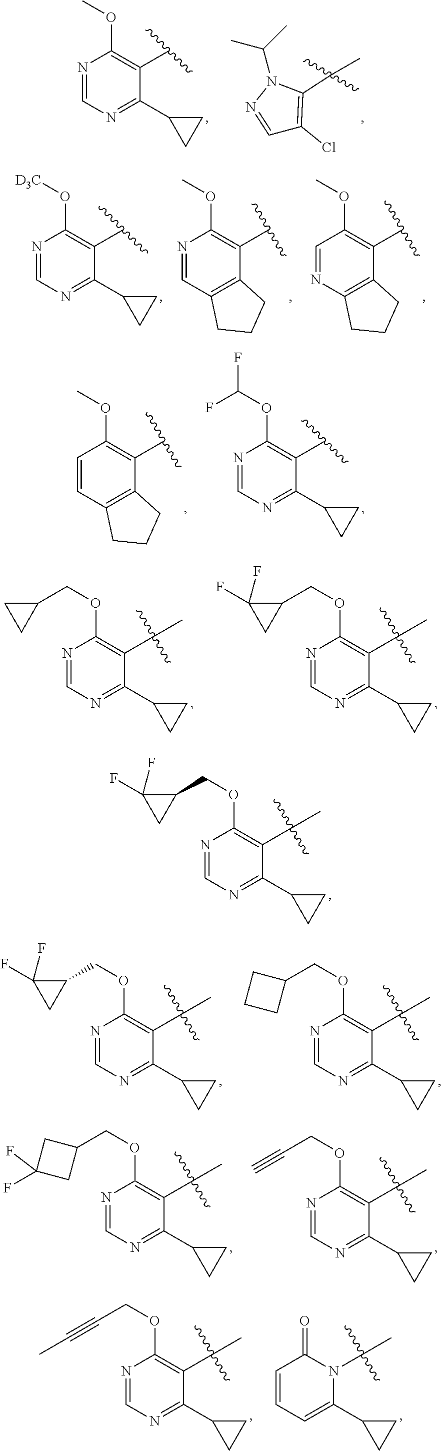

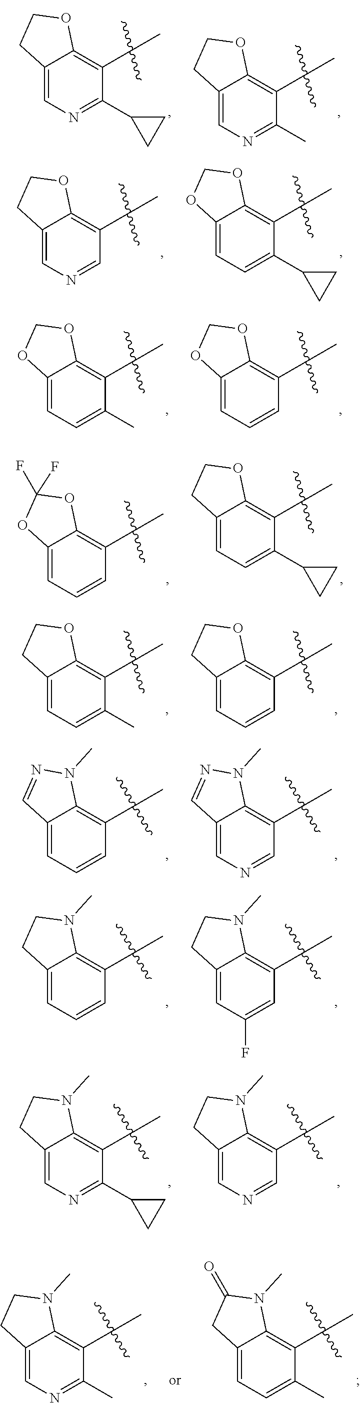

R⁵ is selected from

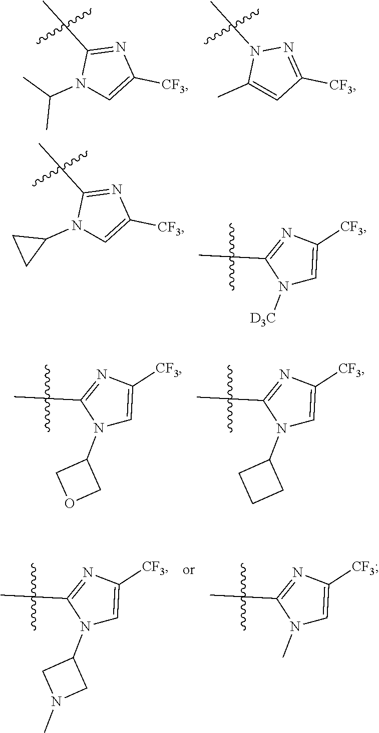

R⁵ is selected from

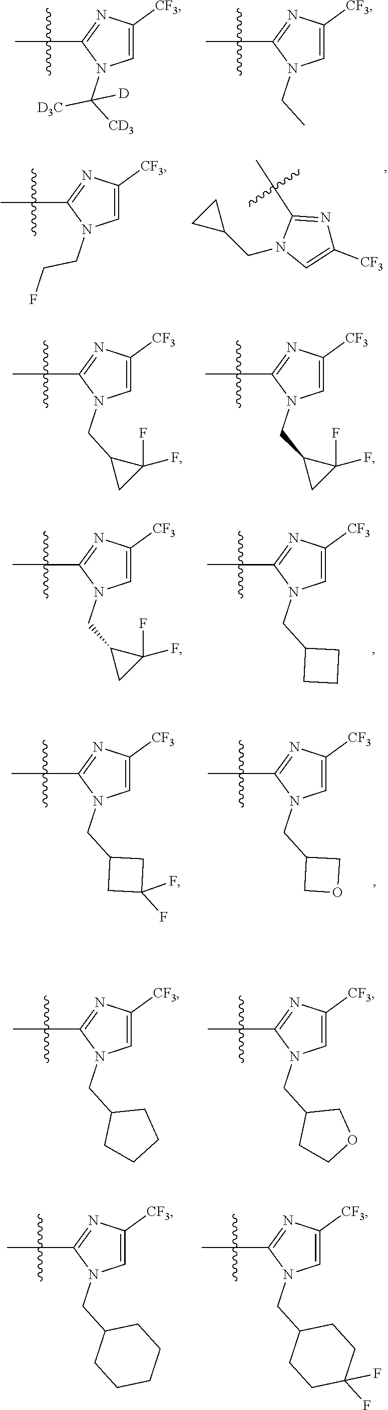

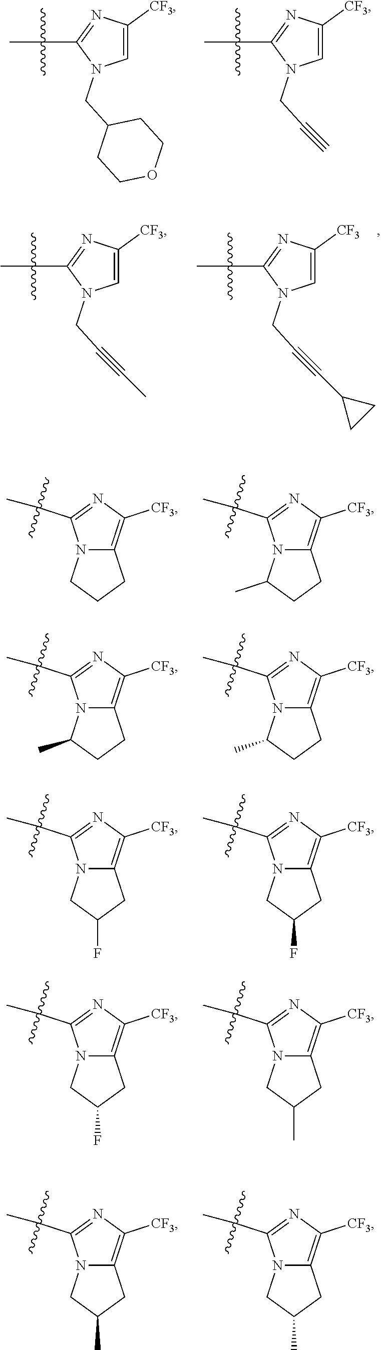

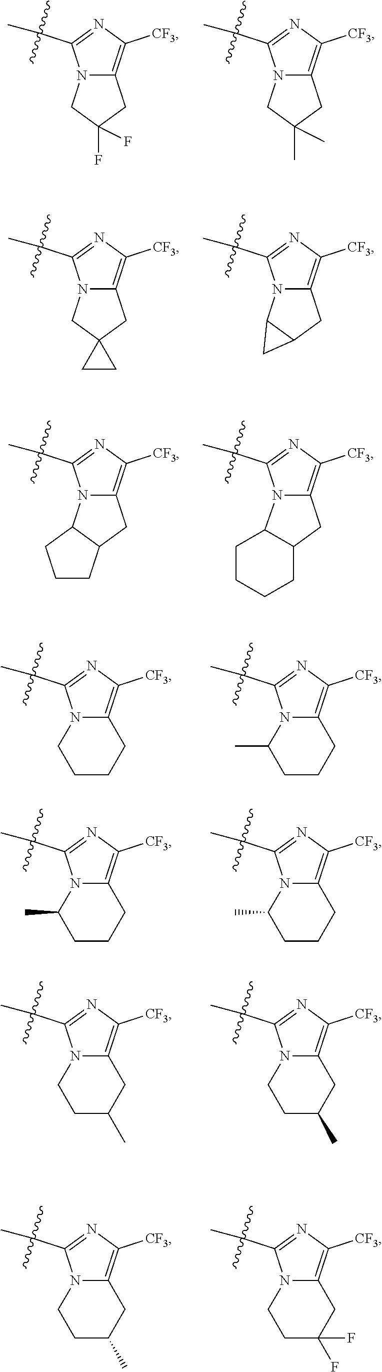

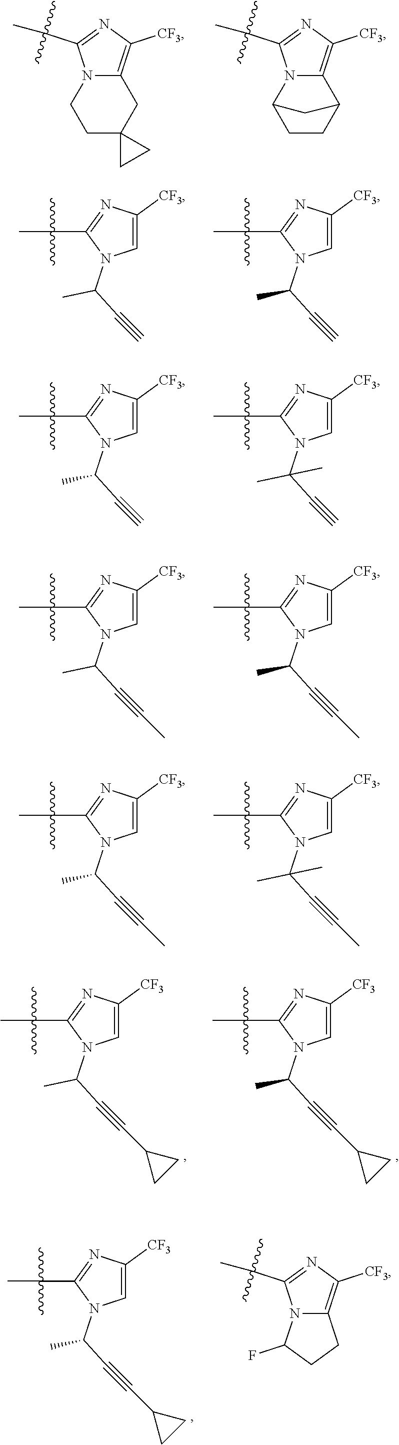

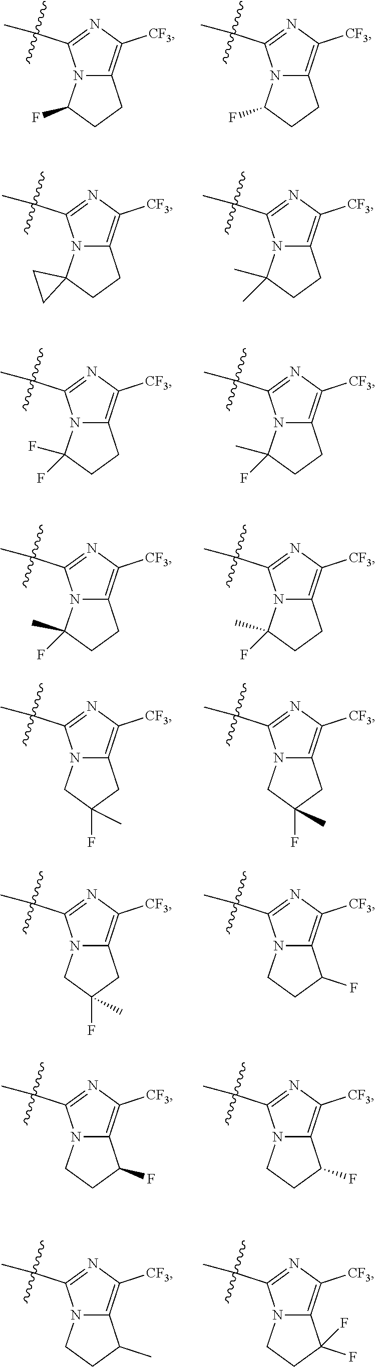

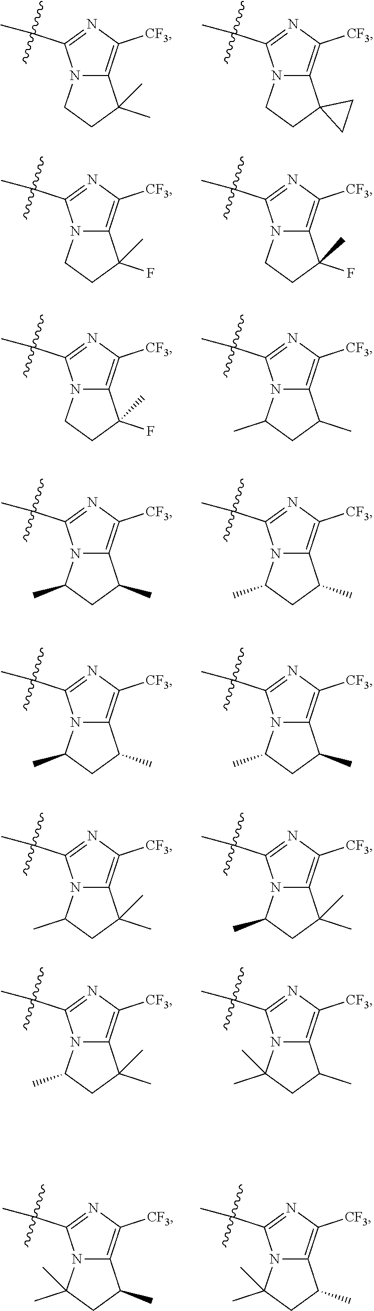

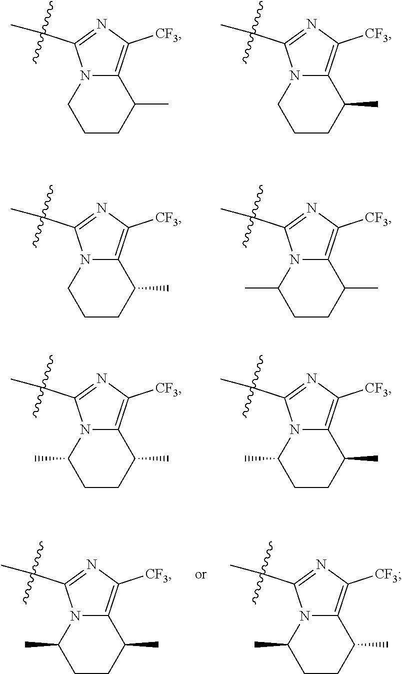

R⁸ is selected from F or Cl;
the definitions of the remaining substituents are the same as those in the first, second, third, or fourth solution of the present invention.

The present invention relates to the following compound or a stereoisomer, tautomer, deuterated substance, solvate, prodrug, metabolite, pharmaceutically acceptable salt or eutectic crystal thereof, wherein the compound is selected from one of the structures shown in Table E.

TABLE E

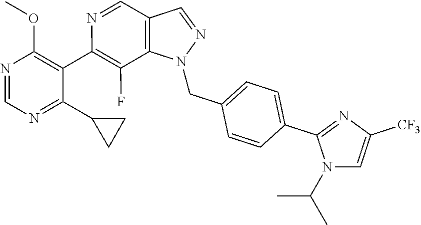 [SMILES: COc1ncnc(C2CC2)c1-c1ncc2cnn(Cc3ccc(-c4nc(C(F)(F)F)cn4C(C)C)cc3)c2c1F]

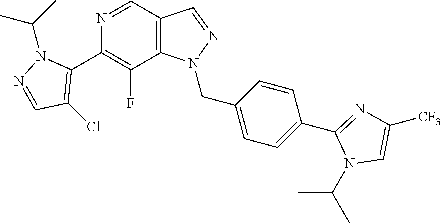 [SMILES: CC(C)n1cc(C(F)(F)F)nc1-c1ccc(Cn2ncc3cnc(-c4c(Cl)cnn4C(C)C)c(F)c32)cc1]

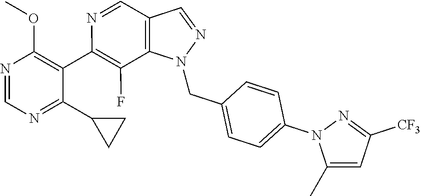 [SMILES: COc1ncnc(C2CC2)c1-c1ncc2cnn(Cc3ccc(-n4nc(C(F)(F)F)cc4C)cc3)c2c1F]

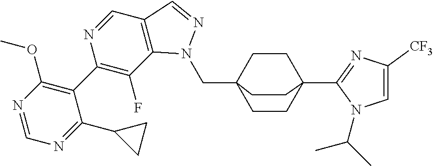 [SMILES: COc1ncnc(C2CC2)c1-c1ncc2cnn(CC34CCC(c5nc(C(F)(F)F)cn5C(C)C)(CC3)CC4)c2c1F]

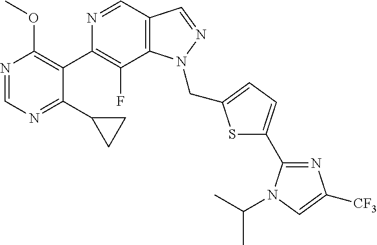 [SMILES: COc1ncnc(C2CC2)c1-c1ncc2cnn(Cc3ccc(-c4nc(C(F)(F)F)cn4C(C)C)s3)c2c1F]

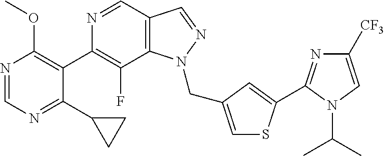 [SMILES: COc1ncnc(C2CC2)c1-c1ncc2cnn(Cc3csc(-c4nc(C(F)(F)F)cn4C(C)C)c3)c2c1F]

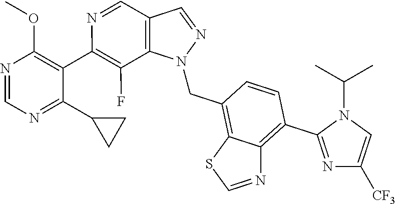 [SMILES: COc1ncnc(C2CC2)c1-c1ncc2cnn(Cc3ccc(-c4nc(C(F)(F)F)cn4C(C)C)c4ncsc34)c2c1F]

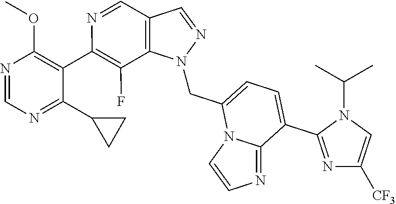 [SMILES: COc1ncnc(C2CC2)c1-c1ncc2cnn(Cc3ccc(-c4nc(C(F)(F)F)cn4C(C)C)c4nccn34)c2c1F]

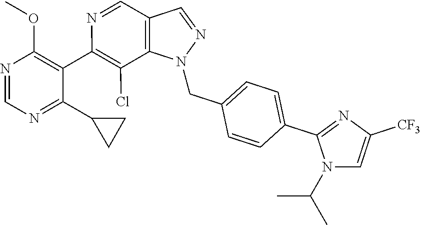 [SMILES: COc1ncnc(C2CC2)c1-c1ncc2cnn(Cc3ccc(-c4nc(C(F)(F)F)cn4C(C)C)cc3)c2c1Cl]

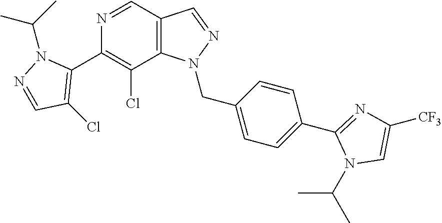 [SMILES: CC(C)n1cc(C(F)(F)F)nc1-c1ccc(Cn2ncc3cnc(-c4c(Cl)cnn4C(C)C)c(Cl)c32)cc1]

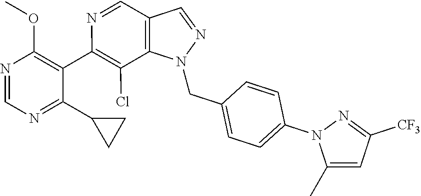 [SMILES: COc1ncnc(C2CC2)c1-c1ncc2cnn(Cc3ccc(-n4nc(C(F)(F)F)cc4C)cc3)c2c1Cl]

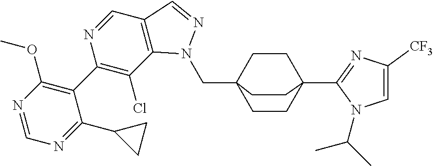 [SMILES: COc1ncnc(C2CC2)c1-c1ncc2cnn(CC34CCC(c5nc(C(F)(F)F)cn5C(C)C)(CC3)CC4)c2c1Cl]

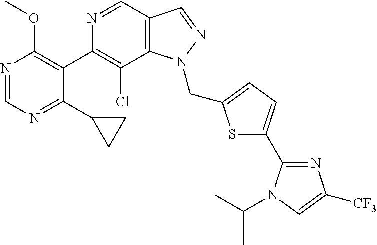 [SMILES: COc1ncnc(C2CC2)c1-c1ncc2cnn(Cc3ccc(-c4nc(C(F)(F)F)cn4C(C)C)s3)c2c1Cl]

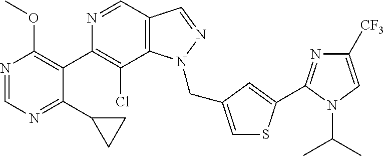 [SMILES: COc1ncnc(C2CC2)c1-c1ncc2cnn(Cc3csc(-c4nc(C(F)(F)F)cn4C(C)C)c3)c2c1Cl]

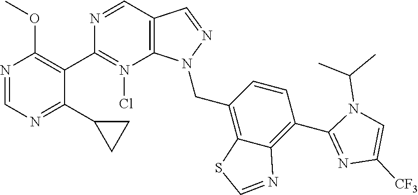

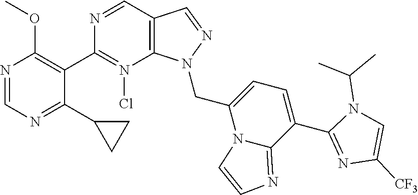

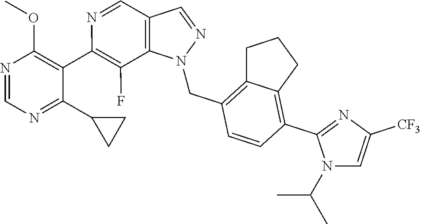 [SMILES: COc1ncnc(C2CC2)c1-c1ncc2cnn(Cc3ccc(-c4nc(C(F)(F)F)cn4C(C)C)c4c3CCC4)c2c1F]

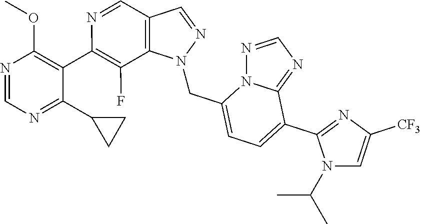 [SMILES: COc1ncnc(C2CC2)c1-c1ncc2cnn(Cc3ccc(-c4nc(C(F)(F)F)cn4C(C)C)c4ncnn34)c2c1F]

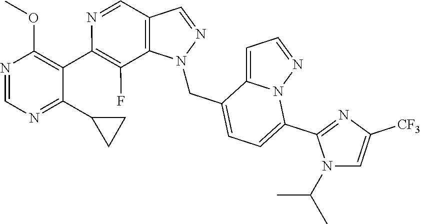 [SMILES: COc1ncnc(C2CC2)c1-c1ncc2cnn(Cc3ccc(-c4nc(C(F)(F)F)cn4C(C)C)n4nccc34)c2c1F]

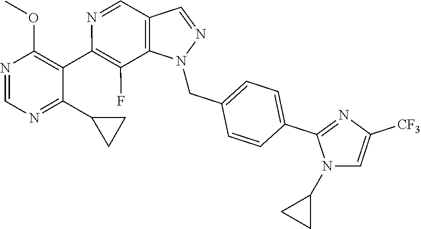 [SMILES: COc1ncnc(C2CC2)c1-c1ncc2cnn(Cc3ccc(-c4nc(C(F)(F)F)cn4C4CC4)cc3)c2c1F]

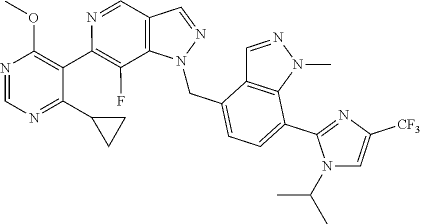 [SMILES: COc1ncnc(C2CC2)c1-c1ncc2cnn(Cc3ccc(-c4nc(C(F)(F)F)cn4C(C)C)c4c3cnn4C)c2c1F]

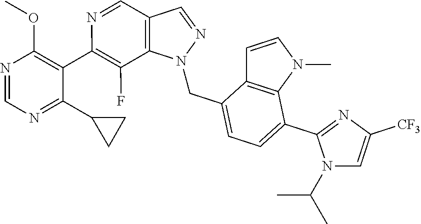 [SMILES: COc1ncnc(C2CC2)c1-c1ncc2cnn(Cc3ccc(-c4nc(C(F)(F)F)cn4C(C)C)c4c3ccn4C)c2c1F]

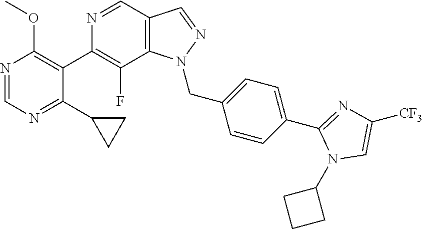 [SMILES: COc1ncnc(C2CC2)c1-c1ncc2cnn(Cc3ccc(-c4nc(C(F)(F)F)cn4C4CCC4)cc3)c2c1F]

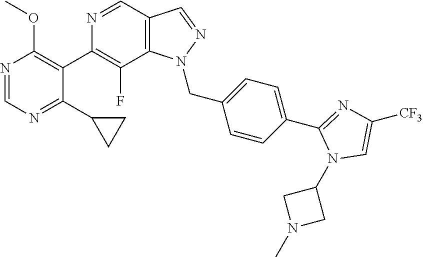 [SMILES: COc1ncnc(C2CC2)c1-c1ncc2cnn(Cc3ccc(-c4nc(C(F)(F)F)cn4C4CN(C)C4)cc3)c2c1F]

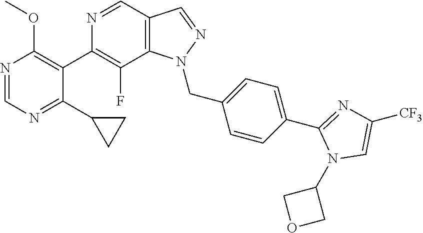 [SMILES: COc1ncnc(C2CC2)c1-c1ncc2cnn(Cc3ccc(-c4nc(C(F)(F)F)cn4C4COC4)cc3)c2c1F]

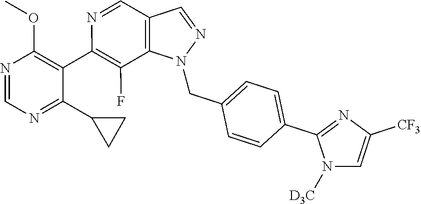 [SMILES: [2H]C([2H])([2H])n1cc(C(F)(F)F)nc1-c1ccc(Cn2ncc3cnc(-c4c(OC)ncnc4C4CC4)c(F)c32)cc1]

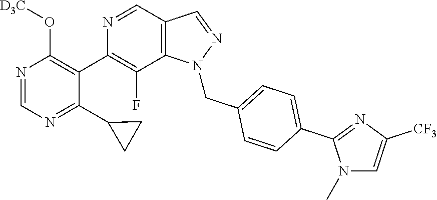 [SMILES: [2H]C([2H])([2H])Oc1ncnc(C2CC2)c1-c1ncc2cnn(Cc3ccc(-c4nc(C(F)(F)F)cn4C)cc3)c2c1F]

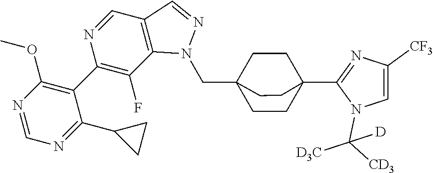 [SMILES: [2H]C([2H])([2H])C([2H])(n1cc(C(F)(F)F)nc1C12CCC(Cn3ncc4cnc(-c5c(OC)ncnc5C5CC5)c(F)c43)(CC1)CC2)C([2H])([2H])[2H]]

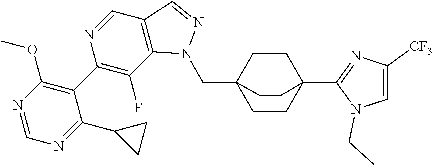 [SMILES: CCn1cc(C(F)(F)F)nc1C12CCC(Cn3ncc4cnc(-c5c(OC)ncnc5C5CC5)c(F)c43)(CC1)CC2]

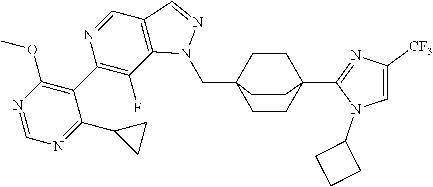 [SMILES: COc1ncnc(C2CC2)c1-c1ncc2cnn(CC34CCC(c5nc(C(F)(F)F)cn5C5CCC5)(CC3)CC4)c2c1F]

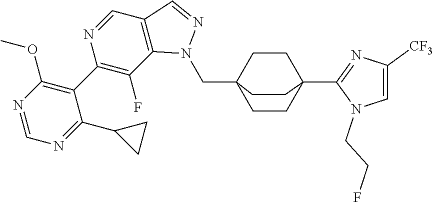 [SMILES: COc1ncnc(C2CC2)c1-c1ncc2cnn(CC34CCC(c5nc(C(F)(F)F)cn5CCF)(CC3)CC4)c2c1F]

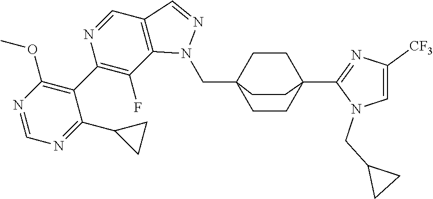 [SMILES: COc1ncnc(C2CC2)c1-c1ncc2cnn(CC34CCC(c5nc(C(F)(F)F)cn5CC5CC5)(CC3)CC4)c2c1F]

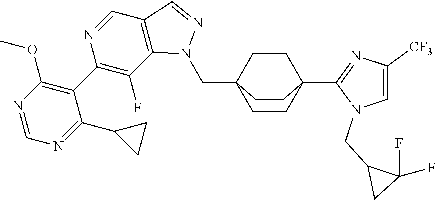 [SMILES: COc1ncnc(C2CC2)c1-c1ncc2cnn(CC34CCC(c5nc(C(F)(F)F)cn5CC5CC5(F)F)(CC3)CC4)c2c1F]

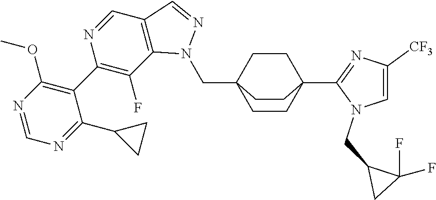 [SMILES: COc1ncnc(C2CC2)c1-c1ncc2cnn(CC34CCC(c5nc(C(F)(F)F)cn5C[C@@H]5CC5(F)F)(CC3)CC4)c2c1F]

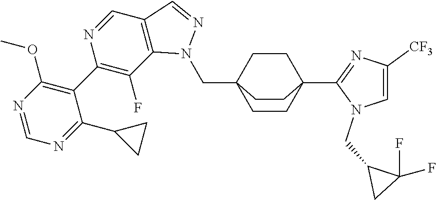 [SMILES: COc1ncnc(C2CC2)c1-c1ncc2cnn(CC34CCC(c5nc(C(F)(F)F)cn5C[C@H]5CC5(F)F)(CC3)CC4)c2c1F]

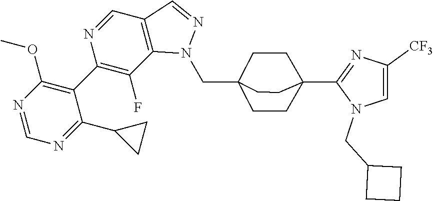 [SMILES: COc1ncnc(C2CC2)c1-c1ncc2cnn(CC34CCC(c5nc(C(F)(F)F)cn5CC5CCC5)(CC3)CC4)c2c1F]

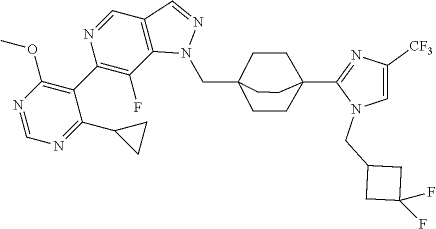 [SMILES: COc1ncnc(C2CC2)c1-c1ncc2cnn(CC34CCC(c5nc(C(F)(F)F)cn5CC5CC(F)(F)C5)(CC3)CC4)c2c1F]

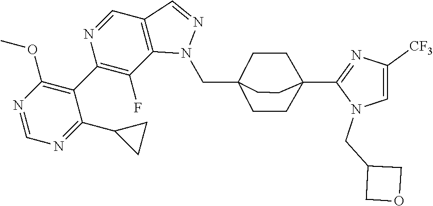 [SMILES: COc1ncnc(C2CC2)c1-c1ncc2cnn(CC34CCC(c5nc(C(F)(F)F)cn5CC5COC5)(CC3)CC4)c2c1F]

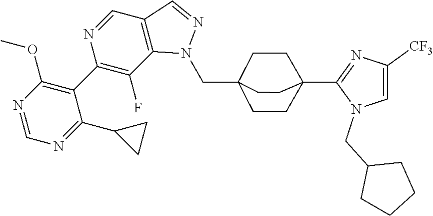 [SMILES: COc1ncnc(C2CC2)c1-c1ncc2cnn(CC34CCC(c5nc(C(F)(F)F)cn5CC5CCCC5)(CC3)CC4)c2c1F]

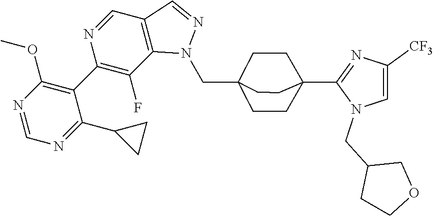 [SMILES: COc1ncnc(C2CC2)c1-c1ncc2cnn(CC34CCC(c5nc(C(F)(F)F)cn5CC5CCOC5)(CC3)CC4)c2c1F]

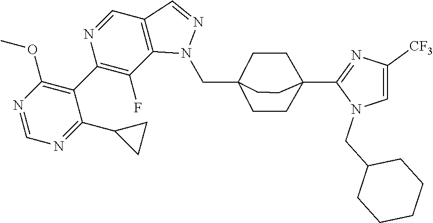 [SMILES: COc1ncnc(C2CC2)c1-c1ncc2cnn(CC34CCC(c5nc(C(F)(F)F)cn5CC5CCCCC5)(CC3)CC4)c2c1F]

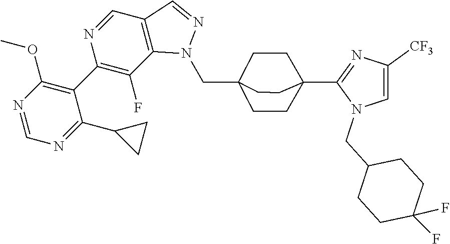 [SMILES: COc1ncnc(C2CC2)c1-c1ncc2cnn(CC34CCC(c5nc(C(F)(F)F)cn5CC5CCC(F)(F)CC5)(CC3)CC4)c2c1F]

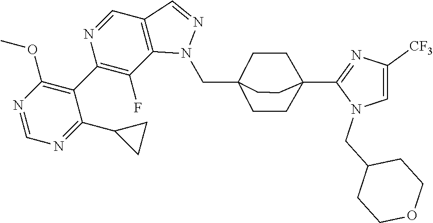 [SMILES: COc1ncnc(C2CC2)c1-c1ncc2cnn(CC34CCC(c5nc(C(F)(F)F)cn5CC5CCOCC5)(CC3)CC4)c2c1F]

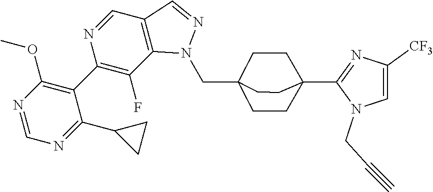 [SMILES: C#CCn1cc(C(F)(F)F)nc1C12CCC(Cn3ncc4cnc(-c5c(OC)ncnc5C5CC5)c(F)c43)(CC1)CC2]

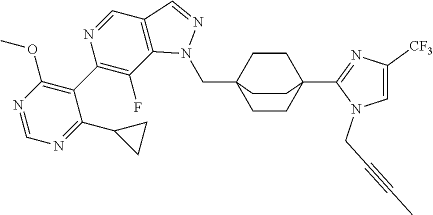 [SMILES: CC#CCn1cc(C(F)(F)F)nc1C12CCC(Cn3ncc4cnc(-c5c(OC)ncnc5C5CC5)c(F)c43)(CC1)CC2]

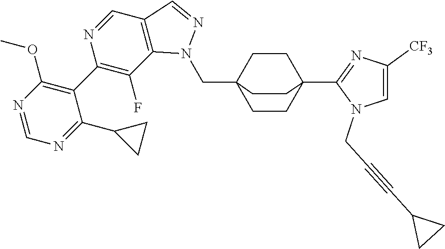 [SMILES: COc1ncnc(C2CC2)c1-c1ncc2cnn(CC34CCC(c5nc(C(F)(F)F)cn5CC#CC5CC5)(CC3)CC4)c2c1F]

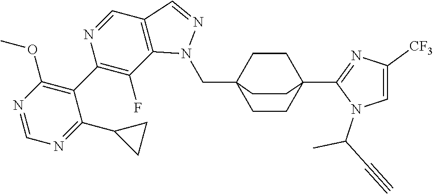 [SMILES: C#CC(C)n1cc(C(F)(F)F)nc1C12CCC(Cn3ncc4cnc(-c5c(OC)ncnc5C5CC5)c(F)c43)(CC1)CC2]

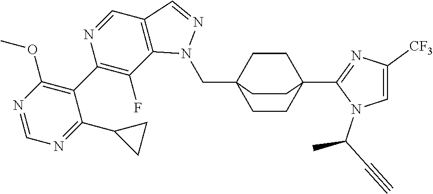 [SMILES: C#C[C@@H](C)n1cc(C(F)(F)F)nc1C12CCC(Cn3ncc4cnc(-c5c(OC)ncnc5C5CC5)c(F)c43)(CC1)CC2]

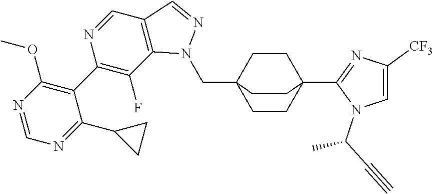 [SMILES: C#C[C@H](C)n1cc(C(F)(F)F)nc1C12CCC(Cn3ncc4cnc(-c5c(OC)ncnc5C5CC5)c(F)c43)(CC1)CC2]

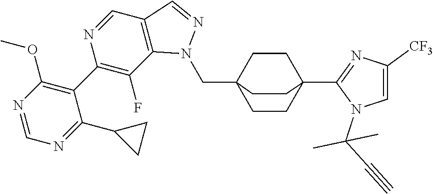 [SMILES: C#CC(C)(C)n1cc(C(F)(F)F)nc1C12CCC(Cn3ncc4cnc(-c5c(OC)ncnc5C5CC5)c(F)c43)(CC1)CC2]

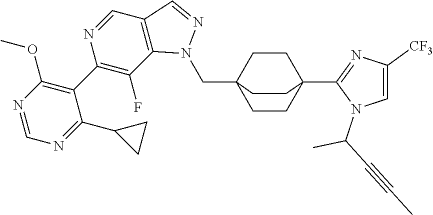 [SMILES: CC#CC(C)n1cc(C(F)(F)F)nc1C12CCC(Cn3ncc4cnc(-c5c(OC)ncnc5C5CC5)c(F)c43)(CC1)CC2]

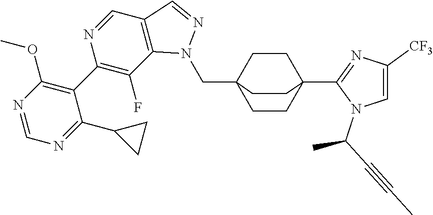 [SMILES: CC#C[C@@H](C)n1cc(C(F)(F)F)nc1C12CCC(Cn3ncc4cnc(-c5c(OC)ncnc5C5CC5)c(F)c43)(CC1)CC2]

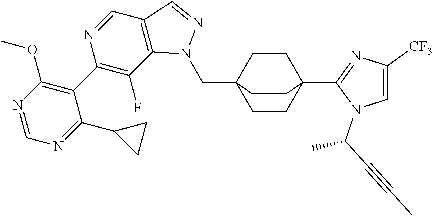 [SMILES: CC#C[C@H](C)n1cc(C(F)(F)F)nc1C12CCC(Cn3ncc4cnc(-c5c(OC)ncnc5C5CC5)c(F)c43)(CC1)CC2]

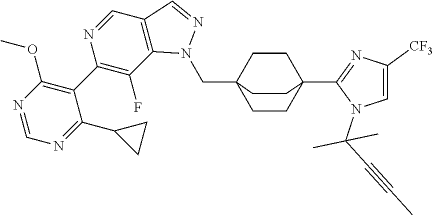 [SMILES: CC#CC(C)(C)n1cc(C(F)(F)F)nc1C12CCC(Cn3ncc4cnc(-c5c(OC)ncnc5C5CC5)c(F)c43)(CC1)CC2]

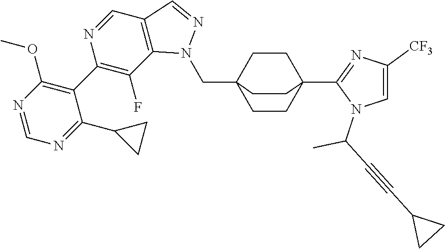 [SMILES: COc1ncnc(C2CC2)c1-c1ncc2cnn(CC34CCC(c5nc(C(F)(F)F)cn5C(C)C#CC5CC5)(CC3)CC4)c2c1F]

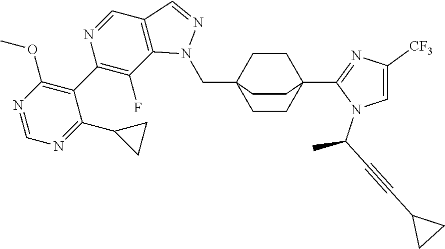 [SMILES: COc1ncnc(C2CC2)c1-c1ncc2cnn(CC34CCC(c5nc(C(F)(F)F)cn5[C@H](C)C#CC5CC5)(CC3)CC4)c2c1F]

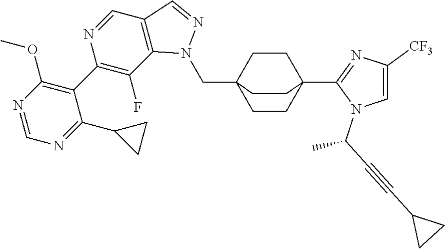 [SMILES: COc1ncnc(C2CC2)c1-c1ncc2cnn(CC34CCC(c5nc(C(F)(F)F)cn5[C@@H](C)C#CC5CC5)(CC3)CC4)c2c1F]

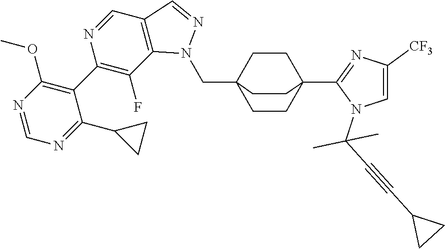 [SMILES: COc1ncnc(C2CC2)c1-c1ncc2cnn(CC34CCC(c5nc(C(F)(F)F)cn5C(C)(C)C#CC5CC5)(CC3)CC4)c2c1F]

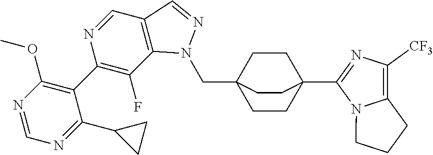 [SMILES: COc1ncnc(C2CC2)c1-c1ncc2cnn(CC34CCC(c5nc(C(F)(F)F)c6n5CCC6)(CC3)CC4)c2c1F]

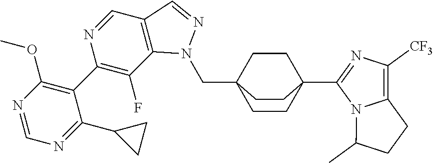 [SMILES: COc1ncnc(C2CC2)c1-c1ncc2cnn(CC34CCC(c5nc(C(F)(F)F)c6n5C(C)CC6)(CC3)CC4)c2c1F]

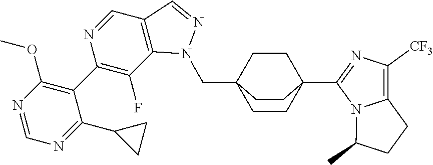 [SMILES: COc1ncnc(C2CC2)c1-c1ncc2cnn(CC34CCC(c5nc(C(F)(F)F)c6n5[C@H](C)CC6)(CC3)CC4)c2c1F]

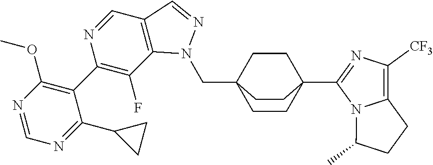 [SMILES: COc1ncnc(C2CC2)c1-c1ncc2cnn(CC34CCC(c5nc(C(F)(F)F)c6n5[C@@H](C)CC6)(CC3)CC4)c2c1F]

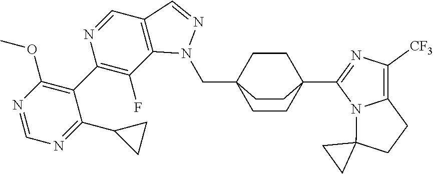 [SMILES: COc1ncnc(C2CC2)c1-c1ncc2cnn(CC34CCC(c5nc(C(F)(F)F)c6n5C5(CC6)CC5)(CC3)CC4)c2c1F]

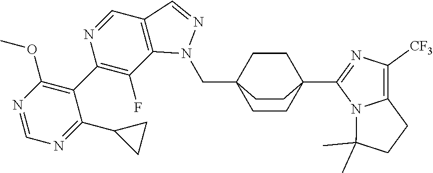 [SMILES: COc1ncnc(C2CC2)c1-c1ncc2cnn(CC34CCC(c5nc(C(F)(F)F)c6n5C(C)(C)CC6)(CC3)CC4)c2c1F]

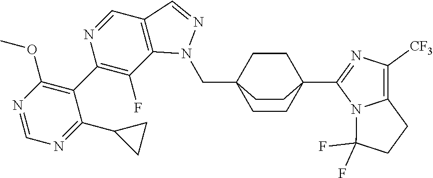 [SMILES: COc1ncnc(C2CC2)c1-c1ncc2cnn(CC34CCC(c5nc(C(F)(F)F)c6n5C(F)(F)CC6)(CC3)CC4)c2c1F]

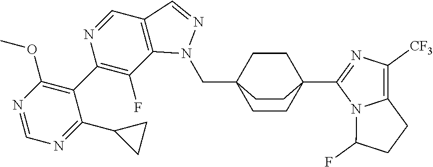 [SMILES: COc1ncnc(C2CC2)c1-c1ncc2cnn(CC34CCC(c5nc(C(F)(F)F)c6n5C(F)CC6)(CC3)CC4)c2c1F]

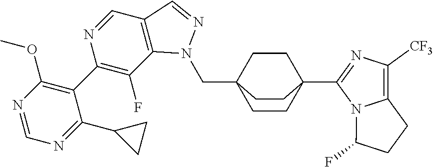 [SMILES: COc1ncnc(C2CC2)c1-c1ncc2cnn(CC34CCC(c5nc(C(F)(F)F)c6n5[C@@H](F)CC6)(CC3)CC4)c2c1F]

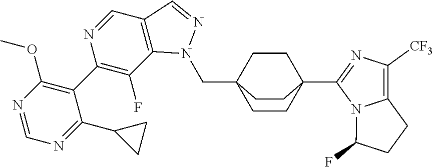 [SMILES: COc1ncnc(C2CC2)c1-c1ncc2cnn(CC34CCC(c5nc(C(F)(F)F)c6n5[C@H](F)CC6)(CC3)CC4)c2c1F]

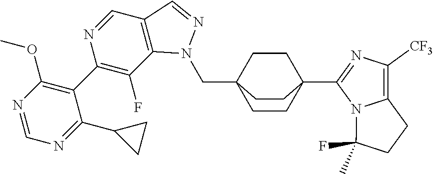 [SMILES: COc1ncnc(C2CC2)c1-c1ncc2cnn(CC34CCC(c5nc(C(F)(F)F)c6n5[C@](C)(F)CC6)(CC3)CC4)c2c1F]

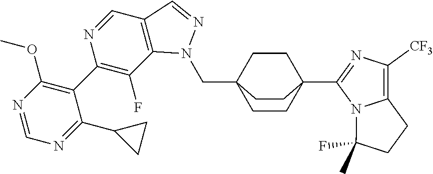 [SMILES: COc1ncnc(C2CC2)c1-c1ncc2cnn(CC34CCC(c5nc(C(F)(F)F)c6n5[C@@](C)(F)CC6)(CC3)CC4)c2c1F]

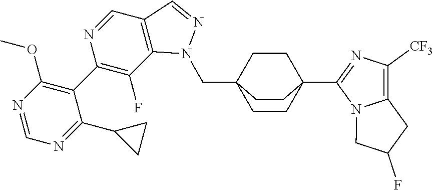 [SMILES: COc1ncnc(C2CC2)c1-c1ncc2cnn(CC34CCC(c5nc(C(F)(F)F)c6n5CC(F)C6)(CC3)CC4)c2c1F]

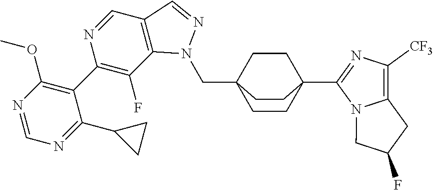 [SMILES: COc1ncnc(C2CC2)c1-c1ncc2cnn(CC34CCC(c5nc(C(F)(F)F)c6n5C[C@H](F)C6)(CC3)CC4)c2c1F]

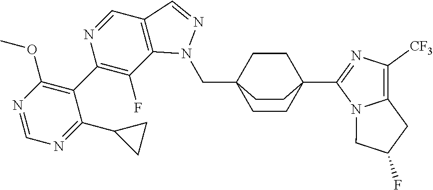 [SMILES: COc1ncnc(C2CC2)c1-c1ncc2cnn(CC34CCC(c5nc(C(F)(F)F)c6n5C[C@@H](F)C6)(CC3)CC4)c2c1F]

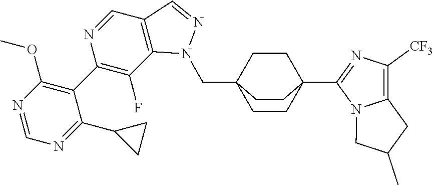 [SMILES: COc1ncnc(C2CC2)c1-c1ncc2cnn(CC34CCC(c5nc(C(F)(F)F)c6n5CC(C)C6)(CC3)CC4)c2c1F]

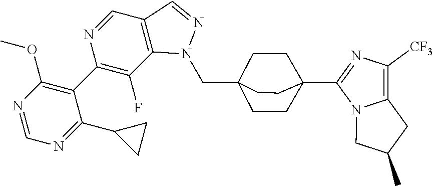 [SMILES: COc1ncnc(C2CC2)c1-c1ncc2cnn(CC34CCC(c5nc(C(F)(F)F)c6n5C[C@H](C)C6)(CC3)CC4)c2c1F]

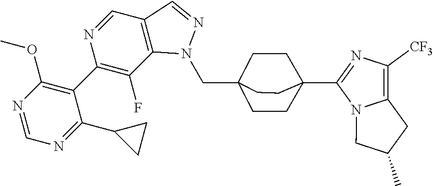 [SMILES: COc1ncnc(C2CC2)c1-c1ncc2cnn(CC34CCC(c5nc(C(F)(F)F)c6n5C[C@@H](C)C6)(CC3)CC4)c2c1F]

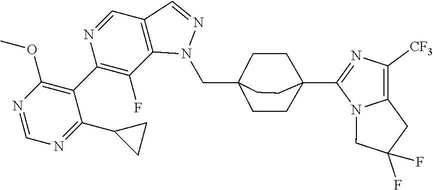 [SMILES: COc1ncnc(C2CC2)c1-c1ncc2cnn(CC34CCC(c5nc(C(F)(F)F)c6n5CC(F)(F)C6)(CC3)CC4)c2c1F]

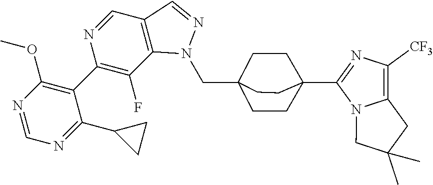

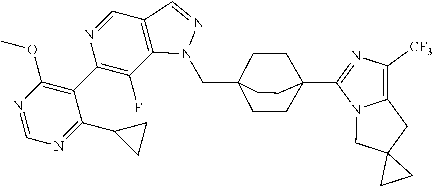 [SMILES: COc1ncnc(C2CC2)c1-c1ncc2cnn(CC34CCC(c5nc(C(F)(F)F)c6n5CC5(CC5)C6)(CC3)CC4)c2c1F]

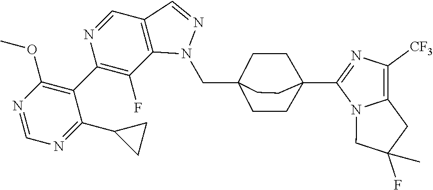

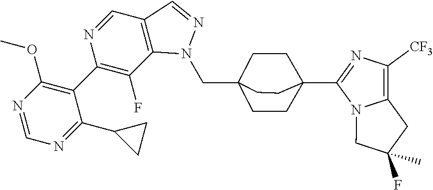

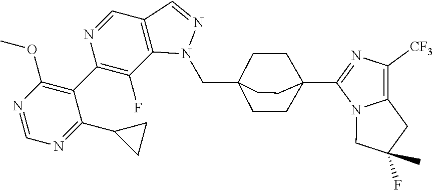

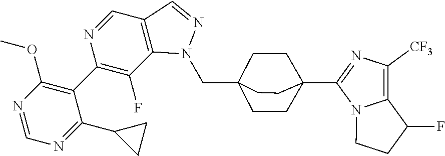 [SMILES: COc1ncnc(C2CC2)c1-c1ncc2cnn(CC34CCC(c5nc(C(F)(F)F)c6n5CCC6F)(CC3)CC4)c2c1F]

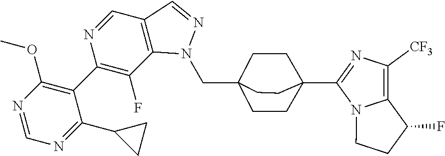 [SMILES: COc1ncnc(C2CC2)c1-c1ncc2cnn(CC34CCC(c5nc(C(F)(F)F)c6n5CC[C@H]6F)(CC3)CC4)c2c1F]

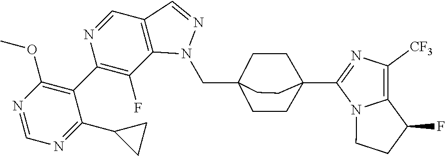 [SMILES: COc1ncnc(C2CC2)c1-c1ncc2cnn(CC34CCC(c5nc(C(F)(F)F)c6n5CC[C@@H]6F)(CC3)CC4)c2c1F]

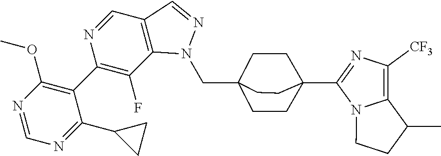

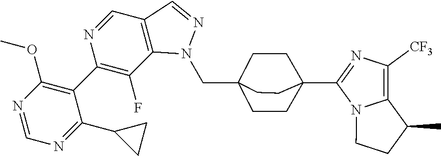

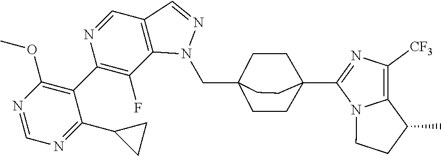

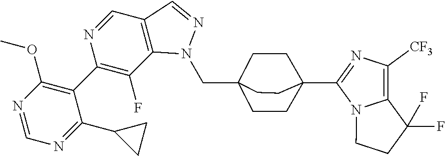 [SMILES: COc1ncnc(C2CC2)c1-c1ncc2cnn(CC34CCC(c5nc(C(F)(F)F)c6n5CCC6(F)F)(CC3)CC4)c2c1F]

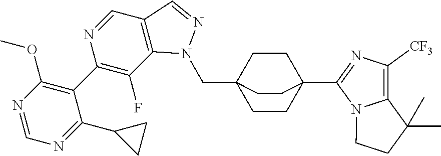

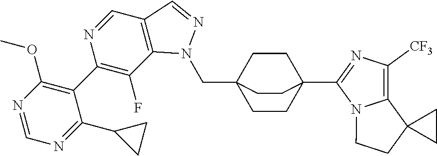 [SMILES: COc1ncnc(C2CC2)c1-c1ncc2cnn(CC34CCC(c5nc(C(F)(F)F)c6n5CCC65CC5)(CC3)CC4)c2c1F]

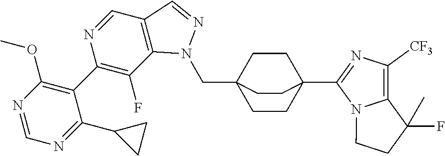 [SMILES: COc1ncnc(C2CC2)c1-c1ncc2cnn(CC34CCC(c5nc(C(F)(F)F)c6n5CCC6(C)F)(CC3)CC4)c2c1F]

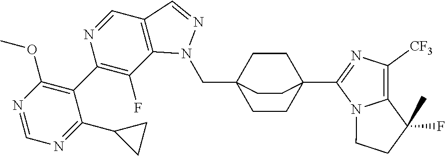 [SMILES: COc1ncnc(C2CC2)c1-c1ncc2cnn(CC34CCC(c5nc(C(F)(F)F)c6n5CC[C@@]6(C)F)(CC3)CC4)c2c1F]

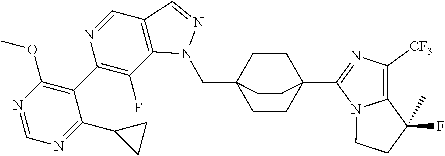 [SMILES: COc1ncnc(C2CC2)c1-c1ncc2cnn(CC34CCC(c5nc(C(F)(F)F)c6n5CC[C@]6(C)F)(CC3)CC4)c2c1F]

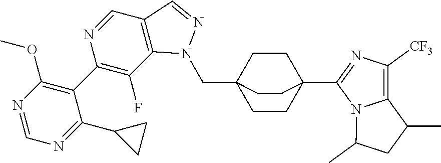

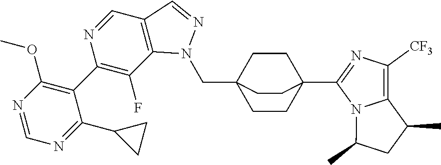

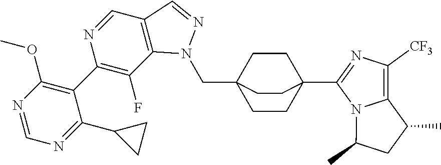

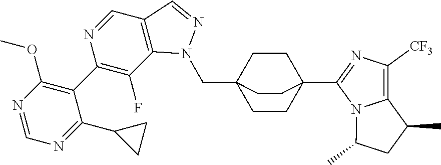

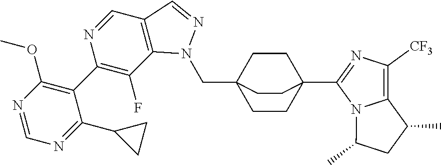

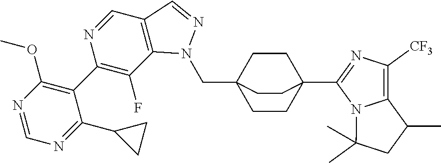

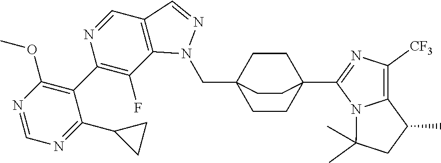

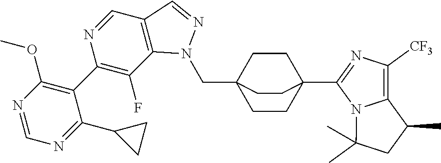

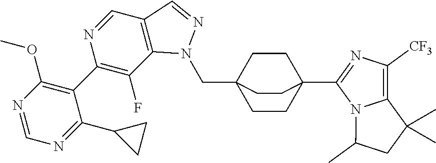

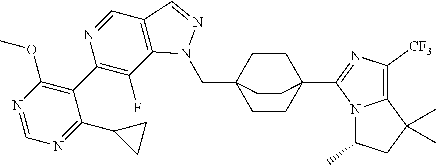

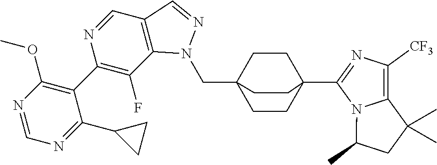

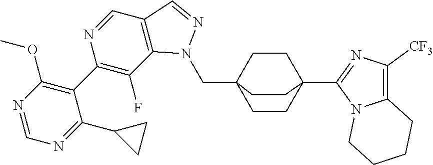 [SMILES: COc1ncnc(C2CC2)c1-c1ncc2cnn(CC34CCC(c5nc(C(F)(F)F)c6n5CCCC6)(CC3)CC4)c2c1F]

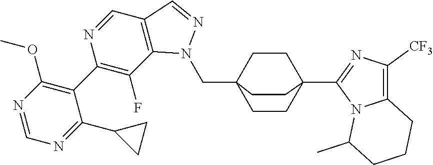 [SMILES: COc1ncnc(C2CC2)c1-c1ncc2cnn(CC34CCC(c5nc(C(F)(F)F)c6n5C(C)CCC6)(CC3)CC4)c2c1F]

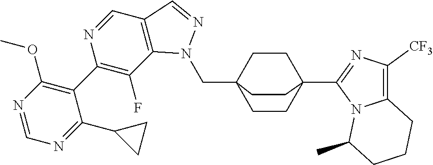 [SMILES: COc1ncnc(C2CC2)c1-c1ncc2cnn(CC34CCC(c5nc(C(F)(F)F)c6n5[C@H](C)CCC6)(CC3)CC4)c2c1F]

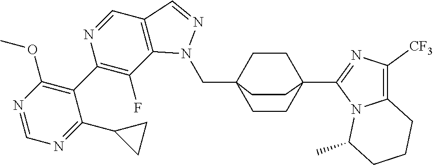 [SMILES: COc1ncnc(C2CC2)c1-c1ncc2cnn(CC34CCC(c5nc(C(F)(F)F)c6n5[C@@H](C)CCC6)(CC3)CC4)c2c1F]

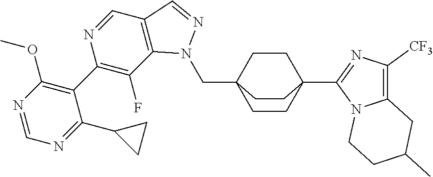

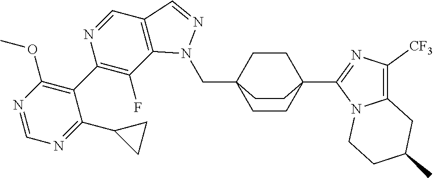

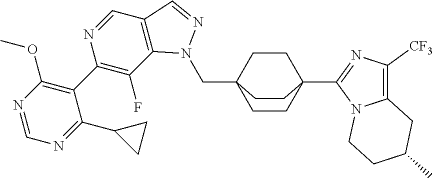

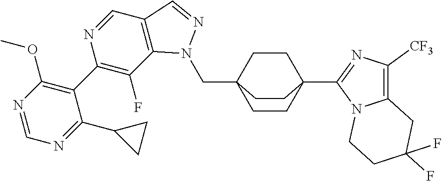 [SMILES: COc1ncnc(C2CC2)c1-c1ncc2cnn(CC34CCC(c5nc(C(F)(F)F)c6n5CCC(F)(F)C6)(CC3)CC4)c2c1F]

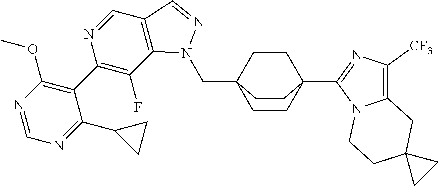 [SMILES: COc1ncnc(C2CC2)c1-c1ncc2cnn(CC34CCC(c5nc(C(F)(F)F)c6n5CCC5(CC5)C6)(CC3)CC4)c2c1F]

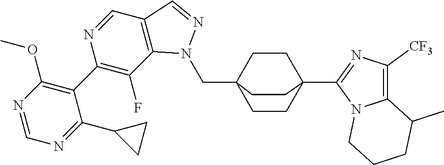

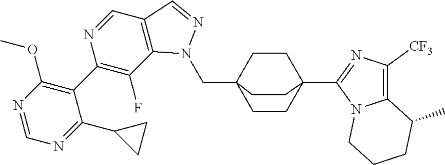

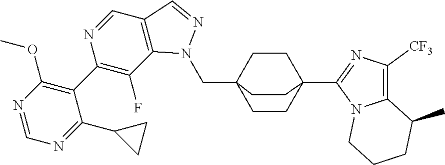

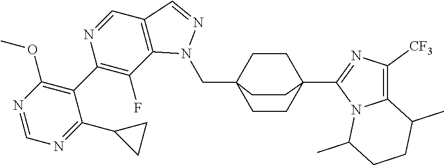

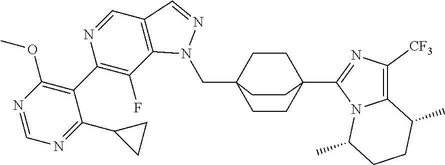

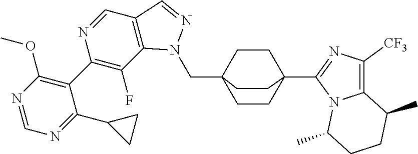

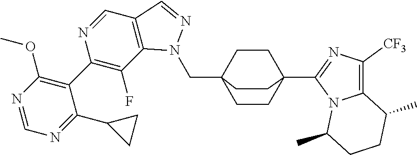

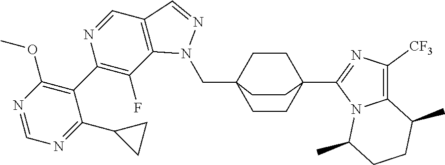

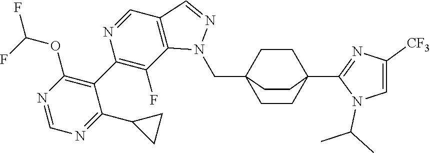 [SMILES: CC(C)n1cc(C(F)(F)F)nc1C12CCC(Cn3ncc4cnc(-c5c(OC(F)F)ncnc5C5CC5)c(F)c43)(CC1)CC2]

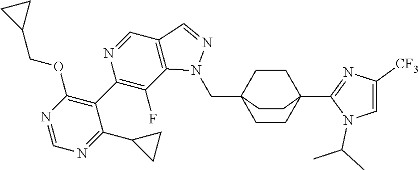 [SMILES: CC(C)n1cc(C(F)(F)F)nc1C12CCC(Cn3ncc4cnc(-c5c(OCC6CC6)ncnc5C5CC5)c(F)c43)(CC1)CC2]

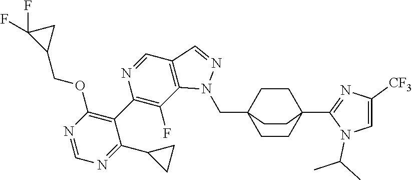 [SMILES: CC(C)n1cc(C(F)(F)F)nc1C12CCC(Cn3ncc4cnc(-c5c(OCC6CC6(F)F)ncnc5C5CC5)c(F)c43)(CC1)CC2]

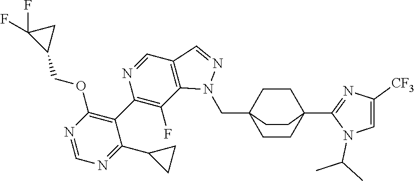 [SMILES: CC(C)n1cc(C(F)(F)F)nc1C12CCC(Cn3ncc4cnc(-c5c(OC[C@H]6CC6(F)F)ncnc5C5CC5)c(F)c43)(CC1)CC2]

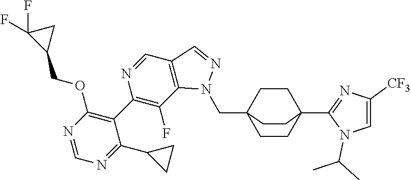 [SMILES: CC(C)n1cc(C(F)(F)F)nc1C12CCC(Cn3ncc4cnc(-c5c(OC[C@@H]6CC6(F)F)ncnc5C5CC5)c(F)c43)(CC1)CC2]

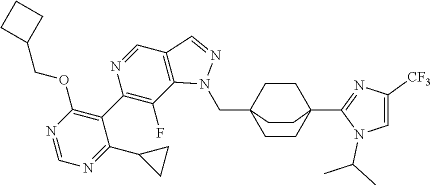 [SMILES: CC(C)n1cc(C(F)(F)F)nc1C12CCC(Cn3ncc4cnc(-c5c(OCC6CCC6)ncnc5C5CC5)c(F)c43)(CC1)CC2]

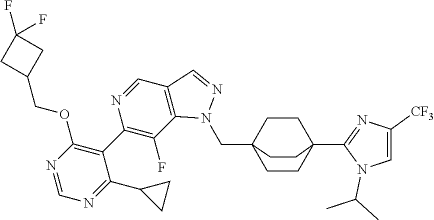 [SMILES: CC(C)n1cc(C(F)(F)F)nc1C12CCC(Cn3ncc4cnc(-c5c(OCC6CC(F)(F)C6)ncnc5C5CC5)c(F)c43)(CC1)CC2]

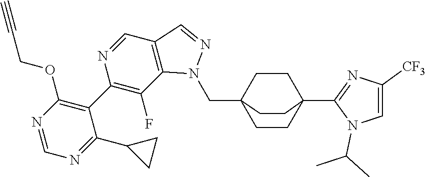 [SMILES: C#CCOc1ncnc(C2CC2)c1-c1ncc2cnn(CC34CCC(c5nc(C(F)(F)F)cn5C(C)C)(CC3)CC4)c2c1F]

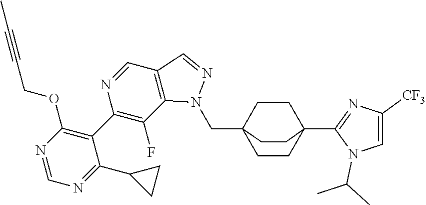 [SMILES: CC#CCOc1ncnc(C2CC2)c1-c1ncc2cnn(CC34CCC(c5nc(C(F)(F)F)cn5C(C)C)(CC3)CC4)c2c1F]

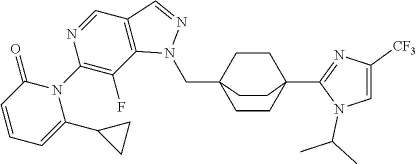 [SMILES: CC(C)n1cc(C(F)(F)F)nc1C12CCC(Cn3ncc4cnc(-n5c(C6CC6)cccc5=O)c(F)c43)(CC1)CC2]

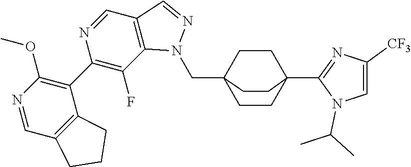 [SMILES: COc1ncc2c(c1-c1ncc3cnn(CC45CCC(c6nc(C(F)(F)F)cn6C(C)C)(CC4)CC5)c3c1F)CCC2]

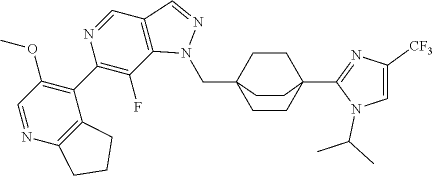 [SMILES: COc1cnc2c(c1-c1ncc3cnn(CC45CCC(c6nc(C(F)(F)F)cn6C(C)C)(CC4)CC5)c3c1F)CCC2]

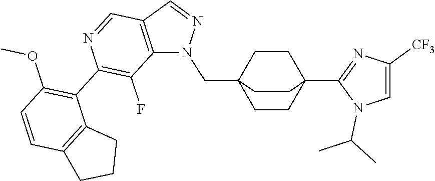 [SMILES: COc1ccc2c(c1-c1ncc3cnn(CC45CCC(c6nc(C(F)(F)F)cn6C(C)C)(CC4)CC5)c3c1F)CCC2]

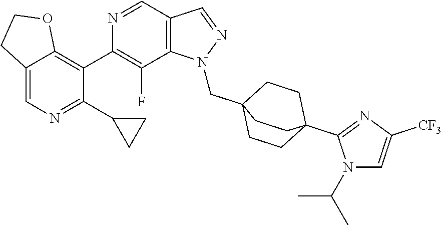 [SMILES: CC(C)n1cc(C(F)(F)F)nc1C12CCC(Cn3ncc4cnc(-c5c(C6CC6)ncc6c5OCC6)c(F)c43)(CC1)CC2]

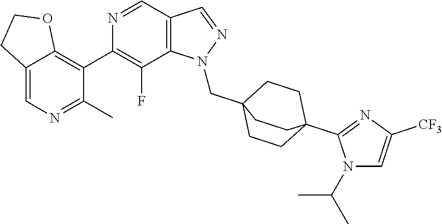 [SMILES: Cc1ncc2c(c1-c1ncc3cnn(CC45CCC(c6nc(C(F)(F)F)cn6C(C)C)(CC4)CC5)c3c1F)OCC2]

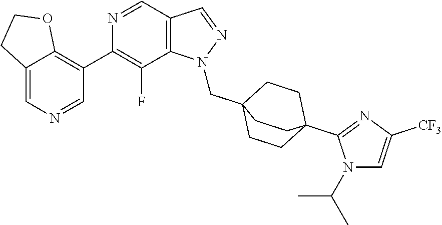 [SMILES: CC(C)n1cc(C(F)(F)F)nc1C12CCC(Cn3ncc4cnc(-c5cncc6c5OCC6)c(F)c43)(CC1)CC2]

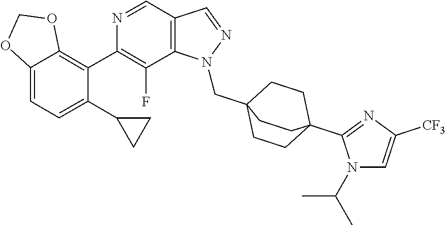 [SMILES: CC(C)n1cc(C(F)(F)F)nc1C12CCC(Cn3ncc4cnc(-c5c(C6CC6)ccc6c5OCO6)c(F)c43)(CC1)CC2]

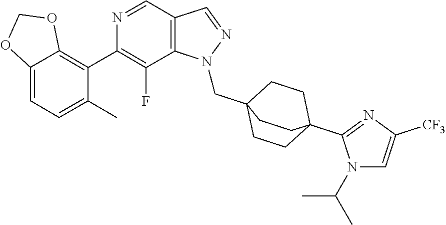 [SMILES: Cc1ccc2c(c1-c1ncc3cnn(CC45CCC(c6nc(C(F)(F)F)cn6C(C)C)(CC4)CC5)c3c1F)OCO2]

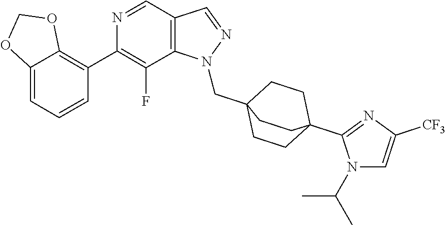 [SMILES: CC(C)n1cc(C(F)(F)F)nc1C12CCC(Cn3ncc4cnc(-c5cccc6c5OCO6)c(F)c43)(CC1)CC2]

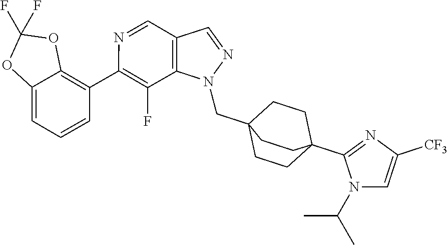 [SMILES: CC(C)n1cc(C(F)(F)F)nc1C12CCC(Cn3ncc4cnc(-c5cccc6c5OC(F)(F)O6)c(F)c43)(CC1)CC2]

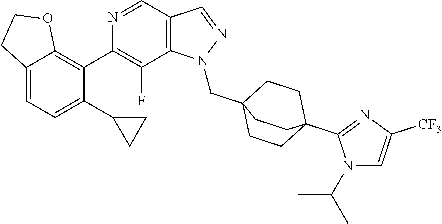 [SMILES: CC(C)n1cc(C(F)(F)F)nc1C12CCC(Cn3ncc4cnc(-c5c(C6CC6)ccc6c5OCC6)c(F)c43)(CC1)CC2]

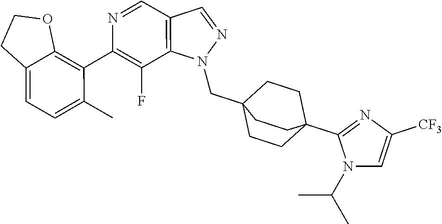 [SMILES: Cc1ccc2c(c1-c1ncc3cnn(CC45CCC(c6nc(C(F)(F)F)cn6C(C)C)(CC4)CC5)c3c1F)OCC2]

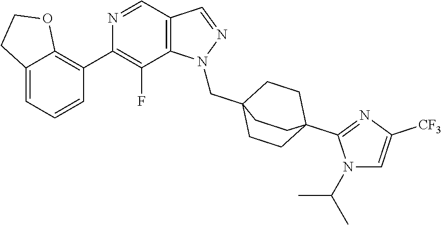 [SMILES: CC(C)n1cc(C(F)(F)F)nc1C12CCC(Cn3ncc4cnc(-c5cccc6c5OCC6)c(F)c43)(CC1)CC2]

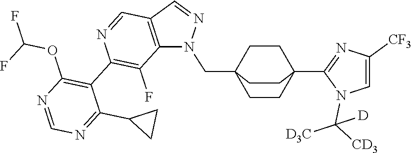 [SMILES: [2H]C([2H])([2H])C([2H])(n1cc(C(F)(F)F)nc1C12CCC(Cn3ncc4cnc(-c5c(OC(F)F)ncnc5C5CC5)c(F)c43)(CC1)CC2)C([2H])([2H])[2H]]

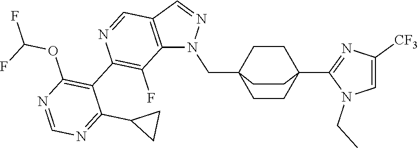 [SMILES: CCn1cc(C(F)(F)F)nc1C12CCC(Cn3ncc4cnc(-c5c(OC(F)F)ncnc5C5CC5)c(F)c43)(CC1)CC2]

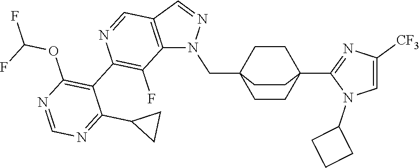 [SMILES: Fc1c(-c2c(OC(F)F)ncnc2C2CC2)ncc2cnn(CC34CCC(c5nc(C(F)(F)F)cn5C5CCC5)(CC3)CC4)c12]

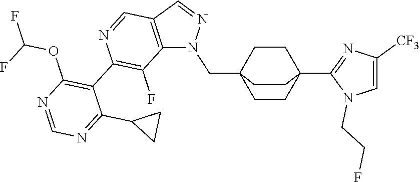 [SMILES: FCCn1cc(C(F)(F)F)nc1C12CCC(Cn3ncc4cnc(-c5c(OC(F)F)ncnc5C5CC5)c(F)c43)(CC1)CC2]

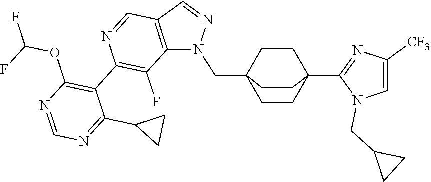 [SMILES: Fc1c(-c2c(OC(F)F)ncnc2C2CC2)ncc2cnn(CC34CCC(c5nc(C(F)(F)F)cn5CC5CC5)(CC3)CC4)c12]

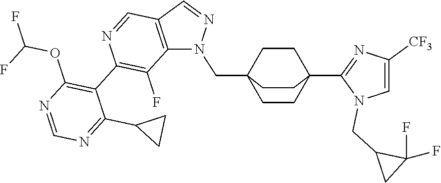 [SMILES: Fc1c(-c2c(OC(F)F)ncnc2C2CC2)ncc2cnn(CC34CCC(c5nc(C(F)(F)F)cn5CC5CC5(F)F)(CC3)CC4)c12]

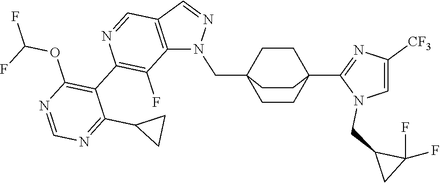 [SMILES: Fc1c(-c2c(OC(F)F)ncnc2C2CC2)ncc2cnn(CC34CCC(c5nc(C(F)(F)F)cn5C[C@@H]5CC5(F)F)(CC3)CC4)c12]

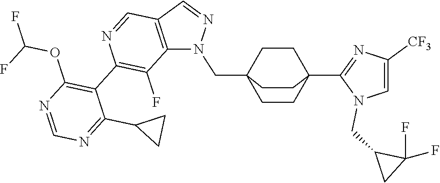 [SMILES: Fc1c(-c2c(OC(F)F)ncnc2C2CC2)ncc2cnn(CC34CCC(c5nc(C(F)(F)F)cn5C[C@H]5CC5(F)F)(CC3)CC4)c12]

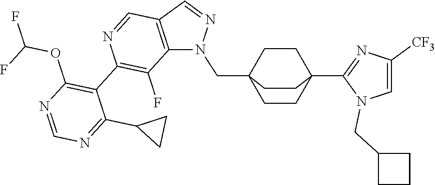 [SMILES: Fc1c(-c2c(OC(F)F)ncnc2C2CC2)ncc2cnn(CC34CCC(c5nc(C(F)(F)F)cn5CC5CCC5)(CC3)CC4)c12]

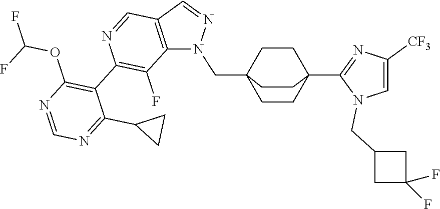 [SMILES: Fc1c(-c2c(OC(F)F)ncnc2C2CC2)ncc2cnn(CC34CCC(c5nc(C(F)(F)F)cn5CC5CC(F)(F)C5)(CC3)CC4)c12]

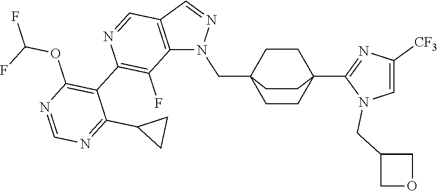 [SMILES: Fc1c(-c2c(OC(F)F)ncnc2C2CC2)ncc2cnn(CC34CCC(c5nc(C(F)(F)F)cn5CC5COC5)(CC3)CC4)c12]

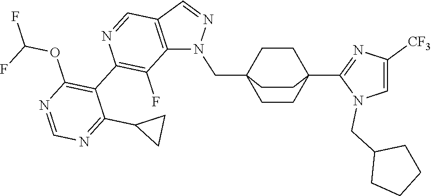 [SMILES: Fc1c(-c2c(OC(F)F)ncnc2C2CC2)ncc2cnn(CC34CCC(c5nc(C(F)(F)F)cn5CC5CCCC5)(CC3)CC4)c12]

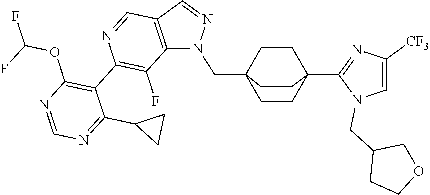 [SMILES: Fc1c(-c2c(OC(F)F)ncnc2C2CC2)ncc2cnn(CC34CCC(c5nc(C(F)(F)F)cn5CC5CCOC5)(CC3)CC4)c12]

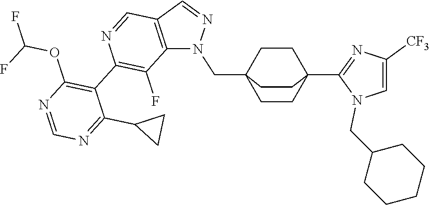 [SMILES: Fc1c(-c2c(OC(F)F)ncnc2C2CC2)ncc2cnn(CC34CCC(c5nc(C(F)(F)F)cn5CC5CCCCC5)(CC3)CC4)c12]

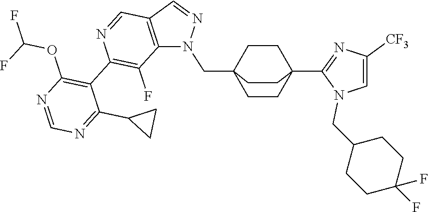 [SMILES: Fc1c(-c2c(OC(F)F)ncnc2C2CC2)ncc2cnn(CC34CCC(c5nc(C(F)(F)F)cn5CC5CCC(F)(F)CC5)(CC3)CC4)c12]

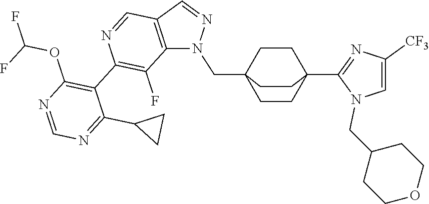 [SMILES: Fc1c(-c2c(OC(F)F)ncnc2C2CC2)ncc2cnn(CC34CCC(c5nc(C(F)(F)F)cn5CC5CCOCC5)(CC3)CC4)c12]

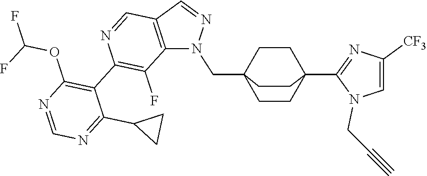 [SMILES: C#CCn1cc(C(F)(F)F)nc1C12CCC(Cn3ncc4cnc(-c5c(OC(F)F)ncnc5C5CC5)c(F)c43)(CC1)CC2]

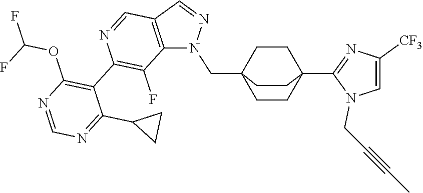 [SMILES: CC#CCn1cc(C(F)(F)F)nc1C12CCC(Cn3ncc4cnc(-c5c(OC(F)F)ncnc5C5CC5)c(F)c43)(CC1)CC2]

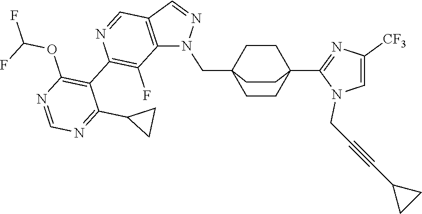 [SMILES: Fc1c(-c2c(OC(F)F)ncnc2C2CC2)ncc2cnn(CC34CCC(c5nc(C(F)(F)F)cn5CC#CC5CC5)(CC3)CC4)c12]

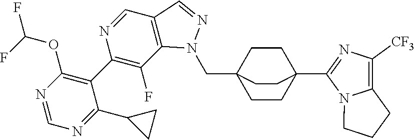 [SMILES: Fc1c(-c2c(OC(F)F)ncnc2C2CC2)ncc2cnn(CC34CCC(c5nc(C(F)(F)F)c6n5CCC6)(CC3)CC4)c12]

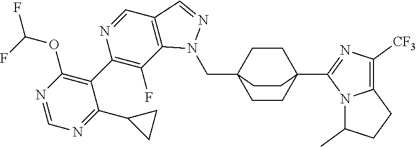 [SMILES: CC1CCc2c(C(F)(F)F)nc(C34CCC(Cn5ncc6cnc(-c7c(OC(F)F)ncnc7C7CC7)c(F)c65)(CC3)CC4)n21]

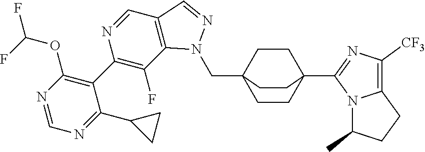 [SMILES: C[C@@H]1CCc2c(C(F)(F)F)nc(C34CCC(Cn5ncc6cnc(-c7c(OC(F)F)ncnc7C7CC7)c(F)c65)(CC3)CC4)n21]

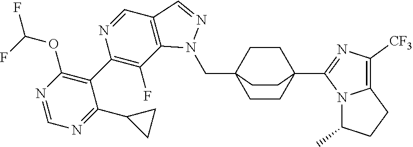 [SMILES: C[C@H]1CCc2c(C(F)(F)F)nc(C34CCC(Cn5ncc6cnc(-c7c(OC(F)F)ncnc7C7CC7)c(F)c65)(CC3)CC4)n21]

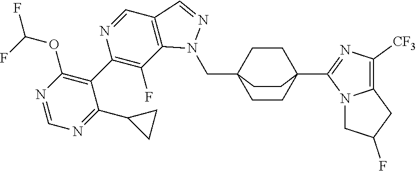 [SMILES: Fc1c(-c2c(OC(F)F)ncnc2C2CC2)ncc2cnn(CC34CCC(c5nc(C(F)(F)F)c6n5CC(F)C6)(CC3)CC4)c12]

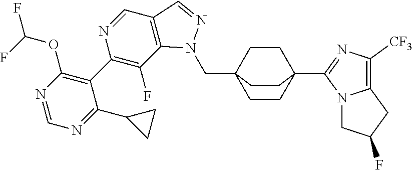 [SMILES: Fc1c(-c2c(OC(F)F)ncnc2C2CC2)ncc2cnn(CC34CCC(c5nc(C(F)(F)F)c6n5C[C@H](F)C6)(CC3)CC4)c12]

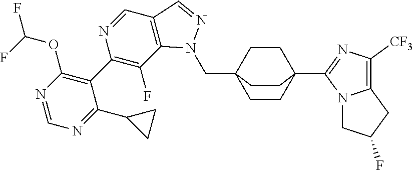 [SMILES: Fc1c(-c2c(OC(F)F)ncnc2C2CC2)ncc2cnn(CC34CCC(c5nc(C(F)(F)F)c6n5C[C@@H](F)C6)(CC3)CC4)c12]

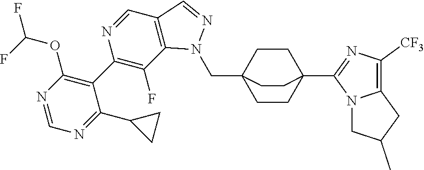 [SMILES: CC1Cc2c(C(F)(F)F)nc(C34CCC(Cn5ncc6cnc(-c7c(OC(F)F)ncnc7C7CC7)c(F)c65)(CC3)CC4)n2C1]

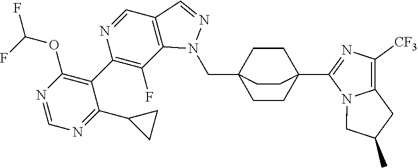 [SMILES: C[C@@H]1Cc2c(C(F)(F)F)nc(C34CCC(Cn5ncc6cnc(-c7c(OC(F)F)ncnc7C7CC7)c(F)c65)(CC3)CC4)n2C1]

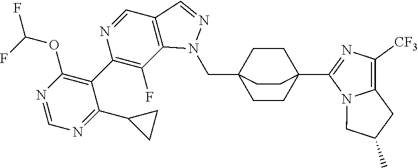 [SMILES: C[C@H]1Cc2c(C(F)(F)F)nc(C34CCC(Cn5ncc6cnc(-c7c(OC(F)F)ncnc7C7CC7)c(F)c65)(CC3)CC4)n2C1]

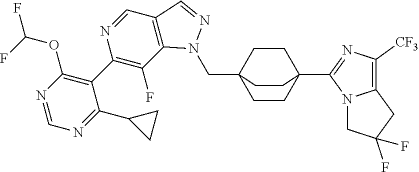 [SMILES: Fc1c(-c2c(OC(F)F)ncnc2C2CC2)ncc2cnn(CC34CCC(c5nc(C(F)(F)F)c6n5CC(F)(F)C6)(CC3)CC4)c12]

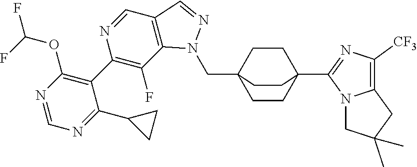 [SMILES: CC1(C)Cc2c(C(F)(F)F)nc(C34CCC(Cn5ncc6cnc(-c7c(OC(F)F)ncnc7C7CC7)c(F)c65)(CC3)CC4)n2C1]

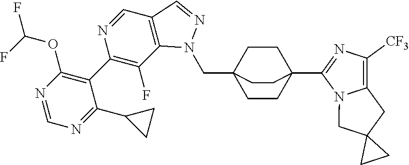 [SMILES: Fc1c(-c2c(OC(F)F)ncnc2C2CC2)ncc2cnn(CC34CCC(c5nc(C(F)(F)F)c6n5CC5(CC5)C6)(CC3)CC4)c12]

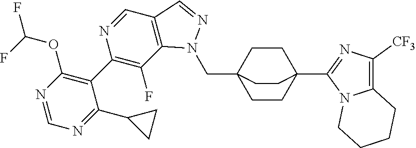 [SMILES: Fc1c(-c2c(OC(F)F)ncnc2C2CC2)ncc2cnn(CC34CCC(c5nc(C(F)(F)F)c6n5CCCC6)(CC3)CC4)c12]

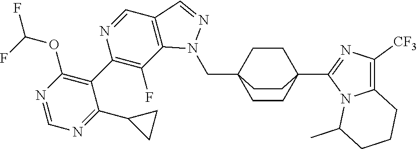 [SMILES: CC1CCCc2c(C(F)(F)F)nc(C34CCC(Cn5ncc6cnc(-c7c(OC(F)F)ncnc7C7CC7)c(F)c65)(CC3)CC4)n21]

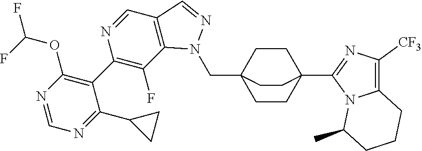 [SMILES: C[C@@H]1CCCc2c(C(F)(F)F)nc(C34CCC(Cn5ncc6cnc(-c7c(OC(F)F)ncnc7C7CC7)c(F)c65)(CC3)CC4)n21]

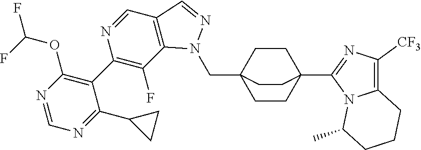 [SMILES: C[C@H]1CCCc2c(C(F)(F)F)nc(C34CCC(Cn5ncc6cnc(-c7c(OC(F)F)ncnc7C7CC7)c(F)c65)(CC3)CC4)n21]

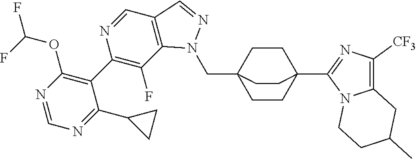 [SMILES: CC1CCn2c(C34CCC(Cn5ncc6cnc(-c7c(OC(F)F)ncnc7C7CC7)c(F)c65)(CC3)CC4)nc(C(F)(F)F)c2C1]

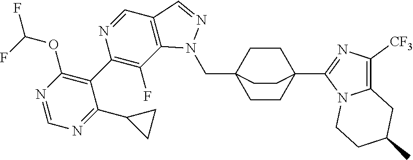 [SMILES: C[C@H]1CCn2c(C34CCC(Cn5ncc6cnc(-c7c(OC(F)F)ncnc7C7CC7)c(F)c65)(CC3)CC4)nc(C(F)(F)F)c2C1]

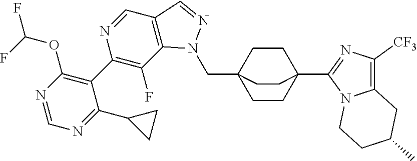 [SMILES: C[C@@H]1CCn2c(C34CCC(Cn5ncc6cnc(-c7c(OC(F)F)ncnc7C7CC7)c(F)c65)(CC3)CC4)nc(C(F)(F)F)c2C1]

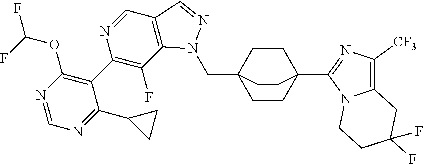 [SMILES: Fc1c(-c2c(OC(F)F)ncnc2C2CC2)ncc2cnn(CC34CCC(c5nc(C(F)(F)F)c6n5CCC(F)(F)C6)(CC3)CC4)c12]

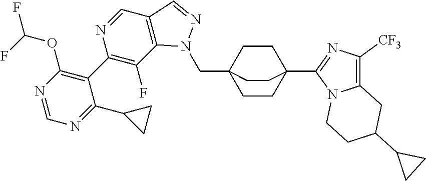 [SMILES: Fc1c(-c2c(OC(F)F)ncnc2C2CC2)ncc2cnn(CC34CCC(c5nc(C(F)(F)F)c6n5CCC(C5CC5)C6)(CC3)CC4)c12]

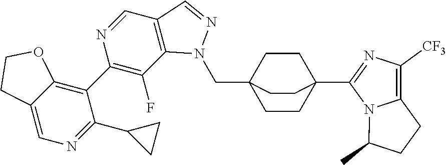 [SMILES: C[C@@H]1CCc2c(C(F)(F)F)nc(C34CCC(Cn5ncc6cnc(-c7c(C8CC8)ncc8c7OCC8)c(F)c65)(CC3)CC4)n21]

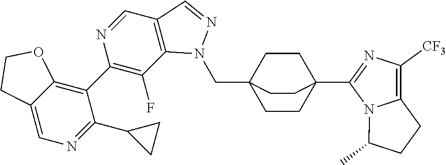 [SMILES: C[C@H]1CCc2c(C(F)(F)F)nc(C34CCC(Cn5ncc6cnc(-c7c(C8CC8)ncc8c7OCC8)c(F)c65)(CC3)CC4)n21]

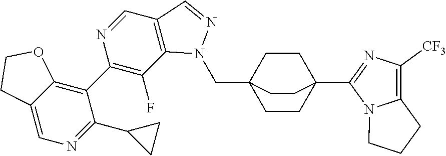 [SMILES: Fc1c(-c2c(C3CC3)ncc3c2OCC3)ncc2cnn(CC34CCC(c5nc(C(F)(F)F)c6n5CCC6)(CC3)CC4)c12]

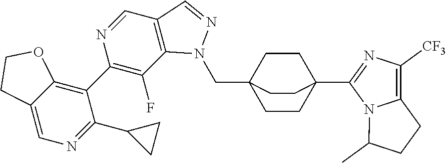 [SMILES: CC1CCc2c(C(F)(F)F)nc(C34CCC(Cn5ncc6cnc(-c7c(C8CC8)ncc8c7OCC8)c(F)c65)(CC3)CC4)n21]

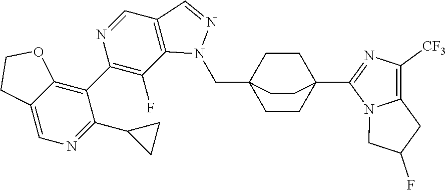 [SMILES: Fc1c(-c2c(C3CC3)ncc3c2OCC3)ncc2cnn(CC34CCC(c5nc(C(F)(F)F)c6n5CC(F)C6)(CC3)CC4)c12]

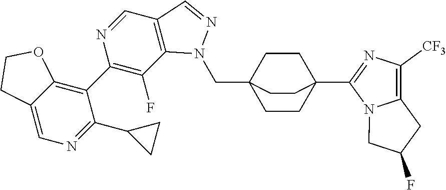 [SMILES: Fc1c(-c2c(C3CC3)ncc3c2OCC3)ncc2cnn(CC34CCC(c5nc(C(F)(F)F)c6n5C[C@H](F)C6)(CC3)CC4)c12]

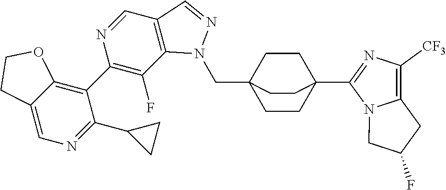 [SMILES: Fc1c(-c2c(C3CC3)ncc3c2OCC3)ncc2cnn(CC34CCC(c5nc(C(F)(F)F)c6n5C[C@@H](F)C6)(CC3)CC4)c12]

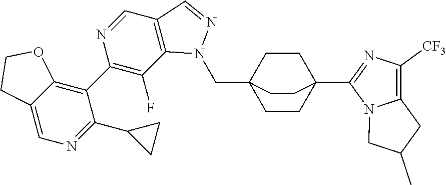 [SMILES: CC1Cc2c(C(F)(F)F)nc(C34CCC(Cn5ncc6cnc(-c7c(C8CC8)ncc8c7OCC8)c(F)c65)(CC3)CC4)n2C1]

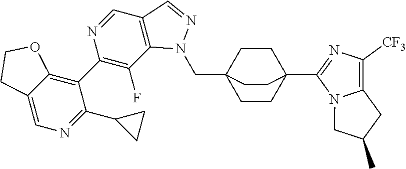 [SMILES: C[C@@H]1Cc2c(C(F)(F)F)nc(C34CCC(Cn5ncc6cnc(-c7c(C8CC8)ncc8c7OCC8)c(F)c65)(CC3)CC4)n2C1]

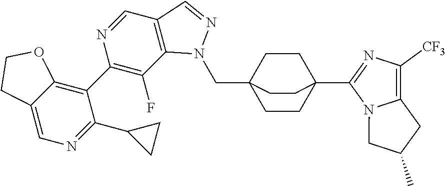 [SMILES: C[C@H]1Cc2c(C(F)(F)F)nc(C34CCC(Cn5ncc6cnc(-c7c(C8CC8)ncc8c7OCC8)c(F)c65)(CC3)CC4)n2C1]

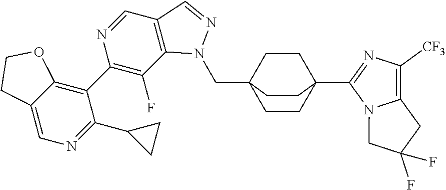 [SMILES: Fc1c(-c2c(C3CC3)ncc3c2OCC3)ncc2cnn(CC34CCC(c5nc(C(F)(F)F)c6n5CC(F)(F)C6)(CC3)CC4)c12]

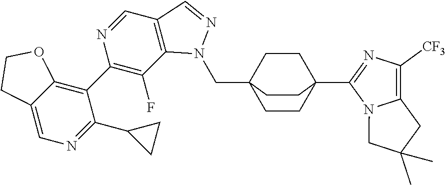 [SMILES: CC1(C)Cc2c(C(F)(F)F)nc(C34CCC(Cn5ncc6cnc(-c7c(C8CC8)ncc8c7OCC8)c(F)c65)(CC3)CC4)n2C1]

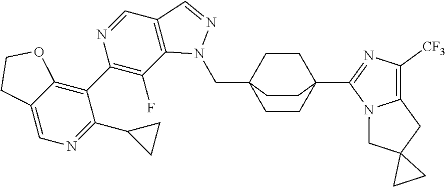 [SMILES: Fc1c(-c2c(C3CC3)ncc3c2OCC3)ncc2cnn(CC34CCC(c5nc(C(F)(F)F)c6n5CC5(CC5)C6)(CC3)CC4)c12]

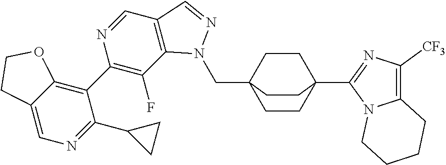 [SMILES: Fc1c(-c2c(C3CC3)ncc3c2OCC3)ncc2cnn(CC34CCC(c5nc(C(F)(F)F)c6n5CCCC6)(CC3)CC4)c12]

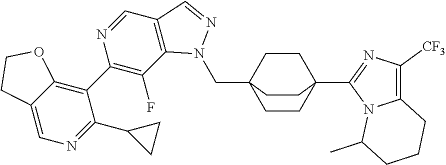 [SMILES: CC1CCCc2c(C(F)(F)F)nc(C34CCC(Cn5ncc6cnc(-c7c(C8CC8)ncc8c7OCC8)c(F)c65)(CC3)CC4)n21]

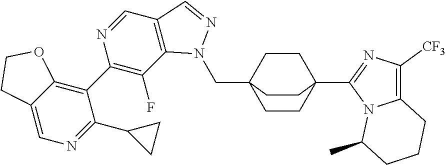 [SMILES: C[C@@H]1CCCc2c(C(F)(F)F)nc(C34CCC(Cn5ncc6cnc(-c7c(C8CC8)ncc8c7OCC8)c(F)c65)(CC3)CC4)n21]

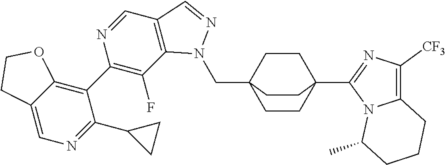 [SMILES: C[C@H]1CCCc2c(C(F)(F)F)nc(C34CCC(Cn5ncc6cnc(-c7c(C8CC8)ncc8c7OCC8)c(F)c65)(CC3)CC4)n21]

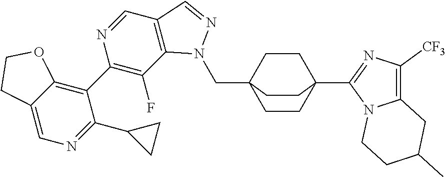 [SMILES: CC1CCn2c(C34CCC(Cn5ncc6cnc(-c7c(C8CC8)ncc8c7OCC8)c(F)c65)(CC3)CC4)nc(C(F)(F)F)c2C1]

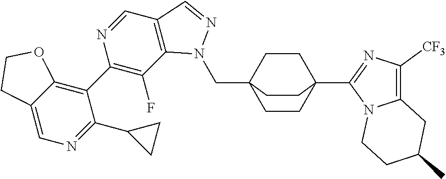 [SMILES: C[C@H]1CCn2c(C34CCC(Cn5ncc6cnc(-c7c(C8CC8)ncc8c7OCC8)c(F)c65)(CC3)CC4)nc(C(F)(F)F)c2C1]

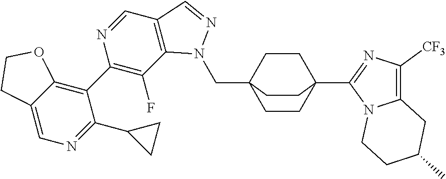 [SMILES: C[C@@H]1CCn2c(C34CCC(Cn5ncc6cnc(-c7c(C8CC8)ncc8c7OCC8)c(F)c65)(CC3)CC4)nc(C(F)(F)F)c2C1]

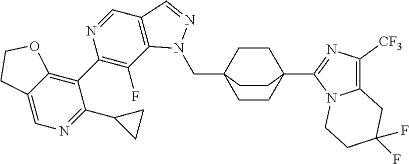 [SMILES: Fc1c(-c2c(C3CC3)ncc3c2OCC3)ncc2cnn(CC34CCC(c5nc(C(F)(F)F)c6n5CCC(F)(F)C6)(CC3)CC4)c12]

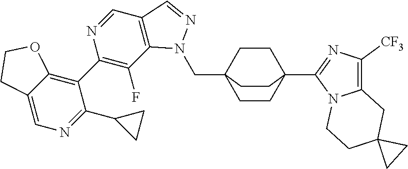 [SMILES: Fc1c(-c2c(C3CC3)ncc3c2OCC3)ncc2cnn(CC34CCC(c5nc(C(F)(F)F)c6n5CCC5(CC5)C6)(CC3)CC4)c12]

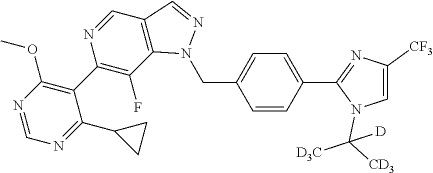 [SMILES: [2H]C([2H])([2H])C([2H])(n1cc(C(F)(F)F)nc1-c1ccc(Cn2ncc3cnc(-c4c(OC)ncnc4C4CC4)c(F)c32)cc1)C([2H])([2H])[2H]]

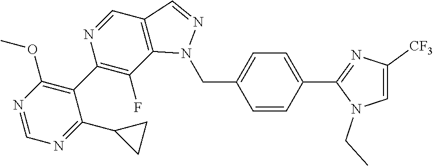 [SMILES: CCn1cc(C(F)(F)F)nc1-c1ccc(Cn2ncc3cnc(-c4c(OC)ncnc4C4CC4)c(F)c32)cc1]

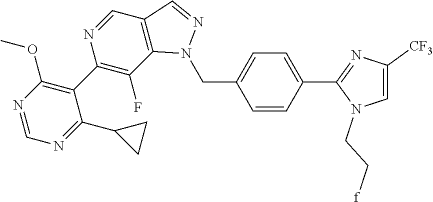

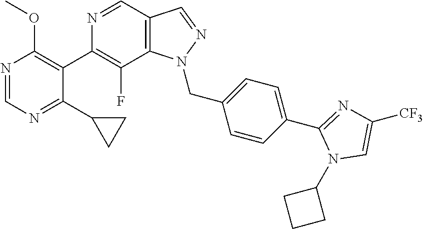 [SMILES: COc1ncnc(C2CC2)c1-c1ncc2cnn(Cc3ccc(-c4nc(C(F)(F)F)cn4C4CCC4)cc3)c2c1F]

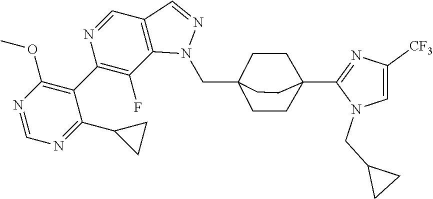 [SMILES: COc1ncnc(C2CC2)c1-c1ncc2cnn(CC34CCC(c5nc(C(F)(F)F)cn5CC5CC5)(CC3)CC4)c2c1F]

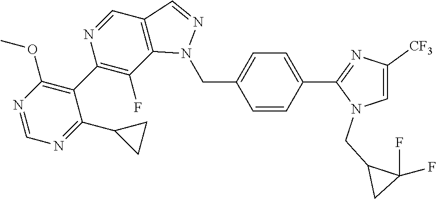 [SMILES: COc1ncnc(C2CC2)c1-c1ncc2cnn(Cc3ccc(-c4nc(C(F)(F)F)cn4CC4CC4(F)F)cc3)c2c1F]

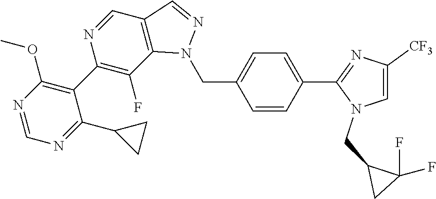 [SMILES: COc1ncnc(C2CC2)c1-c1ncc2cnn(Cc3ccc(-c4nc(C(F)(F)F)cn4C[C@@H]4CC4(F)F)cc3)c2c1F]

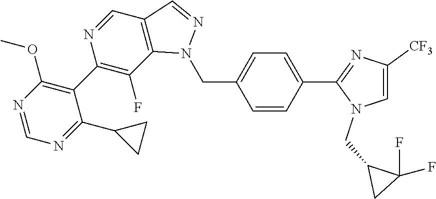 [SMILES: COc1ncnc(C2CC2)c1-c1ncc2cnn(Cc3ccc(-c4nc(C(F)(F)F)cn4C[C@H]4CC4(F)F)cc3)c2c1F]

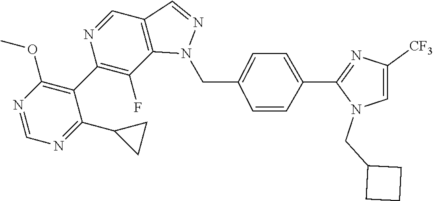 [SMILES: COc1ncnc(C2CC2)c1-c1ncc2cnn(Cc3ccc(-c4nc(C(F)(F)F)cn4CC4CCC4)cc3)c2c1F]

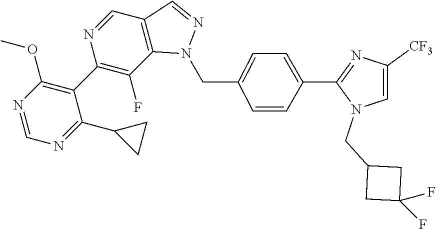 [SMILES: COc1ncnc(C2CC2)c1-c1ncc2cnn(Cc3ccc(-c4nc(C(F)(F)F)cn4CC4CC(F)(F)C4)cc3)c2c1F]

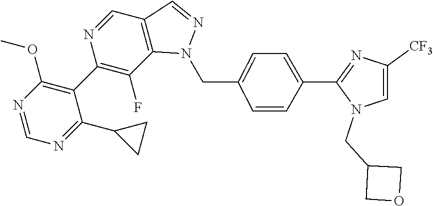 [SMILES: COc1ncnc(C2CC2)c1-c1ncc2cnn(Cc3ccc(-c4nc(C(F)(F)F)cn4CC4COC4)cc3)c2c1F]

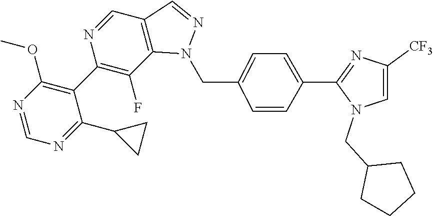 [SMILES: COc1ncnc(C2CC2)c1-c1ncc2cnn(Cc3ccc(-c4nc(C(F)(F)F)cn4CC4CCCC4)cc3)c2c1F]

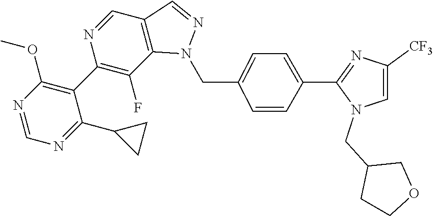 [SMILES: COc1ncnc(C2CC2)c1-c1ncc2cnn(Cc3ccc(-c4nc(C(F)(F)F)cn4CC4CCOC4)cc3)c2c1F]

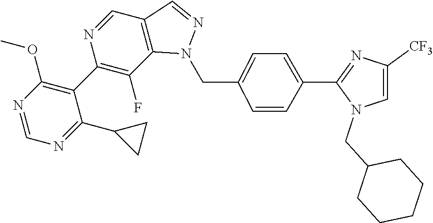 [SMILES: COc1ncnc(C2CC2)c1-c1ncc2cnn(Cc3ccc(-c4nc(C(F)(F)F)cn4CC4CCCCC4)cc3)c2c1F]

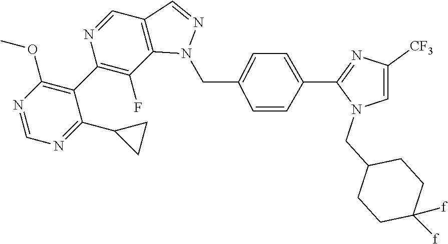

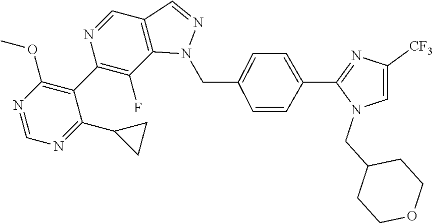 [SMILES: COc1ncnc(C2CC2)c1-c1ncc2cnn(Cc3ccc(-c4nc(C(F)(F)F)cn4CC4CCOCC4)cc3)c2c1F]

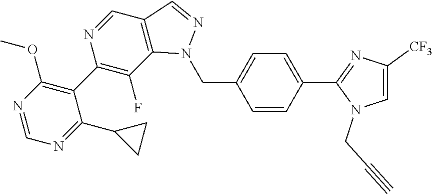 [SMILES: C#CCn1cc(C(F)(F)F)nc1-c1ccc(Cn2ncc3cnc(-c4c(OC)ncnc4C4CC4)c(F)c32)cc1]

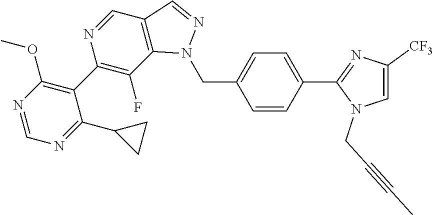 [SMILES: CC#CCn1cc(C(F)(F)F)nc1-c1ccc(Cn2ncc3cnc(-c4c(OC)ncnc4C4CC4)c(F)c32)cc1]

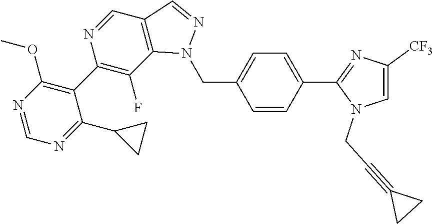

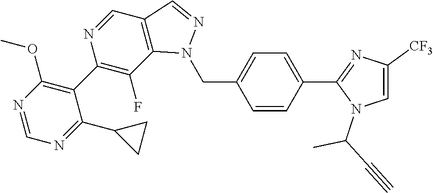 [SMILES: C#CC(C)n1cc(C(F)(F)F)nc1-c1ccc(Cn2ncc3cnc(-c4c(OC)ncnc4C4CC4)c(F)c32)cc1]

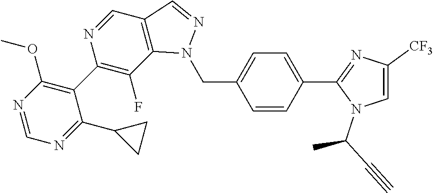 [SMILES: C#C[C@@H](C)n1cc(C(F)(F)F)nc1-c1ccc(Cn2ncc3cnc(-c4c(OC)ncnc4C4CC4)c(F)c32)cc1]

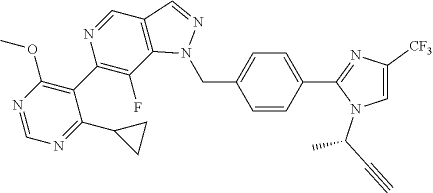 [SMILES: C#C[C@H](C)n1cc(C(F)(F)F)nc1-c1ccc(Cn2ncc3cnc(-c4c(OC)ncnc4C4CC4)c(F)c32)cc1]

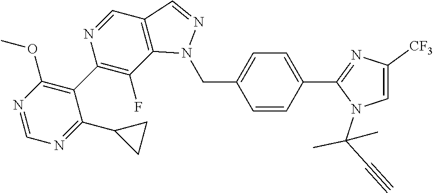 [SMILES: C#CC(C)(C)n1cc(C(F)(F)F)nc1-c1ccc(Cn2ncc3cnc(-c4c(OC)ncnc4C4CC4)c(F)c32)cc1]

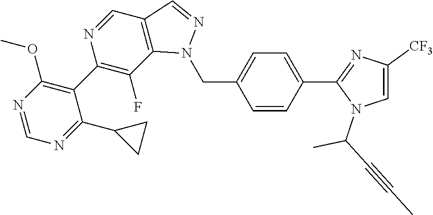 [SMILES: CC#CC(C)n1cc(C(F)(F)F)nc1-c1ccc(Cn2ncc3cnc(-c4c(OC)ncnc4C4CC4)c(F)c32)cc1]

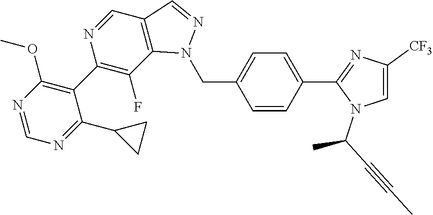 [SMILES: CC#C[C@@H](C)n1cc(C(F)(F)F)nc1-c1ccc(Cn2ncc3cnc(-c4c(OC)ncnc4C4CC4)c(F)c32)cc1]

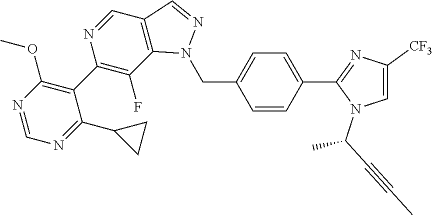 [SMILES: CC#C[C@H](C)n1cc(C(F)(F)F)nc1-c1ccc(Cn2ncc3cnc(-c4c(OC)ncnc4C4CC4)c(F)c32)cc1]

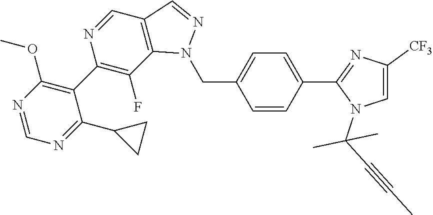 [SMILES: CC#CC(C)(C)n1cc(C(F)(F)F)nc1-c1ccc(Cn2ncc3cnc(-c4c(OC)ncnc4C4CC4)c(F)c32)cc1]

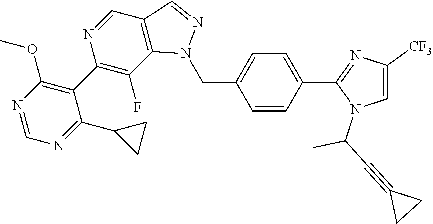

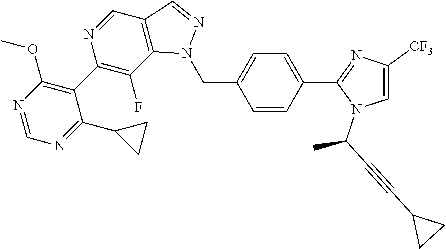 [SMILES: COc1ncnc(C2CC2)c1-c1ncc2cnn(Cc3ccc(-c4nc(C(F)(F)F)cn4[C@H](C)C#CC4CC4)cc3)c2c1F]

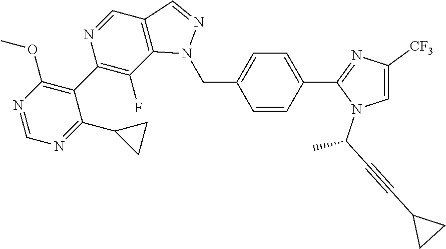 [SMILES: COc1ncnc(C2CC2)c1-c1ncc2cnn(Cc3ccc(-c4nc(C(F)(F)F)cn4[C@@H](C)C#CC4CC4)cc3)c2c1F]

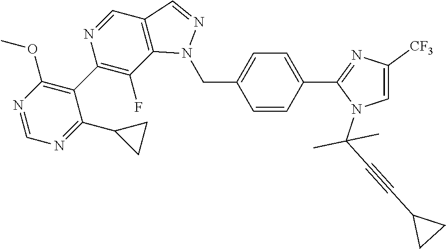 [SMILES: COc1ncnc(C2CC2)c1-c1ncc2cnn(Cc3ccc(-c4nc(C(F)(F)F)cn4C(C)(C)C#CC4CC4)cc3)c2c1F]

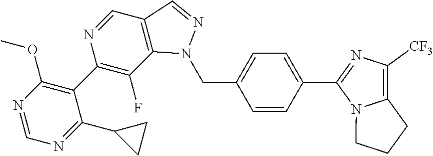 [SMILES: COc1ncnc(C2CC2)c1-c1ncc2cnn(Cc3ccc(-c4nc(C(F)(F)F)c5n4CCC5)cc3)c2c1F]

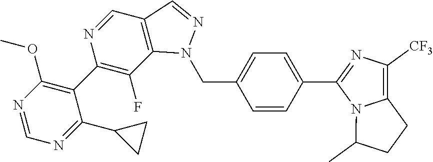 [SMILES: COc1ncnc(C2CC2)c1-c1ncc2cnn(Cc3ccc(-c4nc(C(F)(F)F)c5n4C(C)CC5)cc3)c2c1F]

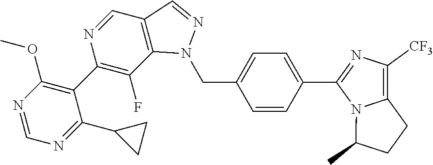 [SMILES: COc1ncnc(C2CC2)c1-c1ncc2cnn(Cc3ccc(-c4nc(C(F)(F)F)c5n4[C@H](C)CC5)cc3)c2c1F]

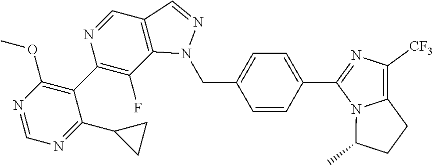 [SMILES: COc1ncnc(C2CC2)c1-c1ncc2cnn(Cc3ccc(-c4nc(C(F)(F)F)c5n4[C@@H](C)CC5)cc3)c2c1F]

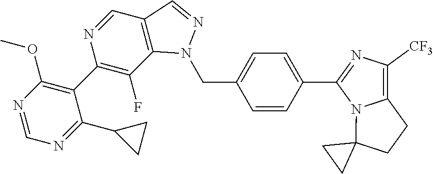 [SMILES: COc1ncnc(C2CC2)c1-c1ncc2cnn(Cc3ccc(-c4nc(C(F)(F)F)c5n4C4(CC5)CC4)cc3)c2c1F]

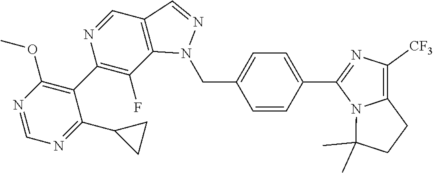 [SMILES: COc1ncnc(C2CC2)c1-c1ncc2cnn(Cc3ccc(-c4nc(C(F)(F)F)c5n4C(C)(C)CC5)cc3)c2c1F]

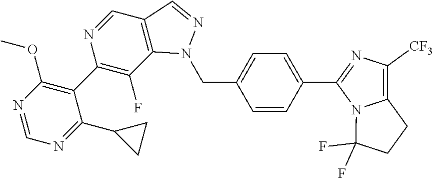 [SMILES: COc1ncnc(C2CC2)c1-c1ncc2cnn(Cc3ccc(-c4nc(C(F)(F)F)c5n4C(F)(F)CC5)cc3)c2c1F]

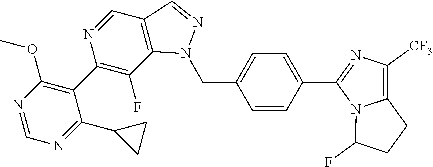 [SMILES: COc1ncnc(C2CC2)c1-c1ncc2cnn(Cc3ccc(-c4nc(C(F)(F)F)c5n4C(F)CC5)cc3)c2c1F]

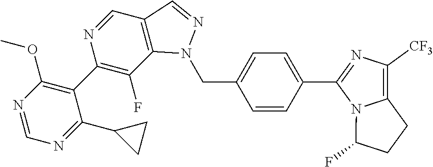 [SMILES: COc1ncnc(C2CC2)c1-c1ncc2cnn(Cc3ccc(-c4nc(C(F)(F)F)c5n4[C@@H](F)CC5)cc3)c2c1F]

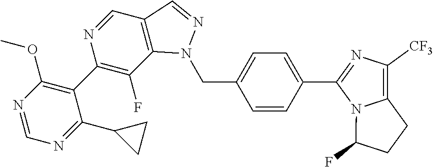 [SMILES: COc1ncnc(C2CC2)c1-c1ncc2cnn(Cc3ccc(-c4nc(C(F)(F)F)c5n4[C@H](F)CC5)cc3)c2c1F]

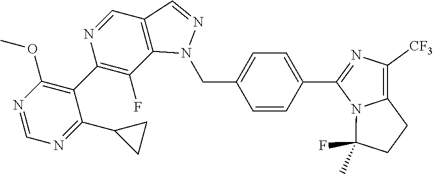 [SMILES: COc1ncnc(C2CC2)c1-c1ncc2cnn(Cc3ccc(-c4nc(C(F)(F)F)c5n4[C@](C)(F)CC5)cc3)c2c1F]

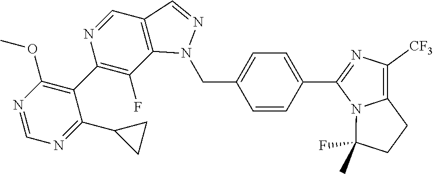 [SMILES: COc1ncnc(C2CC2)c1-c1ncc2cnn(Cc3ccc(-c4nc(C(F)(F)F)c5n4[C@@](C)(F)CC5)cc3)c2c1F]

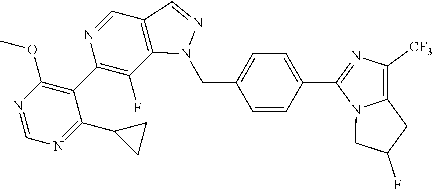 [SMILES: COc1ncnc(C2CC2)c1-c1ncc2cnn(Cc3ccc(-c4nc(C(F)(F)F)c5n4CC(F)C5)cc3)c2c1F]

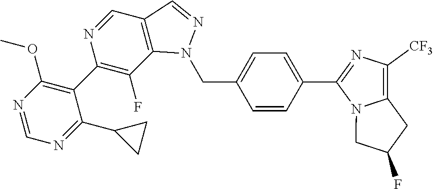 [SMILES: COc1ncnc(C2CC2)c1-c1ncc2cnn(Cc3ccc(-c4nc(C(F)(F)F)c5n4C[C@H](F)C5)cc3)c2c1F]

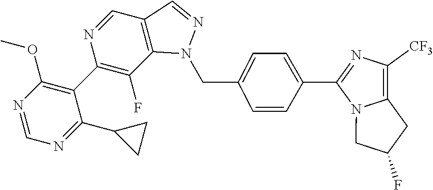 [SMILES: COc1ncnc(C2CC2)c1-c1ncc2cnn(Cc3ccc(-c4nc(C(F)(F)F)c5n4C[C@@H](F)C5)cc3)c2c1F]

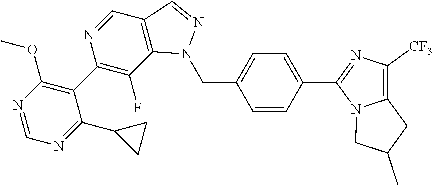 [SMILES: COc1ncnc(C2CC2)c1-c1ncc2cnn(Cc3ccc(-c4nc(C(F)(F)F)c5n4CC(C)C5)cc3)c2c1F]

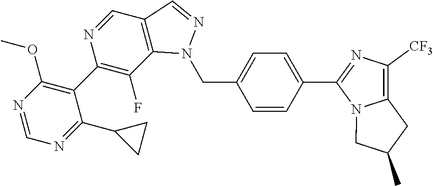 [SMILES: COc1ncnc(C2CC2)c1-c1ncc2cnn(Cc3ccc(-c4nc(C(F)(F)F)c5n4C[C@H](C)C5)cc3)c2c1F]

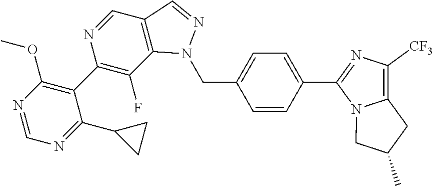 [SMILES: COc1ncnc(C2CC2)c1-c1ncc2cnn(Cc3ccc(-c4nc(C(F)(F)F)c5n4C[C@@H](C)C5)cc3)c2c1F]

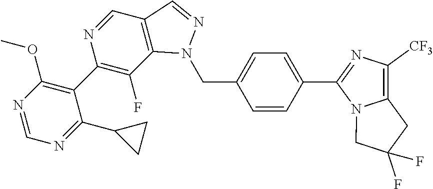 [SMILES: COc1ncnc(C2CC2)c1-c1ncc2cnn(Cc3ccc(-c4nc(C(F)(F)F)c5n4CC(F)(F)C5)cc3)c2c1F]

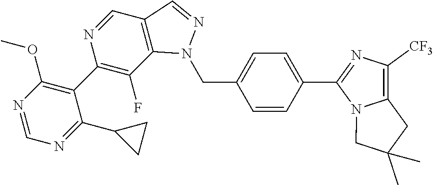

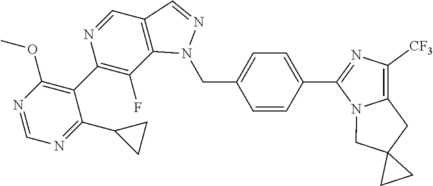 [SMILES: COc1ncnc(C2CC2)c1-c1ncc2cnn(Cc3ccc(-c4nc(C(F)(F)F)c5n4CC4(CC4)C5)cc3)c2c1F]

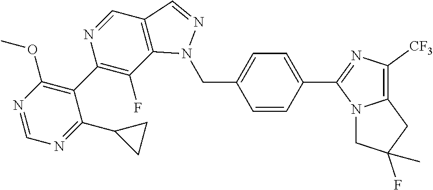

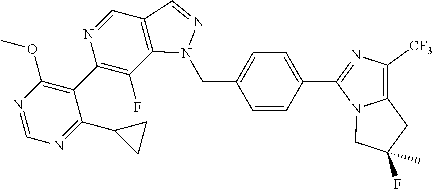

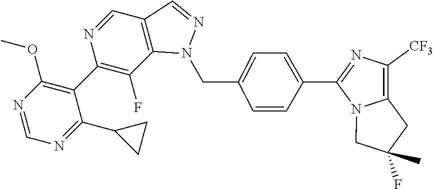

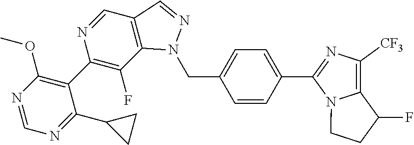 [SMILES: COc1ncnc(C2CC2)c1-c1ncc2cnn(Cc3ccc(-c4nc(C(F)(F)F)c5n4CCC5F)cc3)c2c1F]

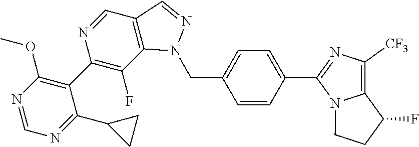 [SMILES: COc1ncnc(C2CC2)c1-c1ncc2cnn(Cc3ccc(-c4nc(C(F)(F)F)c5n4CC[C@H]5F)cc3)c2c1F]

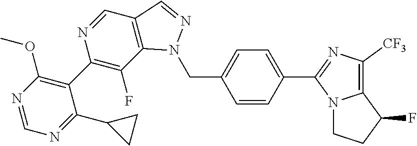 [SMILES: COc1ncnc(C2CC2)c1-c1ncc2cnn(Cc3ccc(-c4nc(C(F)(F)F)c5n4CC[C@@H]5F)cc3)c2c1F]

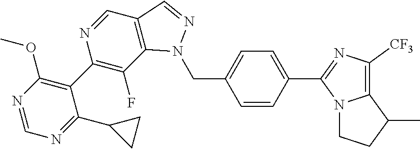

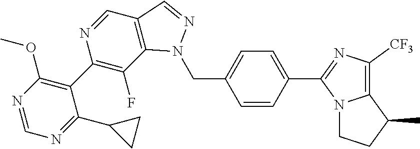

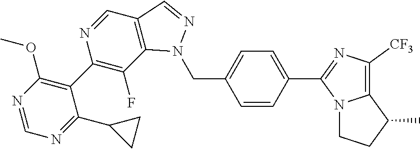

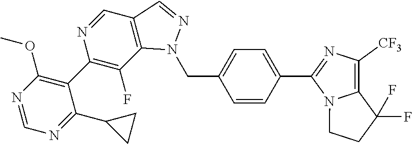 [SMILES: COc1ncnc(C2CC2)c1-c1ncc2cnn(Cc3ccc(-c4nc(C(F)(F)F)c5n4CCC5(F)F)cc3)c2c1F]

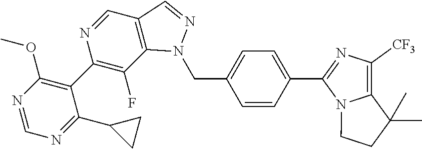

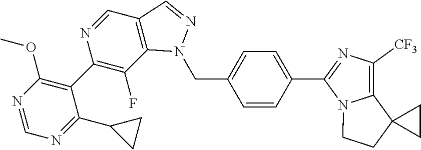 [SMILES: COc1ncnc(C2CC2)c1-c1ncc2cnn(Cc3ccc(-c4nc(C(F)(F)F)c5n4CCC54CC4)cc3)c2c1F]

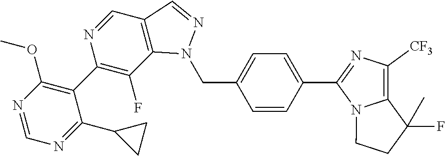 [SMILES: COc1ncnc(C2CC2)c1-c1ncc2cnn(Cc3ccc(-c4nc(C(F)(F)F)c5n4CCC5(C)F)cc3)c2c1F]

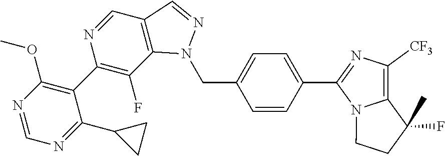 [SMILES: COc1ncnc(C2CC2)c1-c1ncc2cnn(Cc3ccc(-c4nc(C(F)(F)F)c5n4CC[C@@]5(C)F)cc3)c2c1F]

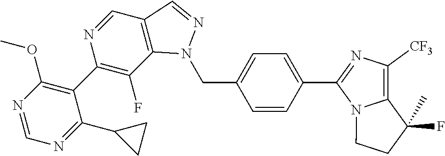 [SMILES: COc1ncnc(C2CC2)c1-c1ncc2cnn(Cc3ccc(-c4nc(C(F)(F)F)c5n4CC[C@]5(C)F)cc3)c2c1F]

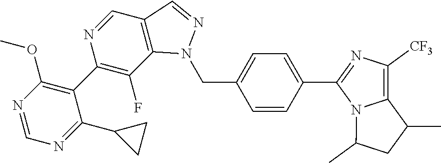

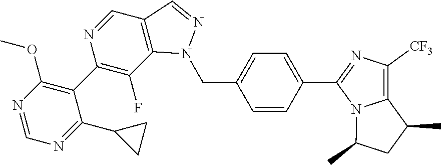

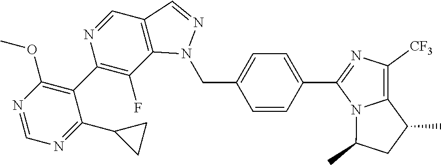

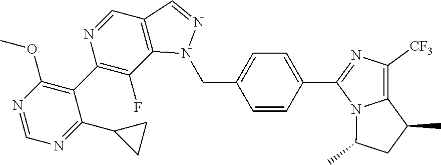

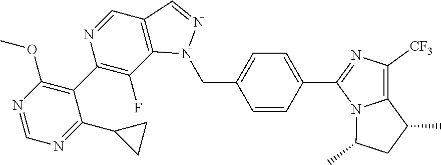

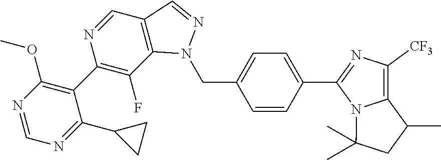

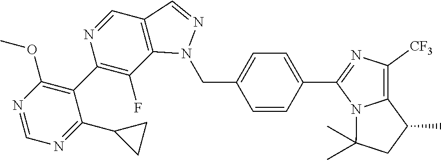

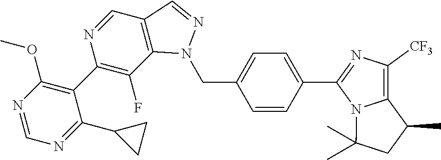

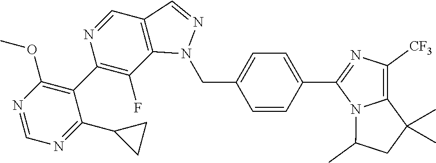

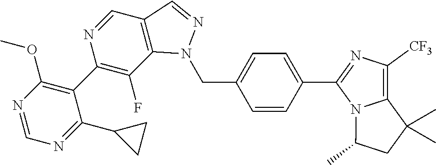

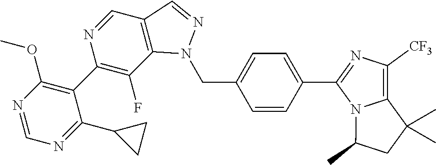

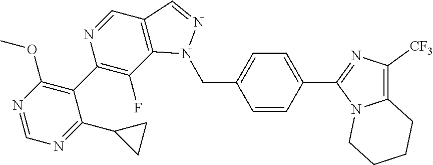 [SMILES: COc1ncnc(C2CC2)c1-c1ncc2cnn(Cc3ccc(-c4nc(C(F)(F)F)c5n4CCCC5)cc3)c2c1F]

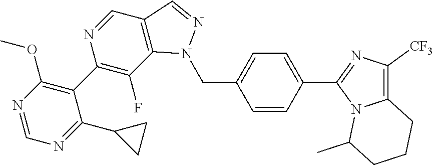 [SMILES: COc1ncnc(C2CC2)c1-c1ncc2cnn(Cc3ccc(-c4nc(C(F)(F)F)c5n4C(C)CCC5)cc3)c2c1F]

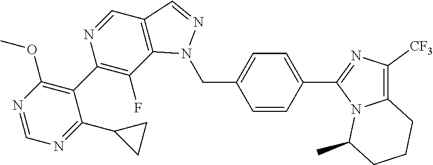 [SMILES: COc1ncnc(C2CC2)c1-c1ncc2cnn(Cc3ccc(-c4nc(C(F)(F)F)c5n4[C@H](C)CCC5)cc3)c2c1F]

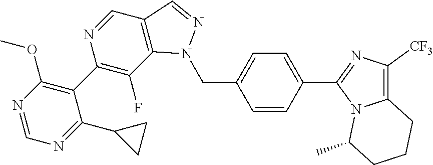 [SMILES: COc1ncnc(C2CC2)c1-c1ncc2cnn(Cc3ccc(-c4nc(C(F)(F)F)c5n4[C@@H](C)CCC5)cc3)c2c1F]

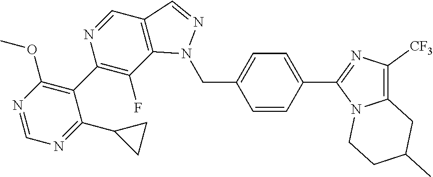

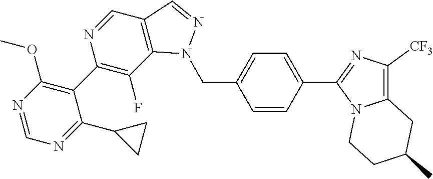

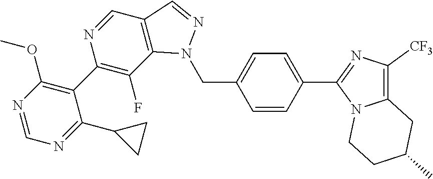

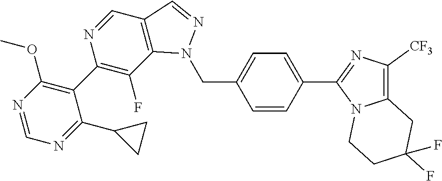 [SMILES: COc1ncnc(C2CC2)c1-c1ncc2cnn(Cc3ccc(-c4nc(C(F)(F)F)c5n4CCC(F)(F)C5)cc3)c2c1F]

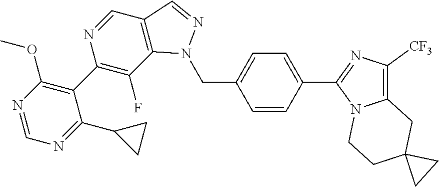 [SMILES: COc1ncnc(C2CC2)c1-c1ncc2cnn(Cc3ccc(-c4nc(C(F)(F)F)c5n4CCC4(CC4)C5)cc3)c2c1F]

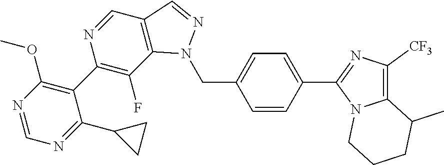

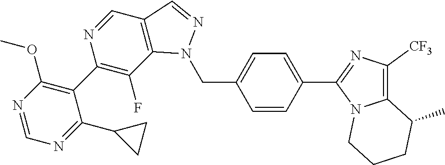

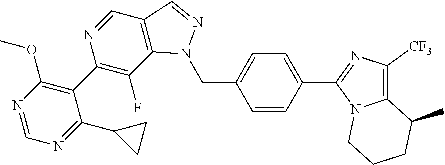

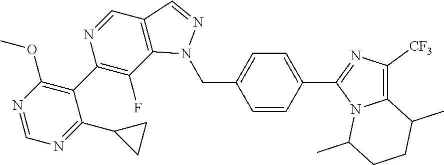

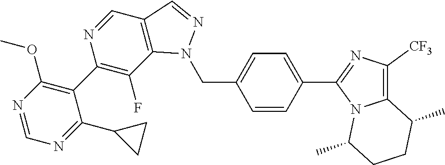

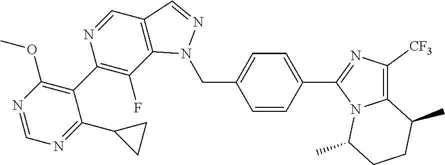

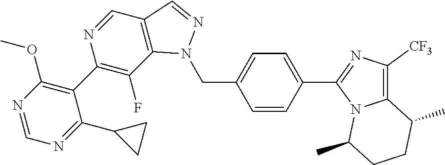

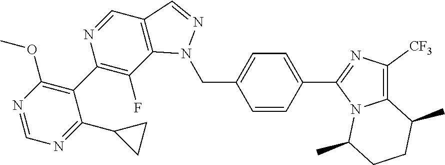

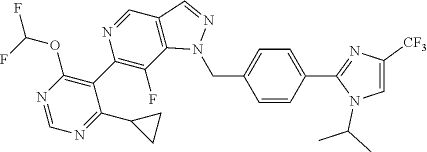 [SMILES: CC(C)n1cc(C(F)(F)F)nc1-c1ccc(Cn2ncc3cnc(-c4c(OC(F)F)ncnc4C4CC4)c(F)c32)cc1]

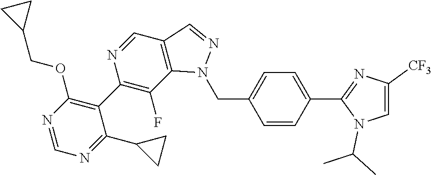 [SMILES: CC(C)n1cc(C(F)(F)F)nc1-c1ccc(Cn2ncc3cnc(-c4c(OCC5CC5)ncnc4C4CC4)c(F)c32)cc1]

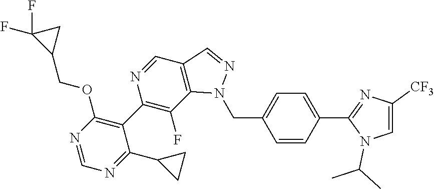 [SMILES: CC(C)n1cc(C(F)(F)F)nc1-c1ccc(Cn2ncc3cnc(-c4c(OCC5CC5(F)F)ncnc4C4CC4)c(F)c32)cc1]

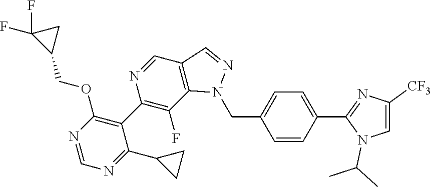 [SMILES: CC(C)n1cc(C(F)(F)F)nc1-c1ccc(Cn2ncc3cnc(-c4c(OC[C@H]5CC5(F)F)ncnc4C4CC4)c(F)c32)cc1]

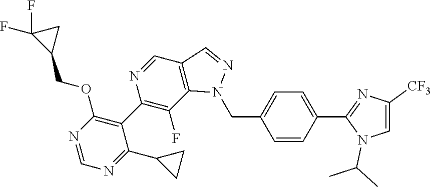 [SMILES: CC(C)n1cc(C(F)(F)F)nc1-c1ccc(Cn2ncc3cnc(-c4c(OC[C@@H]5CC5(F)F)ncnc4C4CC4)c(F)c32)cc1]

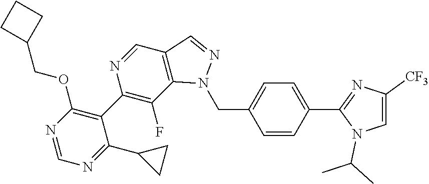 [SMILES: CC(C)n1cc(C(F)(F)F)nc1-c1ccc(Cn2ncc3cnc(-c4c(OCC5CCC5)ncnc4C4CC4)c(F)c32)cc1]

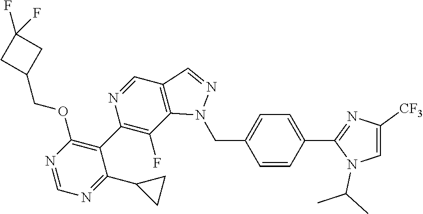 [SMILES: CC(C)n1cc(C(F)(F)F)nc1-c1ccc(Cn2ncc3cnc(-c4c(OCC5CC(F)(F)C5)ncnc4C4CC4)c(F)c32)cc1]

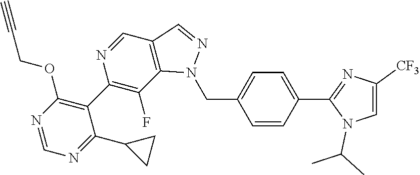 [SMILES: C#CCOc1ncnc(C2CC2)c1-c1ncc2cnn(Cc3ccc(-c4nc(C(F)(F)F)cn4C(C)C)cc3)c2c1F]

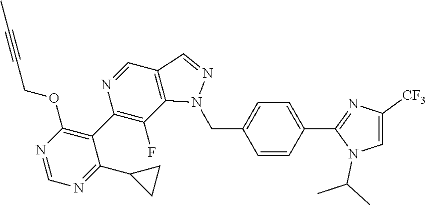 [SMILES: CC#CCOc1ncnc(C2CC2)c1-c1ncc2cnn(Cc3ccc(-c4nc(C(F)(F)F)cn4C(C)C)cc3)c2c1F]

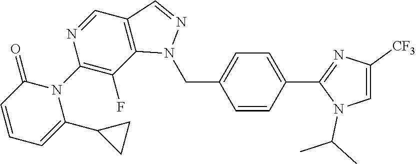 [SMILES: CC(C)n1cc(C(F)(F)F)nc1-c1ccc(Cn2ncc3cnc(-n4c(C5CC5)cccc4=O)c(F)c32)cc1]

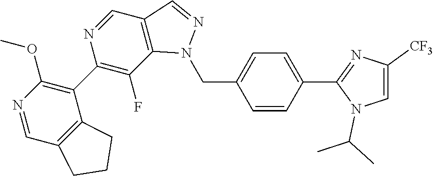 [SMILES: COc1ncc2c(c1-c1ncc3cnn(Cc4ccc(-c5nc(C(F)(F)F)cn5C(C)C)cc4)c3c1F)CCC2]

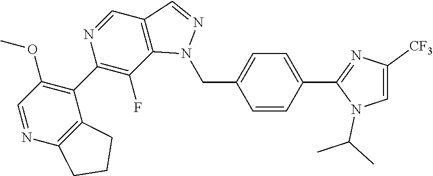 [SMILES: COc1cnc2c(c1-c1ncc3cnn(Cc4ccc(-c5nc(C(F)(F)F)cn5C(C)C)cc4)c3c1F)CCC2]

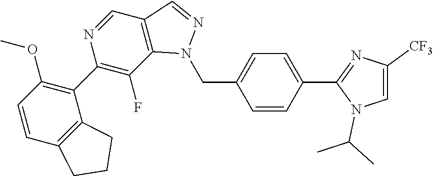 [SMILES: COc1ccc2c(c1-c1ncc3cnn(Cc4ccc(-c5nc(C(F)(F)F)cn5C(C)C)cc4)c3c1F)CCC2]

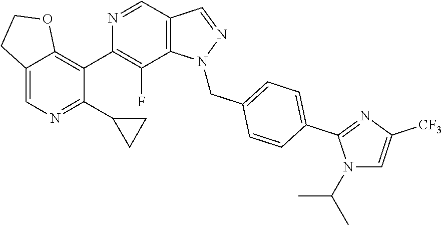 [SMILES: CC(C)n1cc(C(F)(F)F)nc1-c1ccc(Cn2ncc3cnc(-c4c(C5CC5)ncc5c4OCC5)c(F)c32)cc1]

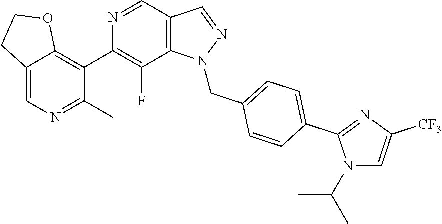 [SMILES: Cc1ncc2c(c1-c1ncc3cnn(Cc4ccc(-c5nc(C(F)(F)F)cn5C(C)C)cc4)c3c1F)OCC2]

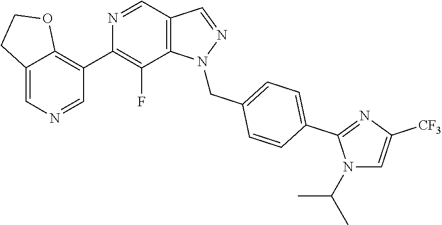 [SMILES: CC(C)n1cc(C(F)(F)F)nc1-c1ccc(Cn2ncc3cnc(-c4cncc5c4OCC5)c(F)c32)cc1]

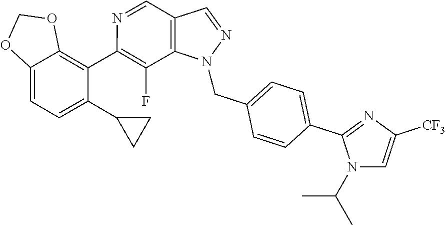 [SMILES: CC(C)n1cc(C(F)(F)F)nc1-c1ccc(Cn2ncc3cnc(-c4c(C5CC5)ccc5c4OCO5)c(F)c32)cc1]

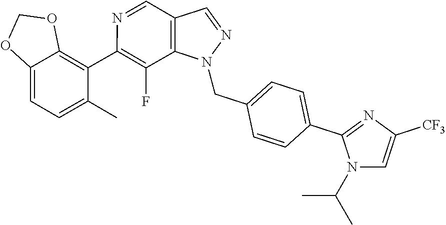 [SMILES: Cc1ccc2c(c1-c1ncc3cnn(Cc4ccc(-c5nc(C(F)(F)F)cn5C(C)C)cc4)c3c1F)OCO2]

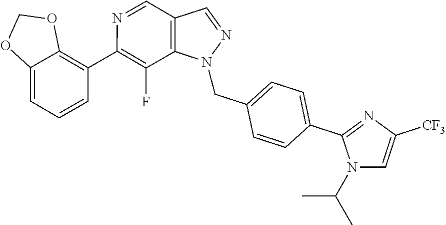 [SMILES: CC(C)n1cc(C(F)(F)F)nc1-c1ccc(Cn2ncc3cnc(-c4cccc5c4OCO5)c(F)c32)cc1]

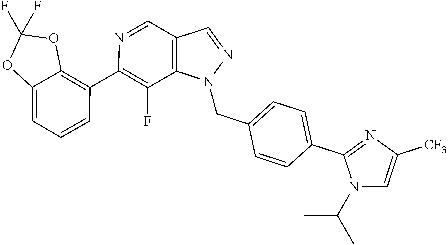 [SMILES: CC(C)n1cc(C(F)(F)F)nc1-c1ccc(Cn2ncc3cnc(-c4cccc5c4OC(F)(F)O5)c(F)c32)cc1]

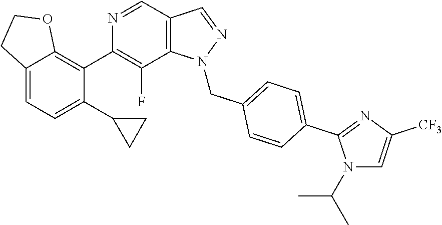 [SMILES: CC(C)n1cc(C(F)(F)F)nc1-c1ccc(Cn2ncc3cnc(-c4c(C5CC5)ccc5c4OCC5)c(F)c32)cc1]

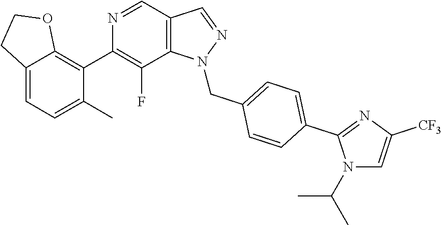 [SMILES: Cc1ccc2c(c1-c1ncc3cnn(Cc4ccc(-c5nc(C(F)(F)F)cn5C(C)C)cc4)c3c1F)OCC2]

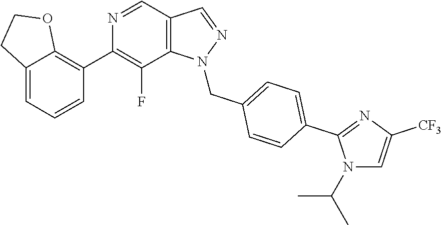 [SMILES: CC(C)n1cc(C(F)(F)F)nc1-c1ccc(Cn2ncc3cnc(-c4cccc5c4OCC5)c(F)c32)cc1]

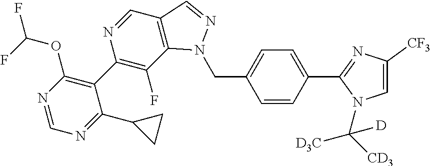 [SMILES: [2H]C([2H])([2H])C([2H])(n1cc(C(F)(F)F)nc1-c1ccc(Cn2ncc3cnc(-c4c(OC(F)F)ncnc4C4CC4)c(F)c32)cc1)C([2H])([2H])[2H]]

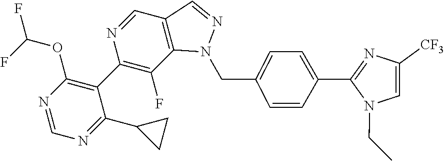 [SMILES: CCn1cc(C(F)(F)F)nc1-c1ccc(Cn2ncc3cnc(-c4c(OC(F)F)ncnc4C4CC4)c(F)c32)cc1]

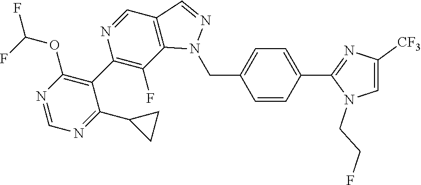 [SMILES: FCCn1cc(C(F)(F)F)nc1-c1ccc(Cn2ncc3cnc(-c4c(OC(F)F)ncnc4C4CC4)c(F)c32)cc1]

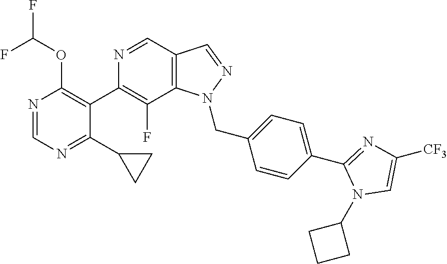 [SMILES: Fc1c(-c2c(OC(F)F)ncnc2C2CC2)ncc2cnn(Cc3ccc(-c4nc(C(F)(F)F)cn4C4CCC4)cc3)c12]

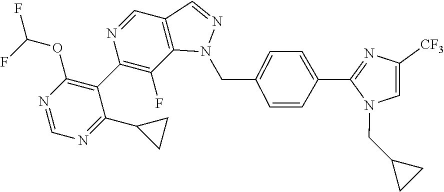 [SMILES: Fc1c(-c2c(OC(F)F)ncnc2C2CC2)ncc2cnn(Cc3ccc(-c4nc(C(F)(F)F)cn4CC4CC4)cc3)c12]

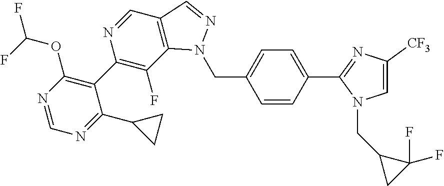 [SMILES: Fc1c(-c2c(OC(F)F)ncnc2C2CC2)ncc2cnn(Cc3ccc(-c4nc(C(F)(F)F)cn4CC4CC4(F)F)cc3)c12]

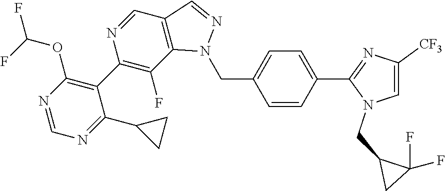 [SMILES: Fc1c(-c2c(OC(F)F)ncnc2C2CC2)ncc2cnn(Cc3ccc(-c4nc(C(F)(F)F)cn4C[C@@H]4CC4(F)F)cc3)c12]

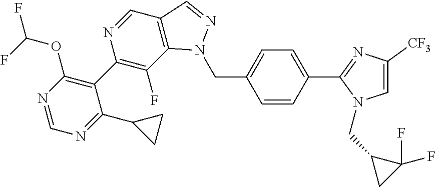 [SMILES: Fc1c(-c2c(OC(F)F)ncnc2C2CC2)ncc2cnn(Cc3ccc(-c4nc(C(F)(F)F)cn4C[C@H]4CC4(F)F)cc3)c12]

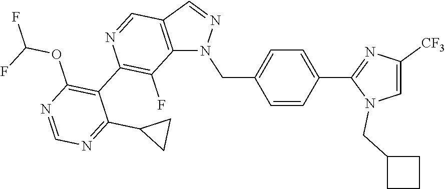 [SMILES: Fc1c(-c2c(OC(F)F)ncnc2C2CC2)ncc2cnn(Cc3ccc(-c4nc(C(F)(F)F)cn4CC4CCC4)cc3)c12]

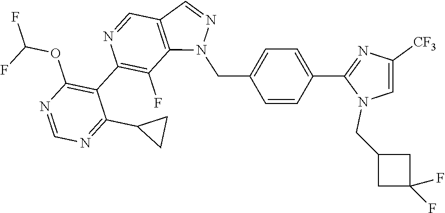 [SMILES: Fc1c(-c2c(OC(F)F)ncnc2C2CC2)ncc2cnn(Cc3ccc(-c4nc(C(F)(F)F)cn4CC4CC(F)(F)C4)cc3)c12]

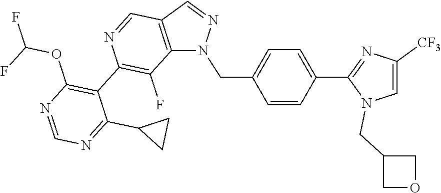 [SMILES: Fc1c(-c2c(OC(F)F)ncnc2C2CC2)ncc2cnn(Cc3ccc(-c4nc(C(F)(F)F)cn4CC4COC4)cc3)c12]

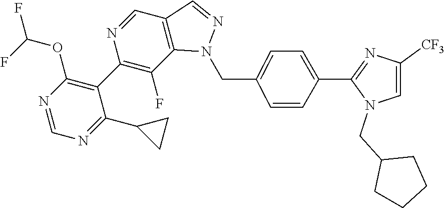 [SMILES: Fc1c(-c2c(OC(F)F)ncnc2C2CC2)ncc2cnn(Cc3ccc(-c4nc(C(F)(F)F)cn4CC4CCCC4)cc3)c12]

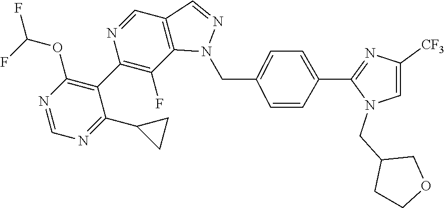 [SMILES: Fc1c(-c2c(OC(F)F)ncnc2C2CC2)ncc2cnn(Cc3ccc(-c4nc(C(F)(F)F)cn4CC4CCOC4)cc3)c12]

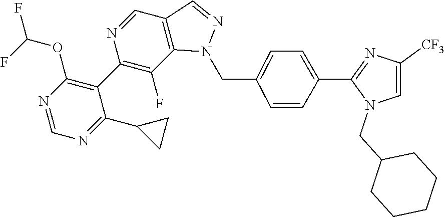 [SMILES: Fc1c(-c2c(OC(F)F)ncnc2C2CC2)ncc2cnn(Cc3ccc(-c4nc(C(F)(F)F)cn4CC4CCCCC4)cc3)c12]

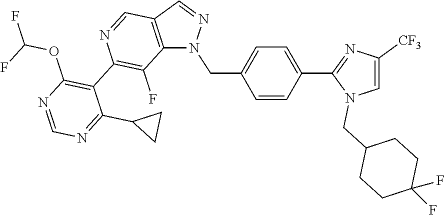 [SMILES: Fc1c(-c2c(OC(F)F)ncnc2C2CC2)ncc2cnn(Cc3ccc(-c4nc(C(F)(F)F)cn4CC4CCC(F)(F)CC4)cc3)c12]

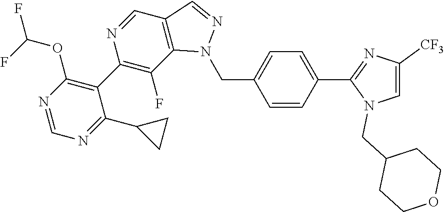 [SMILES: Fc1c(-c2c(OC(F)F)ncnc2C2CC2)ncc2cnn(Cc3ccc(-c4nc(C(F)(F)F)cn4CC4CCOCC4)cc3)c12]

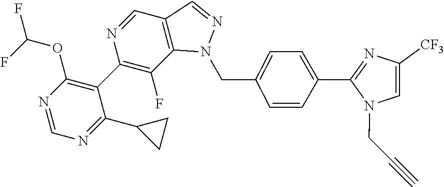 [SMILES: C#CCn1cc(C(F)(F)F)nc1-c1ccc(Cn2ncc3cnc(-c4c(OC(F)F)ncnc4C4CC4)c(F)c32)cc1]

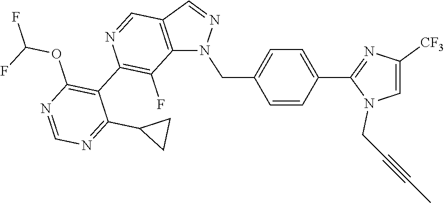 [SMILES: CC#CCn1cc(C(F)(F)F)nc1-c1ccc(Cn2ncc3cnc(-c4c(OC(F)F)ncnc4C4CC4)c(F)c32)cc1]

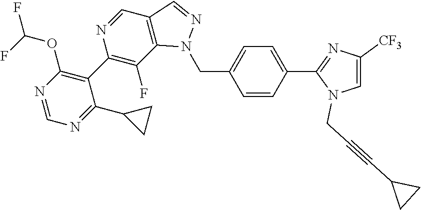 [SMILES: Fc1c(-c2c(OC(F)F)ncnc2C2CC2)ncc2cnn(Cc3ccc(-c4nc(C(F)(F)F)cn4CC#CC4CC4)cc3)c12]

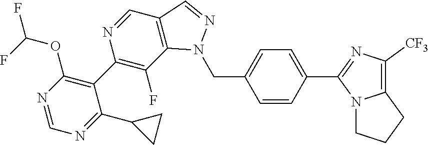 [SMILES: Fc1c(-c2c(OC(F)F)ncnc2C2CC2)ncc2cnn(Cc3ccc(-c4nc(C(F)(F)F)c5n4CCC5)cc3)c12]

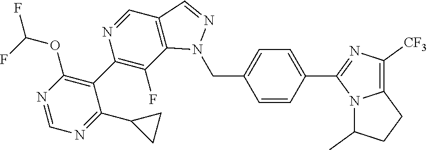 [SMILES: CC1CCc2c(C(F)(F)F)nc(-c3ccc(Cn4ncc5cnc(-c6c(OC(F)F)ncnc6C6CC6)c(F)c54)cc3)n21]

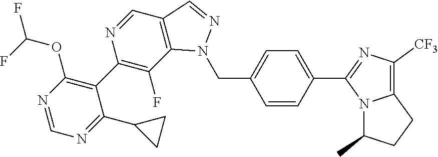 [SMILES: C[C@@H]1CCc2c(C(F)(F)F)nc(-c3ccc(Cn4ncc5cnc(-c6c(OC(F)F)ncnc6C6CC6)c(F)c54)cc3)n21]

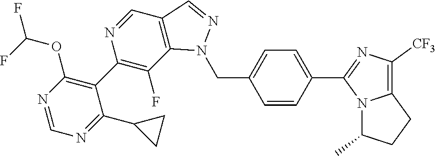 [SMILES: C[C@H]1CCc2c(C(F)(F)F)nc(-c3ccc(Cn4ncc5cnc(-c6c(OC(F)F)ncnc6C6CC6)c(F)c54)cc3)n21]

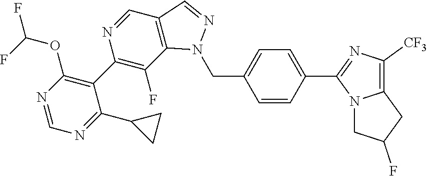 [SMILES: Fc1c(-c2c(OC(F)F)ncnc2C2CC2)ncc2cnn(Cc3ccc(-c4nc(C(F)(F)F)c5n4CC(F)C5)cc3)c12]

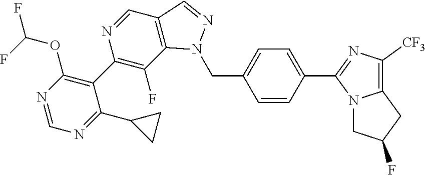 [SMILES: Fc1c(-c2c(OC(F)F)ncnc2C2CC2)ncc2cnn(Cc3ccc(-c4nc(C(F)(F)F)c5n4C[C@H](F)C5)cc3)c12]

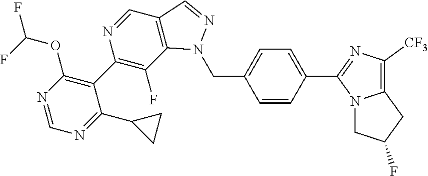 [SMILES: Fc1c(-c2c(OC(F)F)ncnc2C2CC2)ncc2cnn(Cc3ccc(-c4nc(C(F)(F)F)c5n4C[C@@H](F)C5)cc3)c12]

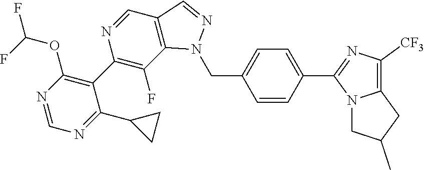 [SMILES: CC1Cc2c(C(F)(F)F)nc(-c3ccc(Cn4ncc5cnc(-c6c(OC(F)F)ncnc6C6CC6)c(F)c54)cc3)n2C1]

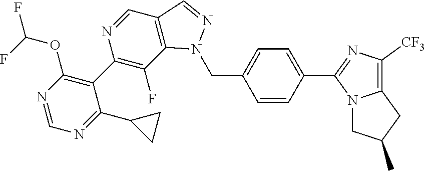 [SMILES: C[C@@H]1Cc2c(C(F)(F)F)nc(-c3ccc(Cn4ncc5cnc(-c6c(OC(F)F)ncnc6C6CC6)c(F)c54)cc3)n2C1]

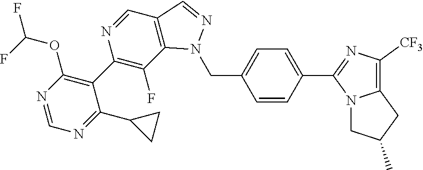 [SMILES: C[C@H]1Cc2c(C(F)(F)F)nc(-c3ccc(Cn4ncc5cnc(-c6c(OC(F)F)ncnc6C6CC6)c(F)c54)cc3)n2C1]

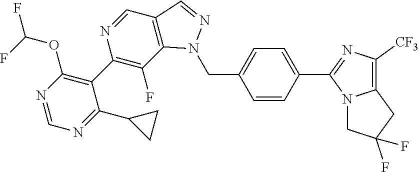 [SMILES: Fc1c(-c2c(OC(F)F)ncnc2C2CC2)ncc2cnn(Cc3ccc(-c4nc(C(F)(F)F)c5n4CC(F)(F)C5)cc3)c12]

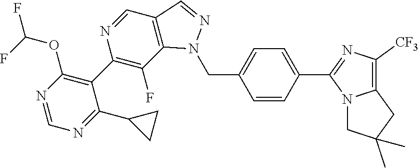 [SMILES: CC1(C)Cc2c(C(F)(F)F)nc(-c3ccc(Cn4ncc5cnc(-c6c(OC(F)F)ncnc6C6CC6)c(F)c54)cc3)n2C1]

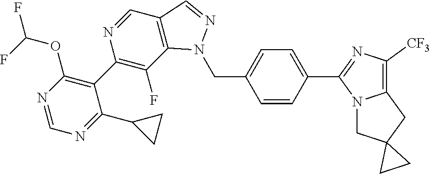 [SMILES: Fc1c(-c2c(OC(F)F)ncnc2C2CC2)ncc2cnn(Cc3ccc(-c4nc(C(F)(F)F)c5n4CC4(CC4)C5)cc3)c12]

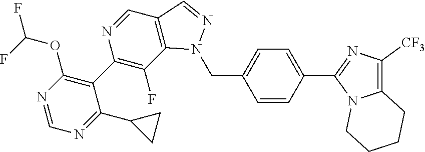 [SMILES: Fc1c(-c2c(OC(F)F)ncnc2C2CC2)ncc2cnn(Cc3ccc(-c4nc(C(F)(F)F)c5n4CCCC5)cc3)c12]

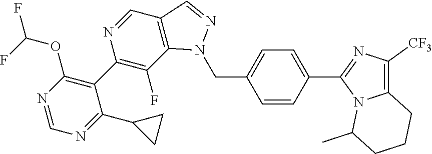 [SMILES: CC1CCCc2c(C(F)(F)F)nc(-c3ccc(Cn4ncc5cnc(-c6c(OC(F)F)ncnc6C6CC6)c(F)c54)cc3)n21]

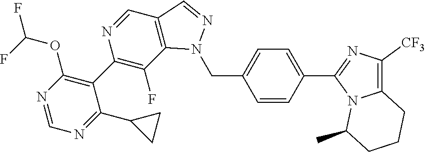 [SMILES: C[C@@H]1CCCc2c(C(F)(F)F)nc(-c3ccc(Cn4ncc5cnc(-c6c(OC(F)F)ncnc6C6CC6)c(F)c54)cc3)n21]

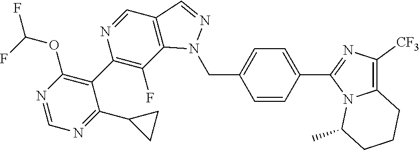 [SMILES: C[C@H]1CCCc2c(C(F)(F)F)nc(-c3ccc(Cn4ncc5cnc(-c6c(OC(F)F)ncnc6C6CC6)c(F)c54)cc3)n21]

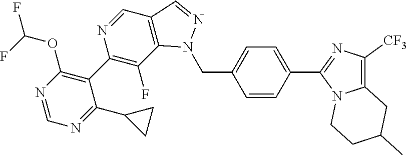 [SMILES: CC1CCn2c(-c3ccc(Cn4ncc5cnc(-c6c(OC(F)F)ncnc6C6CC6)c(F)c54)cc3)nc(C(F)(F)F)c2C1]

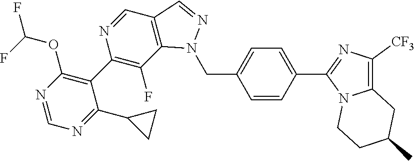 [SMILES: C[C@H]1CCn2c(-c3ccc(Cn4ncc5cnc(-c6c(OC(F)F)ncnc6C6CC6)c(F)c54)cc3)nc(C(F)(F)F)c2C1]

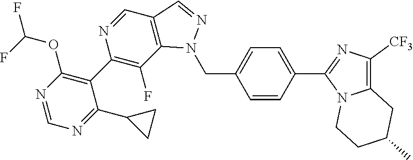 [SMILES: C[C@@H]1CCn2c(-c3ccc(Cn4ncc5cnc(-c6c(OC(F)F)ncnc6C6CC6)c(F)c54)cc3)nc(C(F)(F)F)c2C1]

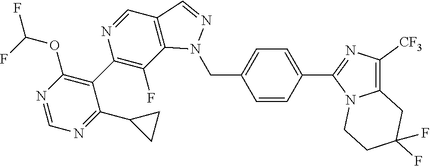 [SMILES: Fc1c(-c2c(OC(F)F)ncnc2C2CC2)ncc2cnn(Cc3ccc(-c4nc(C(F)(F)F)c5n4CCC(F)(F)C5)cc3)c12]

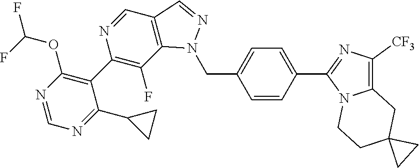 [SMILES: Fc1c(-c2c(OC(F)F)ncnc2C2CC2)ncc2cnn(Cc3ccc(-c4nc(C(F)(F)F)c5n4CCC4(CC4)C5)cc3)c12]

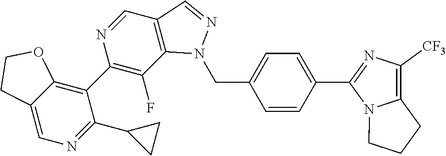 [SMILES: Fc1c(-c2c(C3CC3)ncc3c2OCC3)ncc2cnn(Cc3ccc(-c4nc(C(F)(F)F)c5n4CCC5)cc3)c12]

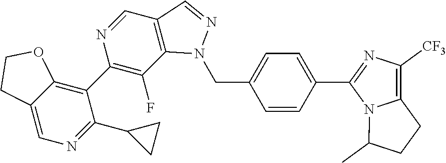 [SMILES: CC1CCc2c(C(F)(F)F)nc(-c3ccc(Cn4ncc5cnc(-c6c(C7CC7)ncc7c6OCC7)c(F)c54)cc3)n21]

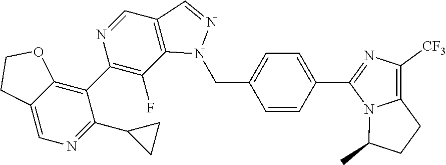 [SMILES: C[C@@H]1CCc2c(C(F)(F)F)nc(-c3ccc(Cn4ncc5cnc(-c6c(C7CC7)ncc7c6OCC7)c(F)c54)cc3)n21]

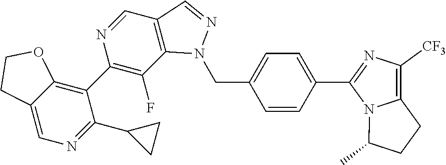 [SMILES: C[C@H]1CCc2c(C(F)(F)F)nc(-c3ccc(Cn4ncc5cnc(-c6c(C7CC7)ncc7c6OCC7)c(F)c54)cc3)n21]

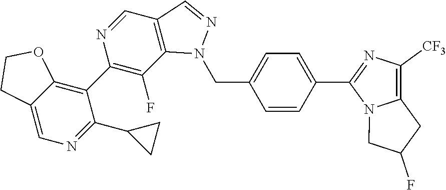 [SMILES: Fc1c(-c2c(C3CC3)ncc3c2OCC3)ncc2cnn(Cc3ccc(-c4nc(C(F)(F)F)c5n4CC(F)C5)cc3)c12]

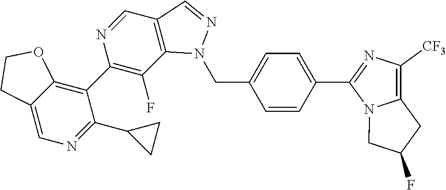 [SMILES: Fc1c(-c2c(C3CC3)ncc3c2OCC3)ncc2cnn(Cc3ccc(-c4nc(C(F)(F)F)c5n4C[C@H](F)C5)cc3)c12]

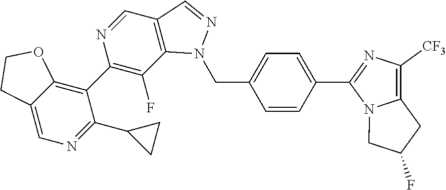 [SMILES: Fc1c(-c2c(C3CC3)ncc3c2OCC3)ncc2cnn(Cc3ccc(-c4nc(C(F)(F)F)c5n4C[C@@H](F)C5)cc3)c12]

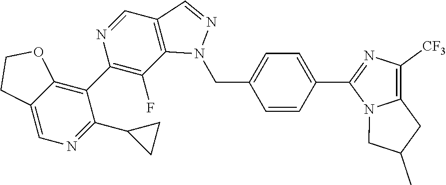 [SMILES: CC1Cc2c(C(F)(F)F)nc(-c3ccc(Cn4ncc5cnc(-c6c(C7CC7)ncc7c6OCC7)c(F)c54)cc3)n2C1]

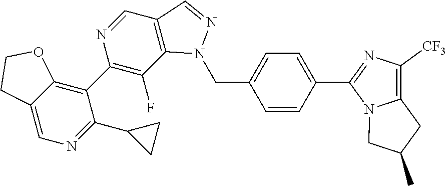 [SMILES: C[C@@H]1Cc2c(C(F)(F)F)nc(-c3ccc(Cn4ncc5cnc(-c6c(C7CC7)ncc7c6OCC7)c(F)c54)cc3)n2C1]

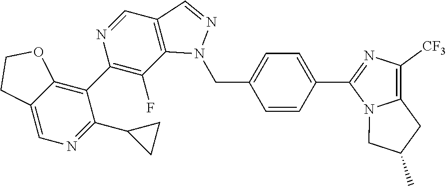 [SMILES: C[C@H]1Cc2c(C(F)(F)F)nc(-c3ccc(Cn4ncc5cnc(-c6c(C7CC7)ncc7c6OCC7)c(F)c54)cc3)n2C1]

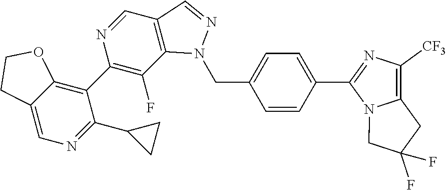 [SMILES: Fc1c(-c2c(C3CC3)ncc3c2OCC3)ncc2cnn(Cc3ccc(-c4nc(C(F)(F)F)c5n4CC(F)(F)C5)cc3)c12]

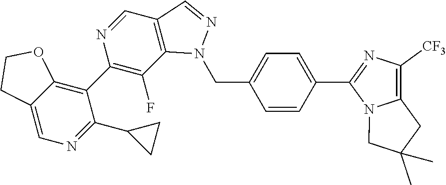 [SMILES: CC1(C)Cc2c(C(F)(F)F)nc(-c3ccc(Cn4ncc5cnc(-c6c(C7CC7)ncc7c6OCC7)c(F)c54)cc3)n2C1]

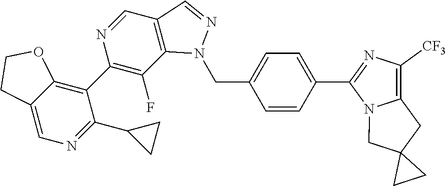 [SMILES: Fc1c(-c2c(C3CC3)ncc3c2OCC3)ncc2cnn(Cc3ccc(-c4nc(C(F)(F)F)c5n4CC4(CC4)C5)cc3)c12]

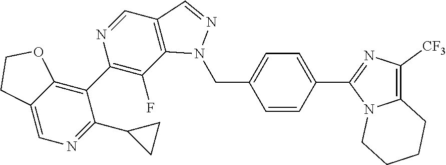 [SMILES: Fc1c(-c2c(C3CC3)ncc3c2OCC3)ncc2cnn(Cc3ccc(-c4nc(C(F)(F)F)c5n4CCCC5)cc3)c12]

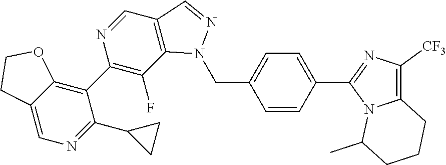 [SMILES: CC1CCCc2c(C(F)(F)F)nc(-c3ccc(Cn4ncc5cnc(-c6c(C7CC7)ncc7c6OCC7)c(F)c54)cc3)n21]

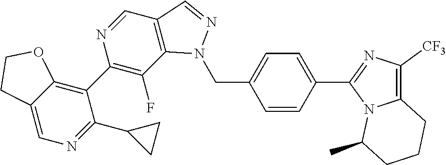 [SMILES: C[C@@H]1CCCc2c(C(F)(F)F)nc(-c3ccc(Cn4ncc5cnc(-c6c(C7CC7)ncc7c6OCC7)c(F)c54)cc3)n21]

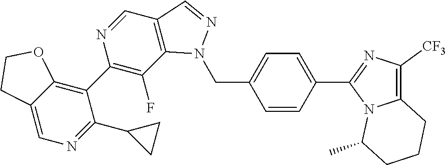 [SMILES: C[C@H]1CCCc2c(C(F)(F)F)nc(-c3ccc(Cn4ncc5cnc(-c6c(C7CC7)ncc7c6OCC7)c(F)c54)cc3)n21]

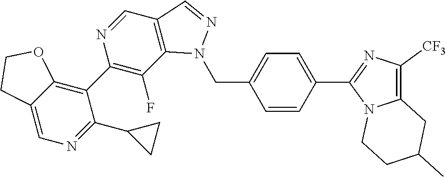 [SMILES: CC1CCn2c(-c3ccc(Cn4ncc5cnc(-c6c(C7CC7)ncc7c6OCC7)c(F)c54)cc3)nc(C(F)(F)F)c2C1]

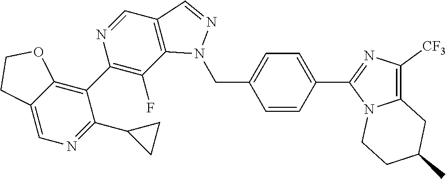 [SMILES: C[C@H]1CCn2c(-c3ccc(Cn4ncc5cnc(-c6c(C7CC7)ncc7c6OCC7)c(F)c54)cc3)nc(C(F)(F)F)c2C1]

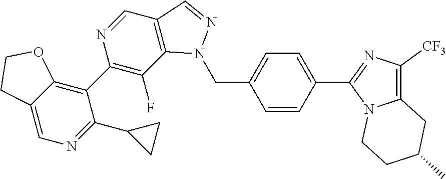 [SMILES: C[C@@H]1CCn2c(-c3ccc(Cn4ncc5cnc(-c6c(C7CC7)ncc7c6OCC7)c(F)c54)cc3)nc(C(F)(F)F)c2C1]

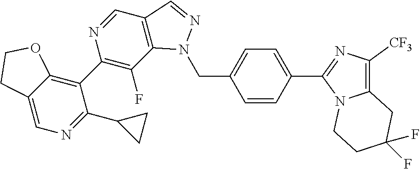 [SMILES: Fc1c(-c2c(C3CC3)ncc3c2OCC3)ncc2cnn(Cc3ccc(-c4nc(C(F)(F)F)c5n4CCC(F)(F)C5)cc3)c12]

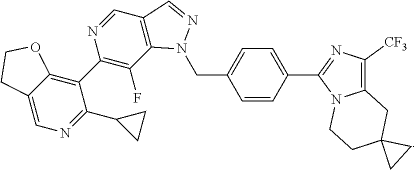 [SMILES: Fc1c(-c2c(C3CC3)ncc3c2OCC3)ncc2cnn(Cc3ccc(-c4nc(C(F)(F)F)c5n4CCC4(CC4)C5)cc3)c12]

The present invention relates to a pharmaceutical composition comprising the compound or a stereoisomer, tautomer, deuterated substance, solvate, prodrug, metabolite, pharmaceutically acceptable salt or eutectic crystal thereof according to the present invention, and a pharmaceutically acceptable carrier.

The present invention relates to an application of the compound or a stereoisomer, tautomer, deuterated substance, solvate, prodrug, metabolite, pharmaceutically acceptable salt or eutectic crystal thereof according to the present invention in the preparation of a medicine for treating a disease related to USP activity or expression, preferably in the preparation of a medicine for treating a tumor disease.

The present invention relates to an application of any of the above-mentioned compounds or stereoisomers, deuterated substances, solvates, prodrugs, metabolites, pharmaceutically acceptable salts or eutectic crystals thereof, or the above pharmaceutical composition in the preparation of a medicine for treating a disease related to USP1 activity or expression, wherein the disease is preferably selected from tumors.

The present invention relates to a pharmaceutical composition or pharmaceutical preparation comprising a therapeutically effective amount of the compound or a stereoisomer, deuterated substance, solvate, prodrug, metabolite, pharmaceutically acceptable salt or eutectic crystal thereof according to the present invention, and a pharmaceutical excipient. The pharmaceutical composition can be in a unit preparation form (the amount of the active drug in the unit preparation is also referred to as the “preparation specification”).

The present invention further provides a method for treating a disease in a mammal, the method comprising administering to the mammal a therapeutically effective amount of the compound, or the stereoisomer, deuterated substance, solvate, prodrug, metabolite, pharmaceutically acceptable salt or eutectic crystal thereof or the pharmaceutical composition according to the present invention. In some embodiments, the mammal mentioned in the present invention includes human.

The term “effective amount” or “therapeutically effective amount” in the present application refers to a sufficient amount of the compound disclosed in the present application that is administered to alleviate, to some extent, one or more symptoms of a disease or condition being treated (for example, a disease related to USP1 activity or expression, such as tumor). In some embodiments, the outcome is the reduction and/or remission of signs, symptoms or causes of the disease, or any other desired change in the biological system. For example, an “effective amount” in terms of the therapeutic use is an amount of the composition comprising the compound disclosed in the present application that is required to provide clinically significant reduction of the symptoms of the disease. Examples of therapeutically effective amounts include, but are not limited to 1-1500 mg, 1-1400 mg, 1-1200 mg, 1-600 mg, 2-600 mg, 3-600 mg, 4-600 mg, 5-600 mg, 6-600 mg, 10-600 mg, 20-600 mg, 25-600 mg, 30-600 mg, 40-600 mg, 50-600 mg, 60-600 mg, 70-600 mg, 75-600 mg, 80-600 mg, 90-600 mg, 100-600 mg, 200-600 mg, 1-500 mg, 2-500 mg, 3-500 mg, 4-500 mg, 5-500 mg, 6-500 mg, 10-500 mg, 20-500 mg, 25-500 mg, 30-500 mg, 40-500 mg, 50-500 mg, 60-500 mg, 70-500 mg, 75-500 mg, 80-500 mg, 90-500 mg, 100-500 mg, 125-500 mg, 150-500 mg, 200-500 mg, 250-500 mg, 300-500 mg, 400-500 mg, 5-400 mg, 10-400 mg, 20-400 mg, 25-400 mg, 30-400 mg, 40-400 mg, 50-400 mg, 60-400 mg, 70-400 mg, 75-400 mg, 80-400 mg, 90-400 mg, 100-400 mg, 125-400 mg, 150-400 mg, 200-400 mg, 250-400 mg, 300-400 mg, 1-300 mg, 2-300 mg, 5-300 mg, 10-300 mg, 20-300 mg, 25-300 mg, 30-300 mg, 40-300 mg, 50-300 mg, 60-300 mg, 70-300 mg, 75-300 mg, 80-300 mg, 90-300 mg, 100-300 mg, 125-300 mg, 150-300 mg, 200-300 mg, 250-300 mg, 1-200 mg, 2-200 mg, 5-200 mg, 10-200 mg, 20-200 mg, 25-200 mg, 30-200 mg, 40-200 mg, 50-200 mg, 60-200 mg, 70-200 mg, 75-200 mg, 80-200 mg, 90-200 mg, 100-200 mg, 125-200 mg, 150-200 mg, 100-1000 mg, 200-1000 mg, 400-1000 mg, 100-800 mg, 200-800 mg, and 400-800 mg.

In some embodiments, the pharmaceutical composition comprises the compound or a stereoisomer, deuterated substance, solvate, prodrug, metabolite, pharmaceutically acceptable salt or eutectic crystal thereof according to the present invention in an amount including but not limited to 1-1500 mg, 1-1400 mg, 1-1200 mg, 1-600 mg, 20-400 mg, 25-200 mg, 1 mg, 5 mg, 10 mg, 15 mg, 20 mg, 25 mg, 30 mg, 35 mg, 40 mg, 45 mg, 50 mg, 55 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 300 mg, 400 mg, 480 mg, and 500 mg.

There is provided a method for treating a disease in a mammal, the method comprising administering to a subject a therapeutically effective amount of the compound, or the stereoisomer, deuterated substance, solvate, prodrug, metabolite, pharmaceutically acceptable salt, or eutectic crystal thereof according to the present invention, wherein the therapeutically effective amount is preferably 1-1500 mg, and the disease is preferably tumors.

There is provided a method for treating a disease in a mammal, comprising administering the compound or a stereoisomer, deuterated substance, solvate, prodrug, metabolite, pharmaceutically acceptable salt or eutectic crystal thereof according to the present invention, as a drug, to a subject at a daily dose of 1-1500 mg/day, wherein the daily dose may be a single dose or divided doses. In some embodiments, the daily dose includes, but is not limited to, 10-1500 mg/day, 10-800 mg/day, 25-800 mg/day, 50-800 mg/day, 100-800 mg/day, 200-800 mg/day, 25-400 mg/day, 50-400 mg/day, 100-400 mg/day, and 200-400 mg/day, and in some embodiments, the daily dose includes, but is not limited to, 10 mg/day, 20 mg/day, 25 mg/day, 50 mg/day, 100 mg/day, 125 mg/day, 150 mg/day, 200 mg/day, 400 mg/day, 600 mg/day, 800 mg/day, 1000 mg/day, and 1200 mg/day.

The present invention relates to a kit, which can comprise a composition in a single dose or a multi-dose form. The kit comprises the compound or a stereoisomer, deuterated substance, solvate, prodrug, metabolite, pharmaceutically acceptable salt or eutectic crystal thereof according to the present invention, wherein the amount of the compound or a stereoisomer, deuterated substance, solvate, prodrug, metabolite, pharmaceutically acceptable salt or eutectic crystal thereof according to the present invention is the same as the amount thereof in the above-mentioned pharmaceutical composition.

In the present invention, the amount of the compound or a stereoisomer, deuterated substance, solvate, prodrug, metabolite, pharmaceutically acceptable salt or eutectic crystal thereof according to the present invention is calculated in the form of free base in each case.

### Synthesis Method I:

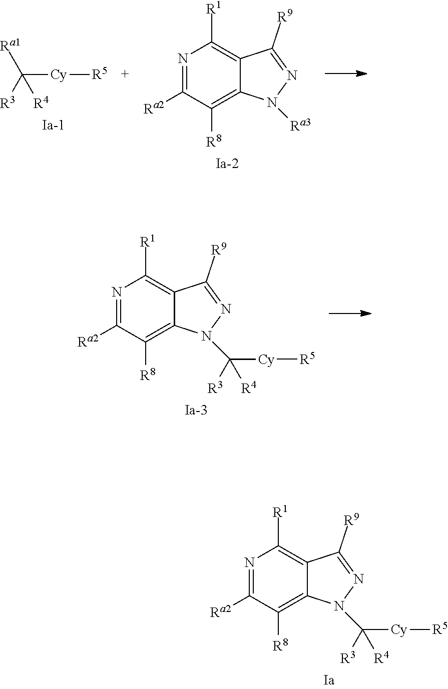

R^{a1} is selected from halogen or OH;
R^{a2} is selected from halogen, preferably from Br or Cl;
R^{a3} is selected from an amino protecting group or H;
the definitions of the remaining groups are the same as in the description;
the compound shown in general formula (Ia-1) and the compound shown in general formula (Ia-2) are subjected to a substitution reaction to obtain a compound shown in general formula (Ia-3), or the compound shown in general formula (Ia-3) is obtained by removing the amino protecting group from the compound shown in general formula (Ia-2) and then performing a substitution reaction by reacting same with the compound shown in general formula (Ia-1);
the compound shown in general formula (Ia-3) is subjected to a coupling reaction to obtain a compound shown in general formula (Ia).

Unless stated to the contrary, the terms used in the description and the claims have the following meanings.

The carbon, hydrogen, oxygen, sulfur, nitrogen or F, Cl, Br, I involved in the groups and compounds of the present invention all comprise their isotopes, and the carbon, hydrogen, oxygen, sulfur or nitrogen involved in the groups and compounds of the present invention is optionally further substituted with one or more of their corresponding isotopes, wherein the isotopes of carbon comprise ¹²C, ¹³C and ¹⁴C, the isotopes of hydrogen comprise protium (H), deuterium (D, also known as heavy hydrogen), tritium (T, also known as superheavy hydrogen), the isotopes of oxygen comprise ¹⁶O, ¹⁷O and ¹⁸O, the isotopes of sulfur comprise ³²S, ³³S, ³⁴S and ³⁶S, the isotopes of nitrogen comprise ¹⁴N and ¹⁵N, the isotopes of fluorine comprise ¹⁷F and ¹⁹F, the isotopes of chlorine comprise ³⁵Cl and ³⁷Cl, and the isotopes of bromine comprise ⁷⁹Br and ⁸¹Br.

“Halogen” refers to F, Cl, Br, or I.

“Halogen-substituted” refers to a substitution with F, Cl, Br, or I, including but not limited to a substitution with 1 to 10 substituents selected from F, Cl, Br, or I, or a substitution with 1 to 6 substituents selected from F, Cl, Br, or I, or a substitution with 1 to 4 substituents selected from F, Cl, Br, or I. “Halogen-substituted” is referred to simply as “halo”.

“Alkyl” refers to a substituted or unsubstituted linear or branched saturated aliphatic hydrocarbyl group, including but not limited to an alkyl group of 1 to 20 carbon atoms, an alkyl group of 1 to 8 carbon atoms, an alkyl group of 1 to 6 carbon atoms, or an alkyl group of 1 to 4 carbon atoms. Non-limiting examples include methyl, ethyl, n-propyl, isopropyl, n-butyl, sec-butyl, neobutyl, tert-butyl, n-pentyl, isoamyl, neopentyl, n-hexyl and various branched isomers thereof. The definition of the “alkyl” herein is consistent with this definition. Alkyl can be monovalent, divalent, trivalent or tetravalent.

“Alkylene” refers to a substituted or unsubstituted linear or branched divalent saturated hydrocarbyl group, including —(CH₂)ᵥ— (v is an integer from 1 to 10), and examples of alkylene include, but are not limited to, methylene, ethylene, propylene, butylene, etc.

“Cycloalkyl” refers to a substituted or unsubstituted saturated carbocyclic hydrocarbyl group, usually having from 3 to 10 carbon atoms, and non-limiting examples include cyclopropyl, cyclobutyl, cyclopentyl, cyclohexyl, cycloheptyl, etc. The “cycloalkyl” herein is as defined above. Cycloalkyl can be monovalent, divalent, trivalent or tetravalent.

“Heterocycloalkyl” refers to a substituted or unsubstituted saturated heteroatom-containing cyclic hydrocarbyl group, including but not limited to 3 to 10 atoms, 3 to 8 atoms, or 1 to 3 heteroatoms selected from N, O or S. N and S selectively substituted in the heterocycloalkyl ring can be oxidized to various oxidation states. Heterocycloalkyl can be connected to a heteroatom or a carbon atom; heterocycloalkyl can be connected to an aromatic ring or a non-aromatic ring; and heterocycloalkyl can be connected to a bridged ring or a spiro ring. Non-limiting examples include oxiranyl, azacyclopropyl, oxacyclobutyl, azetidinyl, tetrahydrofuryl, tetrahydro-2H-pyranyl, dioxolanyl, dioxanyl, pyrrolidyl, piperidyl, imidazolidinyl, oxazolidinyl, oxazinanyl, morpholinyl, hexahydropyrimidyl or piperazinyl. Heterocycloalkyl can be monovalent, divalent, trivalent or tetravalent.

“Alkenyl” refers to a substituted or unsubstituted linear or branched unsaturated hydrocarbyl group, having at least 1, usually 1, 2 or 3 carbon-carbon double bonds, with a main chain including but not limited to 2 to 10, 2 to 6, or 2 to 4 carbon atoms. Examples of alkenyl include, but are not limited to, vinyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene, 1,4-hexadiene, etc. The definition of the “alkenyl” herein is consistent with this definition. Alkenyl can be monovalent, divalent, trivalent or tetravalent.

“Alkynyl” refers to a substituted or unsubstituted linear or branched monovalent unsaturated hydrocarbyl group, having at least 1, usually 1, 2 or 3 carbon-carbon triple bonds, with a main chain including 2 to 10 carbon atoms, including but not limited to a main chain including 2 to 6 carbon atoms, or a main chain including 2 to 4 carbon atoms. Examples of alkynyl include, but are not limited to, ethynyl, propargyl, 1-propynyl, 2-propynyl, 1-butynyl, 2-butynyl, 3-butynyl, 1-pentynyl, 2-pentynyl, 3-pentynyl, 4-pentynyl, 1-methyl-1-butynyl, 2-methyl-1-butynyl, 2-methyl-3-butynyl, 1-hexynyl, 2-hexynyl, 3-hexynyl, 4-hexynyl, 5-hexynyl, 1-methyl-1-pentynyl, 2-methyl-1-pentynyl, 1-heptynyl, 2-heptynyl, 3-heptynyl, 4-heptynyl, 1-octynyl, 3-octynyl, 1-nonynyl, 3-nonynyl, 1-decynyl, 4-decynyl, etc. Alkynyl can be monovalent, divalent, trivalent or tetravalent.

“Alkoxy” refers to a substituted or unsubstituted —O-alkyl group. Non-limiting examples include methoxy, ethoxy, n-propoxy, isopropoxy, n-butoxy, sec-butoxy, tert-butoxy, n-pentoxy, n-hexyloxy, cyclopropoxy and cyclobutoxy.

“Carbocyclyl” or “carbocycle” refers to a substituted or unsubstituted saturated or unsaturated aromatic ring or non-aromatic ring, wherein the aromatic ring or non-aromatic ring can be a 3- to 8-membered monocyclic ring, a 4- to 12-membered bicyclic ring or a 10- to 15-membered tricyclic ring system. Carbocyclyl can be connected to an aromatic ring or a non-aromatic ring, wherein the aromatic ring or non-aromatic ring is optionally a monocyclic ring, a bridged ring or a spiro ring. Non-limiting examples include cyclopropane, cyclobutane, cyclopentane, cyclohexane, cycloheptane, 1-cyclopentyl-1-enyl, 1-cyclopentyl-2-enyl, 1-cyclopentyl-3-enyl, cyclohexyl, 1-cyclohexyl-2-enyl, 1-cyclohexyl-3-enyl, cyclohexenyl, a benzene ring, a naphthalene ring,

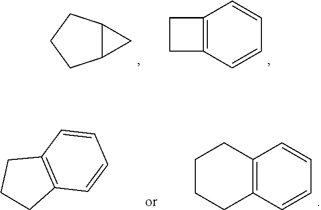

“Carbocyclyl” or “carbocycle” can be monovalent, divalent, trivalent or tetravalent.

“Heterocyclyl” or “heterocycle” refers to a substituted or unsubstituted saturated or unsaturated aromatic ring or non-aromatic ring, wherein the aromatic ring or non-aromatic ring can be a 3- to 8-membered monocyclic ring, a 4- to 12-membered bicyclic ring or a 10- to 15-membered tricyclic ring system, and contains one or more (including but not limited to 2, 3, 4 or 5) heteroatoms selected from N, O or S, and the selectively substituted N and S in the heterocyclyl ring can be oxidized to various oxidation states. Heterocyclyl can be connected to a heteroatom or a carbon atom; heterocyclyl can be connected to an aromatic ring or a non-aromatic ring; and heterocyclyl can be connected to a bridged ring or a spiro ring. Non-limiting examples include oxiranyl, azacyclopropyl, oxetanyl, azetidinyl, 1,3-dioxolanyl, 1,4-dioxolanyl, 1,3-dioxanyl, azacycloheptyl, pyridyl, furyl, thienyl, pyranyl, N-alkylpyrrolyl, pyrimidyl, pyrazinyl, pyridazinyl, imidazolyl, piperidyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, tetrahydrothiazolyl, tetrahydropyranyl, benzoimidazolyl, benzopyridyl, pyrrolopyridyl, benzodihydrofuryl, pyrrolyl, pyrazolyl, thiazolyl, oxazolyl, pyrazinyl, indazolyl, benzothienyl, benzofuryl, benzopyrrolyl, benzoimidazolyl, benzothiazolyl, benzoxazolyl, benzopyridyl, benzopyrimidyl, benzopyrazinyl, piperazinyl, azabicyclo[3.2.1]octanyl, azabicyclo[5.2.0]nonanyl, oxatricyclo[5.3.1.1]dodecyl, azaadamantyl, oxaspiro[3.3]heptanyl,

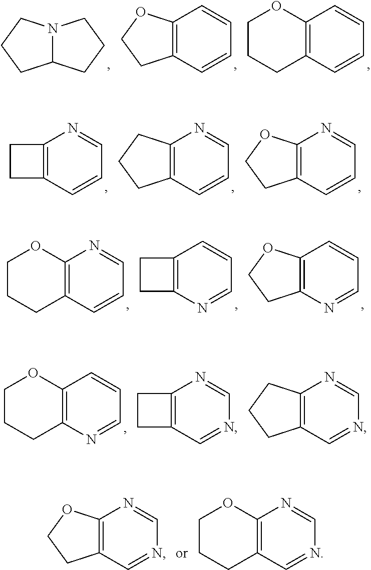

“Heterocyclyl” or “heterocycle” can be monovalent, divalent, trivalent or tetravalent.

“Spiro ring” or “spiro ring group” refers to a polycyclic group that shares one atom (called a spiro atom) between substituted or unsubstituted monocyclic rings. The number of ring atoms in the spiro ring system includes but is not limited to 5 to 20, 6 to 14, 6 to 12, or 6 to 10, wherein one or more rings may contain 0 or more (including but not limited to 1, 2, 3 or 4) double bonds, and can optionally contain 0 to 5 heteroatoms selected from N, O or S(═O)ₙ.

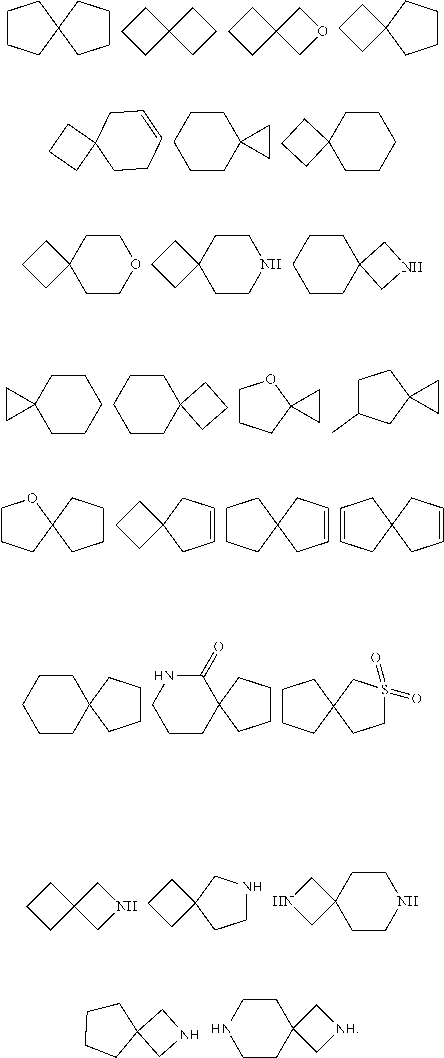

“Spiro ring” or “spiro ring group” can be monovalent, divalent, trivalent or tetravalent.

“Fused ring” or “fused ring group” refers to a polycyclic group in which each ring in the system shares an adjacent pair of atoms with other rings in the system, wherein one or more rings may contain 0 or more (including but not limited to 1, 2, 3 or 4) double bonds, and may be substituted or unsubstituted, and each ring in the fused ring system may contain 0 to 5 heteroatoms or groups containing heteroatoms (including but not limited to N, S(═O)ₙ or O, wherein n is 0, 1 or 2). The number of ring atoms in the fused ring system includes but is not limited to 5 to 20, 5 to 14, 5 to 12, or 5 to 10. Non-limiting examples include:

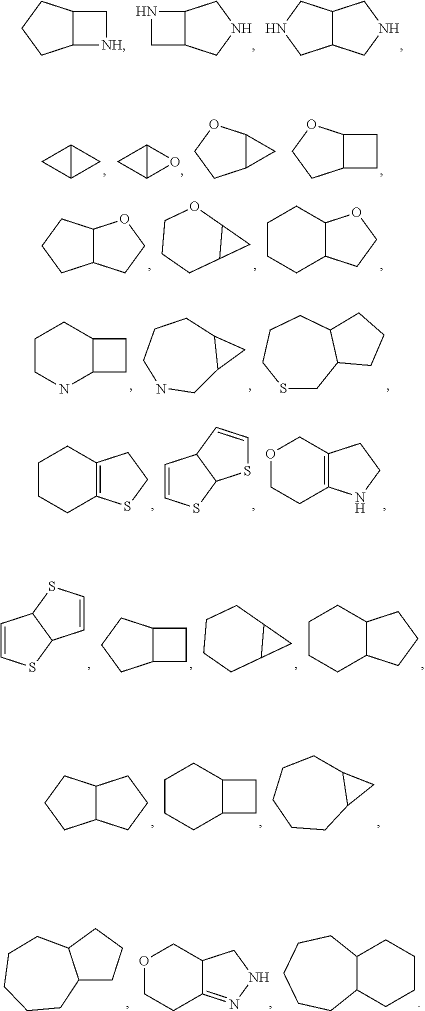

“Fused ring” or “fused ring group” can be monovalent, divalent, trivalent or tetravalent.

“Bridged ring” or “bridged ring group” refers to a substituted or unsubstituted polycyclic group containing any two atoms that are not directly connected, and may contain 0 or more double bonds. Any ring in the fused ring system may contain 0 to 5 groups selected from heteroatoms or groups containing heteroatoms (including but not limited to N, S(═O)n or O, wherein n is 0, 1 or 2). The number of ring atoms includes but is not limited to 5 to 20, 5 to 14, 5 to 12 or 5 to 10. Non-limiting examples include

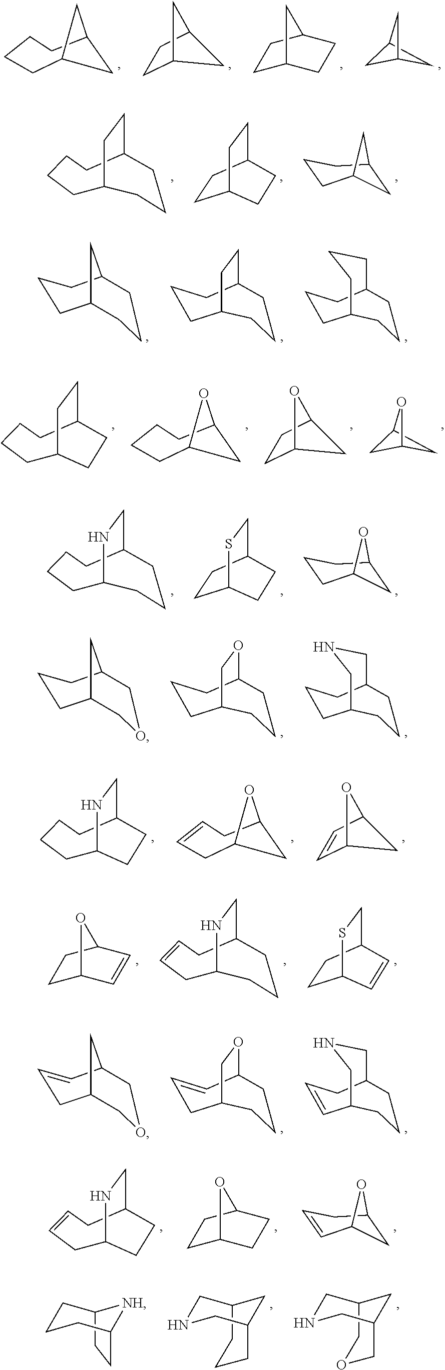

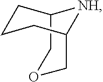

cubane and adamantane. “Bridged ring” or “bridged ring group” can be monovalent, divalent, trivalent or tetravalent.

“Carbospiro ring”, “spiro ring carbocyclyl”, “spirocarbocyclyl” or “carbospiro ring group” refers to a “spiro ring” with a ring system consisting only of carbon atoms. The definition of the “carbospiro ring”, “spiro ring carbocyclyl”, “spirocarbocyclyl” or “carbospiro ring group” herein is consistent with that of a spiro ring.

“Carbo-fused ring”, “fused ring carbocyclyl”, “fused carbocyclyl” or “carbo-fused ring group” refers to a “fused ring” with a ring system consisting only of carbon atoms. The definition of the “carbo-fused ring”, “fused ring carbocyclyl”, “fused carbocyclyl” or “carbo-fused ring group” herein is consistent with that of a fused ring.

“Carbo-bridged ring”, “bridged ring carbocyclyl”, “bridged carbocyclyl” or “carbo-bridged ring group” refers to a “bridged ring” with a ring system consisting only of carbon atoms. The definition of the “carbo-bridged ring”, “bridged ring carbocyclyl”, “bridged carbocyclyl” or “carbo-bridged ring group” herein is consistent with that of a bridged ring.

“Mono-heterocyclic ring”, “monocyclic heterocyclyl” or “mono-heterocyclic ring group” refers to “heterocyclyl” or “heterocycle” with a monocyclic system. The definition of the “heterocyclyl”, “monocyclic heterocyclyl” or “mono-heterocyclic ring group” herein is consistent with that of heterocycle.

“Fused-heterocyclic ring”, “fused-heterocyclic ring group”, “fused ring heterocyclyl” or “fused-heterocyclic ring group” refers to a “fused ring” containing a heteroatom. The definition of the “fused-heterocyclic ring”, “fused-heterocyclic ring group”, “fused ring heterocyclyl” or “fused-heterocyclic ring group” herein is consistent with that of a fused ring.

“Spiro-heterocyclic ring”, “spiro-heterocyclic ring group”, “spiro ring heterocyclyl” or “spiro-heterocyclic ring group” refers to a “spiro ring” containing a heteroatom. The definition of the “spiro-heterocyclic ring”, “spiro-heterocyclic ring group”, “spiro ring heterocyclyl” or “spiro-heterocyclic ring group” herein is consistent with that of a spiro ring.

“Bridged-heterocyclic ring”, “bridged-heterocyclic ring group”, “bridged ring heterocyclyl” or “bridged-heterocyclic ring group” refers to a “bridged ring” containing a heteroatom. The definition of the “bridged-heterocyclic ring”, “bridged-heterocyclic ring group”, “bridged ring heterocyclyl” or “bridged-heterocyclic ring group” herein is consistent with that of a bridged ring.

“Aryl” or “aromatic ring” refers to a substituted or unsubstituted aromatic hydrocarbyl group with a monocyclic ring or a fused ring, wherein the number of ring atoms in the aromatic ring includes but is not limited to 6 to 18, 6 to 12 or 6 to 10 carbon atoms. The aryl ring can be fused to a saturated or unsaturated carbocycle or heterocycle, wherein the ring connected to the parent structure is an aryl ring. Non-limiting examples include a benzene ring, a naphthalene ring, or

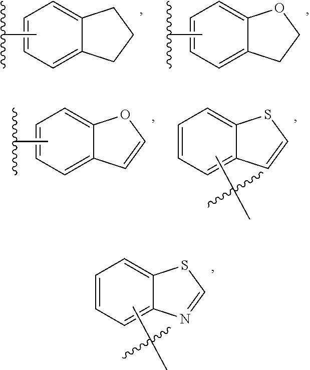

and “aryl” or “aromatic ring” may be monovalent, divalent, trivalent or tetravalent. When divalent, trivalent or tetravalent, the point of connection is on the aryl ring.

“Heteroaryl” or “heteroaromatic ring” refers to a substituted or unsubstituted aromatic hydrocarbyl group containing 1 to 5 heteroatoms or groups containing heteroatoms (including but not limited to N, O or S(═O)n, wherein n is 0, 1 or 2), wherein the number of ring atoms in the heteroaromatic ring includes but is not limited to 5-15, 5-10 or 5-6. Non-limiting examples of heteroaryl include, but are not limited to pyridyl, furyl, thienyl, pyridyl, pyranyl, N-alkylpyrrolyl, pyrimidyl, pyrazinyl, pyridazinyl, imidazolyl, benzopyrazole, benzoimidazole, benzopyridine, pyrrolopyridine, etc. The heteroaryl ring may be fused to a saturated or unsaturated carbocycle or heterocycle, wherein the ring connected to the parent structure is a heteroaryl ring. Non-limiting examples include

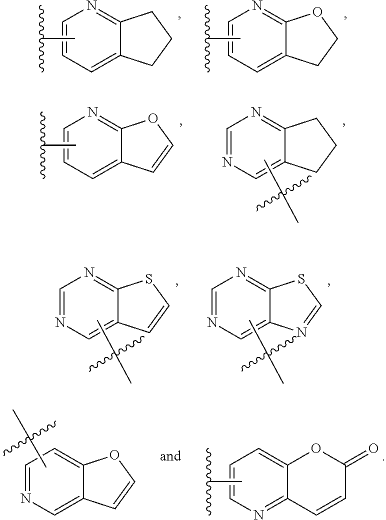

The definition of the “heteroaryl” herein is consistent with this definition. Heteroaryl can be monovalent, divalent, trivalent or tetravalent. When divalent, trivalent or tetravalent, the point of connection is on the heteroaryl ring.

“Substitution” or “substituted” refers to a substitution with 1 or more (including but not limited to 2, 3, 4 or 5) substituents including but not limited to H, F, Cl, Br, I, alkyl, cycloalkyl, alkoxy, haloalkyl, mercaptan, hydroxyl, nitro, mercapto, amino, cyano, isocyano, aryl, heteroaryl, heterocyclyl, bridged ring group, spiro ring group, fused ring group, hydroxyalkyl, ═O, carbonyl, aldehyde, carboxylic acid, carboxylate, —(CH₂)ₘ—C(═O)—R^{a}, —O—(CH₂)ₘ—C(═O)—R^{a}, —(CH₂)ₘ—C(═O)—NR^{b}R^{c}, —(CH₂)ₘS(═O)ₙR^{a}, —(CH₂)ₘ-alkenyl-R^{a}, OR^{d} or —(CH₂)ₘ-alkynyl-R^{a} (wherein m and n are 0, 1 or 2), arylthio, thiocarbonyl, silyl or —NR^{b}R^{c} and the like, wherein R^{b} and R^{c} are independently selected from H, hydroxyl, amino, carbonyl, alkyl, alkoxy, cycloalkyl, heterocyclyl, aryl, heteroaryl, sulfonyl, or trifluoromethylsulfonyl. Alternatively, R^{b} and R^{c} may form a five- or six-membered cycloalkyl or heterocyclyl; each R^{a} or R^{d} is independently selected from aryl, heteroaryl, alkyl, alkoxy, cycloalkyl, heterocyclyl, carbonyl, ester group, bridged ring group, spiro ring group or fused ring group.

“Containing 1 to 4 heteroatoms selected from O, S, and N” refers to containing 1, 2, 3, or 4 heteroatoms selected from O, S, and N.

“Substituted with 0 to X substituents” refers to substituted with 0, 1, 2, 3 . . . X substituents, wherein X is selected from any integer between 1 and 10. For example, “substituted with 0 to 4 substituents” refers to substituted with 0, 1, 2, 3 or 4 substituents. For example, “substituted with 0 to 5 substituents” refers to substituted with 0, 1, 2, 3, 4 or 5 substituents. For example, “bridged-heterocyclic ring is optionally further substituted with 0 to 4 substituents selected from H or F” means that the bridged-heterocyclic ring is optionally further substituted with 0, 1, 2, 3 or 4 substituents selected from H or F.

An X- to Y-membered ring (X is selected from an integer less than Y and greater than or equal to 3, and Y is selected from any integer between 4 and 12) includes X-, X+1-, X+2-, X+3-, X+4-, . . . , to Y-membered rings. Rings include heterocycle, carbocycle, an aromatic ring, aryl, heteroaryl, cycloalkyl, a mono-heterocyclic ring, a fused-heterocyclic ring, a spiro-heterocyclic ring or a bridged-heterocyclic ring. For example, a “4- to 7-membered mono-heterocyclic ring” refers to a 4-, 5-, 6- or 7-membered mono-heterocyclic ring, and a “5- to 10-membered fused-heterocyclic ring” refers to a 5-, 6-, 7-, 8-, 9- or 10-membered fused-heterocyclic ring.

The term “optional” or “optionally” refers to that the events or circumstances subsequently described may but not necessarily occur, and the description includes the occasions where the events or circumstances occur or do not occur. For example, “alkyl optionally substituted with F” means that the alkyl may but not necessarily be substituted with F, and the description includes the case where the alkyl is substituted with F and the case where the alkyl is not substituted with F.

The term “stereoisomer” refers to an isomer produced as a result of different spatial arrangement of atoms in molecules, including cis-trans isomers, enantiomers and conformational isomers.

The term “tautomer” refers to a functional group isomer produced by the rapid movement of an atom in two positions in a molecule, such as keto-enol isomerization and amide-imino alcohol isomerization.

The term “preparation specification” refers to the weight of the active drug contained in each vial, tablet or other unit preparation.

The term “subject” refers to a human or another mammal.

### DETAILED DESCRIPTION

The technical solutions of the present invention will be described in detail by the following examples, but the scope of protection of the present invention includes but is not limited thereto.

The structures of the compounds are determined by nuclear magnetic resonance (NMR) or (and) mass spectrometry (MS). The NMR shift (δ) is given in the unit of 10-6 (ppm). NMR is determined with Bruker Avance III 400 and Bruker Avance 300; the solvent for determination is deuterated dimethyl sulfoxide (DMSO-d6), deuterated chloroform (CDCl3) and deuterated methanol (CD3OD); and the internal standard is tetramethylsilane (TMS);
MS is determined with Agilent 6120B (ESI) and Agilent 6120B (APCI);
HPLC is determined with Agilent 1260DAD high pressure liquid chromatograph (Zorbax SB-C18 100×4.6 mm, 3.5 μM);
Yantai Huanghai HSGF254 or Qingdao GF254 silica gel plate is used as a thin layer chromatography silica plate, and the silica gel plate for the thin layer chromatography (TLC) is of the specification of 0.15 mm-0.20 mm, and the specification when separating and purifying a product by thin layer chromatography is 0.4 mm-0.5 mm;
and for the column chromatography, Yantai Huanghai silica gel of 200-300 mesh silica gel is generally used as a carrier.

The known starting materials of the present invention can be synthesized by or according to methods known in the art, or can be purchased from Titan Technology Co., Ltd., Energy Chemical Co., Ltd., Shanghai Demo Co., Ltd., Chengdu Kelong Chemical Co., Ltd., Accela ChemBio Co., Ltd., J&K Scientific Co., Ltd. and other companies.

Tf: trifluoromethylsulfonyl; Ts: p-toluenesulfonyl; TMS: trimethylsilyl; SEM:

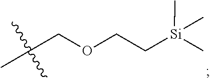 [SMILES: *COCC[Si](C)(C)C]

### THP:

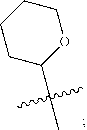 [SMILES: *C1CCCCO1]

Boc: tert-butoxycarbonyl; Ms:

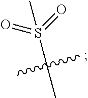 [SMILES: *S(C)(=O)=O]

### TBS:

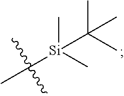 [SMILES: *[Si](C)(C)C(C)(C)C]

MTBE: methyl tert-butyl ether; Bn: benzyl; DIPEA: N,N-diisopropylethylamine; DMAc: N,N-dimethylacetamide; DMF: N,N-dimethylformamide; DMSO: dimethyl sulfoxide; DCM: dichloromethane; Cbz:

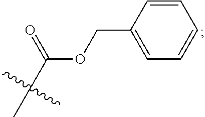 [SMILES: *C(=O)OCc1ccccc1]

LDA: lithium diisopropylamide;
Pd(dppf)Cl₂·CH₂Cl₂: CAS 95464-05-4; XPhos Pd G2: CAS 1310584-14-5

### Example 1

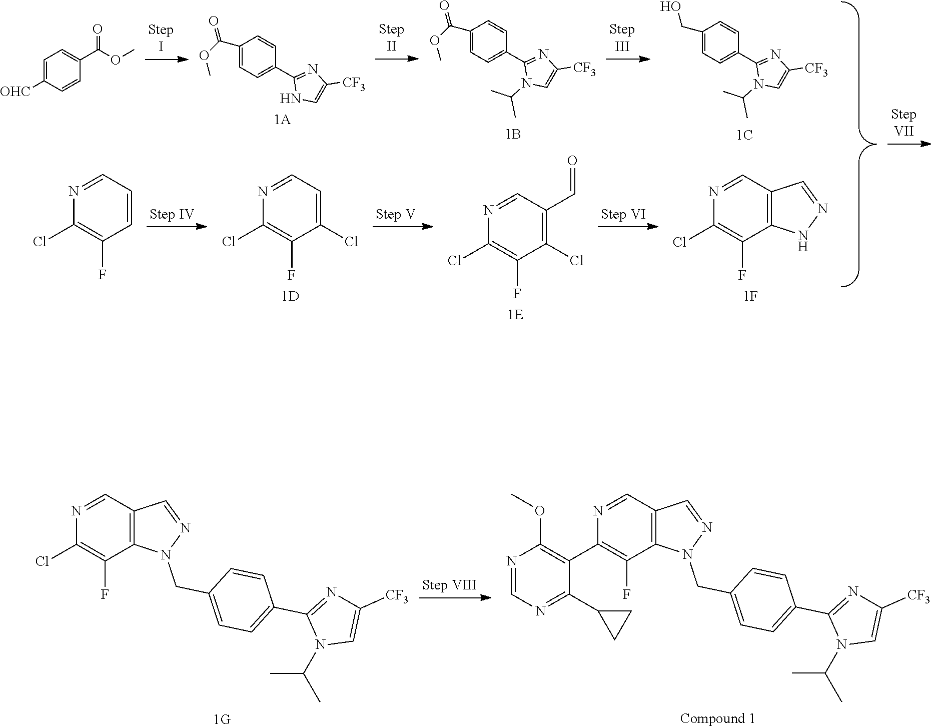

### Step I: Preparation of 1A

Sodium acetate (4.15 g, 50.55 mmol) was added to a solution of 1,1-dibromo-3,3,3-trifluoroacetone (12.4 g, 45.95 mmol) in water (24 mL), heated at 100° C. for 1 h, and cooled to room temperature, a solution of methyl 4-formylbenzoate (8.3 g, 50.55 mmol) in methanol (180 mL) and aqueous ammonia (44 mL) was added, and the mixture was stirred at room temperature for 40 minutes, reacted at 100° C. for 2 h, cooled to room temperature, quenched by adding water (50 mL), and extracted with ethyl acetate (60 mL×3). The organic phases were combined, dried over anhydrous sodium sulfate and then concentrated under reduced pressure, and the residue was purified by silica gel column chromatography (ethyl acetate/petroleum ether (V/V)=1/10-1/1) to obtain 1A (8 g, yield: 64.43%).

LCMS m/z=271.1 [M+H]⁺.

### Step II: Preparation of 1B

1A (17 g, 62.91 mmol) was dissolved in 200 mL of DMF, isopropyl iodide (32.08 g, 188.73 mmol) and cesium carbonate (61.49 g, 188.75 mmol) were added in sequence, and the mixture was reacted at 90° C. overnight. The reaction product was cooled to room temperature and extracted by adding water and ethyl acetate, the organic layer was washed with an aqueous saturated sodium chloride solution, dried over anhydrous sodium sulfate, and concentrated under reduced pressure, and the residue was purified by silica gel column chromatography (ethyl acetate/petroleum ether (V/V)=1/20-1/1) to obtain 1B (11.8 g, yield: 60%).

LCMS m/z=313.1 [M+H]⁺.

### Step III: Preparation of 1C

1B (11.8 g, 37.79 mmol) was dissolved in 150 mL of THF, lithium borohydride (4.12 g, 188.95 mmol) was added, and the mixture was heated to 50° C. and stirred for 2 h. After cooling to room temperature, an aqueous saturated ammonium chloride solution was added in an ice-water bath to quench the reaction, the reaction product was extracted by adding water and ethyl acetate, and the organic layer was dried over anhydrous sodium sulfate, concentrated under reduced pressure to obtain 1C (10 g).

LCMS m/z=285.1 [M+H]⁺.

### Step IV: Preparation of 1D

2-Chloro-3-fluoropyridine (5 g, 38.03 mmol) was dissolved in THF (25 mL), the mixture was cooled to −70° C. under nitrogen protection, and a THF solution of lithium bis(trimethylsilyl)amide (57 mL, 1N, 57 mmol) was dropwise added slowly; after the addition was complete, the mixture was heated to −30° C. and stirred for 1 h, hexachloroethane (10.80 g, 45.64 mmol) was added at −30° C., and after the addition was complete, the mixture was naturally heated to room temperature and stirred overnight. Sequentially, 20 mL of an aqueous saturated ammonium chloride solution was added to quench the reaction, 100 mL of water and 100 mL of ethyl acetate were added, and after layering, the organic layer was washed with 100 mL of an aqueous saturated sodium chloride solution, dried over anhydrous sodium sulfate, and concentrated under reduced pressure. The residue was purified by silica gel column chromatography (dichloromethane/petroleum ether (V/V)=0/1-1/4) to obtain 1D (2 g, yield 32%).

¹H NMR (400 MHz, CDCl₃) δ 8.13 (d, 1H), 7.34 (t, 1H).

### Step V: Preparation of 1E

1D (2 g, 12.02 mmol) was dissolved in THF (10 mL), the mixture was cooled to −70° C. under nitrogen protection, and a THF solution of LDA (14.4 mL, 1N, 14.42 mmol) was dropwise added slowly at −70° C. The mixture was stirred at −70° C. for 1 h, DMF (1.76 g, 24.04 mmol) was then dropwise added slowly, and the mixture was stirred for 30 min. 10 mL of an aqueous saturated ammonium chloride solution was added to quench the reaction, the mixture was heated to room temperature, 20 mL of water and 20 mL of ethyl acetate were added, and after layering, the organic layer was washed with 20 mL of an aqueous saturated sodium chloride solution, dried over anhydrous sodium sulfate, and concentrated under reduced pressure. The residue was purified by silica gel column chromatography (ethyl acetate/petroleum ether (V/V)=0/1-1/4) to obtain 1E (0.56 g, yield: 24%).

¹H NMR (400 MHz, CDCl₃) δ 10.40 (s, 1H), 8.69 (s, 1H).

### Step VI: Preparation of 1F

1E (0.55 g, 2.84 mmol) was dissolved in n-butanol (10 mL), and hydrazine hydrate (0.89 g, 14.2 mmol, wt %=80%) was added and stirred at 100° C. for 8 h. After cooling to room temperature, 20 mL of water and 20 mL of ethyl acetate were added, and after layering, the organic phase was dried over anhydrous sodium sulfate, and concentrated under reduced pressure. The residue was purified by silica gel column chromatography (ethyl acetate/petroleum ether (V/V)=1/10-1/2) to obtain 1F (0.28 g, yield: 57%)

LCMS m/z=172.1 [M+H]⁺.

### Step VII: Preparation of 1G

1F (0.09 g, 0.52 mmol), 1C (0.15 g, 0.52 mmol) and tri-n-butylphosphine (0.21 g, 1.04 mmol) were added to THF (5 mL) in sequence, and diethyl azodicarboxylate (0.18 g, 1.03 mmol) was added dropwise under nitrogen protection at room temperature and stirred at room temperature for 1 h. The solvent was removed by concentration under reduced pressure, and the residue was purified by silica gel column chromatography (ethyl acetate/petroleum ether (V/V)=1/10-1/2) to obtain 1G (0.15 g, yield: 66%).

LCMS m/z=438.1 [M+H]⁺.

### Step VIII: Preparation of Compound 1

1G (0.13 g, 0.30 mmol) was dissolved in a mixed solvent of 1,4-dioxane (4 mL) and water (1 mL), and (4-cyclopropyl-6-methoxypyrimidin-5-yl)boronic acid (0.12 g, 0.62 mmol), potassium phosphate (0.13 g, 0.60 mmol), and XPhos G3 (CAS: 1445085-55-1, 0.025 g, 0.030 mmol) were added in sequence and heated to 90° C. under nitrogen protection and stirred overnight. After cooling to room temperature, 10 mL of ethyl acetate and 10 mL of water were added, and after layering, the organic layer was dried over anhydrous sodium sulfate and concentrated under reduced pressure. The residue was purified by silica gel column chromatography (ethyl acetate/petroleum ether (V/V)=1/5-1/1) to obtain Compound 1 (80 mg, yield: 48%).

LCMS m/z=552.2 [M+H]⁺.

¹H NMR (400 MHz, DMSO-d₆) δ 9.08 (d, 1H), 8.71 (s, 1H), 8.54 (d, 1H), 8.18-8.13 (m, 1H), 7.60-7.50 (m, 2H), 7.41-7.33 (m, 2H), 5.84 (s, 2H), 4.50-4.32 (m, 1H), 3.83 (s, 3H), 1.74-1.64 (m, 1H), 1.38 (d, 6H), 1.15-1.06 (m, 1H), 1.04-0.96 (m, 1H), 0.94-0.79 (m, 2H).

### Example 2

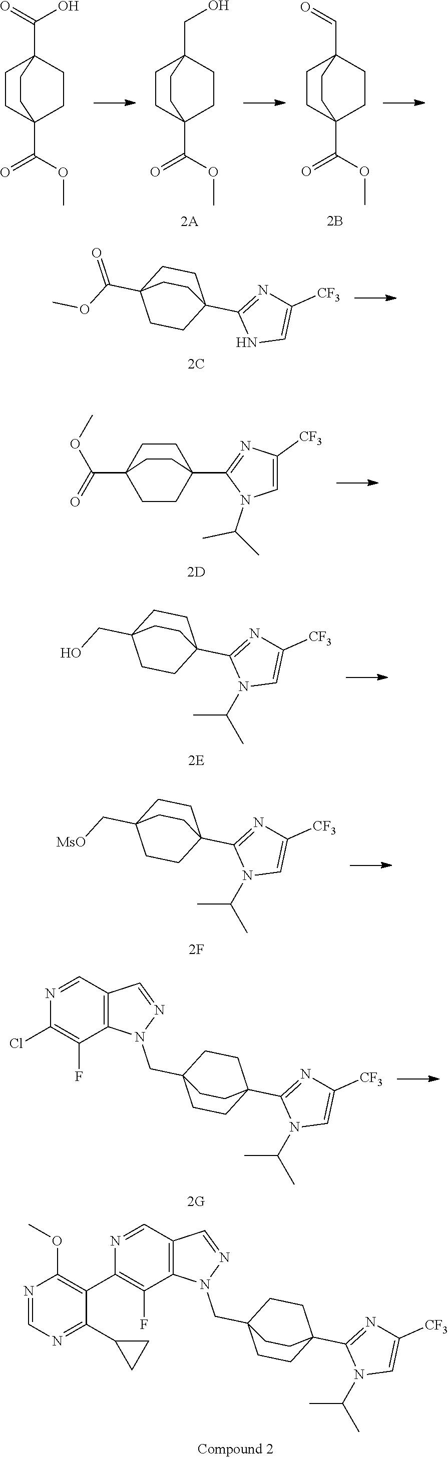

### Step I: Preparation of 2A

4-(Methoxycarbonyl)bicyclo[2.2.2]octane-1-carboxylic acid (5 g, 23.58 mmol) was dissolved in THF (60 mL), and a borane tetrahydrofuran solution (47 mL, 1M) was dropwise added slowly at 0° C. and stirred at room temperature overnight. Methanol was added at 0° C. for quenching, the resulting product was concentrated under reduced pressure, and the residue was purified by silica gel column chromatography (ethyl acetate/petroleum ether (V/V)=1/20-1/2) to obtain 2A (3.8 g, yield: 81%).

LCMS m/z=199.2 [M+H]⁺.

### Step II: Preparation of 2B

2A (3.8 g, 19.16 mmol) was dissolved in DCM (100 mL), and Dess-Martin periodinane (16.25 g, 38.32 mmol) was added at 0° C. in portions, and reacted at room temperature for 3 h. TLC monitoring showed that the reaction was complete. The resulting product was concentrated under reduced pressure, and the residue was purified by silica gel column chromatography (ethyl acetate/petroleum ether (V/V)=1/20-1/2) to obtain 2B (3.02 g, yield: 80%).

LCMS m/z=197.1 [M+H]⁺.

### Step III: Preparation of 2C

1,1-Dibromo-3,3,3-trifluoroacetone (4.98 g, 18.49 mmol, CAS: 431-67-4) was placed in a 250 mL single-mouth flask, and 30 mL of water and sodium acetate (1.89 g, 23.09 mmol) were added and reacted at 90° C. for 1 h. The resulting product was cooled to room temperature, and 60 mL of methanol, 2B (3.02 g, 15.39 mmol), and concentrated aqueous ammonia (15 mL) were added in sequence and reacted at 90° C. for 2 h. The resulting product was cooled to room temperature, the reaction liquid was poured into 200 mL of water and filtered, the filter cake was washed with water (2×10 mL), and the filter cake was dried under reduced pressure to obtain 2C (2.9 g, yield: 62%).

LCMS m/z=303.1 [M+H]⁺.

¹H NMR (400 MHz, DMSO-d₆) δ 12.36-12.16 (m, 1H), 7.68-7.55 (m, 1H), 3.59 (s, 3H), 1.90-1.72 (m, 12H).

### Step IV: Preparation of 2D

2C (2.0 g, 6.62 mmol) was dissolved in 30 mL of DMF at room temperature, isopropyl iodide (4.50 g, 26.48 mmol) and cesium carbonate (6.47 g, 19.86 mmol) were added in sequence, and the mixture was reacted at 90° C. overnight. The reaction product was cooled to room temperature and extracted by adding water and ethyl acetate, the organic layer was washed with an aqueous saturated sodium chloride solution, dried over anhydrous sodium sulfate, and concentrated under reduced pressure, and the residue was purified by silica gel column chromatography (ethyl acetate/petroleum ether (V/V)=1/20-1/1) to obtain 2D (1.2 g, yield: 52%).

LCMS m/z=345.2 [M+H]⁺.

### Step V: Preparation of 2E

2D (1.2 g, 3.48 mmol) was dissolved in 20 mL of THF, lithium borohydride (0.38 g, 17.40 mmol) was added, and the mixture was stirred at 60° C. for 2 h. After cooling to room temperature, an aqueous saturated ammonium chloride solution was added to quench the reaction, the reaction product was extracted by adding water and ethyl acetate, and the organic layer was dried over anhydrous sodium sulfate, concentrated under reduced pressure to obtain 2E (0.9 g, yield: 81%).

LCMS m/z=317.2 [M+H]⁺.

### Step VI: Preparation of 2F

At 0° C., methylsufonyl chloride (64 mg, 0.56 mmol) was added to a solution of 2E (0.12 g, 0.37 mmol) and triethylamine (0.11 g, 1.09 mmol) in dichloromethane (15 mL), which was stirred at room temperature for 1 h. 20 mL of an aqueous saturated sodium bicarbonate solution was added for quenching, and dichloromethane (20 mL×3) was used for extraction. The organic phases were combined, dried over anhydrous sodium sulfate, concentrated under reduced pressure, and purified by silica gel column chromatography (ethyl acetate/petroleum ether (V/V)=1/100-1/20) to obtain 2F (0.11 g, yield 74%).

LCMS m/z=395.1 [M+H]⁺.

### Step VII: Preparation of 2G

1F (0.1 g, 0.58 mmol) and 2F (0.23 g, 0.58 mmol) were dissolved in DMF (5 mL), and cesium carbonate (0.38 g, 1.17 mmol) and sodium iodide (0.087 g, 0.58 mmol) were added and reacted at 140° C. for 2 h. The reaction product was cooled to room temperature, 20 mL of water and 20 mL of ethyl acetate were added for quenching, the organic layer was washed with an aqueous saturated sodium chloride solution (10 mL×3), dried over anhydrous sodium sulfate, and concentrated under reduced pressure, and the residue was purified by silica gel column chromatography (ethyl acetate/petroleum ether (V/V)=1/10-1/2) to obtain 2G (0.060 g, yield: 22%).

LCMS m/z=470.2 [M+H]⁺.

### Step VIII: Preparation of Compound 2

2G (0.06 g, 0.13 mmol), (4-cyclopropyl-6-methoxypyrimidin-5-yl)boronic acid (0.050 g, 0.26 mmol), XPhos G3 (0.011 g, 0.013 mmol, CAS: 1445085-55-1), and potassium phosphate (0.056 g, 0.27 mmol) were placed in a 50 mL single-mouth flask, 1,4-dioxane (4 mL) and water (1 mL) were added, and after displacement with nitrogen three times, the mixture was heated to 90° C. and stirred overnight. After cooling to room temperature, 20 mL of water and 20 mL of ethyl acetate were added for quenching, the organic layer was dried over anhydrous sodium sulfate and then concentrated under reduced pressure, the residue was purified by silica gel column chromatography (ethyl acetate/petroleum ether (V/V)=1/10-1/1), and after concentration, the residue was purified by secondary silica gel column chromatography (dichloromethane/methanol (V/V)=100/0-100/3) to obtain Compound 2 (30 mg, yield: 39%).

LCMS m/z=584.2 [M+H]⁺.

¹H NMR (400 MHz, CD₃OD) δ 8.97 (d, 1H), 8.64 (s, 1H), 8.38 (d, 1H), 7.65-7.58 (m, 1H), 4.93-4.84 (m, 1H), 4.39 (s, 2H), 3.93 (s, 3H), 2.06-1.94 (m, 6H), 1.74-1.59 (m, 7H), 1.41 (d, 6H), 1.27-1.20 (m, 1H), 1.16-1.09 (m, 1H), 0.99-0.86 (m, 2H).

### Example 3

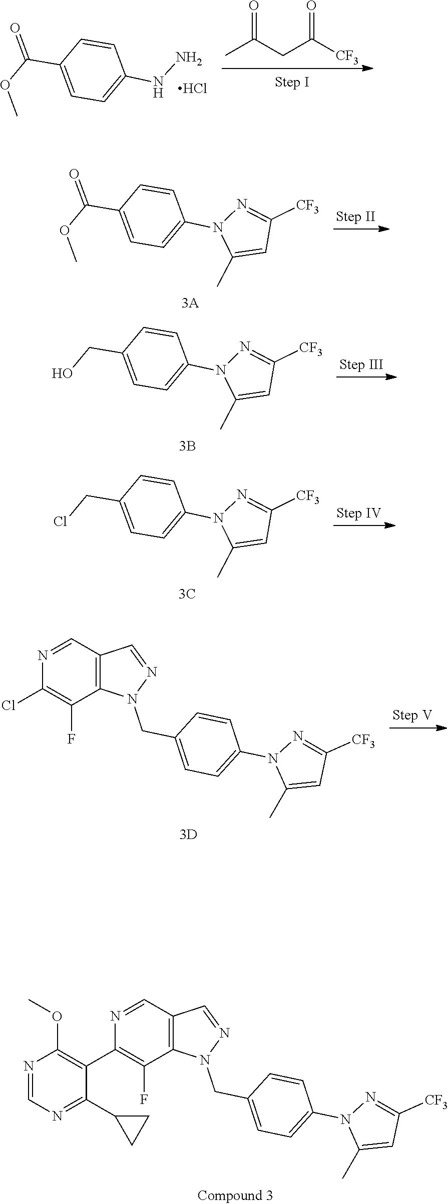

### Step I: Preparation of 3A

Methyl 4-hydrazinobenzoate hydrochloride (2.02 g, 10.0 mmol) was dissolved in hexafluoroisopropanol (10 mL), 1,1,1-trifluoro-2,4-pentanedione (1.54 g, 10.0 mmol) was added, triethylamine (2.23 g, 22.00 mmol) was dropwise added slowly under nitrogen protection in an ice bath, and the mixture was reacted at room temperature overnight. The reaction was quenched by adding water (50 mL) and extracted by adding ethyl acetate (60 mL×3), the organic phases were combined, dried over anhydrous sodium sulfate and then concentrated under reduced pressure, and the residue was purified by silica gel column chromatography (ethyl acetate/petroleum ether (V/V)=1/10-1/5) to obtain 3A (2.4 g, yield: 84%).

LCMS m/z=285.1 [M+H]⁺.

### Step II: Preparation of 3B

3A (2.4 g, 8.44 mmol) was dissolved in 50 mL of THF, and lithium borohydride (1.84 g, 84.4 mmol) was added slowly in an ice-water bath and stirred at 50° C. for 4 h. After the reaction was complete, the reaction product was cooled to room temperature, the reaction flask was placed in an ice-water bath, the reaction was quenched by slowly adding an aqueous saturated ammonium chloride solution, 20 mL of water was added, ethyl acetate (60 mL×3) was added for extraction, the organic layers were collected, dried over anhydrous sodium sulfate, and filtered, and the filtrate was concentrated under reduced pressure to obtain 3B (2.0 g, yield: 92%).

LCMS m/z=257.2 [M+H]⁺.

### Step III: Preparation of 3C

Thionyl dichloride (8 mL, 110.28 mmol) was added to a solution of 3B (1.0 g, 3.9 mmol) in dichloromethane (10 mL) and stirred at room temperature for 5 h, the mixture was then concentrated under reduced pressure, and an aqueous saturated sodium bicarbonate solution and ethyl acetate were added to the resulting crude product; after layering, the organic phase was collected, dried over anhydrous sodium sulfate, and filtered, and the filtrate was concentrated under reduced pressure to obtain 3C (1.0 g, yield: 93%).

LCMS m/z=275.0 [M+H]⁺.

### Step IV: Preparation of 3D

3C (106 mg, 0.39 mmol) and 1F (60 mg, 0.35 mmol) were dissolved in a solution of N,N-dimethylformamide (2 mL), and cesium carbonate (342 mg, 1.05 mmol) was added and stirred at room temperature overnight. After the reaction was complete, the reaction product was filtered by diatomaceous earth to remove the residue, the filtrate was diluted by adding 10 mL of ethyl acetate and washed by adding water (10 mL×3), the organic phases were collected and concentrated under reduced pressure, the residue was purified by silica gel column chromatography (ethyl acetate/petroleum ether (V/V)=0/1-1/1) to obtain 3D (65 mg, yield 45%).

LCMS m/z=410.1 [M+H]⁺.

Step V: Preparation of Compound 3 trifluoroacetate

3D (65 mg, 0.16 mmol) was dissolved in a mixed solvent of 1,4-dioxane (10 mL) and water (1 mL), and (4-cyclopropyl-6-methoxypyrimidin-5-yl)boronic acid (46.6 mg, 0.24 mmol), potassium phosphate (101.9 mg, 0.48 mmol), and XPhos G3 (CAS: 1445085-55-1, 13.5 mg, 0.016 mmol) were added in sequence and heated to 90° C. under nitrogen protection and stirred overnight. After the reaction was complete, a large amount of solvent was removed by concentration, 10 mL of water was added thereto, and ethyl acetate (10 mL×3) was added for extraction. The organic phases were combined, dried over anhydrous sodium sulfate, and concentrated under reduced pressure, the crude product was purified by preparative HPLC (instrument: waters 2767 preparative liquid chromatographic instrument; chromatographic column: XBridge @Prep C18 (30 mm×150 mm); composition of mobile phase: mobile phase A: acetonitrile, mobile phase B: water (containing 0.1% trifluoroacetic acid)), and the preparation liquid was collected and freeze-dried to obtain Compound 3 trifluoroacetate (10 mg, yield: 12%).

LCMS m/z=524.2 [M+H]⁺.

¹H NMR (400 MHz, DMSO-d₆) δ 9.08 (d, 1H), 8.71 (s, 1H), 8.55 (d, 1H), 7.60-7.53 (m, 2H), 7.44-7.37 (m, 2H), 6.74 (s, 1H), 5.85 (s, 2H), 3.83 (s, 3H), 2.31 (s, 3H), 1.73-1.63 (m, 1H), 1.15-1.06 (m, 1H), 1.04-0.96 (m, 1H), 0.95-0.80 (m, 2H).

### Example 4

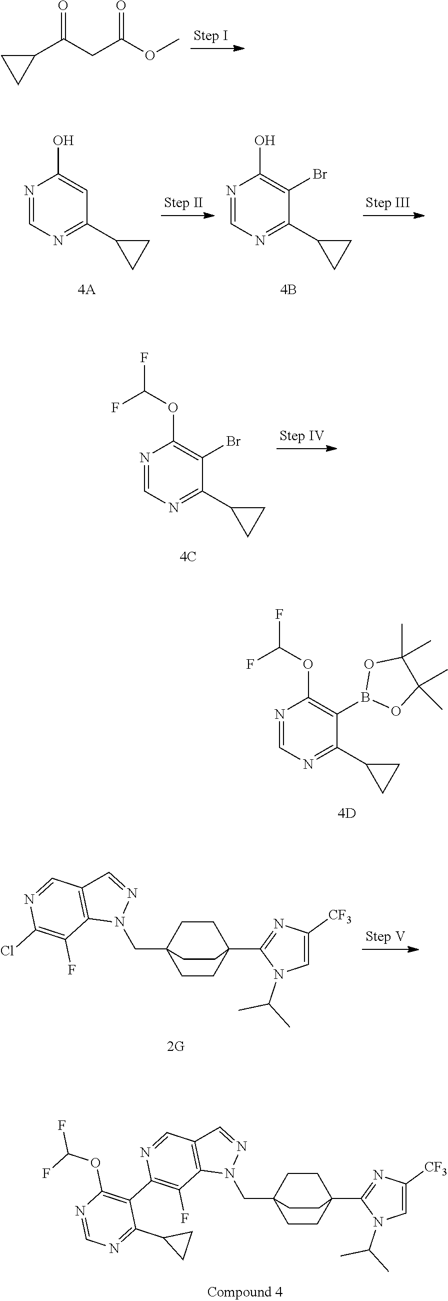

### Step I: Preparation of 4A

Methyl 3-cyclopropyl-3-oxopropionate (20 g, 140.66 mmol) and formamidine acetate (29.29 g, 281.32 mmol) were dissolved in methanol (400 mL), and sodium methoxide (53.19 g, 984.62 mmol) was added and reacted at room temperature for 2 days. The pH was adjusted to 7 with acetic acid, suction filtration was performed by diatomaceous earth, the filtrate was concentrated under reduced pressure, and the residue was purified by silica gel column chromatography (ethyl acetate/petroleum ether (V/V)=0/1-1/3); (methanol/dichloromethane (V/V)=0/1-1/10) to obtain 4A (9 g, yield: 47%).

LCMS m/z=137.1 [M+H]⁺.

### Step II: Preparation of 4B

4A (8.5 g, 62.43 mmol) was dissolved in acetonitrile (100 mL), and NBS (11.67 g, 65.60 mmol) was added and reacted at room temperature for 1 h. After extraction by adding water and ethyl acetate, the organic layer was washed with an aqueous saturated sodium chloride solution, dried over anhydrous sodium sulfate, and concentrated under reduced pressure, and the residue was purified by silica gel column chromatography (ethyl acetate/petroleum ether (V/V)=0/1-1/1) to obtain 4B (9.1 g, yield: 67%).

LCMS m/z=215.0 [M+H]⁺.

### Step III: Preparation of 4C

4B (6 g, 27.90 mmol) was dissolved in acetonitrile (200 mL), sodium hydride (2.2 g, 54.90 mmol) was added, stirred at room temperature and reacted for 45 min, and 2-fluorosulfonyl difluoroacetic acid (8.94 g, 50.22 mmol) was added and reacted at room temperature for 2 h. After extraction by adding water and ethyl acetate, the organic layer was washed with an aqueous saturated sodium chloride solution, dried over anhydrous sodium sulfate, and concentrated under reduced pressure, and the residue was purified by silica gel column chromatography (ethyl acetate/petroleum ether (V/V)=0/1-1/2) to obtain 4C (4.0 g, yield: 54%).

LCMS m/z=265.0 [M+H]⁺.

### Step IV: Preparation of 4D

4C (1.0 g, 3.77 mmol) was dissolved in 1,4-dioxane (15 mL), bis(pinacolato)diboron (1.05 g, 4.16 mmol), Pd(dppf)Cl₂·CH₂Cl₂ (0.31 g, 0.38 mmol), and potassium acetate (0.74 g, 7.54 mmol) were added in sequence, and the mixture was reacted under nitrogen protection at 90° C. overnight. The reaction product was cooled to room temperature and extracted by adding water and ethyl acetate, the organic layer was washed with an aqueous saturated sodium chloride solution, dried over anhydrous sodium sulfate, and concentrated under reduced pressure, and the residue was purified by silica gel column chromatography (ethyl acetate/petroleum ether (V/V)=1/1-1/20) to obtain 4D (420 mg, yield: 35%).

LCMS m/z=313.0 [M+H]⁺.

### Step V: Preparation of Compound 4

2G (100 mg, 0.21 mmol), 4D (85.21 mg, 0.27 mmol), Pd(dppf)Cl₂·CH₂Cl₂ (17.15 mg, 0.021 mmol), and potassium carbonate (87.07 mg, 0.63 mmol) were placed in a 25 mL single-mouth flask, 1,4-dioxane (4 mL) and water (1 mL) were added, and after displacement with nitrogen three times, the mixture was stirred at 90° C. for 3 h. After cooling to room temperature, 20 mL of water and 20 mL of ethyl acetate were added for extraction, the organic layer was dried over anhydrous sodium sulfate and then concentrated under reduced pressure, the residue was purified by silica gel column chromatography (ethyl acetate/petroleum ether (V/V)=1/10-1/1), and the resulting crude product was further subjected to separation and purification by preparative HPLC (instrument: waters 2767 preparative liquid chromatographic instrument; chromatographic column: SunFire @Prep C18 (19 mm×150 mm); composition of mobile phase: mobile phase A: acetonitrile, mobile phase B: water (containing 5 mM ammonium acetate)) to obtain Compound 4 (20 mg, yield: 15%).

LCMS m/z=620.3 [M+H]⁺.

¹H NMR (400 MHz, DMSO-d₆) δ 9.10 (d, 1H), 8.85 (s, 1H), 8.52 (d, 1H), 8.03-7.63 (m, 2H), 4.88-4.74 (m, 1H), 4.39-4.26 (m, 2H), 1.95-1.79 (m, 7H), 1.61-1.46 (m, 6H), 1.32 (d, 6H), 1.23-1.18 (m, 1H), 1.13-0.92 (m, 3H).

### Example 5

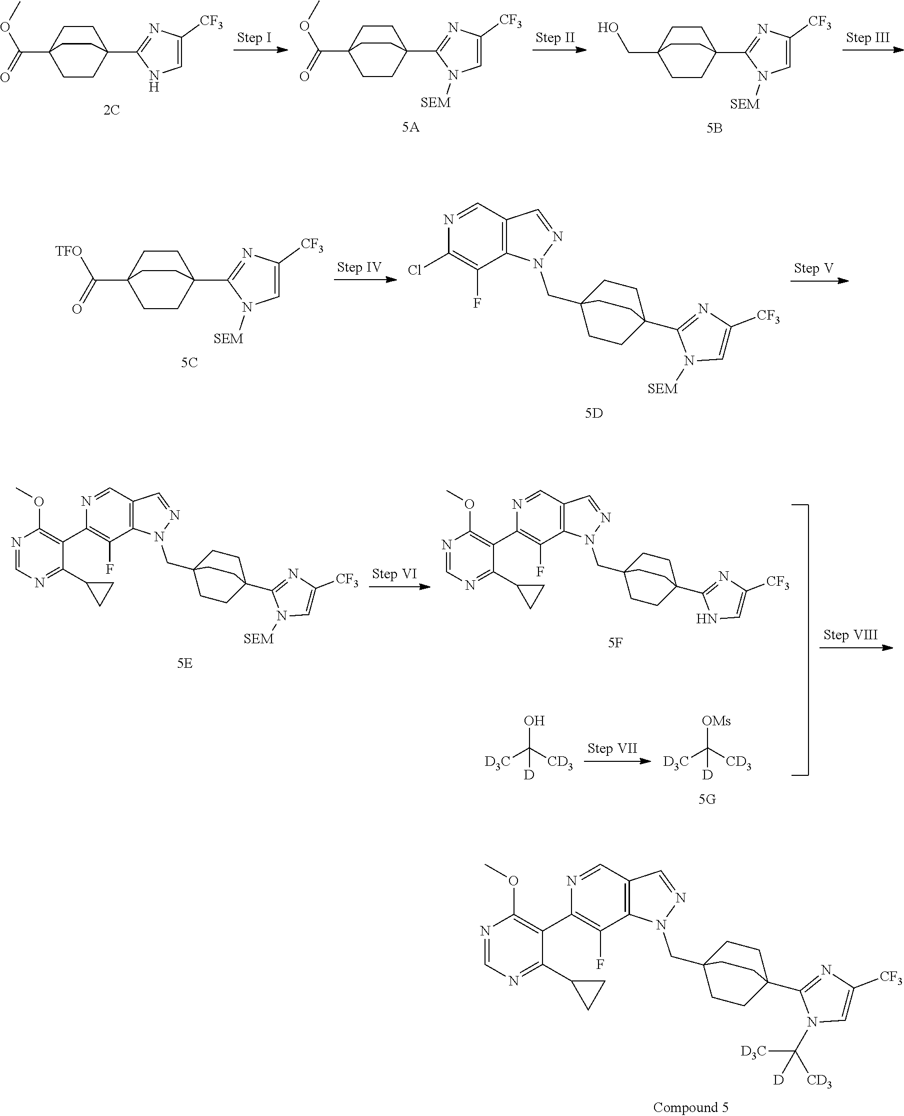

### Step I: Preparation of 5A

2C (4 g, 13.23 mmol) was dissolved in DMF (20 mL), NaH (0.79 g, 19.84 mmol, wt %=60%) was slowly added in an ice bath and stirred in the ice bath for 10 min, 2-(trimethylsilyl)ethoxymethyl chloride (3.31 g, 19.85 mmol) was then added, and the mixture was naturally heated to room temperature and stirred for 30 min. 10 mL of an aqueous saturated ammonium chloride solution was added to quench the reaction, 20 mL of ethyl acetate was added for extraction, the organic layer was washed with an aqueous saturated sodium chloride solution (10 mL×3), dried over anhydrous sodium sulfate, and concentrated under reduced pressure, and the residue was purified by silica gel column chromatography (ethyl acetate/petroleum ether (V/V)=1/10-1/3) to obtain 5A (5.2 g, yield: 90%).

### Step II: Preparation of 5B

5A (5.2 g, 12.02 mmol) was dissolved in THF (25 mL), lithium borohydride (1.31 g, 59.86 mmol) was added at room temperature, and after the addition was complete, the mixture was stirred at 60° C. for 2 h. After cooling to room temperature, 10 mL of an aqueous saturated ammonium chloride solution was dropwise added slowly to quench the reaction, the reaction product was extracted by adding 20 ml of ethyl acetate, and the organic layer was dried over anhydrous sodium sulfate and concentrated under reduced pressure to obtain 5B (5 g, yield: 100%).

LCMS m/z=405.2 [M+H]⁺.

### Step III: Preparation of 5C

5B (4.81 g, 11.89 mmol) was dissolved in DCM (30 mL), pyridine (1.41 g, 17.84 mmol) was added, trifluoromethanesulfonic anhydride (4.03 g, 14.29 mmol) was dropwise added slowly in an ice bath, and the mixture was naturally heated to room temperature and stirred for 1 h. 30 mL of dichloromethane and 50 mL of water were added for extraction, and the organic layer was washed once with 30 ml of an aqueous saturated sodium chloride solution, dried over anhydrous sodium sulfate, and concentrated under reduced pressure to obtain 5C.

### Step IV: Preparation of 5D

1F (1.7 g, 9.91 mmol) and 5C from the previous step were dissolved in DMF (30 mL), and in a water bath at room temperature, cesium carbonate (9.69 g, 29.73 mmol) was added. After the addition was complete, the mixture was stirred for 1 h, 50 mL of ethyl acetate and 50 mL of water were added for extraction, the organic layer was washed with an aqueous saturated sodium chloride solution (30 mL×3), dried over anhydrous sodium sulfate, and concentrated under reduced pressure, and the residue was purified by silica gel column chromatography (ethyl acetate/petroleum ether (V/V)=1/10-1/2) to obtain 5D (2.3 g, yield: 41%).

LCMS m/z=558.2 [M+H]⁺.

### Step V: Preparation of 5E

5D (1 g, 1.79 mmol) and (4-cyclopropyl-6-methoxypyrimidin-5-yl)boronic acid (0.69 g, 3.56 mmol) were dissolved in a mixed solvent of 1,4-dioxane (10 mL) and water (2 mL), and XPhos G3 (0.15 g, 0.18 mmol) and potassium phosphate (0.76 g, 3.58 mmol) were added in sequence; and after the addition was complete, displacement with nitrogen was performed three times, and the mixture was heated to 90° C. and stirred overnight. After cooling to room temperature, 20 ml of ethyl acetate and 10 ml of water were added for extraction, the organic layer was dried over anhydrous sodium sulfate and concentrated under reduced pressure, and the residue was purified by column chromatography (ethyl acetate/petroleum ether (V/V)=1/10-1/1) to obtain 5E (1.1 g, yield: 91%).

### Step VI: Preparation of 5F

5E (1.1 g, 1.64 mmol) was dissolved in THF (15 mL), tetrabutylammonium fluoride (4.29 g, 16.4 mmol) was added, and the mixture was heated to 70° C. and stirred for 6 h. After cooling to room temperature, 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/3-1/0) to obtain 5F (0.8 g, yield 90%).

LCMS m/z=542.2 [M+H]⁺.

### Step VII: Preparation of 5G

Isopropanol-D7 (2 g, 29.79 mmol) was dissolved in DCM (20 mL), triethylamine (6.03 g, 59.59 mmol) was added in an ice bath, methylsufonyl chloride (4.10 g, 35.79 mmol) was dropwise added slowly, and after the addition was complete, the mixture was naturally heated to room temperature and stirred for 20 min. 30 mL of water and 30 mL of dichloromethane were added for extraction, and the organic layer was then washed with 20 mL of a saturated sodium bicarbonate solution and 20 mL of an aqueous sodium chloride solution in sequence, dried over anhydrous sodium sulfate, and concentrated under reduced pressure to obtain 5G (4 g).

### Step VIII: Preparation of Compound 5

5F (0.1 g, 0.18 mmol) and 5G (0.13 g, 0.90 mmol) were dissolved in DMF (5 mL), and cesium carbonate (0.29 g, 0.90 mmol) and sodium iodide (0.027 g, 0.18 mmol) were added in sequence and stirred at 80° C. overnight. After cooling to room temperature, 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 5 (60 mg, yield 56%).

LCMS m/z=591.3 [M+H]⁺.

¹H NMR (400 MHz, DMSO-d₆) δ 9.05 (d, 1H), 8.72 (s, 1H), 8.48 (d, 1H), 7.93-7.85 (m, 1H), 4.38-4.25 (m, 2H), 3.86 (s, 3H), 1.96-1.82 (m, 6H), 1.78-1.68 (m, 1H), 1.62-1.45 (m, 6H), 1.18-0.98 (m, 2H), 0.97-0.81 (m, 2H).

### Example 6

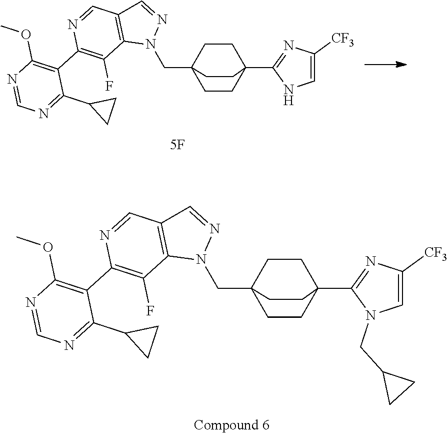 [SMILES: COc1ncnc(C2CC2)c1-c1ncc2cnn(CC34CCC(c5nc(C(F)(F)F)c[nH]5)(CC3)CC4)c2c1F ; COc1ncnc(C2CC2)c1-c1ncc2cnn(CC34CCC(c5nc(C(F)(F)F)cn5CC5CC5)(CC3)CC4)c2c1F]

5F (0.1 g, 0.18 mmol) and bromomethylcyclopropane (0.049 g, 0.36 mmol) were dissolved in DMF (5 mL), and cesium carbonate (0.12 g, 0.36 mmol) was added and stirred at 60° C. for 3 h. After cooling to room temperature, 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 6 (60 mg, yield 56%).

LCMS m/z=596.3 [M+H]⁺.

¹H NMR (400 MHz, DMSO-d₆) δ 9.04 (d, 1H), 8.72 (s, 1H), 8.48 (d, 1H), 7.79-7.71 (m, 1H), 4.39-4.24 (m, 2H), 3.94 (d, 2H), 3.86 (s, 3H), 1.96-1.82 (m, 6H), 1.79-1.68 (m, 1H), 1.61-1.45 (m, 6H), 1.22-1.08 (m, 2H), 1.05-0.98 (m, 1H), 0.97-0.80 (m, 2H), 0.59-0.49 (m, 2H), 0.46-0.37 (m, 2H).

### Example 7

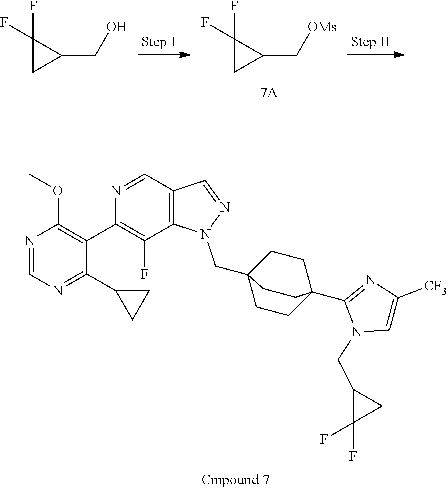 [SMILES: OCC1CC1(F)F ; CS(=O)(=O)OCC1CC1(F)F ; COc1ncnc(C2CC2)c1-c1ncc2cnn(CC34CCC(c5nc(C(F)(F)F)cn5CC5CC5(F)F)(CC3)CC4)c2c1F]

### Step I: Preparation of 7A

2,2-Difluorocyclopropyl methanol (1 g, 9.28 mmol) was dissolved in DCM (20 mL), triethylamine (1.88 g, 18.54 mmol) was added in an ice bath, methylsufonyl chloride (1.28 g, 11.11 mmol) was dropwise added slowly, and the mixture was naturally heated to room temperature and stirred for 20 min. 30 mL of water and 30 mL of dichloromethane were added for extraction, and the organic layer was then washed with 20 mL of an aqueous saturated sodium bicarbonate solution and 20 mL of an aqueous sodium chloride solution in sequence, dried over anhydrous sodium sulfate, and concentrated under reduced pressure to obtain 7A (1.6 g).

### Step II: Preparation of Compound 7

5F (0.1 g, 0.18 mmol) and 7A (0.067 g, 0.36 mmol) were dissolved in DMF (5 mL), and cesium carbonate (0.12 g, 0.36 mmol) and sodium iodide (0.027 g, 0.18 mmol) were added in sequence and stirred at 60° C. for 3 h. After cooling to room temperature, 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 7 (65 mg, yield 57%).

LCMS m/z=632.7 [M+H]⁺.

¹H NMR (400 MHz, DMSO-d₆) δ 9.04 (d, 1H), 8.72 (s, 1H), 8.48 (d, 1H), 7.73-7.67 (m, 1H), 4.39-4.26 (m, 3H), 4.19-4.08 (m, 1H), 3.86 (s, 3H), 2.28-2.15 (m, 1H), 1.96-1.83 (m, 6H), 1.77-1.62 (m, 3H), 1.59-1.45 (m, 6H), 1.18-1.09 (m, 1H), 1.06-0.97 (m, 1H), 0.97-0.83 (m, 2H).

### Example 8

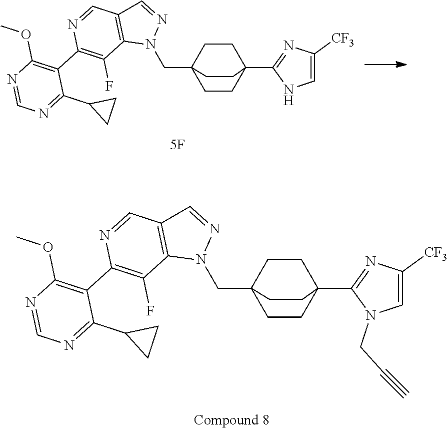 [SMILES: COc1ncnc(C2CC2)c1-c1ncc2cnn(CC34CCC(c5nc(C(F)(F)F)c[nH]5)(CC3)CC4)c2c1F ; C#CCn1cc(C(F)(F)F)nc1C12CCC(Cn3ncc4cnc(-c5c(OC)ncnc5C5CC5)c(F)c43)(CC1)CC2]

5F (0.1 g, 0.18 mmol) and 3-bromopropyne (0.043 g, 0.36 mmol) were dissolved in DMF (5 mL), and cesium carbonate (0.12 g, 0.36 mmol) was added and stirred at room temperature for 3 h. 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 8 (90 mg, yield 86%).

LCMS m/z=580.2 [M+H]⁺.

¹H NMR (400 MHz, DMSO-d₆) δ 9.05 (d, 1H), 8.72 (s, 1H), 8.48 (d, 1H), 7.76-7.69 (m, 1H), 5.03 (d, 2H), 4.41-4.24 (m, 2H), 3.86 (s, 3H), 3.53 (t, 1H), 1.96-1.83 (m, 6H), 1.79-1.69 (m, 1H), 1.60-1.43 (m, 6H), 1.18-1.09 (m, 1H), 1.06-0.98 (m, 1H), 0.98-0.82 (m, 2H).

### Example 9

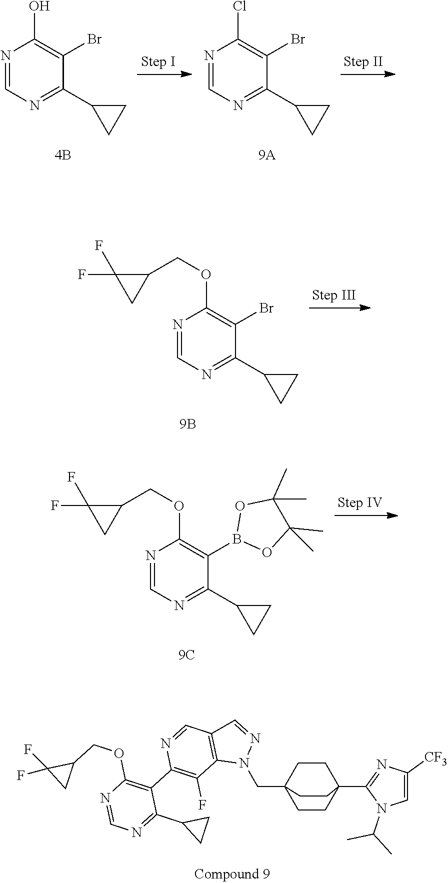 [SMILES: Oc1ncnc(C2CC2)c1Br ; Clc1ncnc(C2CC2)c1Br ; FC1(F)CC1COc1ncnc(C2CC2)c1Br ; CC1(C)OB(c2c(OCC3CC3(F)F)ncnc2C2CC2)OC1(C)C ; CC(C)n1cc(C(F)(F)F)nc1C12CCC(Cn3ncc4cnc(-c5c(OCC6CC6(F)F)ncnc5C5CC5)c(F)c43)(CC1)CC2]

### Step I: Preparation of 9A

4B (1 g, 4.65 mmol) was dissolved in acetonitrile (10 mL), phosphorus oxychloride (7.13 g, 46.50 mmol) was added slowly at room temperature, and after the addition was complete, the mixture was stirred at 80° C. for 4 h. After cooling to room temperature, the reaction liquid was poured into ice-water, the pH was adjusted to alkalinity with an aqueous saturated sodium bicarbonate solution, 30 mL of ethyl acetate was added for extraction, and the organic layer was dried over anhydrous sodium sulfate and then concentrated under reduced pressure to obtain 9A.

### Step II: Preparation of 9B

2,2-Difluorocyclopropyl methanol (0.93 g, 8.60 mmol) was dissolved in THF (10 mL), and sodium hydride (0.25 g, 6.3 mmol, wt %=60%) was added slowly in an ice bath; and after the addition was complete, the mixture was stirred in an ice bath for 20 min, 9A (1 g, 4.28 mmol) was slowly added, and the mixture was naturally heated to room temperature and stirred for 20 min. 10 mL of an aqueous saturated ammonium chloride solution was added to quench the reaction, 20 mL of ethyl acetate and 20 mL of water were added for extraction, and the organic layer was dried over anhydrous sodium sulfate and concentrated under reduced pressure to obtain 9B (1.4 g).

LCMS m/z=305.1 [M+H]⁺.

### Step III: Preparation of 9C

9B (1.3 g, 4.26 mmol) was dissolved in THF (10 mL), and a solution of isopropylmagnesium chloride in tetrahydrofuran (3.2 mL, 6.39 mmol, 2N) was dropwise added slowly in an ice bath and stirred at room temperature for 1.5 h. At 0° C., 2 mL of a solution of isopropyl pinacol borate (1.19 g, 6.39 mmol) in THF was dropwise added slowly and stirred at room temperature for 1 h. 20 mL of water was added to quench the reaction, 20 mL of ethyl acetate was added for extraction, the organic layer was dried over anhydrous sodium sulfate and concentrated under reduced pressure, and the residue was purified by silica gel column chromatography (ethyl acetate/petroleum ether (V/V)=0/1-1/10) to obtain 9C (0.23 g, yield: 15%).

LCMS m/z=353.2 [M+H]⁺.

### Step IV: Preparation of Compound 9

2G (0.11 g, 0.23 mmol) and 9C (0.12 g, 0.34 mmol) were dissolved in a mixed solvent of 1,4-dioxane (4 mL) and water (1 mL), XPhos G3 (0.019 g, 0.023 mmol) and potassium phosphate (0.098 g, 0.46 mmol) were added in sequence; and after the addition was complete, displacement with nitrogen was performed three times, and the mixture was stirred at 90° C. overnight. After cooling to room temperature, 20 mL of ethyl acetate and 10 mL of water were added for extraction, the organic layer was dried over anhydrous sodium sulfate and concentrated under reduced pressure, the residue was purified by silica gel column chromatography (ethyl acetate/petroleum ether (V/V)=1/10-1/1), and the resulting crude product was further subjected to separation and purification by preparative HPLC (instrument: waters 2767 preparative liquid chromatographic instrument; chromatographic column: SunFire @Prep C18 (19 mm×150 mm); composition of mobile phase: mobile phase A: acetonitrile, mobile phase B: water (containing 5 mM ammonium acetate)) to obtain Compound 9 (30 mg, yield: 20%).

LCMS m/z=660.90 [M+H]⁺.

¹H NMR (400 MHz, DMSO-d₆) δ 9.06 (d, 1H), 8.71 (d, 1H), 8.49 (d, 1H), 7.91-7.84 (m, 1H), 4.88-4.73 (m, 1H), 4.68-4.14 (m, 4H), 2.17-2.00 (m, 1H), 1.94-1.80 (m, 6H), 1.78-1.69 (m, 1H), 1.65-1.45 (m, 7H), 1.43-1.28 (m, 7H), 1.18-1.10 (m, 1H), 1.05-0.91 (m, 2H), 0.90-0.80 (m, 1H).

### Example 10

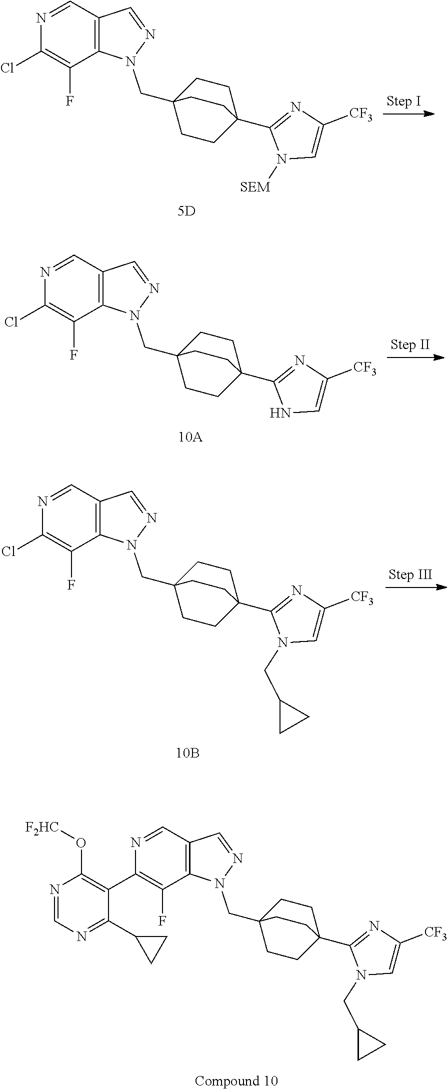 [SMILES: C[Si](C)(C)CCOCn1cc(C(F)(F)F)nc1C12CCC(Cn3ncc4cnc(Cl)c(F)c43)(CC1)CC2 ; Fc1c(Cl)ncc2cnn(CC34CCC(c5nc(C(F)(F)F)c[nH]5)(CC3)CC4)c12 ; Fc1c(Cl)ncc2cnn(CC34CCC(c5nc(C(F)(F)F)cn5CC5CC5)(CC3)CC4)c12 ; Fc1c(-c2c(OC(F)F)ncnc2C2CC2)ncc2cnn(CC34CCC(c5nc(C(F)(F)F)cn5CC5CC5)(CC3)CC4)c12]

### Step I: Preparation of 10A

5D (0.7 g, 1.25 mmol) was dissolved in DCM (4 mL), and trifluoroacetic acid (4 mL) was added and stirred at room temperature overnight. The mixture was concentrated under reduced pressure, 20 mL of dichloromethane was added to the residue, 10 mL of an aqueous saturated sodium bicarbonate solution was added and stirred for 10 min, and after liquid separation, the organic layer was dried over anhydrous sodium sulfate and concentrated under reduced pressure to obtain 10A.

### Step II: Preparation of 10B

10A (0.1 g, 0.23 mmol) was dissolved in DMF (5 mL), and bromomethylcyclopropane (0.062 g, 0.46 mmol) was added and stirred at 60° C. for 3 h. After cooling to room temperature, 20 mL of ethyl acetate and 20 mL of water were added and stirred; and after layering, the organic layer was washed with an aqueous saturated sodium chloride solution (10 mL×3), dried over anhydrous sodium sulfate, and concentrated under reduced pressure, and the residue was purified by silica gel column chromatography (ethyl acetate/petroleum ether (V/V)=1/5-1/2) to obtain 10B (0.1 g, yield: 90%).

### Step III: Preparation of Compound 10

10B (0.1 g, 0.21 mmol) and 4D (0.098 g, 0.32 mmol) were dissolved in a mixed solvent of 1,4-dioxane (4 mL) and water (1 mL), XPhos Pd G2 (0.017 g, 0.021 mmol) and potassium phosphate (0.089 g, 0.42 mmol) were added in sequence, and after displacement with nitrogen three times, the mixture was stirred at 90° C. for 3 h. After cooling to room temperature, 20 ml of ethyl acetate and 10 ml of water were added for extraction, the organic layer was dried over anhydrous sodium sulfate and concentrated under reduced pressure, and the residue was purified by silica gel column chromatography (ethyl acetate/petroleum ether (V/V)=1/10-1/1) to obtain Compound 10 (90 mg, yield: 68%).

LCMS m/z=632.90 [M+H]⁺.

¹H NMR (400 MHz, DMSO-d₆) δ 9.10 (d, 1H), 8.85 (s, 1H), 8.52 (d, 1H), 8.05-7.62 (m, 2H), 4.42-4.24 (m, 2H), 3.93 (d, 2H), 1.94-1.81 (m, 7H), 1.64-1.46 (m, 6H), 1.27-1.18 (m, 2H), 1.18-1.13 (m, 1H), 1.06-0.93 (m, 2H), 0.58-0.50 (m, 2H), 0.45-0.37 (m, 2H).

### Example 11

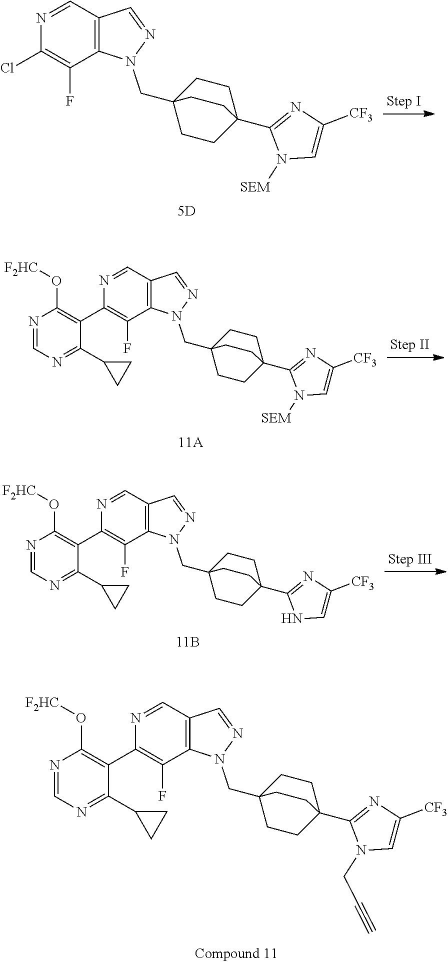 [SMILES: C[Si](C)(C)CCOCn1cc(C(F)(F)F)nc1C12CCC(Cn3ncc4cnc(Cl)c(F)c43)(CC1)CC2 ; C[Si](C)(C)CCOCn1cc(C(F)(F)F)nc1C12CCC(Cn3ncc4cnc(-c5c(OC(F)F)ncnc5C5CC5)c(F)c43)(CC1)CC2 ; Fc1c(-c2c(OC(F)F)ncnc2C2CC2)ncc2cnn(CC34CCC(c5nc(C(F)(F)F)c[nH]5)(CC3)CC4)c12 ; C#CCn1cc(C(F)(F)F)nc1C12CCC(Cn3ncc4cnc(-c5c(OC(F)F)ncnc5C5CC5)c(F)c43)(CC1)CC2]

### Step I: Preparation of 11A

5D (0.100 g, 0.18 mmol) and 4D (0.084 g, 0.27 mmol) were dissolved in a mixed solvent of 1,4-dioxane (4 mL) and water (1 mL), potassium phosphate (0.076 g, 0.36 mmol) and XPhos Pd G2 (0.014 g, 0.018 mmol) were added in sequence, and after displacement with nitrogen three times, the mixture was stirred at 90° C. overnight. After cooling to room temperature, 20 mL of ethyl acetate and 10 mL of water were added for extraction, the organic layer was dried over anhydrous sodium sulfate and concentrated under reduced pressure, and the residue was purified by silica gel column chromatography (ethyl acetate/petroleum ether (V/V)=1/10-1/2) to obtain 11A (0.13 g, yield: 99%).

### Step II: Preparation of 11B

11A (0.13 g, 0.18 mmol) was dissolved in DCM (4 mL), and trifluoroacetic acid (4 mL) was added at room temperature and stirred at room temperature overnight. The mixture was concentrated under reduced pressure, 20 mL of dichloromethane was added to the residue, 10 mL of an aqueous saturated sodium bicarbonate solution was added and stirred for 10 min, and after liquid separation, the organic layer was dried over anhydrous sodium sulfate and concentrated under reduced pressure to obtain 11B.

### Step III: Preparation of Compound 11

11B (0.11 g, 0.19 mmol) and 3-bromopropyne (0.045 g, 0.38 mmol) were dissolved in DMF (5 mL), and cesium carbonate (0.12 g, 0.38 mmol) was added and stirred at room temperature for 3 h. 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by silica gel column chromatography (ethyl acetate/petroleum ether (V/V)=1/5-1/1) to obtain Compound 11 (70 mg, yield 60%).

LCMS m/z=616.80 [M+H]⁺.

¹H NMR (400 MHz, DMSO-d₆) δ 9.10 (d, 1H), 8.85 (s, 1H), 8.52 (d, 1H), 8.03-7.62 (m, 2H), 5.02 (d, 2H), 4.40-4.26 (m, 2H), 3.51 (t, 1H), 1.95-1.81 (m, 7H), 1.63-1.41 (m, 6H), 1.30-1.20 (m, 1H), 1.12-1.01 (m, 2H), 1.01-0.92 (m, 1H).

### Example 12

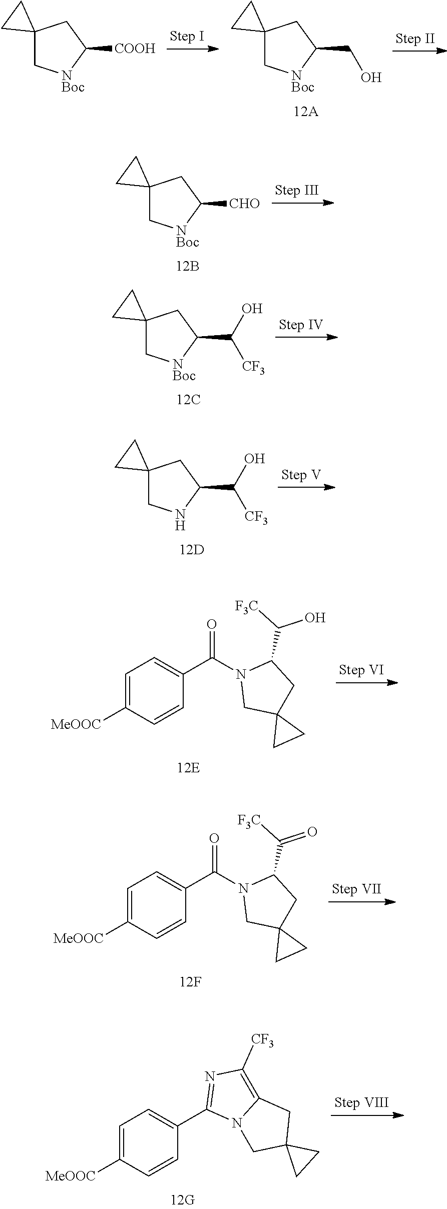

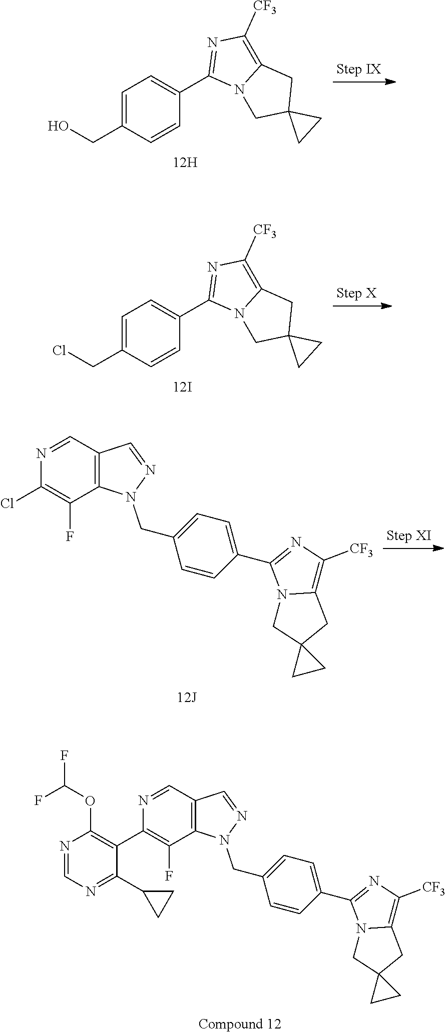 [SMILES: OCc1ccc(-c2nc(C(F)(F)F)c3n2CC2(CC2)C3)cc1 ; FC(F)(F)c1nc(-c2ccc(CCl)cc2)n2c1CC1(CC1)C2 ; Fc1c(Cl)ncc2cnn(Cc3ccc(-c4nc(C(F)(F)F)c5n4CC4(CC4)C5)cc3)c12 ; Fc1c(-c2c(OC(F)F)ncnc2C2CC2)ncc2cnn(Cc3ccc(-c4nc(C(F)(F)F)c5n4CC4(CC4)C5)cc3)c12]

### Step I: Preparation of 12A

(S)-5-(tert-butyloxycarboryl)-5-azaspiro[2.4]heptane-6-carboxylic acid (10 g, 41.49 mmol) was dissolved in THF (100 mL), and a borane tetrahydrofuran solution (62 mL, 1M) was dropwise added slowly at 0° C. and stirred at room temperature for 2 h. Methanol was added at 0° C. for quenching, the mixture was concentrated under reduced pressure, ethyl acetate and water were added to the residue for extraction, and the organic phase was washed with an aqueous saturated sodium chloride solution, dried over anhydrous sodium sulfate, and concentrated under reduced pressure to obtain 12A (9.42 g).

### Step II: Preparation of 12B

12A (9.42 g, 41.44 mmol) was dissolved in DMSO (60 mL), and 2-iodoxybenzoic acid (12.76 g, 45.58 mmol) was added and stirred at room temperature for 2 h. 2-Iodoxybenzoic acid (6 g, 21.41 mmol) was added and reacted at 40° C. for 1 h. 100 mL of ethyl acetate was added and stirred at room temperature for 10 min. After filtration, the filter cake was washed with ethyl acetate (3×30 mL), 100 mL of an aqueous saturated sodium thiosulfate solution was added to the filtrate and then stirred for 10 min, 50 mL of a saturated sodium bicarbonate solution was added for extraction, the organic phase was washed with an aqueous saturated sodium chloride solution three times, dried over anhydrous sodium sulfate, dried over anhydrous sodium sulfate, and concentrated under reduced pressure, and the residue was purified by silica gel column chromatography (ethyl acetate/petroleum ether (V/V)=0/1-1/5) to obtain 12B (8 g, yield: 85%).

### Step III: Preparation of 12C

At 0° C., (trifluoromethyl)trimethylsilane (4.73 g, 33.29 mmol) was added to a solution of 12B (5 g, 22.19 mmol) in tetrahydrofuran (50 mL). After stirring at 0° C. for 10 min, tetrabutylammonium fluoride (4.66 mL, 4.66 mmol, 1M solution in THF) was added and stirred at 0° C. for 1 h, tetrabutylammonium fluoride (5 mL, 5 mmol, 1M solution in THF) was then added, the mixture was stirred at room temperature for 2 h, an aqueous saturated ammonium chloride solution (100 mL) was then added for quenching, and the reaction product was extracted with ethyl acetate (100 mL×3). The organic phases were combined, dried over anhydrous sodium sulfate and then concentrated under reduced pressure, and the residue was purified by silica gel column chromatography (ethyl acetate/petroleum ether (V/V)=1/100-1/5) to obtain 12C (5.9 g, yield: 90%).

LCMS m/z=240.2 [M-55]⁺.

Step IV: Preparation of 12D hydrochloride

12C (1.73 g, 5.86 mmol) was dissolved in a hydrochloric acid-dioxane solution (30 mL, 4 M) and stirred at room temperature for 2 h. After the reaction was complete, 12D hydrochloride (1.34 g) was obtained by concentration under reduced pressure.

### Step V: Preparation of 12E

2-(7-Azobenzotriazole)-N,N,N′,N′-tetramethylurea hexafluorophosphate (4.16 g, 10.95 mmol) was added to 12D hydrochloride (1.69 g, 7.30 mmol) and 4-(methoxycarbonyl)benzoic acid (1.45 g, 8.03 mmol) in N,N-dimethylformamide (30 mL) and stirred at room temperature for 5 min. N,N-diisopropylethylamine (2.83 g, 21.9 mmol) was then added, 200 mL of ethyl acetate and 100 mL of water were added. After layering, the organic layer was dried under reduced pressure, and the residue was purified by silica gel column chromatography (ethyl acetate/petroleum ether (V/V)=1/100-1/5) to obtain 12E (2.5 g, yield 95%).

LCMS m/z=358.2 [M+H]⁺.

### Step VI: Preparation of 12F

12E (2.5 g, 7.00 mmol) was dissolved in dichloromethane (30 mL), and Dess-Martin periodinane (4.45 g, 10.5 mmol) was added at 0° C. in portions, and reacted at room temperature for 3 h. After direct concentration under reduced pressure, the residue was purified by silica gel column chromatography (ethyl acetate/petroleum ether (V/V)=1/100-1/5) to obtain 12F (2 g, yield: 80%).

### Step VII: Preparation of 12G

Ammonium trifluoroacetate (33.12 g, 252.73 mmol) was added to 12F (2 g, 5.63 mmol) and reacted at 130° C. for 2 h. After cooling to room temperature, 100 mL of water and ethyl acetate (100 mL×3) were added for extraction, the organic phases were combined, dried over anhydrous sodium sulfate and then concentrated under reduced pressure, and the residue was purified by silica gel column chromatography (ethyl acetate/petroleum ether (V/V)=1/100-1/2) to obtain 12G (1.4 g, yield: 73%).

LCMS m/z=337.1 [M+H]⁺.

### Step VIII: Preparation of 12H

At 0° C., diisobutylaluminium hydride (10.4 mL, 10.4 mmol, 1.0 M solution in hexanes) was dropwise added to 12G (1.4 g, 4.16 mmol) in tetrahydrofuran solution (20 mL), and the mixture was naturally heated to room temperature and reacted for 3 h. 60 mL of water and ethyl acetate (60 mL×3) were added for extraction, the organic phases were combined, dried over anhydrous sodium sulfate, and concentrated under reduced pressure, and the residue was purified by silica gel column chromatography (ethyl acetate/petroleum ether (V/V)=1/100-1/2) to obtain 12H (0.95 g, yield: 74%).

LCMS m/z=309.2 [M+H]⁺.

### Step IX: Preparation of 121

12H (0.95 g, 3.08 mmol) was dissolved in 10 mL of dichloromethane, and thionyl chloride (1.83 g, 15.40 mmol) was dropwise added slowly in an ice-water bath and stirred in the ice-water bath for 2 h. Saturated sodium bicarbonate (20 mL) was added for quenching, dichloromethane (30 mL×3) was added for extraction, and the organic phases were combined, dried over anhydrous sodium sulfate, and concentrated under reduced pressure to obtain 121 (0.95 g).

LCMS m/z=327.3 [M+H]⁺.

### Step X: Preparation of 12J

Cesium carbonate (1495.51 mg, 4.59 mmol) was added to a solution of 121 (500 mg, 1.53 mmol) and 1F (275.61 mg, 1.61 mmol) in N,N-dimethylformamide (10 mL) and reacted at room temperature for 3 h. After extraction by adding water and ethyl acetate, the organic phase was washed with an aqueous saturated sodium chloride solution, dried over anhydrous sodium sulfate, and concentrated under reduced pressure, and the residue was purified by silica gel column chromatography (ethyl acetate/petroleum ether (V/V)=0/1-2/1) to obtain 12J (330 mg, yield: 46%).

LCMS m/z=462.1 [M+H]⁺.

### Step XI: Preparation of 12

12J (100 mg, 0.22 mmol), 4D (103 mg, 0.33 mmol), XPhos Pd G2 (17.31 mg, 0.022 mmol) and potassium phosphate (140.10 mg, 0.66 mmol) were placed in a 25 mL single-mouth flask, 1,4-dioxane (4 mL) and water (1 mL) were added, and after displacement with nitrogen three times, the mixture was stirred at 90° C. for 3 h. After cooling to room temperature, 20 mL of water and 20 mL of ethyl acetate were added for extraction, the organic layer was dried over anhydrous sodium sulfate and then concentrated under reduced pressure, and the residue was purified by silica gel column chromatography (ethyl acetate/petroleum ether (V/V)=0/1-2/1) to obtain Compound 12 (50 mg, yield: 37%).

LCMS m/z=612.7 [M+H]⁺.

¹H NMR (400 MHz, DMSO-d₆) δ 9.12 (d, 1H), 8.84 (s, 1H), 8.56 (d, 1H), 8.03-7.59 (m, 3H), 7.34 (d, 2H), 5.90-5.72 (m, 2H), 4.23 (s, 2H), 2.95 (s, 2H), 1.89-1.76 (m, 1H), 1.21-1.14 (m, 1H), 1.12-0.98 (m, 2H), 0.98-0.89 (m, 1H), 0.78 (s, 4H).

### Example 13

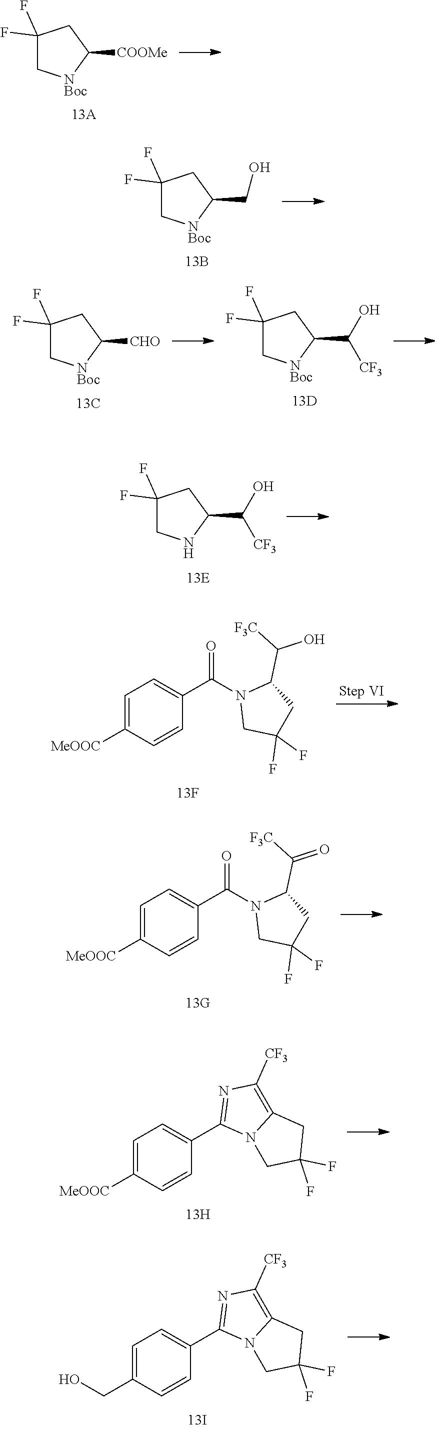

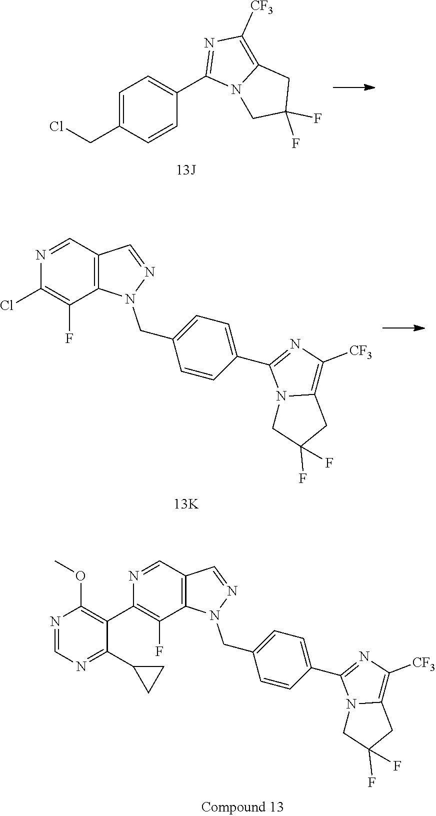 [SMILES: FC1(F)Cc2c(C(F)(F)F)nc(-c3ccc(CCl)cc3)n2C1 ; Fc1c(Cl)ncc2cnn(Cc3ccc(-c4nc(C(F)(F)F)c5n4CC(F)(F)C5)cc3)c12 ; COc1ncnc(C2CC2)c1-c1ncc2cnn(Cc3ccc(-c4nc(C(F)(F)F)c5n4CC(F)(F)C5)cc3)c2c1F]

### Step I: Preparation of 13B

13A (7 g, 26.4 mmol) was dissolved in tetrahydrofuran (100 mL), and lithium borohydride (1.72 g, 79.2 mmol) was added at 0° C. in portions and reacted at room temperature for 3 h. Water (50 mL) was added for quenching, ethyl acetate (40 mL×3) was added for extraction, the organic phases were combined, dried over anhydrous sodium sulfate and then concentrated under reduced pressure, and the residue was purified by silica gel column chromatography (ethyl acetate/petroleum ether (V/V)=1/100-1/10) to obtain 13B (6 g, yield: 96%).

LCMS m/z=182.1 [M-55]⁺.

### Step II: Preparation of 13C

13B (6 g, 25.3 mmol) was dissolved in dichloromethane (100 mL), and Dess-Martin periodinane (16.1 g, 37.95 mmol) was added at 0° C. in portions, and reacted at room temperature for 3 h. A saturated sodium thiosulfate solution (50 mL) was added for quenching, dichloromethane (30 mL×3) was added for extraction, the organic phases were combined, dried over anhydrous sodium sulfate and then concentrated under reduced pressure, and the residue was purified by silica gel column chromatography (ethyl acetate/petroleum ether (V/V)=1/100-1/10) to obtain 13C (3.2 g, yield 54%).

### Step III: Preparation of 13D

At 0° C., (trifluoromethyl)trimethylsilane (2.9 g, 20.4 mmol) was added to a solution of 13C (3.2 g, 13.6 mmol) in tetrahydrofuran (50 mL). After stirring at 0° C. for 10 min, tetrabutylammonium fluoride (0.86 mL, 0.86 mmol, 1M solution in THF) was added and stirred at 0° C. for 1 h, tetrabutylammonium fluoride (2 mL, 2 mmol, 1M solution in THF) was then added, the mixture was stirred at room temperature for 1 h, an aqueous saturated ammonium chloride solution (50 mL) was then added for quenching, and the reaction product was extracted with ethyl acetate (30 mL×3). The organic phases were combined, dried over anhydrous sodium sulfate and then concentrated under reduced pressure, and the residue was purified by silica gel column chromatography (ethyl acetate/petroleum ether (V/V)=1/100-1/10) to obtain 13D (4 g, yield: 96%).

Step IV: Preparation of 13E hydrochloride

13D (4 g, 13.1 mmol) was dissolved in a hydrochloric acid-dioxane solution (50 mL, 4 M) and stirred at room temperature for 2 h. After the reaction was complete, 13E hydrochloride was obtained by concentration under reduced pressure.

### Step V: Preparation of 13F

2-(7-azobenzotriazole)-N,N,N′,N′-tetramethylurea hexafluorophosphate (7.23 g, 19 mmol) was added to 13E (2.6 g, 12.67 mmol) and 4-(methoxycarbonyl)benzoic acid (2.05 g, 11.4 mmol) in N,N-dimethylformamide (20 mL) and stirred at room temperature for 5 min. N,N-diisopropylethylamine (3.27 g, 25.34 mmol) was then added, 100 mL of ethyl acetate and 50 mL of water were added. After layering, the organic layer was dried under reduced pressure, and the residue was purified by silica gel column chromatography (ethyl acetate/petroleum ether (V/V)=1/100-1/5) to obtain 13F (3.3 g, yield 71%).

LCMS m/z=368.4 [M+H]⁺.

### Step VI: Preparation of 13G

13F (2.5 g, 6.81 mmol) was dissolved in dichloromethane (50 mL), and Dess-Martin periodinane (4.33 g, 10.21 mmol) was added at 0° C. in portions, and the mixture was heated to room temperature and reacted for 3 h. A aqueous saturated sodium thiosulfate solution (50 mL) was added for quenching, dichloromethane (30 mL×3) was added for extraction, the organic phases were combined, dried over anhydrous sodium sulfate and then concentrated under reduced pressure, and the residue was purified by silica gel column chromatography (ethyl acetate/petroleum ether (V/V)=1/100-1/10) to obtain 13G (2.4 g, yield 96%).

### Step VII: Preparation of 13H

Ammonium trifluoroacetate (20 g, 76.31 mmol) was added to 13G (2.4 g, 6.57 mmol) in 1,4-dioxane solution (10 mL) and reacted at 130° C. for 2 h. After cooling to room temperature, 50 mL of water and ethyl acetate (30 mL×3) were added for extraction, the organic phases were combined, dried over anhydrous sodium sulfate and then concentrated under reduced pressure, and the residue was purified by silica gel column chromatography (ethyl acetate/petroleum ether (V/V)=1/100-1/10) to obtain 13H (1.1 g, yield: 48%).

LCMS m/z=347.1 [M+H]⁺.

### Step VIII: Preparation of 13I

At 0° C., diisobutylaluminium hydride (9.54 mL, 9.54 mmol, 1.0 M solution in hexanes) was dropwise added to 13H (1.1 g, 3.18 mmol) in tetrahydrofuran solution (55 mL) and reacted at 0° C. for 2 h. 50 mL of water and ethyl acetate (30 mL×3) were added for extraction, the organic phases were combined, dried over anhydrous sodium sulfate, and concentrated under reduced pressure, and the residue was purified by silica gel column chromatography (ethyl acetate/petroleum ether (V/V)=1/100-1/10) to obtain 13I (0.76 g, yield: 75%).

LCMS m/z=319.3 [M+H]⁺.

### Step IX: Preparation of 13J

13I (760 mg, 2.39 mmol) was dissolved in 20 mL of dichloromethane, and thionyl chloride (0.85 g, 7.17 mmol) was dropwise added slowly in an ice-water bath and stirred in the ice-water bath for 2 h. Saturated sodium bicarbonate (20 mL) was added for quenching, dichloromethane (30 mL×3) was added for extraction, and the organic phases were combined, dried over anhydrous sodium sulfate, and concentrated under reduced pressure to obtain 13J (800 mg).

LCMS m/z=337.3 [M+H]⁺.

### Step X: Preparation of 13K

Cesium carbonate (1.45 g, 4.47 mmol) was added to a solution of 13J (500 mg, 1.49 mol) and 1F (268 mg, 1.56 mmol) in N,N-dimethylformamide (10 mL) and stirred at room temperature for 2 h. After extraction by adding water and ethyl acetate, the organic phase was washed with an aqueous saturated sodium chloride solution, dried over anhydrous sodium sulfate, and concentrated under reduced pressure, and the residue was purified by silica gel column chromatography (ethyl acetate/petroleum ether (V/V)=0/1-2/1) to obtain 13K (300 mg, yield: 43%).

LCMS m/z=472.1 [M+H]⁺.

### Step XI: Preparation of Compound 13

13K (0.1 g, 0.21 mmol) was dissolved in a mixed solvent of 1,4-dioxane (10 mL) and water (3 mL), (4-cyclopropyl-6-methoxypyrimidin-5-yl)boronic acid (61 mg, 0.32 mmol), potassium phosphate (0.13 g, 0.60 mmol) and XPhos G2 (CAS: 1310584-14-5, 16.5 mg, 0.021 mmol) were added in sequence, and the mixture was stirred under nitrogen protection at 90° C. for 3 h. After cooling to room temperature, 10 mL of ethyl acetate and 10 mL of water were added, and after layering, the organic layer was dried over anhydrous sodium sulfate, and concentrated under reduced pressure. The residue was purified by silica gel column chromatography (ethyl acetate/petroleum ether (V/V)=1/5-1/1) to obtain 13 (60 mg, yield: 48.8%).

LCMS m/z=586.2 [M+H]⁺.

¹H NMR (400 MHz, DMSO-d₆) δ 9.07 (d, 1H), 8.71 (s, 1H), 8.54 (d, 1H), 7.82 (d, 2H), 7.37 (d, 2H), 5.82 (s, 2H), 4.93 (t, 2H), 3.84 (s, 3H), 3.72 (t, 2H), 1.73-1.64 (m, 1H), 1.16-1.06 (m, 1H), 1.05-0.96 (m, 1H), 0.95-0.78 (m, 2H).

### Example 14

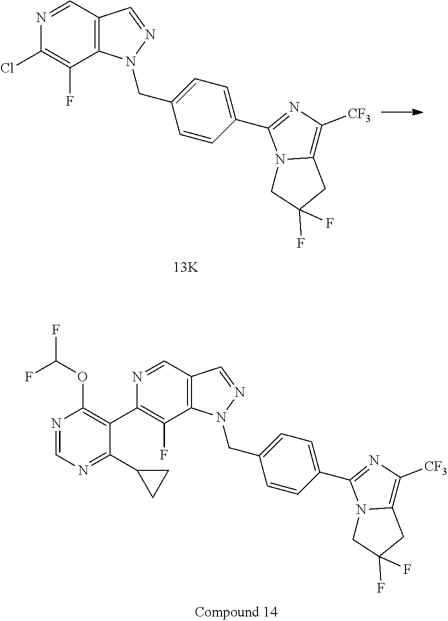 [SMILES: Fc1c(Cl)ncc2cnn(Cc3ccc(-c4nc(C(F)(F)F)c5n4CC(F)(F)C5)cc3)c12 ; Fc1c(-c2c(OC(F)F)ncnc2C2CC2)ncc2cnn(Cc3ccc(-c4nc(C(F)(F)F)c5n4CC(F)(F)C5)cc3)c12]

13K (0.1 g, 0.21 mmol) was dissolved in a mixed solvent of 1,4-dioxane (10 mL) and water (3 mL), 4D (98 mg, 0.32 mmol), potassium phosphate (0.13 g, 0.6 mmol) and XPhos G2 (CAS: 1310584-14-5, 17 mg, 0.021 mmol) were added in sequence, and the mixture was stirred under nitrogen protection at 90° C. for 3 h. After cooling to room temperature, 10 mL of ethyl acetate and 10 mL of water were added, and after layering, the organic layer was dried over anhydrous sodium sulfate, and concentrated under reduced pressure. The residue was purified by silica gel column chromatography (ethyl acetate/petroleum ether (V/V)=1/5-1/1) to obtain Compound 14 (66 mg, yield: 51%).

LCMS m/z=622.7 [M+H]⁺.

¹H NMR (400 MHz, DMSO-d₆) δ 9.12 (d, 1H), 8.84 (s, 1H), 8.57 (d, 1H), 8.00-7.60 (m, 3H), 7.37 (d, 2H), 5.89-5.77 (m, 2H), 4.91 (t, 2H), 3.71 (t, 2H), 1.88-1.78 (m, 1H), 1.21-1.12 (m, 1H), 1.11-0.90 (m, 3H).

### Example 15

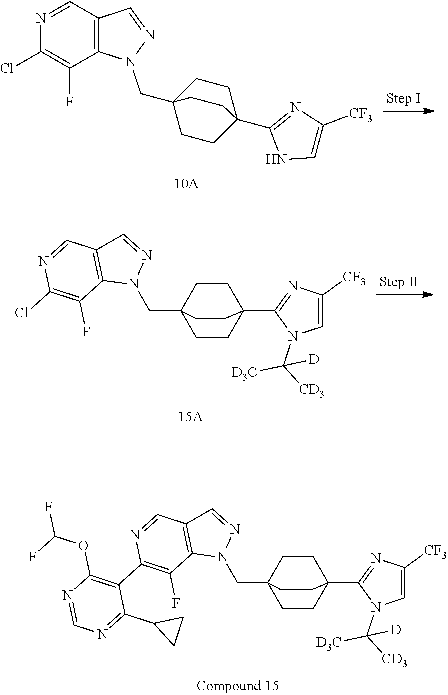 [SMILES: Fc1c(Cl)ncc2cnn(CC34CCC(c5nc(C(F)(F)F)c[nH]5)(CC3)CC4)c12 ; [2H]C([2H])([2H])C([2H])(n1cc(C(F)(F)F)nc1C12CCC(Cn3ncc4cnc(Cl)c(F)c43)(CC1)CC2)C([2H])([2H])[2H] ; [2H]C([2H])([2H])C([2H])(n1cc(C(F)(F)F)nc1C12CCC(Cn3ncc4cnc(-c5c(OC(F)F)ncnc5C5CC5)c(F)c43)(CC1)CC2)C([2H])([2H])[2H]]

### Step I: Preparation of 15A

10A (0.15 g, 0.35 mmol) and 5G (0.25 g, 1.72 mmol) were dissolved in DMF (5 mL), cesium carbonate (0.57 g, 1.75 mmol) and sodium iodide (0.052 g, 0.35 mmol) were added in sequence, and the mixture was stirred at 80° C. overnight. After cooling to room temperature, 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/2) to obtain Compound 15A (0.1 g, yield 60%).

### Step II: Preparation of Compound 15

15A (0.1 g, 0.21 mmol) and 4D (0.098 g, 0.32 mmol) were dissolved in 1,4-dioxane (4 mL) and water (1 mL), XPhos Pd G2 (0.017 g, 0.021 mmol) and potassium phosphate (0.089 g, 0.42 mmol) were added in sequence, and after displacement with nitrogen three times, the mixture was stirred at 90° C. for 3 h. After cooling to room temperature, 20 ml of ethyl acetate and 10 ml of water were added for extraction, the organic layer was dried over anhydrous sodium sulfate and concentrated under reduced pressure, and the residue was purified by column chromatography (ethyl acetate/petroleum ether (V/V)=1/10-1/1) to obtain Compound 15 (70 mg, yield: 53%).

LCMS m/z=627.80 [M+H]⁺.

¹H NMR (400 MHz, DMSO-d₆) δ 9.10 (d, 1H), 8.85 (s, 1H), 8.52 (d, 1H), 8.04-7.62 (m, 2H), 4.40-4.25 (m, 2H), 1.93-1.81 (m, 7H), 1.63-1.43 (m, 6H), 1.24-1.16 (m, 1H), 1.12-1.02 (m, 2H), 1.01-0.93 (m, 1H).

### Example 16

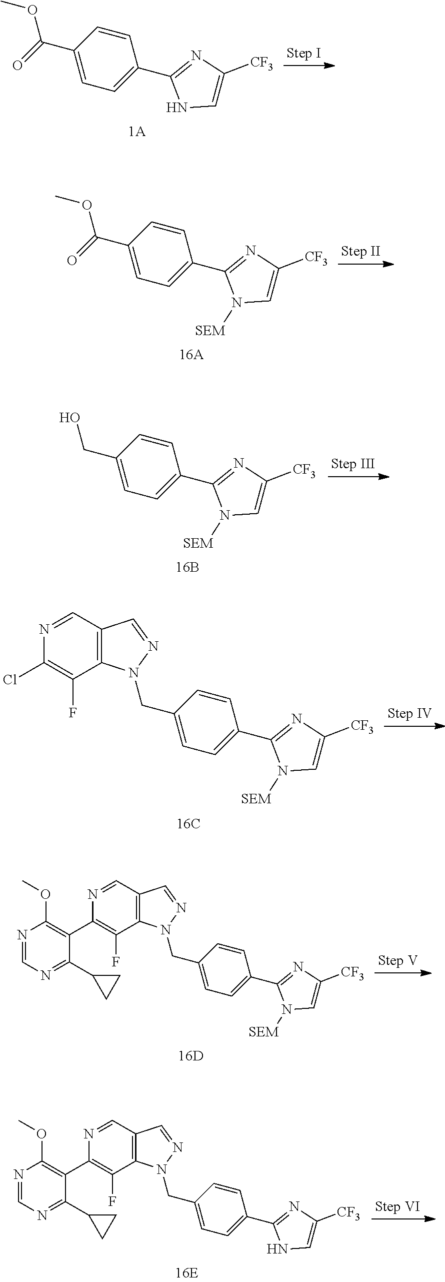 [SMILES: COC(=O)c1ccc(-c2nc(C(F)(F)F)c[nH]2)cc1 ; COC(=O)c1ccc(-c2nc(C(F)(F)F)cn2COCC[Si](C)(C)C)cc1 ; C[Si](C)(C)CCOCn1cc(C(F)(F)F)nc1-c1ccc(CO)cc1 ; C[Si](C)(C)CCOCn1cc(C(F)(F)F)nc1-c1ccc(Cn2ncc3cnc(Cl)c(F)c32)cc1 ; COc1ncnc(C2CC2)c1-c1ncc2cnn(Cc3ccc(-c4nc(C(F)(F)F)cn4COCC[Si](C)(C)C)cc3)c2c1F ; COc1ncnc(C2CC2)c1-c1ncc2cnn(Cc3ccc(-c4nc(C(F)(F)F)c[nH]4)cc3)c2c1F]

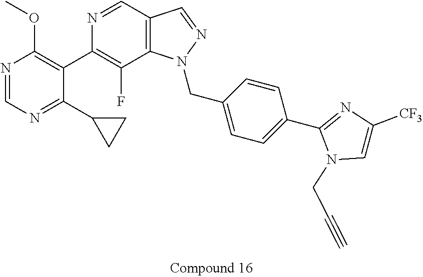 [SMILES: C#CCn1cc(C(F)(F)F)nc1-c1ccc(Cn2ncc3cnc(-c4c(OC)ncnc4C4CC4)c(F)c32)cc1]

### Step I: Preparation of 16A

1A (3 g, 11.10 mmol) was dissolved in 30 mL of DMF, sodium hydride (0.66 g, 16.5 mmol, wt %=60%) was added in an ice-water bath and reacted at 0° C. for 30 min, and 2-(trimethylsilyl)ethoxymethyl chloride (2.77 g, 16.63 mmol) was added and reacted at room temperature for 1 h. After quenching by adding water, water and ethyl acetate were added for extraction, the organic phase was washed with an aqueous saturated sodium chloride solution, dried over anhydrous sodium sulfate, and concentrated under reduced pressure, and the residue was purified by silica gel column chromatography (ethyl acetate/petroleum ether (V/V)=0/1-1/5) to obtain 16A (3.9 g, yield: 87%).

LCMS m/z=401.3 [M+H]⁺.

### Step II: Preparation of 16B

16A (3.9 g, 9.74 mmol) was dissolved in 30 mL of THF, and lithium borohydride (1.06 g, 48.7 mmol) was added slowly in portions and stirred at 50° C. for 3 h. After cooling to room temperature, an aqueous saturated ammonium chloride solution was added in an ice-water bath to quench the reaction, the reaction product was extracted by adding water and ethyl acetate, and the organic layer was dried over anhydrous sodium sulfate, concentrated under reduced pressure to obtain 16B (3.5 g, yield: 96%).

LCMS m/z=373.4 [M+H]⁺.

### Step III: Preparation of 16C

1F (0.5 g, 2.91 mmol), 16B (1.19 g, 3.20 mmol) and tri-n-butyl phosphorus (1.18 g, 5.82 mmol) were added to THF (10 mL), diethyl azodicarboxylate (1.01 g, 5.82 mmol) was dropwise added at room temperature under nitrogen protection and stirred at room temperature for 1 h. After concentration under reduced pressure, the residue was purified by silica gel column chromatography (ethyl acetate/petroleum ether (V/V)=1/10-1/2) to obtain 16C (0.52 g, yield: 34%).

LCMS m/z=526.60 [M+H]⁺.

### Step IV: Preparation of 16D

16C (0.2 g, 0.38 mmol) and (4-cyclopropyl-6-methoxypyrimidin-5-yl)boronic acid (0.13 g, 0.67 mmol) were dissolved in a mixed solvent of 1,4-dioxane (4 mL) and water (1 mL), potassium phosphate (0.16 g, 0.76 mmol) and XPhos G3 (0.032 g, 0.038 mmol) were added in sequence, and after displacement with nitrogen three times, the mixture was stirred at 90° C. for 3 h. After cooling to room temperature, 20 ml of ethyl acetate and 10 ml of water were added for extraction, the organic layer was dried over anhydrous sodium sulfate and concentrated under reduced pressure, and the residue was purified by column chromatography (ethyl acetate/petroleum ether (V/V)=1/10-1/2) to obtain 16D (0.22 g, yield: 90%).

### Step V: Preparation of 16E

16D (0.22 g, 0.34 mmol) was dissolved in DCM (4 mL), and TFA (4 mL) was added and stirred at room temperature overnight. After concentration under reduced pressure, 20 mL of dichloromethane was added, 10 mL of an aqueous saturated sodium bicarbonate solution was added for alkalization, and after liquid separation, the organic layer was dried over anhydrous sodium sulfate and concentrated under reduced pressure to obtain 16E.

### Step VI: Preparation of Compound 16

16E (0.090 g, 0.18 mmol) and 3-bromopropyne (0.043 g, 0.36 mmol) were dissolved in DMF (5 mL), and cesium carbonate (0.12 g, 0.36 mmol) was added and stirred at room temperature for 3 h. 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 16 (70 mg, yield 71%).

LCMS m/z=548.2 [M+H]⁺.

¹H NMR (400 MHz, DMSO-d₆) δ 9.07 (d, 1H), 8.71 (s, 1H), 8.54 (d, 1H), 8.04-8.01 (m, 1H), 7.75-7.70 (m, 2H), 7.42-7.37 (m, 2H), 5.84 (s, 2H), 4.99 (d, 2H), 3.84 (s, 3H), 3.56 (t, 1H), 1.74-1.65 (m, 1H), 1.14-1.07 (m, 1H), 1.04-0.96 (m, 1H), 0.94-0.78 (m, 2H).

### Example 17

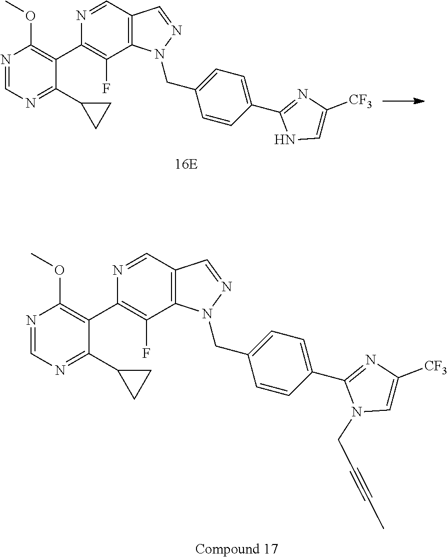 [SMILES: COc1ncnc(C2CC2)c1-c1ncc2cnn(Cc3ccc(-c4nc(C(F)(F)F)c[nH]4)cc3)c2c1F ; CC#CCn1cc(C(F)(F)F)nc1-c1ccc(Cn2ncc3cnc(-c4c(OC)ncnc4C4CC4)c(F)c32)cc1]

16E (0.090 g, 0.18 mmol) and 1-bromo-2-butyne (0.047 g, 0.35 mmol) were dissolved in DMF (5 mL), and cesium carbonate (0.12 g, 0.36 mmol) was added and stirred at room temperature for 3 h. 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 17 (70 mg, yield 69%).

LCMS m/z=562.2 [M+H]⁺.

¹H NMR (400 MHz, DMSO-d₆) δ 9.08 (d, 1H), 8.71 (s, 1H), 8.54 (d, 1H), 8.03-7.99 (m, 1H), 7.75-7.69 (m, 2H), 7.43-7.37 (m, 2H), 5.84 (s, 2H), 4.93-4.88 (m, 2H), 3.84 (s, 3H), 1.75 (t, 3H), 1.73-1.66 (m, 1H), 1.15-1.07 (m, 1H), 1.05-0.97 (m, 1H), 0.94-0.82 (m, 2H).

### Example 18

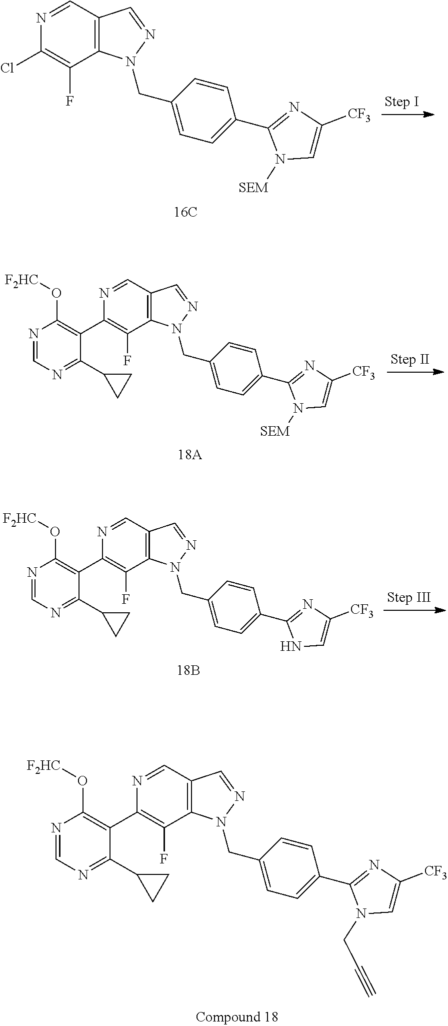 [SMILES: C[Si](C)(C)CCOCn1cc(C(F)(F)F)nc1-c1ccc(Cn2ncc3cnc(Cl)c(F)c32)cc1 ; C[Si](C)(C)CCOCn1cc(C(F)(F)F)nc1-c1ccc(Cn2ncc3cnc(-c4c(OC(F)F)ncnc4C4CC4)c(F)c32)cc1 ; Fc1c(-c2c(OC(F)F)ncnc2C2CC2)ncc2cnn(Cc3ccc(-c4nc(C(F)(F)F)c[nH]4)cc3)c12 ; C#CCn1cc(C(F)(F)F)nc1-c1ccc(Cn2ncc3cnc(-c4c(OC(F)F)ncnc4C4CC4)c(F)c32)cc1]

### Step I: Preparation of 18A

16C (0.3 g, 0.57 mmol) and 4D (0.44 g, 1.41 mmol) were dissolved in 1,4-dioxane (4 mL) and water (1 mL), XPhos Pd G2 (0.045 g, 0.057 mmol) and potassium phosphate (0.36 g, 1.70 mmol) were added in sequence, and after displacement with nitrogen three times, the mixture was stirred at 90° C. for 3 h. After cooling to room temperature, 20 ml of ethyl acetate and 10 ml of water were added for extraction, the organic layer was dried over anhydrous sodium sulfate and concentrated under reduced pressure, and the residue was purified by column chromatography (ethyl acetate/petroleum ether (V/V)=1/10-1/1) to obtain 18A (0.35 g, yield: 91%).

LCMS m/z=676.80 [M+H]⁺.

### Step II: Preparation of 18B

18A (0.35 g, 0.52 mmol) was dissolved in DCM (4 mL), and TFA (4 mL) was added and stirred at room temperature overnight. After concentration under reduced pressure, 20 mL of dichloromethane was added, 10 mL of an aqueous saturated sodium bicarbonate solution was added for alkalization, and after liquid separation, the organic layer was dried over anhydrous sodium sulfate and concentrated under reduced pressure to obtain 18B.

### Step III: Preparation of Compound 18

18B (0.070 g, 0.13 mmol) and 3-bromopropyne (0.031 g, 0.26 mmol) were dissolved in DMF (5 mL), and cesium carbonate (0.085 g, 0.26 mmol) was added and stirred at room temperature for 3 h. 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 18 (30 mg, yield 40%).

LCMS m/z=584.6 [M+H]⁺.

¹H NMR (400 MHz, DMSO-d₆) δ 9.13 (d, 1H), 8.84 (s, 1H), 8.57 (d, 1H), 8.04-8.01 (m, 1H), 7.99-7.62 (m, 3H), 7.44-7.38 (m, 2H), 5.91-5.80 (m, 2H), 4.97 (d, 2H), 3.55 (t, 1H), 1.88-1.78 (m, 1H), 1.21-1.13 (m, 1H), 1.10-0.89 (m, 3H).

### Example 19

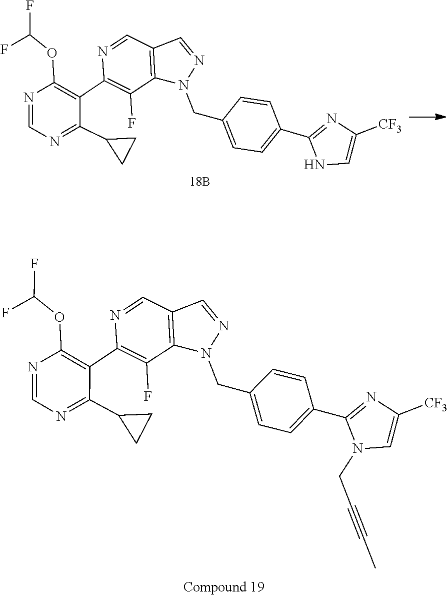 [SMILES: Fc1c(-c2c(OC(F)F)ncnc2C2CC2)ncc2cnn(Cc3ccc(-c4nc(C(F)(F)F)c[nH]4)cc3)c12 ; CC#CCn1cc(C(F)(F)F)nc1-c1ccc(Cn2ncc3cnc(-c4c(OC(F)F)ncnc4C4CC4)c(F)c32)cc1]

18B (0.076 g, 0.14 mmol) and 1-bromo-2-butyne (0.037 g, 0.28 mmol) were dissolved in DMF (5 mL), and cesium carbonate (0.091 g, 0.28 mmol) was added and stirred at room temperature for 3 h. 20 mL of ethyl acetate and 20 mL of water were added for extraction, the organic layer was washed with an aqueous saturated sodium chloride solution (10 mL×3), dried over anhydrous sodium sulfate and concentrated under reduced pressure, and the residue was purified by column chromatography (ethyl acetate/petroleum ether (V/V)=1/5-1/1) to obtain Compound 19 (50 mg, yield 60%).

LCMS m/z=598.80 [M+H]⁺.

¹H NMR (400 MHz, DMSO-d₆) δ 9.13 (d, 1H), 8.85 (s, 1H), 8.57 (d, 1H), 8.02-8.00 (m, 1H), 8.00-7.63 (m, 3H), 7.44-7.38 (m, 2H), 5.91-5.80 (m, 2H), 4.92-4.86 (m, 2H), 1.88-1.80 (m, 1H), 1.76 (t, 3H), 1.23-1.14 (m, 1H), 1.12-0.90 (m, 3H).

### Example 20

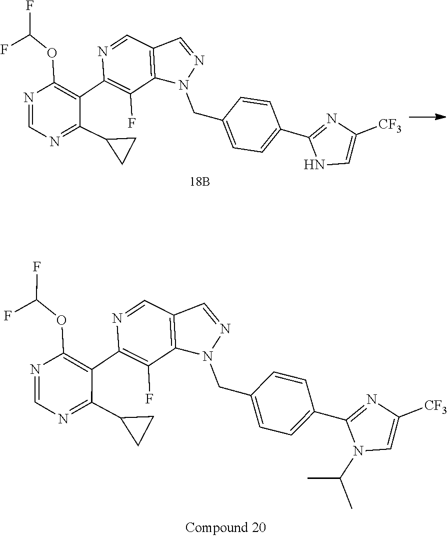 [SMILES: Fc1c(-c2c(OC(F)F)ncnc2C2CC2)ncc2cnn(Cc3ccc(-c4nc(C(F)(F)F)c[nH]4)cc3)c12 ; CC(C)n1cc(C(F)(F)F)nc1-c1ccc(Cn2ncc3cnc(-c4c(OC(F)F)ncnc4C4CC4)c(F)c32)cc1]

18B (0.1 g, 0.18 mmol) and isopropyl iodide (0.15 g, 0.90 mmol) were dissolved in acetonitrile (5 mL), and cesium carbonate (0.29 g, 0.90 mmol) was added and stirred at 60° C. for 16 h. After cooling to room temperature, 20 mL of ethyl acetate and 20 mL of water were added for extraction, the organic layer was washed with an aqueous saturated sodium chloride solution (10 mL), dried over anhydrous sodium sulfate and concentrated under reduced pressure, and the residue was purified by column chromatography (ethyl acetate/petroleum ether (V/V)=1/5-1/1) to obtain Compound 20 (70 mg, yield 66%).

LCMS m/z=588.7 [M+H]⁺.

¹H NMR (400 MHz, DMSO-d₆) δ 9.13 (d, 1H), 8.84 (s, 1H), 8.58 (d, 1H), 8.17-8.14 (m, 1H), 8.00-7.61 (m, 1H), 7.56-7.50 (m, 2H), 7.41-7.35 (m, 2H), 5.92-8.81 (m, 2H), 4.48-4.36 (m, 1H), 1.88-1.80 (m, 1H), 1.40-1.34 (m, 6H), 1.22-1.13 (m, 1H), 1.11-0.87 (m, 3H).

### Example 21

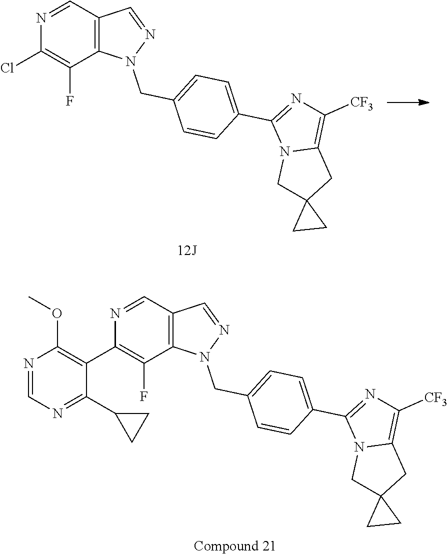 [SMILES: Fc1c(Cl)ncc2cnn(Cc3ccc(-c4nc(C(F)(F)F)c5n4CC4(CC4)C5)cc3)c12 ; COc1ncnc(C2CC2)c1-c1ncc2cnn(Cc3ccc(-c4nc(C(F)(F)F)c5n4CC4(CC4)C5)cc3)c2c1F]

12J (100 mg, 0.22 mmol), (4-cyclopropyl-6-methoxypyrimidin-5-yl)boronic acid (64.02 mg, 0.33 mmol), XPhos Pd G2 (17.31 mg, 0.022 mmol) and potassium phosphate (140.10 mg, 0.66 mmol) were placed in a 25 mL single-mouth flask, 1,4-dioxane (4 mL) and water (1 mL) were added, and after displacement with nitrogen three times, the mixture was stirred at 90° C. for 3 hours. After cooling to room temperature, 20 mL of water and 20 mL of ethyl acetate were added for extraction, the organic layer was dried over anhydrous sodium sulfate and then concentrated under reduced pressure, the residue was purified by silica gel column chromatography (ethyl acetate/petroleum ether (V/V)=0/1-2/1), and the resulting crude product was further subjected to separation and purification by preparative HPLC (instrument: waters 2767 preparative liquid chromatographic instrument; chromatographic column: SunFire @Prep C18 (19 mm×150 mm); composition of mobile phase: mobile phase A: acetonitrile, mobile phase B: water (containing 5 mM ammonium acetate)) to obtain Compound 21 (35 mg, yield: 27%).

LCMS m/z=576.3 [M+H]⁺.

¹H NMR (400 MHz, DMSO-d₆) δ 9.06 (d, 1H), 8.71 (s, 1H), 8.53 (d, 1H), 7.81-7.75 (m, 2H), 7.37-7.31 (m, 2H), 5.80 (s, 2H), 4.25 (s, 2H), 3.83 (s, 3H), 2.97-2.93 (m, 2H), 1.74-1.62 (m, 1H), 1.14-1.06 (m, 1H), 1.04-0.96 (m, 1H), 0.94-0.80 (m, 2H), 0.78 (s, 4H).

### Example 22

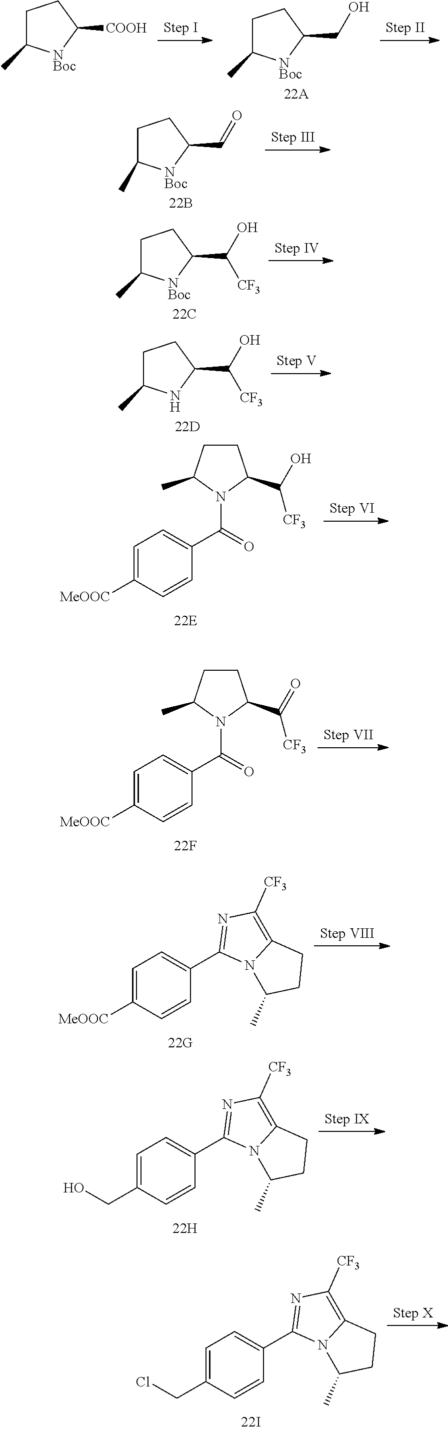

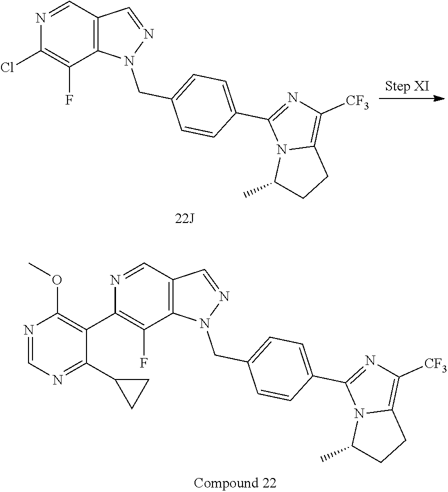 [SMILES: C[C@H]1CCc2c(C(F)(F)F)nc(-c3ccc(Cn4ncc5cnc(Cl)c(F)c54)cc3)n21 ; COc1ncnc(C2CC2)c1-c1ncc2cnn(Cc3ccc(-c4nc(C(F)(F)F)c5n4[C@@H](C)CC5)cc3)c2c1F]

### Step I: Preparation of 22A

(2S,5S)—N-Boc-5-methylpyrrolidine-2-carboxylic acid (3 g, 13.09 mmol) was dissolved in THF (30 mL), and a borane tetrahydrofuran solution (19.63 mL, 1M) was dropwise added slowly at 0° C. and stirred at room temperature for 2 h. Methanol was added at 0° C. for quenching, the mixture was concentrated under reduced pressure, ethyl acetate and water were added to the residue for extraction, and the organic phase was washed with an aqueous saturated sodium chloride solution, dried over anhydrous sodium sulfate, and concentrated under reduced pressure to obtain 22A (2.8 g).

### Step II: Preparation of 22B

22A (2.8 g, 13.01 mmol) was dissolved in DMSO (15 mL), and 2-iodoxybenzoic acid (4.01 g, 14.31 mmol) was added and stirred at 40° C. for 2 h. 60 mL of ethyl acetate was added and stirred at room temperature for 10 min. After filtration, the filter cake was washed with ethyl acetate (3×10 ml), 60 mL of an aqueous saturated sodium thiosulfate solution was added to the filtrate and then stirred for 10 min, 30 mL of a saturated sodium bicarbonate solution was added for extraction, the organic phase was washed with an aqueous saturated sodium chloride solution three times, dried over anhydrous sodium sulfate, dried over anhydrous sodium sulfate, and concentrated under reduced pressure, and the residue was purified by silica gel column chromatography (ethyl acetate/petroleum ether (V/V)=0/1-1/5) to obtain 22B (2 g, yield: 72%).

### Step III: Preparation of 22C

At 0° C., (trifluoromethyl)trimethylsilane (2 g, 14.07 mmol) was added to 22B (2 g, 9.38 mmol) in tetrahydrofuran (20 mL) and stirred at 0° C. for 10 min, tetrabutylammonium fluoride (1.97 mL, 1.97 mmol, 1M solution in THF) was added and stirred at room temperature for 2 h, an aqueous saturated ammonium chloride solution (30 mL) was added for quenching, ethyl acetate (50 mL×3) was added for extraction, the organic phases were combined, dried over anhydrous sodium sulfate and then concentrated under reduced pressure, and the residue was purified by silica gel column chromatography (ethyl acetate/petroleum ether (V/V)=1/100-1/5) to obtain 22C (2.46 g, yield: 92%).

LCMS m/z=228.1 [M-55]⁺.

### Step IV: Preparation of 22D Hydrochloride

22C (2.46 g, 8.68 mmol) was dissolved in a hydrochloric acid-dioxane solution (20 mL, 4 M) and stirred at room temperature for 2 hours. Concentration under reduced pressure afforded Compound 22D hydrochloride (1.8 g).

### Step V: Preparation of 22E

HATU (5.61 g, 14.75 mmol) was added to 22D hydrochloride (1.8 g) and 4-(methoxycarbonyl)benzoic acid (1.86 g, 10.32 mmol) in DMF (20 mL) and stirred at room temperature for 5 min, DIPEA (3.81 g, 29.49 mmol) was then added, and 100 mL of ethyl acetate and 100 mL of water were added. After layering, the organic layer was dried under reduced pressure, and the residue was purified by silica gel column chromatography (ethyl acetate/petroleum ether (V/V)=1/100-1/5) to obtain 22E (3 g, yield 88%).

LCMS m/z=346.1 [M+H]⁺.

### Step VI: Preparation of 22F

22E (2 g, 5.79 mmol) was dissolved in dichloromethane (30 mL), and Dess-Martin periodinane (3.68 g, 8.69 mmol) was added at 0° C. in portions, and reacted at room temperature for 3 h. After direct concentration under reduced pressure, the residue was purified by silica gel column chromatography (ethyl acetate/petroleum ether (V/V)=1/100-1/5) to obtain 22F (1.59 g, yield: 80%).

### Step VII: Preparation of 22G

Ammonium trifluoroacetate (11 g, 83.94 mmol) was added to 22F (1.59 g, 4.63 mmol) and reacted at 130° C. for 2 h. After cooling to room temperature, 50 mL of water and ethyl acetate (50 mL×3) were added for extraction, the organic phases were combined, dried over anhydrous sodium sulfate and then concentrated under reduced pressure, and the residue was purified by silica gel column chromatography (ethyl acetate/petroleum ether (V/V)=1/100-1/2) to obtain 22G (1.3 g, yield: 86%).

LCMS m/z=325.1 [M+H]⁺.

### Step VIII: Preparation of 22H

At 0° C., diisobutylaluminium hydride (10.02 mL, 10.02 mmol, 1.0 M solution in hexanes) was dropwise added to 22G (1.3 g, 4.01 mmol) in tetrahydrofuran solution (20 mL), and the mixture was naturally heated to room temperature and reacted for 3 hours. 60 mL of water was added, ethyl acetate (60 mL×3) was added for extraction, the organic phases were combined, dried over anhydrous sodium sulfate, and concentrated under reduced pressure, and the residue was purified by silica gel column chromatography (ethyl acetate/petroleum ether (V/V)=1/100-1/2) to obtain 22H (1.1 g, yield: 92%).

LCMS m/z=297.2 [M+H]⁺.

### Step IX: Preparation of 221

22H (1.1 g, 3.71 mmol) was dissolved in 20 mL of dichloromethane, and thionyl chloride (2.21 g, 18.55 mmol) was dropwise added slowly in an ice-water bath and reacted at room temperature for 2 h. A aqueous saturated sodium bicarbonate solution (30 mL) was added for quenching, dichloromethane (30 mL×3) was added for extraction, and the organic phases were combined, dried over anhydrous sodium sulfate, and concentrated under reduced pressure to obtain 221 (1 g).

LCMS m/z=315.1 [M+H]⁺.

### Step X: Preparation of 22J

Cesium carbonate (1554.16 mg, 4.77 mmol) was added to a solution of 221 (500 mg, 1.59 mmol) and 1F (286.42 mg, 1.67 mmol) in DMF (10 mL) and reacted at room temperature for 3 h. After extraction by adding water and ethyl acetate, the organic phase was washed with aqueous saturated sodium chloride solution, dried over anhydrous sodium sulfate, and concentrated under reduced pressure, and the residue was purified by silica gel column chromatography (ethyl acetate/petroleum ether (V/V)=0/1-2/1) to obtain Compound 22J (310 mg, yield: 43%).

LCMS m/z=450.1 [M+H]⁺.

### Step XI: Preparation of Compound 22

22J (150 mg, 0.33 mmol) was dissolved in a mixed solvent of 1,4-dioxane (4 mL) and water (1 mL), (4-cyclopropyl-6-methoxypyrimidin-5-yl)boronic acid (96.03 mg, 0.49 mmol), potassium phosphate (210.15 mg, 0.99 mmol) and XPhos G2 (CAS: 1310584-14-5, 25.96 mg, 0.033 mmol) were added in sequence, and the mixture was heated to 90° C. under nitrogen protection and reacted for 3 h. After cooling to room temperature, 20 mL of ethyl acetate and 20 mL of water were added, and after layering, the organic layer was dried over anhydrous sodium sulfate, and concentrated under reduced pressure. The residue was purified by silica gel column chromatography (ethyl acetate/petroleum ether (V/V)=1/5-2/1) to obtain Compound 22 (100 mg, yield: 53%).

LCMS m/z=564.60 [M+H]⁺.

¹H NMR (400 MHz, DMSO-d₆) δ 9.07 (d, 1H), 8.71 (s, 1H), 8.54 (d, 1H), 7.80 (d, 2H), 7.34 (d, 2H), 5.81 (s, 2H), 5.07-4.93 (m, 1H), 3.86-3.79 (m, 3H), 3.09-2.95 (m, 1H), 2.94-2.79 (m, 2H), 2.31-2.20 (m, 1H), 1.73-1.62 (m, 1H), 1.19-1.14 (m, 3H), 1.14-1.06 (m, 1H), 1.03-0.95 (m, 1H), 0.95-0.78 (m, 2H).

### Example 23

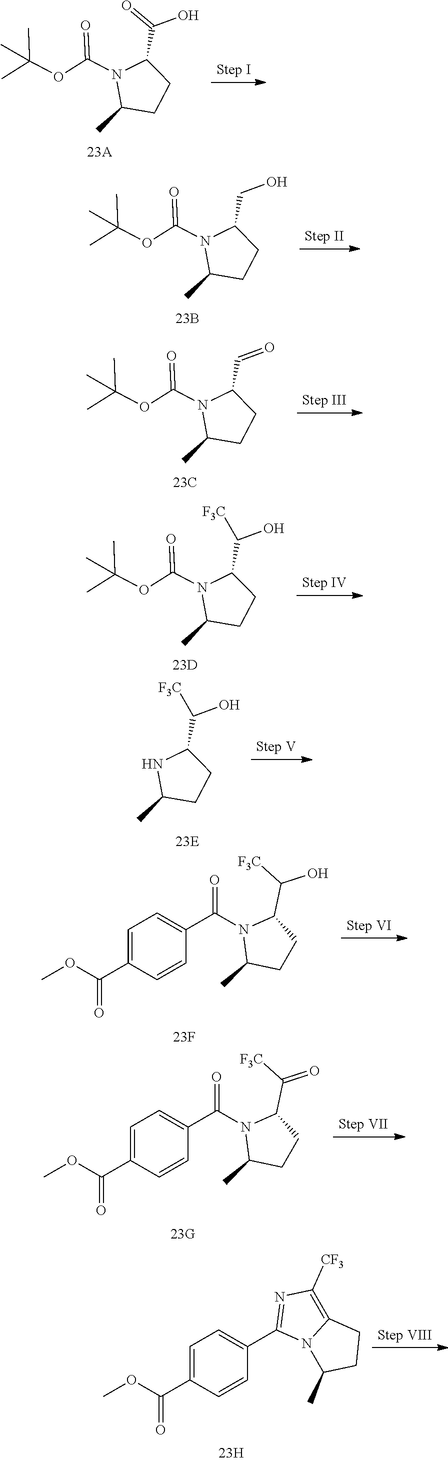

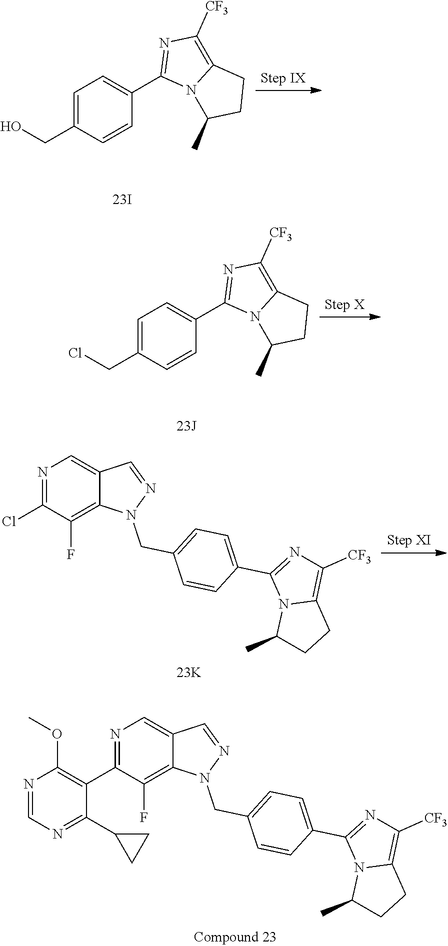 [SMILES: C[C@@H]1CCc2c(C(F)(F)F)nc(-c3ccc(CO)cc3)n21 ; C[C@@H]1CCc2c(C(F)(F)F)nc(-c3ccc(CCl)cc3)n21 ; C[C@@H]1CCc2c(C(F)(F)F)nc(-c3ccc(Cn4ncc5cnc(Cl)c(F)c54)cc3)n21 ; COc1ncnc(C2CC2)c1-c1ncc2cnn(Cc3ccc(-c4nc(C(F)(F)F)c5n4[C@H](C)CC5)cc3)c2c1F]

### Step I: Preparation of 23B

23A (CAS No.: 160033-52-3, 2 g, 8.73 mmol) was dissolved in THF (10 mL), and a borane tetrahydrofuran solution (17 mL, 17 mmol, 1N) was dropwise added in an ice bath and stirred at room temperature for 120 min. 10 mL of methanol was dropwise added to quench the reaction, the reaction product was concentrated under reduced pressure, the residue was dissolved by adding 50 mL of ethyl acetate and washed with 50 mL of a saturated sodium bicarbonate solution and 50 mL of an aqueous saturated sodium chloride solution in sequence, and the organic layer was dried over anhydrous sodium sulfate and concentrated under reduced pressure to obtain 23B.

LCMS m/z=160.1 [M-55]⁺.

### Step II: Preparation of 23C

23B (1.88 g, 8.73 mmol) was dissolved in DMSO (10 mL), and 2-iodoxybenzoic acid (2.93 g, 10.48 mmol) was added and stirred at room temperature for 30 min and at 40° C. for 30 min. After cooling to room temperature, 20 mL of ethyl acetate was added. After filtration, 20 mL of an aqueous saturated sodium thiosulfate solution was added to the filtrate and stirred for 20 min, 10 mL of a saturated sodium bicarbonate solution was then added and stirred for 10 min, and after liquid separation, the organic layer was dried over anhydrous sodium sulfate and then concentrated under reduced pressure to obtain 23C.

### Step III: Preparation of 23D

23C (1.88 g, 8.82 mmol) was dissolved in THF (20 mL), (trifluoromethyl)trimethylsilane (1.88 g, 13.23 mmol) was added, and tetrabutylammonium fluoride (0.69 g, 2.65 mmol, 1N) in tetrahydrofuran solution (2.65 mL) was dropwise added slowly in an ice bath and stirred at room temperature for 2 h. A aqueous saturated ammonium chloride solution (10 mL) was added for quenching, ethyl acetate (20 mL×2) was added for extraction, the organic phases were combined, dried over anhydrous sodium sulfate and then concentrated under reduced pressure, and the residue was purified by silica gel column chromatography (ethyl acetate/petroleum ether (V/V)=1/10-1/2) to obtain 23D (2 g, yield: 80%).

LCMS m/z=228.1 [M-55]⁺.

### Step IV: Preparation of 23E Hydrochloride

23D (2 g, 7.06 mmol) was dissolved in DCM (5 mL), and a hydrochloric acid-dioxane solution (10 mL, 4 M) was added and stirred at room temperature for 2 h. After concentration under reduced pressure, 20 mL of methyl tert-butyl ether was added and stirred for 10 min, and the mixture was then filtered to obtain 23E hydrochloride (1.16 g, yield: 75%).

### Step V: Preparation of 23F

HATU (2.42 g, 6.36 mmol) was added to a solution of 23E hydrochloride (1.16 g, 5.30 mmol) and monomethyl terephthalate (0.95 g, 5.30 mmol) in DMF (15 mL) and stirred at room temperature for 5 min, and DIPEA (1.37 g, 10.60 mmol) was added and stirred for 20 min. 30 mL of ethyl acetate was added, the mixture was washed with an aqueous saturated sodium chloride solution (20 mL×3), the organic layers were concentrated under reduced pressure, and the residue was purified by silica gel column chromatography (ethyl acetate/petroleum ether (V/V)=1/10-1/2) to obtain 23F (1.8 g, yield: 98%).

LCMS m/z=346.1 [M+H]⁺.

### Step VI: Preparation of 23G

23F (1.8 g, 5.21 mmol) was dissolved in DCM (20 mL), and Dess-Martin periodinane (2.65 g, 6.25 mmol) was added at 0° C. in portions and reacted at room temperature for 3 hours. 20 mL of an aqueous saturated sodium thiosulfate solution was added and stirred for 20 min, and 20 mL of a saturated sodium bicarbonate solution was then added and stirred for 10 min. After liquid separation, the organic layer was dried over anhydrous sodium sulfate and concentrated under reduced pressure, and the residue was purified by silica gel column chromatography (ethyl acetate/petroleum ether (V/V)=1/100-1/2) to obtain 23G (1.5 g, yield: 84%).

### Step VII: Preparation of 23H

Ammonium trifluoroacetate (8.59 g, 65.55 mmol) was added to 23G (1.5 g, 4.37 mmol) and reacted at 130° C. for 1.5 h. After cooling to room temperature, 30 mL of water and ethyl acetate (20 mL×2) were added for extraction, the organic phases were combined, dried over anhydrous sodium sulfate and then concentrated under reduced pressure, and the residue was purified by silica gel column chromatography (ethyl acetate/petroleum ether (V/V)=1/100-1/2) to obtain 23H (1 g, yield: 70%).

LCMS m/z=325.3 [M+H]⁺.

### Step VIII: Preparation of 231

At 0° C., diisobutylaluminium hydride (7.7 mL, 7.7 mmol, 1N) in THF solution was dropwise added to a solution of 23H (1 g, 3.08 mmol) in THF (10 mL) and reacted at room temperature for 3 hours. 10 mL of an aqueous saturated ammonium chloride solution was added to quench the reaction, and 30 mL of water and 60 mL of ethyl acetate were added for extraction. After leaving to stand for 20 min, a solid precipitated out, and after filtration, the filtrate was subjected to liquid separation. The organic phase was washed once with 20 mL of an aqueous saturated sodium chloride solution, then dried over anhydrous sodium sulfate, and concentrated under reduced pressure, and the residue was purified by silica gel column chromatography (ethyl acetate/petroleum ether (V/V)=1/100-1/2) to obtain 231 (0.54 g, yield: 59%).

LCMS m/z=297.3 [M+H]⁺.

### Step IX: Preparation of 23J

231 (0.54 g, 1.82 mmol) was dissolved in DCM (4 mL), and thionyl chloride (1.08 g, 9.1 mmol) was dropwise added slowly in an ice-water bath and stirred at room temperature for 2 h. After concentration under reduced pressure, an aqueous saturated sodium bicarbonate solution (10 mL) was added to the residue for alkalization, dichloromethane (30 mL) was added for extraction, and the organic phase was dried over anhydrous sodium sulfate and concentrated under reduced pressure to obtain 23J.

### Step X: Preparation of 23K

Compound 1F (0.11 g, 0.64 mmol) and 23J (0.24 g, 0.76 mmol) were dissolved in DMF (5 mL), and cesium carbonate (0.42 g, 1.28 mmol) was added at room temperature and stirred at 40° C. for 1 h. After cooling to room temperature, 20 mL of ethyl acetate and 20 mL of water were added for extraction, the organic layer was washed with an aqueous saturated sodium chloride solution (10 mL×3), dried over anhydrous sodium sulfate, concentrated under reduced pressure, and purified by column chromatography (ethyl acetate/petroleum ether (V/V)=1/5-1/2) to obtain Compound 23K (0.14 g, yield: 48%).

LCMS m/z=450.3 [M+H]⁺.

### Step XI: Preparation of Compound 23

Compound 23K and (4-cyclopropyl-6-methoxypyrimidin-5-yl)boronic acid (0.12 g, 0.62 mmol) were dissolved in 1,4-dioxane (4 mL) and water (1 mL), and XPhos Pd G2 (0.024 g, 0.031 mmol) and potassium phosphate (0.20 g, 0.93 mmol) were added in sequence. After the addition was complete, displacement with nitrogen was performed three times, and the mixture was heated to 95° C. and stirred for 3 h. After cooling to room temperature, 20 ml of ethyl acetate and 10 ml of water were added for extraction, the organic layer was dried over anhydrous sodium sulfate and concentrated under reduced pressure, and the residue was purified by column chromatography (ethyl acetate/petroleum ether (V/V)=1/10-1/1) to obtain Compound 23 (0.14 g, yield: 80%).

LCMS m/z=564.5 [M+H]⁺.

¹H NMR (400 MHz, DMSO-d₆) δ 9.07 (d, 1H), 8.71 (s, 1H), 8.54 (d, 1H), 7.83-7.76 (m, 2H), 7.37-7.29 (m, 2H), 5.81 (s, 2H), 5.07-4.95 (m, 1H), 3.83 (d, 3H), 3.07-2.94 (m, 1H), 2.92-2.80 (m, 2H), 2.31-2.21 (m, 1H), 1.72-1.62 (m, 1H), 1.18-1.13 (m, 3H), 1.13-1.04 (m, 1H), 1.03-0.95 (m, 1H), 0.94-0.77 (m, 2H).

### BIOLOGICAL TEST EXAMPLES

### Test Example 1: Inhibitory Activity on USP1/UAF1 Enzymology

Ubiquitin rhodamine 110 was used as a substrate to detect the activity of the deubiquitinating enzyme USP1/UAF1 after drug treatment. The total test system was 20 μl, and the test buffer comprises 50 mM Tris-HCl (pH 7.8), 0.5 mM EDTA, 0.01% Bovine Serum Albumin, 1 mM DTT, and 0.01% Tween-20. At the beginning of the experiment, 0.5 nM USP1/UAF1 (Bio-Techne, E-568-050) protein was added to a 384-well plate (PerkinElmer, 6008269), and the test compound diluted in a gradient was then added. After incubation at room temperature for 10 min, 500 nM ubiquitin rhodamine 110 (Bio-Techne, U-555-050) was added, and the mixture was incubated at room temperature for 20 min. Enspire™ Multilabel Reader microplate reader (PerkinElmer) was used for detection at excitation light of 485 nm/emission light of 535 nm. The IC₅₀ value was calculated using GraphPad Prism software.

TABLE 1
Results of inhibitory activity of test
compounds on USP1/UAF1 enzymology

| Compound No. | IC₅₀ (nM) |
|---|---|
| Compound 1 | A |
| Compound 2 | A |
| Compound 3 trifluoroacetate | A |
| Compound 4 | A |
| Compound 8 | A |
| Compound 10 | A |
| Compound 12 | A |
| Compound 14 | A |
| Compound 15 | A |
| Compound 16 | A |
| Compound 18 | A |
| Compound 19 | A |
| Compound 20 | A |
| Compound 21 | A |

Note:
A < 20 nM

Conclusion: The compounds of the present invention have a good inhibitory effect on the enzymatic activity of USP1/UAF1.

### Test Example 2: Pharmacokinetic Test in Beagle Dogs

Experimental objective: a single dose of test compounds was administered to beagle dogs intravenously and intragastrically, and the concentrations of the test compounds in plasma of the beagle dogs were measured to evaluate pharmacokinetic characteristics of the test compounds in the beagle dogs.

Experimental animals: male beagle dogs, about 8-11 kg, were purchased from Beijing Marshall Biotechnology Co., Ltd.

Experimental method: On the day of the experiment, the beagle dogs were randomly grouped according to their body weights. The animals were fasted but with water available for 14 to 18 hours one day before administration, and were fed 4 hours after administration. The administration was performed based on the information in the following table.

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | samples | administration |
| G1 | 3 | Compound of | 1 | 1 | 1 | Plasma | Intravenous |
| | | the present | | | | | administration |
| | | invention | | | | | |
| G2 | 3 | Compound of | 5 | 1 | 5 | Plasma | Intragastric |
| | | the present | | | | | administration |
| | | invention | | | | | |

Vehicle for intravenous administration: 5% DMA+5% Solutol+90% Saline; Vehicle for intragastric administration: 0.5% MC

(DMA: dimethylacetamide; Solutol: polyethylene glycol-60-hydroxystearate; Saline: physiological saline; MC: Methylcellulose solution)

Sampling: Before and after the administration, blood was taken from the jugular veins or limb veins, and placed in an EDTAK2 centrifuge tube. Centrifugation was performed at 5000 rpm at 4° C. for 10 min, and plasma was collected.

Time points for blood collection: 0, 5 min, 15 min, 30 min, 1 h, 2 h, 4 h, 6 h, 8 h, 10 h, 12 h, 24 h, 48 h, and 72 h. Before analysis and detection, all the samples were stored at −60° C. or lower. The samples were analyzed quantitatively by LC-MS/MS.

TABLE 2
Pharmacokinetic parameters of test
compounds in plasma of beagle dogs

| | Mode of | | |
|---|---|---|---|
| Test compound | administration | AUC₀₋ₜ (h · ng · mL⁻¹) | F (%) |
| Compound 2 | i.g. (5 mg/kg) | 6302 | 41.2 |
| Control | i.g. (5 mg/kg) | 994 | 15.8 |
| compound A | | | |

* Note:
i.g. (intragastrically) administration of compounds.

Conclusion: the compound synthesized by using the technique of the present invention has a good oral absorption performance in beagle dogs, and the oral performance is superior to that of control compound A, wherein the control compound A is

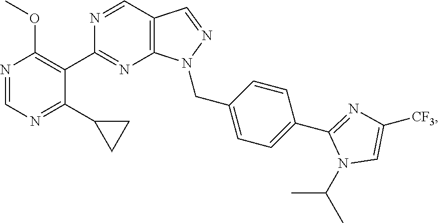 [SMILES: COc1ncnc(C2CC2)c1-c1ncc2cnn(Cc3ccc(-c4nc(C(F)(F)F)cn4C(C)C)cc3)c2n1]

and the preparation method thereof may be found in WO 2020132269.

### Test Example 3: Clone Formation Test of MDA-MB-436 Cells

In this experiment, MDA-MB-436 cells were treated by adding a test compound to detect the ability of cell clone formation. At the beginning of the experiment, MDA-MB-436 cells (ATCC, HTB-130) were added to a 96-well plate (Corning, 3599), with 500 cells per well, and they were subjected to adherent culture overnight. The next day, the compound diluted in a gradient was added, and culture was continued in a CO₂-free incubator at 37° C. On the 7th day, the old culture medium was discarded, and the test compound diluted in a gradient was added again. At the 14th day, the 96-well plate was taken out, the culture liquid was discarded, and cell clones were detected by Crystal violet Assay kit. PHERAstar FSX microplate reader (BMG LABTECH) was used for detection of OD at 570 nm. The IC₅₀ value was calculated using GraphPad Prism software.

TABLE 3
Results of inhibitory activity of test
compounds on MDA-MB-436 cell clones

| Compound No. | IC₅₀ (nM) |
|---|---|
| Compound 1 | A |
| Compound 2 | A |
| Compound 3 trifluoroacetate | A |
| Compound 4 | A |
| Compound 10 | A |
| Compound 15 | A |
| Compound 16 | A |
| Compound 20 | A |
| Compound 22 | A |

Note:
A < 200 nM

Conclusion: The compounds of the present invention have a good inhibitory effect on MDA-MB-436 cell clone formation.

### Test Example 4: Pharmacokinetic Test in Mice

Experimental animals: Male ICR mice, 25-30 g, were purchased from Chengdu Ddossy Experimental Animals Co., Ltd.

Experimental design: On the day of the experiment, the ICR mice were randomly grouped according to their body weights. The animals were fasted with water available for 12 to 14 h one day before the administration, and were fed 4 h after the administration.

| | Administration information | | | | | | |
|---|---|---|---|---|---|---|---|
| | | Administration | Administration | Administration | | | |
| | Test | dosage* | concentration | volume | Collected | Mode of | |
| Group | compound | (mg/kg) | (mg/mL) | (mL/kg) | sample | administration | Vehicle |
| G1 | Compound of | 2.5 | 0.5 | 5 | Plasma | Intravenous | 5% DMA + |
| | the present | | | | | administration | 5% Solutol + |
| | invention | | | | | | 90% Saline |
| G2 | Compound of | 10 | 1 | 10 | Plasma | Oral | 0.5% MC |
| | the present | | | | | (Intragastric | |
| | invention | | | | | administration) | |

(DMA: dimethylacetamide; Solutol: polyethylene glycol-15-hydroxystearate; Saline: physiological saline; MC: methylcellulose)

Before and after the administration, blood was taken from the orbit of the animals at specified time points and placed in an EDTAK2 centrifuge tube. The blood was centrifuged at 5000 rpm and 4° C. for 10 min to collect plasma. The blood collection time points for the intravenous group and intragastric administration group were: 0, 5 min, 15 min, 30 min, 1 h, 2 h, 4 h, 7 h, and 24 h. Before analysis and detection, all samples were stored below −60° C. The samples were analyzed quantitatively by LC-MS/MS.

TABLE 4
Pharmacokinetic parameters of test compounds in plasma of mice

| Test compound | Mode of administration | Oral absorption |
|---|---|---|
| Compound 1 | i.g. (10 mg/kg) | Yes |
| Compound 2 | i.g. (10 mg/kg) | Yes |
| Compound 8 | i.g. (10 mg/kg) | Yes |
| Compound 12 | i.g. (10 mg/kg) | Yes |
| Compound 14 | i.g. (10 mg/kg) | Yes |
| Compound 16 | i.g. (10 mg/kg) | Yes |
| Compound 21 | i.g. (10 mg/kg) | Yes |
| Compound 22 | i.g. (10 mg/kg) | Yes |

* Note:
i.g. (intragastrically) administration of compounds.

Conclusion: The compounds synthesized by using the technique of the present invention have a good oral absorption performance in mice.

1. A compound or a stereoisomer, tautomer, deuterated substance, solvate, prodrug, metabolite, pharmaceutically acceptable salt or eutectic crystal thereof, wherein the compound is selected from a compound shown in general formula (I), 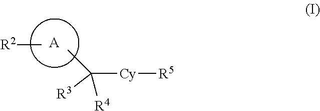
wherein ring A is selected from 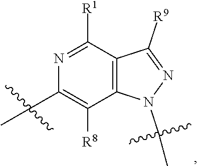
and the left side is connected to R²;
R¹ and R⁹ are each independently selected from H, halogen, cyano, C₁₋₆ alkyl, C₂₋₆ alkenyl, C₂₋₆ alkynyl, and C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, or cyano-substituted C₁₋₆ alkyl;
R⁸ is selected from halogen, cyano, C₁₋₆ alkyl, C₂₋₆ alkenyl, C₂₋₆ alkynyl, and C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, or cyano-substituted C₁₋₆ alkyl;
R² is selected from C₆₋₁₀ carbocycle or 5- to 10-membered heterocycle, wherein the carbocycle or heterocycle is optionally further substituted with 0 to 4 R^{2a};
each R^{2a} is independently selected from H, halogen, OH, ═O, cyano, NH₂, NH(C₁₋₆ alkyl), N(C₁₋₆ alkyl)₂, —SO₂—C₁₋₆ alkyl, —SO₂—C₃₋₆ cycloalkyl, C₁₋₆ alkyl, C₁₋₆ alkoxy, C₁₋₆ alkylthio, —Z—C₃₋₆ cycloalkyl, or —Z-3- to 8-membered heterocycle, wherein the alkyl, cycloalkyl, alkoxy, alkylthio, or heterocycle is optionally further substituted with 0 to 4 substituents selected from H, D, halogen, OH, ═O, cyano, NH₂, C₁₋₆ alkyl, C₂₋₆ alkynyl, C₁₋₆ alkoxy, or C₃₋₆ cycloalkyl, and the heterocycle contains 1 to 4 heteroatoms selected from O, S, and N;
R³ and R⁴ are each independently selected from H, halogen, OH, cyano, NH₂, C₁₋₆ alkyl, C₂₋₆ alkenyl, C₂₋₆ alkynyl, or C₁₋₆ alkoxy, wherein the alkyl, alkenyl, alkynyl, or alkoxy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, or 3- to 8-membered heterocyclyl, and the heterocyclyl contains 1 to 4 heteroatoms selected from O, S, and N;
Cy is selected from C₃₋₁₂ carbocycle or 4- to 12-membered heterocycle, and the Cy is optionally further substituted with 0 to 4 substituents selected from H, halogen, OH, ═O, cyano, NH₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, or 3- to 8-membered heterocyclyl, wherein the heterocycle contains 1 to 4 heteroatoms selected from O, S, and N;
R⁵ is selected from —Z—C₃₋₁₂ carbocycle or —Z-4- to 12-membered heterocycle, and the R⁵ is optionally further substituted with 0 to 4 R^{5a};
each R^{5a} is independently selected from H, halogen, OH, cyano, NH₂, NHC₁₋₆ alkyl, N(C₁₋₆ alkyl)₂, C₁₋₆ alkyl, C₂₋₆ alkynyl, C₁₋₆ alkoxy, —C₁₋₄ alkylene-C₃₋₆ cycloalkyl, —C₁₋₄ alkylene-3- to 6-membered heterocycle, C₃₋₆ cycloalkyl, 3- to 8-membered heterocycle, —CONHC₁₋₆ alkyl, —CONHC₁₋₆ alkoxy, —CONHC₃₋₆ cycloalkyl, —CONH 3- to 8-membered heterocycle, —NHCO—C₁₋₆ alkyl, —NHCO—C₁₋₆ alkoxy, —NHCO—C₃₋₆ cycloalkyl, or —NHCO-3- to 8-membered heterocycle, wherein the alkyl, alkylene, alkynyl, alkoxy, cycloalkyl, or heterocycle is optionally further substituted with 0 to 4 substituents selected from H, D, halogen, OH, ═O, cyano, NH₂, —C(═O)NH₂, —C(═O)NHC₁₋₆ alkyl, —C(═O)N(C₁₋₆ alkyl)₂, C₁₋₆ alkyl, C₂₋₆ alkynyl, C₁₋₆ alkoxy, halogen-substituted C₁₋₆ alkyl, hydroxyl-substituted C₁₋₆ alkyl, cyano-substituted C₁₋₆ alkyl, C₃₋₆ cycloalkyl, halogen-substituted C₃₋₆ cycloalkyl, or 3- to 8-membered heterocycle, and the heterocycle contains 1 to 4 heteroatoms selected from O, S, and N;
Z is selected from a bond, O, S, NH, N(C₁₋₄ alkyl), C(═O)NH, NHC(═O), —OCH₂—, —CH₂O—, or C₁₋₃ alkylene.

2. The compound or the stereoisomer, tautomer, deuterated substance, solvate, prodrug, metabolite, pharmaceutically acceptable salt or eutectic crystal thereof according to claim 1, wherein the compound shown in general formula (I) is selected from a compound shown in general formula (Ia), 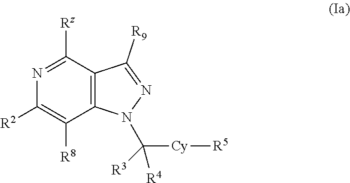
R¹ and R⁹ are each independently selected from H, halogen, cyano, C₁₋₄ alkyl, C₂₋₄ alkenyl, C₂₋₄ alkynyl, and C₁₋₄ alkoxy, wherein the alkyl, alkenyl, alkynyl, or alkoxy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, or cyano-substituted C₁₋₄ alkyl;
R⁸ is selected from halogen, cyano, C₁₋₄ alkyl, C₂₋₄ alkynyl, and C₁₋₄ alkoxy, wherein the alkyl, alkynyl, or alkoxy is optionally further substituted with 0 to 4 substituents selected from H, halogen, OH, cyano, NH₂, C₁₋₄ alkyl, C₂₋₄ alkynyl, and C₁₋₄ alkoxy;
each R^{2a} is independently selected from H, halogen, OH, ═O, cyano, NH₂, NH(C₁₋₄ alkyl), N(C₁₋₄ alkyl)₂, —SO₂—C₁₋₄ alkyl, —SO₂—C₃₋₆ cycloalkyl, C₁₋₄ alkyl, C₁₋₄ alkoxy, C₁₋₄ alkylthio, —Z—C₃₋₆ cycloalkyl, or —Z-3- to 6-membered heterocycle, wherein the alkyl, cycloalkyl, alkoxy, alkylthio, or heterocycle is optionally further substituted with 0 to 4 substituents selected from H, D, halogen, OH, ═O, cyano, NH₂, C₁₋₄ alkyl, C₂₋₄ alkynyl, C₁₋₄ alkoxy, or C₃₋₆ cycloalkyl, and the heterocycle contains 1 to 4 heteroatoms selected from O, S, and N;
R³ and R⁴ are each independently selected from H, halogen, OH, cyano, and C₁₋₄ alkyl;
Cy is selected from C₃₋₆ monocyclic cycloalkyl, C₅₋₁₁ fused cycloalkyl, C₅₋₁₁ spirocycloalkyl, C₅₋₁₁ bridged cycloalkyl, 4- to 7-membered monocyclic heterocycloalkyl, 6- to 11-membered fused heterocycloalkyl, 6- to 11-membered spiroheterocycloalkyl, 6- to 11-membered bridged heterocycloalkyl, benzene ring, benzo C₄₋₆ carbocycle, benzo 4- to 6-membered heterocycle, 5- to 6-membered heteroaromatic ring, or 8- to 10-membered fused heteroaromatic ring, and the Cy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, or 3- to 8-membered heterocycle, wherein the heterocycle contains 1 to 4 heteroatoms selected from O, S, and N;
R⁵ is selected from —Z—C₆₋₁₂ carbocycle or —Z-5- to 12-membered heterocycle, and the R⁵ is optionally further substituted with 0 to 4 R^{5a};
each R^{5a} is independently selected from H, halogen, OH, cyano, NH₂, C₁₋₄ alkyl, C₂₋₄ alkynyl, C₁₋₄ alkoxy, —C₁₋₂ alkylene-C₃₋₆ cycloalkyl, —C₁₋₂ alkylene-3- to 6-membered heterocycle, C₃₋₆ cycloalkyl, or 3- to 6-membered heterocycle, wherein the alkyl, alkylene, alkynyl, alkoxy, cycloalkyl, or heterocycle is optionally further substituted with 0 to 4 substituents selected from H, D, halogen, OH, ═O, cyano, NH₂, —C(═O)NH₂, —C(═O)NHC₁₋₄ alkyl, —C(═O)N(C₁₋₄ alkyl)₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, halogen-substituted C₃₋₆ cycloalkyl, or 3- to 8-membered heterocycle, and the heterocycle contains 1 to 4 heteroatoms selected from O, S, and N.

3. The compound or the stereoisomer, tautomer, deuterated substance, solvate, prodrug, metabolite, pharmaceutically acceptable salt or eutectic crystal thereof according to claim 2, wherein
R¹ and R⁹ are each independently selected from H, F, Cl, Br, I, cyano, methyl, or ethyl;
R⁸ is selected from F, Cl, Br, I, CN, methyl, ethyl, ethynyl, methoxy, or ethoxy;
R² is selected from phenyl, pyridine, pyrimidine, pyrrole, pyrazole, imidazole, furan, thiophene, thiazole, or oxazole, wherein the phenyl, pyridine, pyrimidine, pyrrole, pyrazole, imidazole, furan, thiophene, thiazole, or oxazole is optionally further substituted with 0 to 4 R^{2a};
or R² is selected from 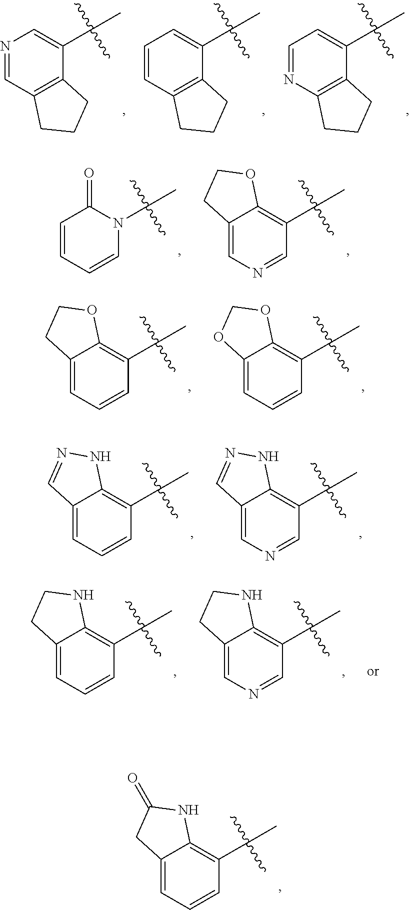
and R² is optionally further substituted with 0 to 4 R^{2a};
each R^{2a} is independently selected from H, F, Cl, Br, I, or cyano, or each R^{2a} is independently selected from one of the following substituted or unsubstituted groups: —SO₂-methyl, —SO₂-ethyl, —SO₂-propyl, —SO₂-isopropyl, —SO₂-cyclopropyl, methyl, ethyl, propyl, isopropyl, methoxy, ethoxy, propoxy, methylthio, ethylthio, propylthio, cyclopropyl, cyclobutyl, cyclopentyl, cyclohexyl, azetidinyl, azacyclopentyl, azacyclohexyl, piperazine, oxetanyl, oxacyclopentyl, oxacyclohexyl, morpholine, —O-azetidinyl, —O-azacyclopentyl, —O-azacyclohexyl, —O-oxetanyl, —O-oxacyclopentyl, —O-oxacyclohexyl, —OCH₂-phenyl, —OCH₂-cyclopropyl, —OCH₂-cyclobutyl, —OCH₂-cyclopentyl, —OCH₂-cyclohexyl, pyrrole, pyrazole, imidazole, thiazole, thiophene, furan, and oxazole, which, when substituted, are optionally further substituted with 0 to 4 substituents selected from H, D, halogen, OH, ═O, cyano, NH₂, C₁₋₄ alkyl, C₂₋₄ alkynyl, C₁₋₄ alkoxy, and C₃₋₆ cycloalkyl;
R³ and R⁴ are each independently selected from H, F, Cl, Br, I, methyl, and ethyl;
Cy is selected from cyclopropyl, cyclobutyl, cyclopentyl, cyclohexyl, bicyclo[2.2.2]octyl, bicyclo[1.1.1]pentyl, cyclopentyl-fused cyclopentyl, cyclobutylspirocyclohexyl, cyclobutylspirocyclopentyl, azetidinyl, azacyclopentyl, azacyclohexyl, azetidinylspirocyclobutyl, azetidinylspirocyclopentyl, azetidinylspirocyclohexyl, azetidinylspiroazetidinyl, azetidinylspiroazacyclopentyl, azetidinylspiroazacyclohexyl, 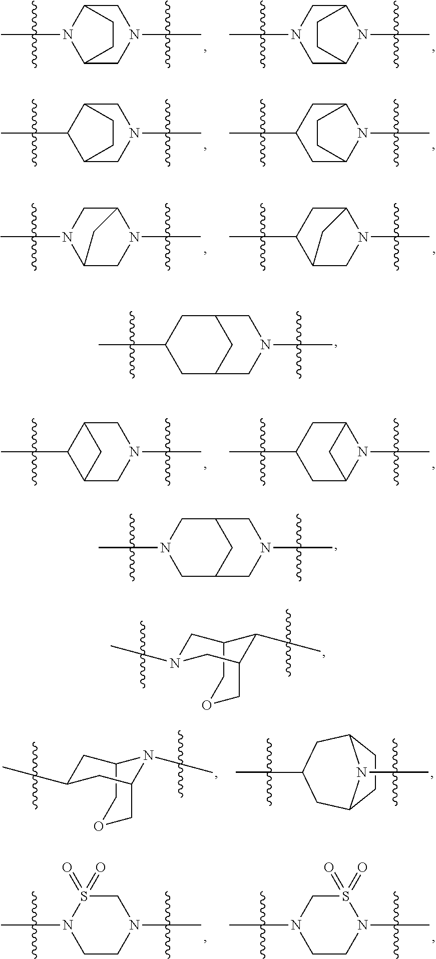
benzene ring, thiophene, furan, pyrrole, thiazole, oxazole, pyrazole, pyrazine, pyrimidine, 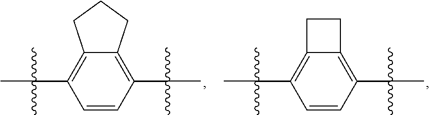 [SMILES: *c1ccc(*)c2c1CCC2 ; *c1ccc(*)c2c1CC2]
benzothiazole, benzothiophene, benzofuran, benzoxazole, benzopyrrole, benzopyrazole, benzimidazole, indolizine, pyridinopyrrole, pyridinoimidazole, pyridinopyrazole, pyrimidoimidazole, pyrimidopyrazole, pyridinotriazole, or 2-pyridone, and the Cy is optionally further substituted with 0 to 4 substituents selected from H, halogen, OH, ═O, cyano, NH₂, C₁₋₄ alkyl, C₂₋₄ alkynyl, C₁₋₄ alkoxy, halogen-substituted C₁₋₄ alkyl, hydroxyl-substituted C₁₋₄ alkyl, cyano-substituted C₁₋₄ alkyl, C₃₋₆ cycloalkyl, or 3- to 8-membered heterocycle, wherein the heterocycle contains 1 to 4 heteroatoms selected from O, S, and N;
R⁵ is selected from benzene ring, pyrazole, pyrrole, imidazole, triazole, pyridine, pyrimidine, pyrazine, or pyridazine, and the R⁵ is optionally further substituted with 0 to 4 R^{5a};
or R⁵ is selected from 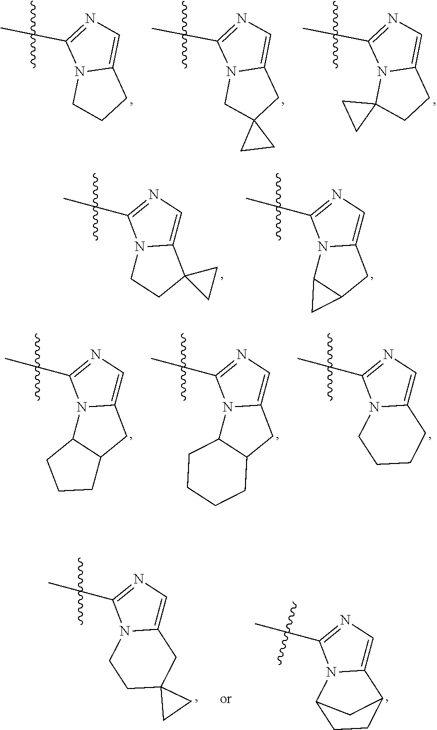
and the R⁵ is optionally further substituted with 0 to 4 R^{5a};
each R^{5a} is independently selected from H, F, Cl, Br, I, cyano, methyl, ethyl, propyl, isopropyl, propargyl, butargyl, methoxy, ethoxy, cyclopropyl, cyclobutyl, cyclopentyl, cyclohexyl, oxetanyl, oxacyclopentyl, oxacyclohexyl, azetidinyl, azacyclopentyl, azacyclohexyl, —CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, or —CH₂-azacyclohexyl, wherein the methyl, ethyl, propyl, isopropyl, propargyl, butargyl, methoxy, ethoxy, cyclopropyl, cyclobutyl, cyclopentyl, cyclohexyl, oxetanyl, oxacyclopentyl, oxacyclohexyl, azetidinyl, azacyclopentyl, or azacyclohexyl is optionally further substituted with 0 to 4 substituents selected from H, D, halogen, OH, ═O, cyano, NH₂, C₁₋₄ alkyl, C₂₋₄ alkynyl, C₁₋₄ alkoxy, C₃₋₆ cycloalkyl, or halogen-substituted C₃₋₆ cycloalkyl.

4. The compound or the stereoisomer, tautomer, deuterated substance, solvate, prodrug, metabolite, pharmaceutically acceptable salt or eutectic crystal thereof according to claim 3, wherein
R¹ and R⁹ are H;
R⁸ is selected from F, Cl, Br, CN, methyl, ethynyl, or methoxy;
R² is selected from pyrrole, pyrazole, imidazole, pyrimidine, 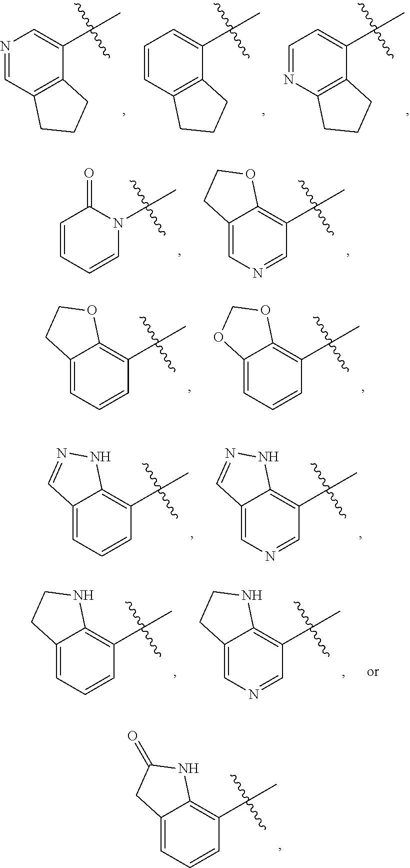
wherein the pyrrole, pyrazole, imidazole, pyrimidine, 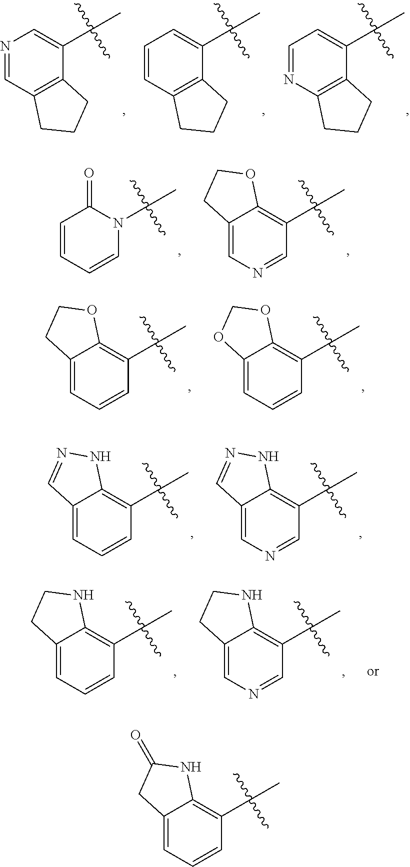
is optionally further substituted with 0 to 4 substituents selected from H, F, Cl, Br, I, cyano, —SO₂-methyl, —SO₂-ethyl, —SO₂-propyl, —SO₂-isopropyl, —SO₂-cyclopropyl, CH₂F, CHF₂, CF₃, —OCH₂F, —OCHF₂, —OCF₃, methyl, ethyl, propyl, isopropyl, methoxy, ethoxy, propoxy, —OCD₃, methylthio, ethylthio, propylthio, cyclopropyl, cyclobutyl, cyclopentyl, cyclohexyl, azetidinyl, azacyclopentyl, azacyclohexyl, piperazine, oxetanyl, oxacyclopentyl, oxacyclohexyl, morpholine, —O-azetidinyl, —O-azacyclopentyl, —O-azacyclohexyl, —O-oxetanyl, —O-oxacyclopentyl, —O-oxacyclohexyl, —OCH₂-phenyl, —OCH₂-cyclopropyl, —OCH₂-cyclobutyl, —OCH₂-cyclopentyl, —OCH₂-cyclohexyl, 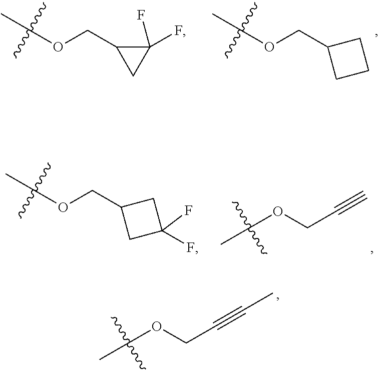
pyrrole, pyrazole, imidazole, thiazole, thiophene, furan, oxazole, 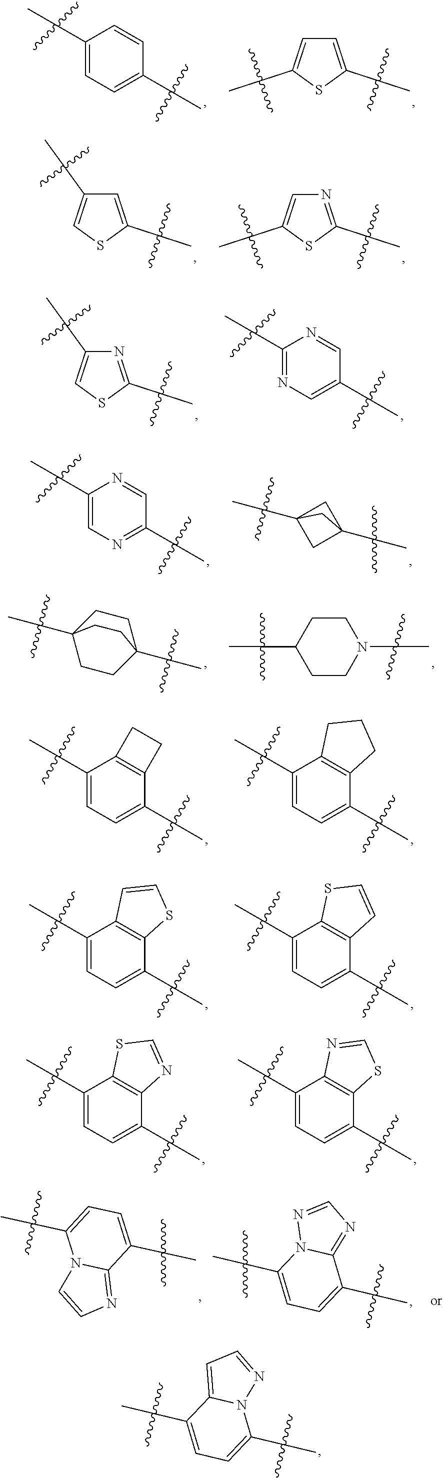
Cy is selected from 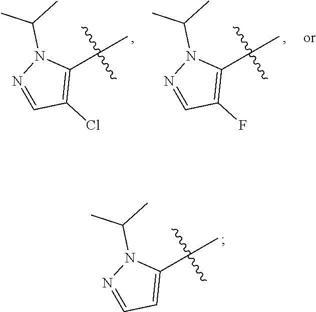
and the Cy is optionally further substituted with 0 to 4 substituents selected from H, F, Cl, Br, I, cyano, CH₂F, CHF₂, CF₃, methyl, ethyl, propyl, isopropyl, methoxy, ethoxy, or cyclopropyl;
alternatively, Cy is selected from 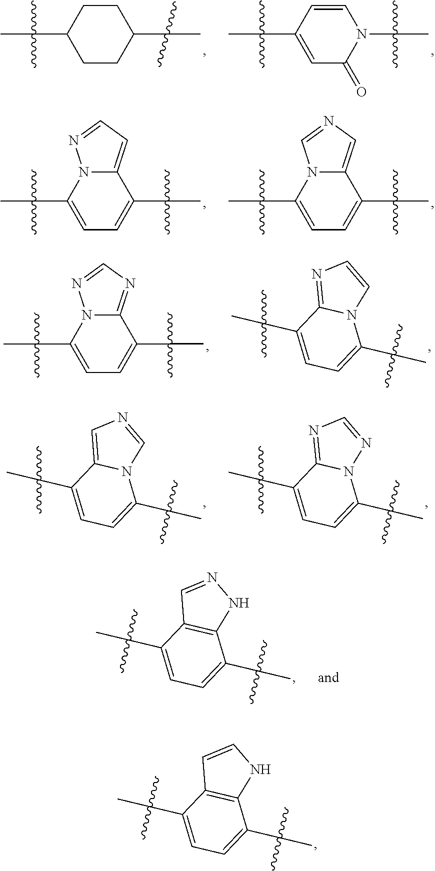
and the Cy is optionally further substituted with 0 to 4 substituents selected from H, F, Cl, Br, I, cyano, CH₂F, CHF₂, CF₃, methyl, ethyl, propyl, isopropyl, methoxy, ethoxy, or cyclopropyl;
R⁵ is selected from pyrrole, pyrazole, or imidazole, and the pyrrole, pyrazole, or imidazole is optionally further substituted with 0 to 4 substituents selected from H, F, Cl, Br, I, cyano, CH₂F, CHF₂, CF₃, CD₃, methyl, ethyl, propyl, isopropyl, —CH₂CH₂F, methoxy, ethoxy, cyclopropyl, cyclobutyl, cyclopentyl, cyclohexyl, oxetanyl, oxacyclopentyl, oxacyclohexyl, azetidinyl, N-methylazetidinyl, azacyclopentyl, azacyclohexyl, 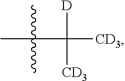 [SMILES: *C([2H])(C([2H])([2H])[2H])C([2H])([2H])[2H]]
—CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, —CH₂-azacyclohexyl, 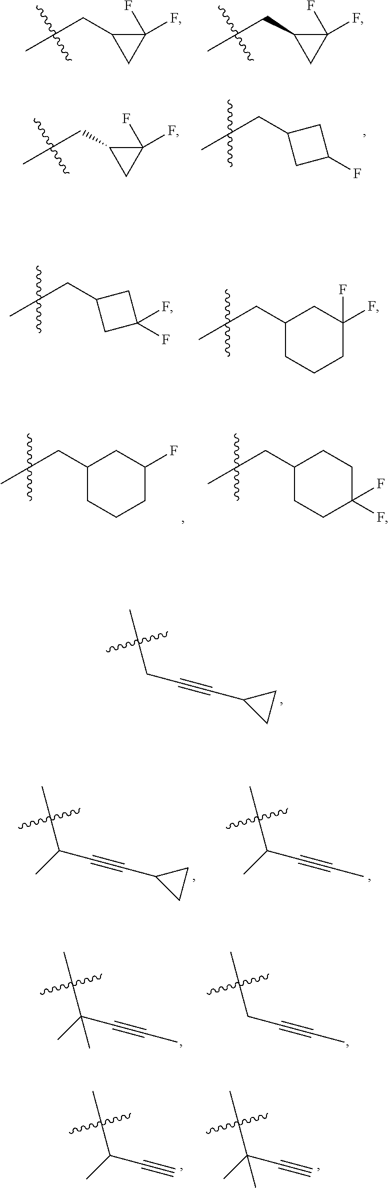
or propargyl;
alternatively, R⁵ is selected from 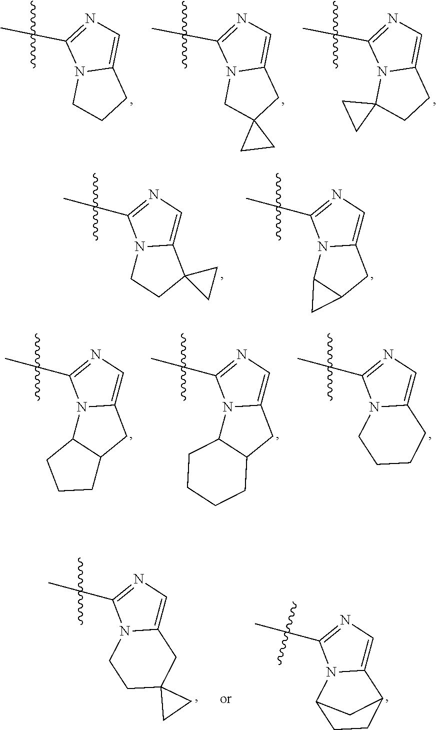
and R⁵ is optionally further substituted with 0 to 4 substituents selected from H, F, Cl, Br, I, cyano, CH₂F, CHF₂, CF₃, CD₃, methyl, ethyl, propyl, isopropyl, —CH₂CH₂F, methoxy, ethoxy, cyclopropyl, cyclobutyl, cyclopentyl, cyclohexyl, oxetanyl, oxacyclopentyl, oxacyclohexyl, azetidinyl, N-methylazetidinyl, azacyclopentyl, azacyclohexyl, 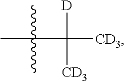 [SMILES: *C([2H])(C([2H])([2H])[2H])C([2H])([2H])[2H]]
—CH₂-cyclopropyl, —CH₂-cyclobutyl, —CH₂-cyclopentyl, —CH₂-cyclohexyl, —CH₂-oxetanyl, —CH₂-oxacyclopentyl, —CH₂-oxacyclohexyl, —CH₂-azetidinyl, —CH₂-azacyclopentyl, —CH₂-azacyclohexyl, 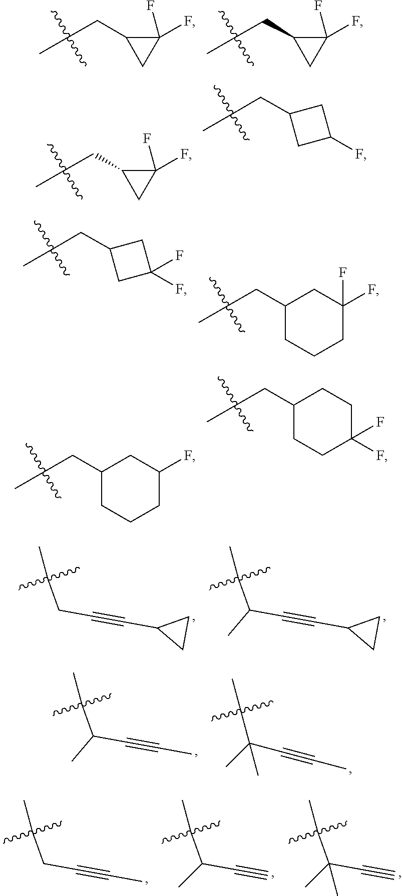
or propargyl.

5. The compound or the stereoisomer, tautomer, deuterated substance, solvate, prodrug, metabolite, pharmaceutically acceptable salt or eutectic crystal thereof according to claim 4, wherein
R² is selected from 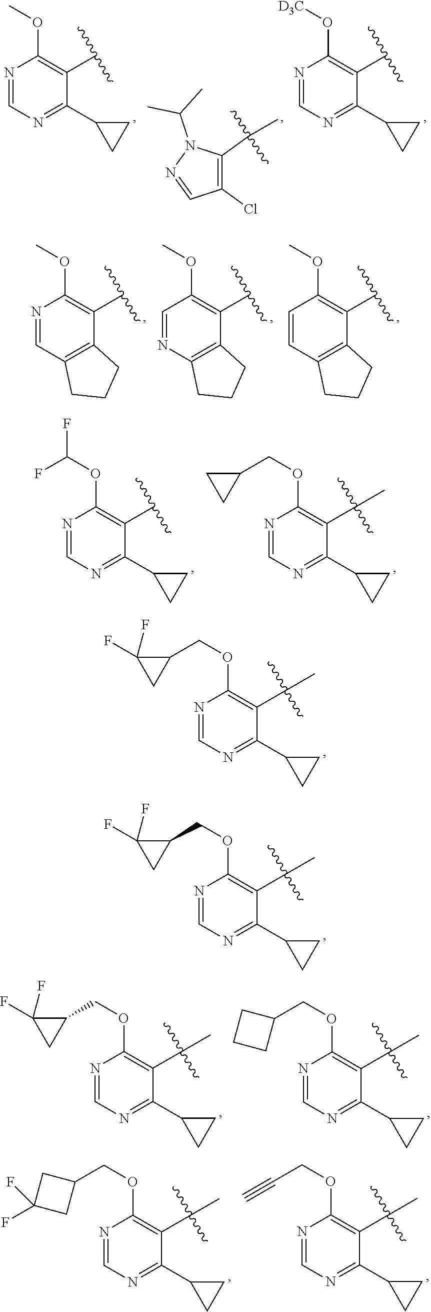 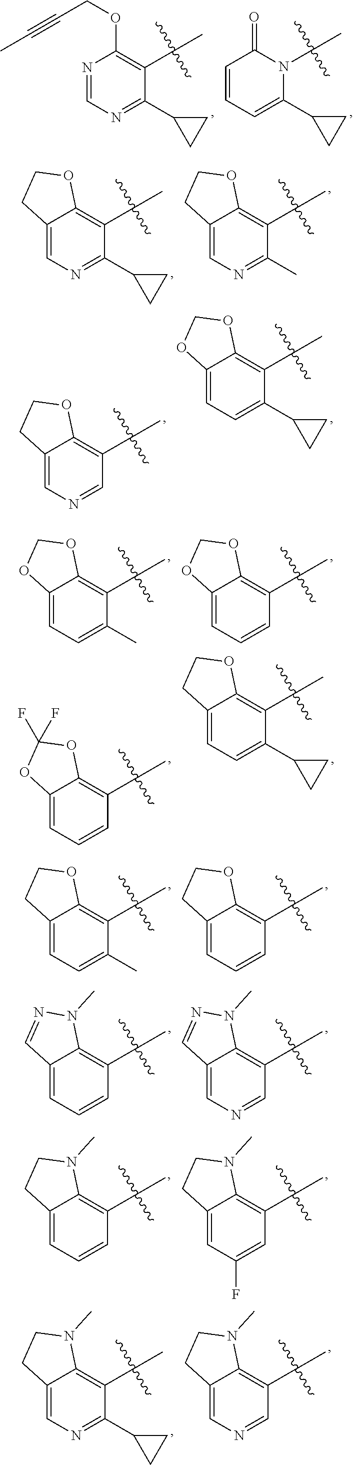 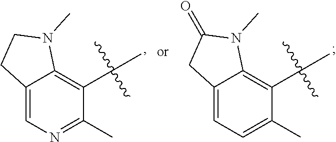 [SMILES: *c1c(C)ncc2c1N(C)CC2 ; *c1c(C)ccc2c1N(C)C(=O)C2]
R⁵ is selected from 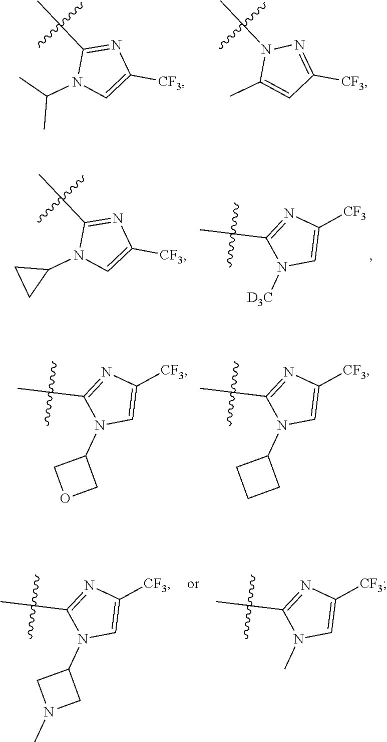
alternatively, R⁵ is selected from 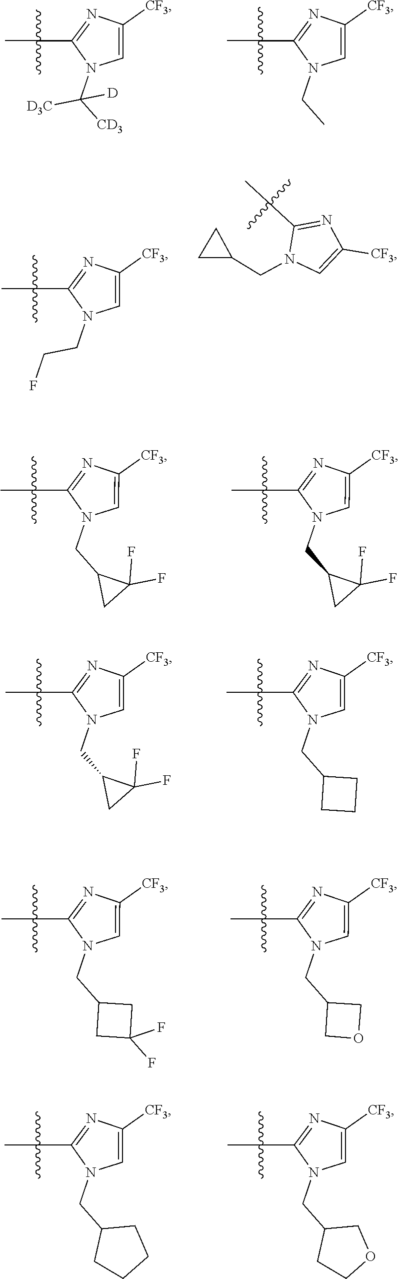 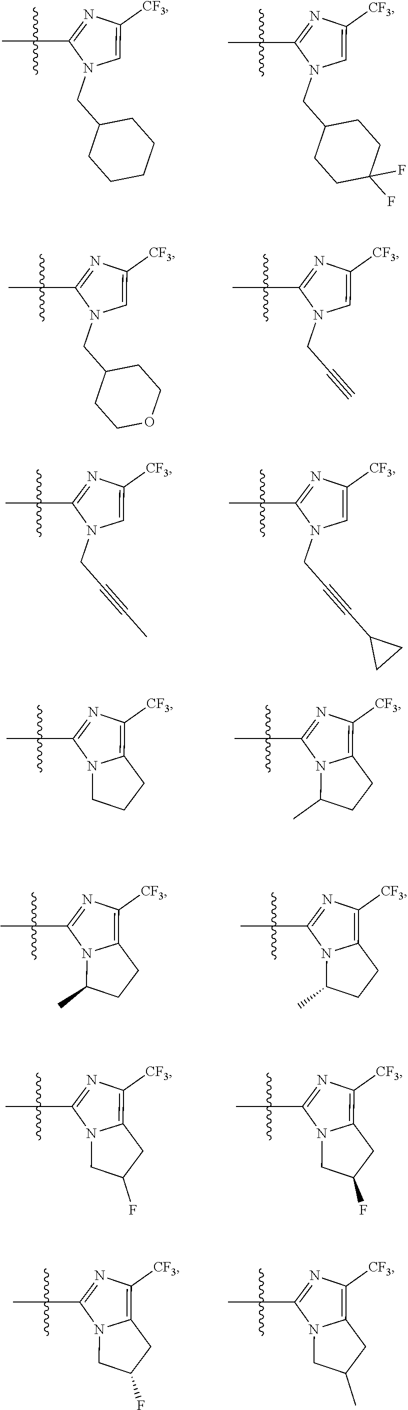 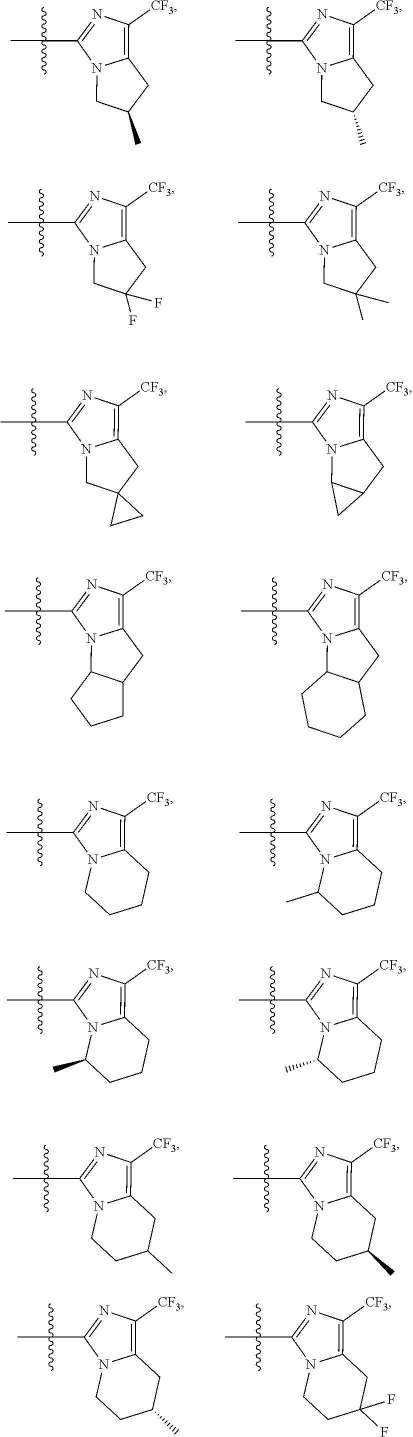 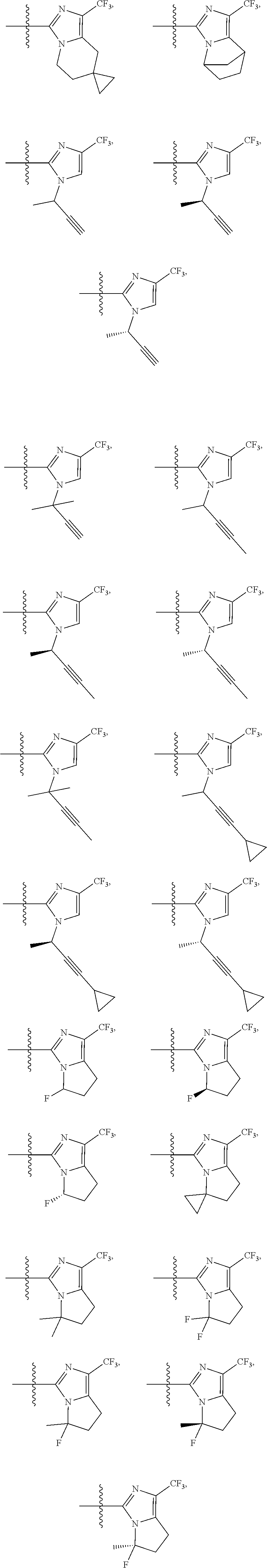 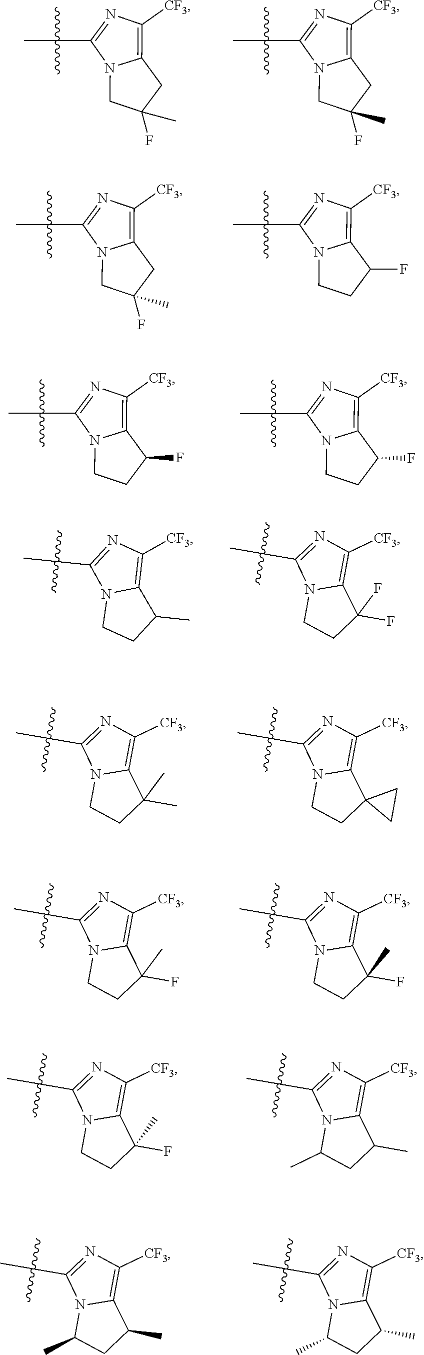 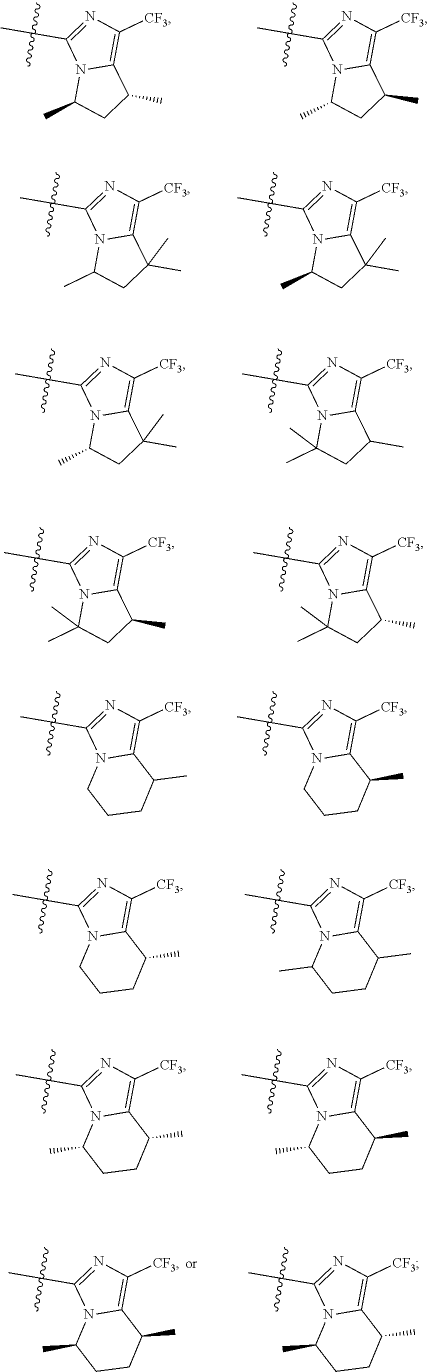
R⁸ is selected from F or C₁.

6. The compound or the stereoisomer, tautomer, deuterated substance, solvate, prodrug, metabolite, pharmaceutically acceptable salt or eutectic crystal thereof according to claim 1, wherein the compound is selected from one of structures shown in Table E.

7. The pharmaceutical composition comprising the compound or the stereoisomer, tautomer, deuterated substance, solvate, prodrug, metabolite, pharmaceutically acceptable salt or eutectic crystal thereof according to claim 1, and a pharmaceutically acceptable carrier.

8. The pharmaceutical composition according to claim 7, wherein the pharmaceutical composition comprises 1-1500 mg of the compound or the stereoisomer, deuterated substance, solvate, prodrug, metabolite, pharmaceutically acceptable salt or eutectic crystal thereof, and a pharmaceutical excipient.

9. A method for treating a disease related to USP1 activity or expression, comprising administering a therapeutically effective amount of the compound or the stereoisomer, tautomer, deuterated substance, solvate, prodrug, metabolite, pharmaceutically acceptable salt or eutectic crystal thereof according to claim 1.

10. The method according to claim 9, wherein the disease is selected from tumors.

11. The method according to claim 9, comprising administering to a subject a therapeutically effective amount of the compound or the stereoisomer, deuterated substance, solvate, prodrug, metabolite, pharmaceutically acceptable salt or eutectic crystal thereof, wherein the therapeutically effective amount is 1-1500 mg.
